(19) United States
(12) Patent Application Publication
(10) Pub. No.: US 2026/0199351 A1
(43) Pub. Date: Jul. 16, 2026

(54) TRIAZOLE COMPOUNDS, PREPARATION METHODS AND MEDICINAL USES THEREOF

(71) Applicant: Jiangsu Hengrui Pharmaceuticals Co., Ltd., Lianyungang, Jiangsu (CN)

(72) Inventors: Hugh Y. Zhu, Rockville, MD (US); Xinlin Du, Rockville, MD (US); Gang Liu, Rockville, MD (US); Jason Rohde, Rockville, MD (US)

(21) Appl. No.: 19/135,373

(22) PCT Filed: Dec. 5, 2023
(86) PCT No.: PCT/CN2023/136398

§ 371 Date: Jun. 3, 2025

Related U.S. Application Data
(60) Provisional application No. 63/386,066, filed on Dec. 5, 2022.
(60) Provisional application No. 63/483,134, filed on Feb. 3, 2023.
(60) Provisional application No. 63/491,803, filed on Mar. 23, 2023.
(60) Provisional application No. 63/497,827, filed on Apr. 24, 2023.

(51) Int. Cl. A61K 31/519; A61K 31/551; C07D 487/04; C07D 519/00

(52) U.S. Cl. CPC A61K 31/519; A61K 31/551; C07D 487/04; C07D 519/00

(57) ABSTRACT

Compounds of formulas useful as WRN inhibitor, the preparation method thereof, pharmaceutical compositions comprising the compounds, and the pharmaceutical uses for the treatment of WRN-mediated diseases or disorders are disclosed.

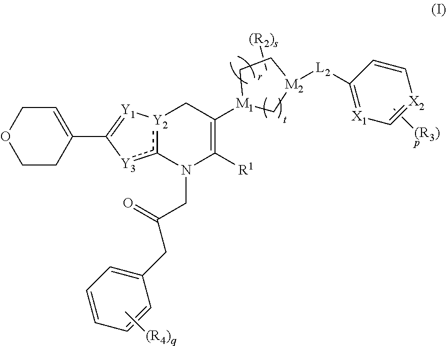

### FIELD OF THE INVENTION

The present invention belongs to the field of medicine, and relates to triazole compound, preparation methods thereof, pharmaceutical compositions comprising the compounds, and medical uses thereof.

### BACKGROUND OF THE INVENTION

Cells in any living organism possess multiple DNA repair machineries to maintain the genome stability and integrity, which is essential for survival. DNA mismatch repair (MMR) is a highly conserved pathway that plays a critical role in maintaining genomic stability by correcting errors occurred during DNA replication, recombination, and repairing processes. (Pecina-Slaus, N., Kafka, A., Salamon, I. & Bukovac, A. Mismatch Repair Pathway, Genome Stability and Cancer. Front Mol. Biosci. 7, 122 (2020)). Deficiency in MMR machinery creates genomic hypermutation and instability, manifested as the high frequency of insertion and deletions at short tracts of repetitive DNA sequences (microsatellites) throughout the genome, a phenomenon known as microsatellite instability (MSI). (Kim, T. M., Laird, P. W. & Park, P. J. The landscape of microsatellite instability in colorectal and endometrial cancer genomes. Cell 155, 858-868 (2013), Vernole, P. et al. Common fragile sites in colon cancer cell lines: role of mismatch repair, RAD51 and poly(ADP-ribose) polymerase-1. Mutat. Res 712, 40-48 (2011)). Microsatellites are generated by DNA polymerase slippage during replication or mismatch repair and are susceptible to misalignment and frame shift mutation, which can be recognized and corrected by MMR process in normal cells. However, in cancer cells lacking normal MMR machinery, errors occurred during replication or repair processes are accumulated and the mutation rate in genome is increased (Lower, S. S., McGurk, M. P., Clark, A. G., et al, Satellite DNA evolution: old ideas, new approaches. Curr. Opin. Genet. Dev., 2018; 49:70-78). High frequency of deletion and insertion in microsatellites lead to MSI, which contributes to the development of 10-30% of ovarian, colon, gastric and endometrial cancers (Aaltonen, L. A. et al. Clues to the pathogenesis of familial colorectal cancer, Science 260, 812-816 (1993), Bonneville R et al., Landscape of Microsatellite Instability Across 39 Cancer Types. JCO Precis Oncol. 1: PO.17.00073 (2017)). While MSI-high (MSI-H) cancer patients show a better overall prognosis and reduced metastatic potential with higher tumor mutation burden and immunogenicity comparing to MSS cancers (Kang, S. et al. The significance of microsatellite instability in colorectal cancer after controlling for clinicopathological factors. Med. (Baltim.) 97, e0019 (2018)), MSI-H tumors tend to develop resistance to immunotherapy and chemotherapy (Le, D. T. et al. Phase II Open-Label Study of Pembrolizumab in Treatment-Refractory, Microsatellite Instability-High/Mismatch Repair-Deficient Metastatic Colorectal Cancer: KEYNOTE-164. J. Clin. Oncol. 38, 11-19 (2020), Overman, M. J. et al. Nivolumab in patients with metastatic DNA mismatch repair-deficient or microsatellite instability-high colorectal cancer (CheckMate 142): an open-label, multicentre, phase 2 study. Lancet Oncol. 18, 1182-1191 (2017), Fuca, G. et al. Ascites and resistance to immune checkpoint inhibition in dMMR/MSI-H metastatic colorectal and gastric cancers. J. Immunother. Cancer 10, 4001 (2022)).

Recently, multiple independent large scale functional genomics screen using more than 300 human cancer cell lines identified Werner syndrome RecQ helicase (WRN) as being selectively required for survival of MSI-H cells (Behan, F. M. et al. Prioritization of cancer therapeutic targets using CRISPR-Cas9 screens. Nature 568, 511-516 (2019), McDonald E. R. et al., Project DRIVE: A Compendium of Cancer Dependencies and Synthetic Lethal Relationships Uncovered by Large-Scale, Deep RNAi Screening. Cell 170(3):577-592 (2017), Chan, E. M. et al. WRN helicase is a synthetic lethal target in microsatellite unstable cancers. Nature 568, 551-556 (2019)). WRN, one of 5 RecQ-like helicases in human, is a multifunctional enzyme with helicase and exonuclease activities and plays important roles in multiple pathways of DNA repair and maintenance of genome integrity including DNA replication, transcription, DNA repair, and telomere maintenance (Bohr, V. A. Rising from the RecQ-age: the role of human RecQ helicases in genome maintenance. Trends Biochem Sci. 33, 609-620 (2008), Singh, D. K., Ahn, B. & Bohr, V. A. Roles of RECQ helicases in recombination based DNA repair, genomic stability and aging. Biogerontology 10, 235-252 (2009), Rossi, M. L., Ghosh, A. K. & Bohr, V. A. Roles of Werner syndrome protein in protection of genome integrity. DNA Repair (Amst.) 9, 331-344 (2010)).

Deletion of WRN leads various defects in cell and genomic structure including cell cycle arrest, DNA breaks, mitotic defects, chromosome shattering, and apoptosis in MSI but not MSS cell lines. (Behan, F. M. et al. Prioritization of cancer therapeutic targets using CRISPR-Cas9 screens. Nature 568, 511-516 (2019), McDonald E. R. et al., Project DRIVE: A Compendium of Cancer Dependencies and Synthetic Lethal Relationships Uncovered by Large-Scale, Deep RNAi Screening. Cell 170(3):577-592 (2017), Chan, E. M. et al. WRN helicase is a synthetic lethal target in microsatellite unstable cancers. Nature 568, 551-556 (2019)). In addition, loss-of-function mutation and deletion studies on WRN further pinpoint the helicase function of WRN as indispensable for the survival MSI cells (Lieb, S. et al. Werner syndrome helicase is a selective vulnerability of microsatellite instability-high tumor cells. Elife 8, e43333 (2019)). These results clearly show that targeting WRN, in particular, the Helicase domain can be a promising strategy for the treatment of for MSI-high cancers.

### SUMMARY OF THE INVENTION

The present invention, in one aspect, provides a compound of formula (I)-(XI), or a pharmaceutically acceptable salt, solvate, or prodrug thereof, including tautomers, cis- or trans-isomers, mesomers, racemates, enantiomers, diastereomers, and mixtures thereof:

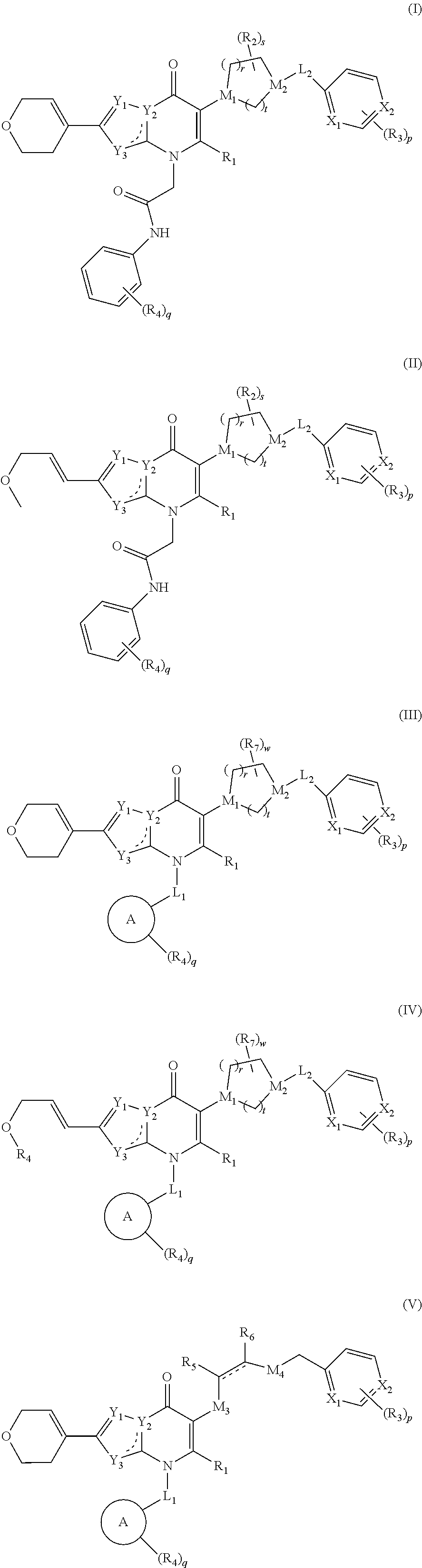

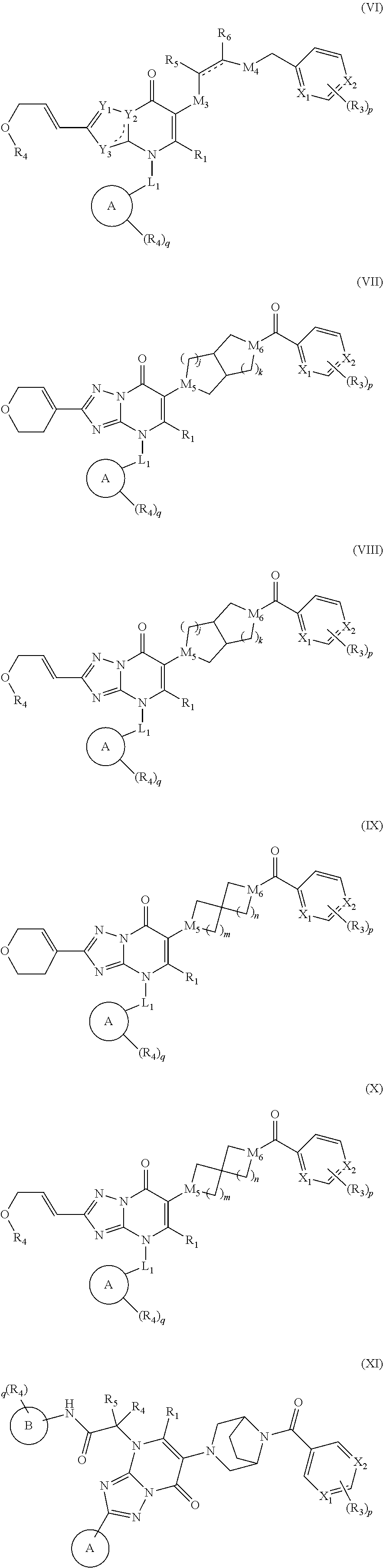

or a tautomer, cis- or trans-isomer, mesomer, racemate, enantiomer, diastereomer, or mixture thereof, or a pharmaceutically acceptable salt, solvate, or prodrug thereof, wherein,
ring A and ring B are each independently selected from aryl or heteroaryl;
Y₁ is CH, NH, N, O or S;
Y₂ is CH, NH, N, O or S;
Y₃ is CH, NH, N, O or S;
X₁ and X₂ are each independently CH or N;
M₁, M₂, M₅, M₆, are each independently CH, NH, N, O or S;
M₃ and M₄ are each independently —NRₐ, —CRₐR_{b};
Rₐ and R_{b} are each independently selected from the group consisting of hydrogen, deuterium, halogen, amino, cyano, oxo, hydroxy, alkyl, alkoxy, haloalkyl and hydroxyalkyl;
Y₂ is CH, NH, N, O or S;
Y₃ is CH, NH, N, O or S;
[symbol] is single bond or double bond, with the proviso that [symbol] are not double bond simultaneously;
R₁, R₃ and R₄ are each independently selected from the group consisting of hydrogen, deuterium, halogen, amino, cyano, oxo, hydroxy, alkyl, alkoxy, haloalkyl and hydroxyalkyl;
two of R₂ together with atom to which they are bound with form a C₃-C₁₀ cycloalkyl or C₃-C₁₀ heterocyclyl,
R₅ and R₆ are each independently selected from the group consisting of hydrogen, deuterium, halogen, amino, cyano, oxo, hydroxy, alkyl, alkoxy, haloalkyl and hydroxyalkyl;
or, R₅ and R₆ together with atom to which they are bound with form a C₃-C₁₀ cycloalkyl, C₃-C₁₀ heterocyclyl, C₆-C₁₄ aryl or C₅-C₁₂ heteroaryl containing 1 to 4 heteroatoms selected from the group consisting of O, S and N;
R₇ is selected from the group consisting of hydrogen, deuterium, halogen, amino, cyano, oxo, hydroxy, alkyl, alkoxy, haloalkyl and hydroxyalkyl;
or, two of R₇ together with atom to which they are bound with form a C₃-C₁₀ cycloalkyl or C₃-C₁₀ heterocyclyl,
L₁ is bond, O, C₁-C₆ alkyl, C₂-C₆ alkenyl, —C₁-C₆ alkyl-NH—, —C₂-C₆ alkenyl-NH—, C₂-C₆ alkynyl, C₁-C₆ alkoxy, C₁-C₆ alkylthio, C₁-C₆ haloalkyl, —C₃-C₁₀ cycloalkyl-CONH—, —C₁-C₆ alkyl-C₃-C₁₀ cycloalkyl-CONH, —C₁-C₆ haloalkyl-NH—, —C₃-C₁₀-heterocyclic-NH-alkyl, —C₃-C₁₀ cycloalkyl-NH—, —C₁-C₆ alkyl-C(NH)—NH, —C₆-C₁₀ heteroaryl-C₁-C₆ alkyl, —S(O)₂—NH— and —C₁-C₆ alkyl-S(O)₂—NH—;
L₂ is bond, O, —C(O), —CONH—, C₁-C₆ alkyl, C₂-C₆ alkenyl, —C₁-C₆ alkyl-NH—, —C₂-C₆ alkenyl-NH—, C₂-C₆ alkynyl, C₁-C₆ alkoxy, C₁-C₆ alkylthio, C₁-C₆ haloalkyl and —CON(Rₑ)—;
Rₑ is selected from the group consisting of hydrogen, deuterium, halogen, amino, cyano, oxo, hydroxy, alkyl, alkoxy, haloalkyl and hydroxyalkyl;
ring A is C₆-C₁₄ aryl or C₅-C₁₂ heteroaryl containing 1 to 4 heteroatoms selected from the group consisting of O, S and N;
m is 0, 1, 2, 3 or 4;
p is 0, 1, 2 or 3;
q is 0, 1, 2 or 3;
r is 0, 1, 2 or 3;
s is 2 or 3;
t is 0, 1, 2 or 3;
w is 0, 1, 2 or 3; and
n is 0, 1, 2 or 3.

In an embodiment, the compound is of formulas (XII)-(XIII), (XIX)-(XXI)

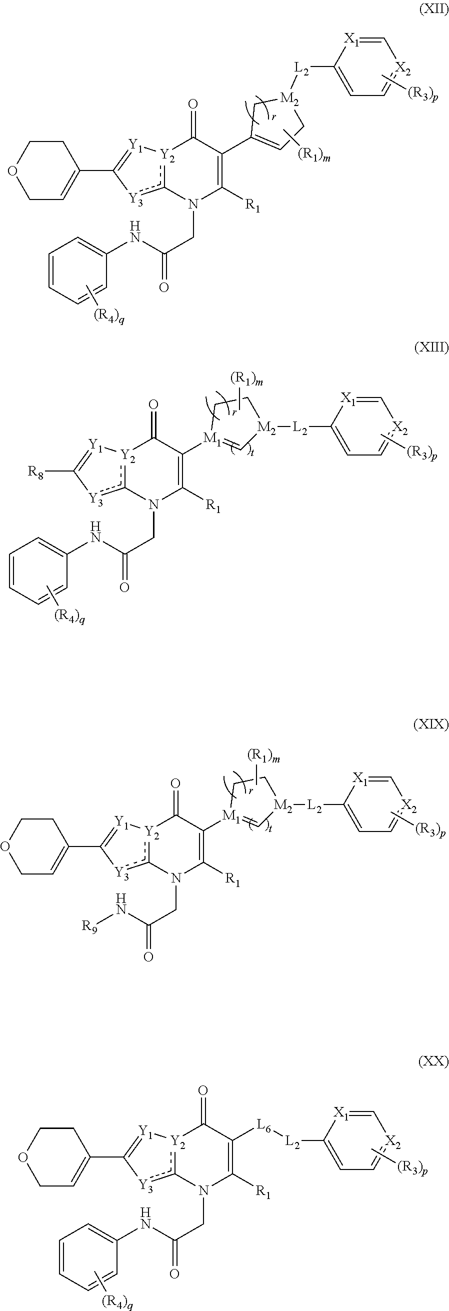

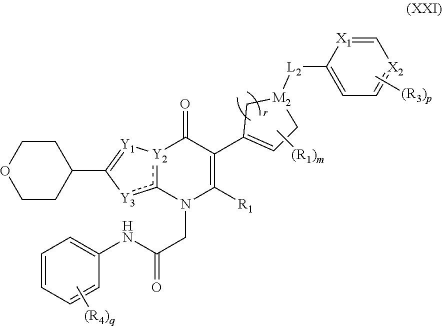

R8 is C₃-C₁₂ fused cycloalkyl, C₂-C₁₀ fused heterocyclyl, wherein the fused cycloalkyl and fused heterocyclyl are each optionally substituted with one or more substituents selected from hydrogen, deuterium, halogen, amino, cyano, hydroxyl, C₁₋₆ alkyl, C₂₋₆ alkenyl, C₂₋₆ alkynyl, C₁₋₆ alkoxy, oxo;
R₉ is bridged C₃-C₁₀ cycloalkyl or bridged C₃-C₁₀ heterocyclyl wherein the bridged cycloalkyl and bridged heterocyclyl are each optionally substituted with one or more substituents selected from hydrogen, deuterium, halogen, amino, cyano, hydroxyl, C₁₋₅ alkyl, C₂₋₆ alkenyl, C₂₋₆ alkynyl, C₁₋₆ alkoxy, oxo, C₁₋₆haloalkyl, C₃₋₅ cycloalkyl;
L6 is bridged C₃-C₁₀ cycloalkyl or bridged C₂-C₁₀ heterocyclyl wherein the bridged cycloalkyl and bridged heterocyclyl are each optionally substituted with one or more substituents selected from hydrogen, deuterium, halogen, amino, cyano, hydroxyl, C₁₋₆ alkyl, C₂₋₆ alkenyl, C₂₋₆ alkynyl, C₁₋₆ alkoxy, oxo, C₁₋₆haloalkyl, C₃₋₅ cycloalkyl;
Y₁, Y₂, Y₃, M₁, M₂, L₂, X₁, X₂, R₁, R₃, R₄, m, p, q, r, t are as defined above.

In an embodiment, R₁, R₃ and R₄ are each independently selected from the group consisting of hydrogen, deuterium, halogen, amino, cyano, oxo, hydroxy, C₁-C₆ alkyl, C₁-C₆ alkoxy, C₁-C₆ haloalkyl, C₁-C₆ hydroxyalkyl and C₂-C₆ alkenyl; In a preferred embodiment, R₁, R₃ and R₄ are each independently selected from the group consisting of hydrogen, deuterium, halogen, amino, cyano, oxo, hydroxy, C₁-C₃ alkyl, —CF₃.

In an embodiment, the compound is of formulas (XXII) or (XXIII),

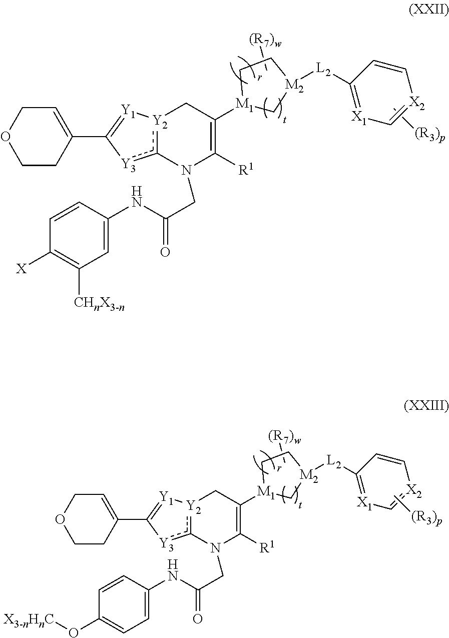

each X is independently selected from F, Cl, Br, I;
Y₁, Y₂, Y₃, M₁, M₂, L₂, X₁, X₂, R₁, R₃, R₇, m, n, p, r, t, w are as defined above.

In an embodiment two of R₂ together with atom to which they are bound with form a bridged C₃-C₁₀ cycloalkyl or bridged C₃-C₁₀ heterocyclyl;

In a preferred embodiment,

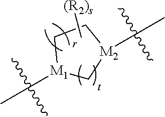

represents

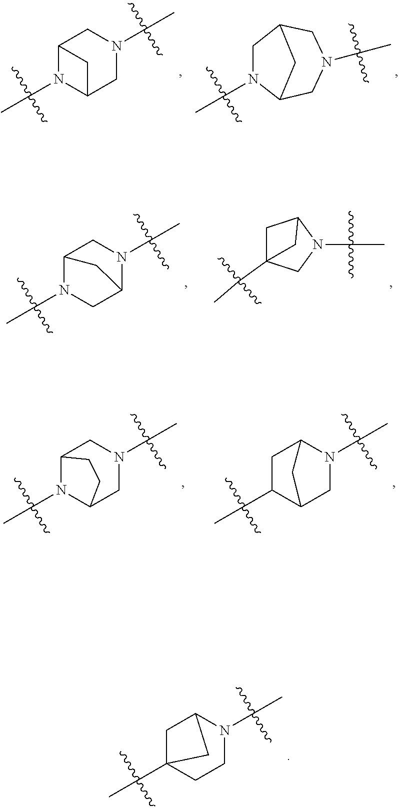

In an embodiment, R₅ and R₆ are each independently selected from the group consisting of hydrogen, deuterium, halogen, amino, cyano, oxo, hydroxy, C₁-C₃ alkyl, C₁-C₃ alkoxy, C₁-C₃ haloalkyl and C₁-C₃ hydroxyalkyl;
or, R₅ and R₆ together with atom to which they are bound with form

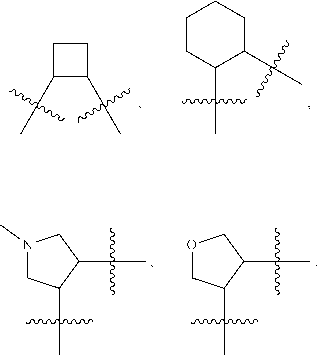

In an embodiment, R₇ is selected from the group consisting of hydrogen, deuterium, halogen, amino, cyano, oxo, hydroxy, C₁-C₃ alkyl, C₁-C₃ alkoxy, C₁-C₃haloalkyl and C₁-C₃ hydroxyalkyl;
or, two of R₇ together with atom to which they are bound with form a bridged C₃-C₁₀ cycloalkyl or bridged C₃-C₁₀ heterocyclyl, preferably,

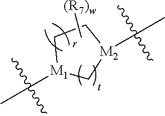

represents

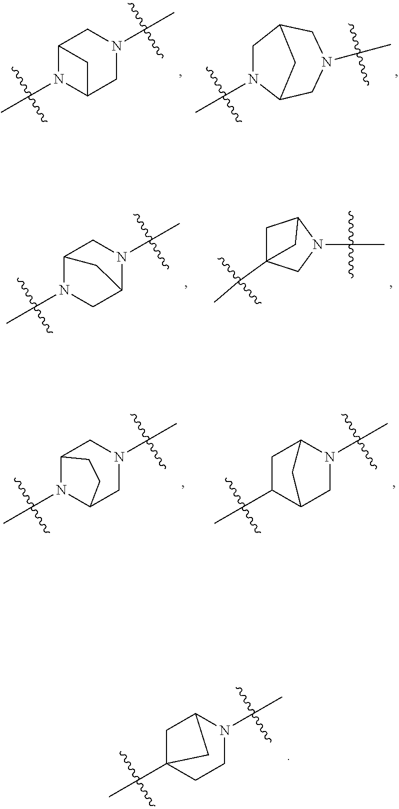

In an embodiment, Rₐ and R_{b} are each independently selected from the group consisting of hydrogen, deuterium, halogen, amino, cyano, oxo, hydroxy, C₁-C₃ alkyl, C₁-C₃ alkoxy, C₁-C₃ haloalkyl and C₁-C₃ hydroxyalkyl; In an embodiment, L₁ is bond, O, C₁-C₃ alkyl, C₂-C₃ alkenyl, —C₁-C₃ alkyl-NH—, —C₂-C₃ alkenyl-NH—, C₂-C₃ alkynyl, C₁-C₃ alkoxy, C₁-C₃ alkylthio, C₁-C₃ haloalkyl, —C₃-C₆ cycloalkyl-CONH—, —C₁-C₃ alkyl-C₃-C₆ cycloalkyl-CONH, —C₁-C₃ haloalkyl-NH—, —C₃-C₆-heterocyclic-NH-alkyl, —C₃-C₆ cycloalkyl-NH—, —C₁-C₆ alkyl-C(NH)—NH, —C₆-C₁₀ heteroaryl-C₁-C₆ alkyl, —S(O)₂—NH— and —C₁-C₆ alkyl-S(O)₂—NH—;

In an embodiment, L₂ is bond, O, —C(O), —CONH—, C₁-C₆ alkyl, C₂-C₆ alkenyl, —C₁-C₃ alkyl-NH—, —C₂-C₃ alkenyl-NH—, C₂-C₃ alkynyl, C₁-C₃ alkoxy, C₁-C₃ alkylthio, C₁-C₃ haloalkyl and —CON(Rₑ)—;
Rₑ is selected from the group consisting of hydrogen, deuterium, halogen, amino, cyano, oxo, hydroxy, C₁-C₃ alkyl, C₁-C₃ alkoxy, C₁-C₃ haloalkyl and C₁-C₃ hydroxyalkyl;

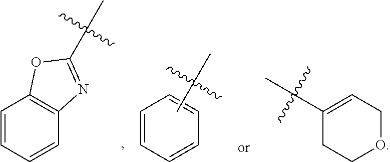

In an embodiment, ring A is.

In an embodiment, ring B is selected from the group consisting of phenyl, pyridyl, pyrrolyl or pyrimidinyl.

In an embodiment,

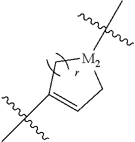

is

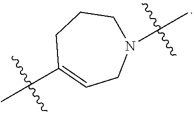 [SMILES: *C1=CCN(*)CCC1]

In an embodiment, R8 is selected from the group consisting of

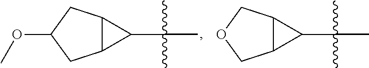

In an embodiment, R9 is

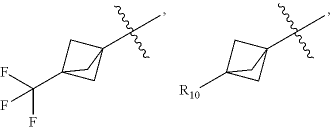

R10 is C₁₋₅ alkyl, C₃₋₅ cycloalkyl.

In an embodiment, L6 is

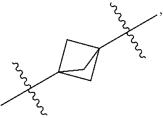 [SMILES: *C12CC(*)(C1)C2]

### or L6-L2 is

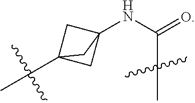 [SMILES: *C(=O)NC12CC(*)(C1)C2]

In an embodiment, in the formula (XXII), X linked to the phenyl is Cl, and/or CHₙX₃₋ₙ is CF₃.

In an embodiment, in the formula (XXIII), CHₙX₃₋ₙ is CF₃.

The present invention also provides a pharmaceutical composition, comprising a therapeutically effective amount of a compound of any of formula (I)-(X), or a tautomer, cis- or trans isomer, mesomer, racemate, enantiomer, diastereomer, or mixture thereof, or a pharmaceutically acceptable salt, solvate, or prodrug thereof, together with one or more pharmaceutically acceptable carriers, diluents or excipients. In another aspect, the present invention relates to a method for treatment of disease mediated by WRN, comprising administering to a subject in need thereof an effective amount of a compound of of any formula (I)-(X), or a tautomer, cis- or trans-isomer, mesomer, racemate, enantiomer, diastereomer, or mixture thereof, or a pharmaceutically acceptable salt solvate, or prodrug thereof, or the pharmaceutical composition comprising the same.

In another aspect, the present invention relates to a method for treating cancer comprising administering to a subject in need thereof an effective amount of a compound of any formula (I)-(X), or a tautomer, cis- or trans-isomer, mesomer, racemate, enantiomer, diastereomer, or mixture thereof, or a pharmaceutically acceptable salt solvate, or prodrug thereof, or the pharmaceutical composition comprising the same.

In a preferred embodiment, the cancer is selected from the group consisting of microsatellite instability-high (MSI-H) or mismatch repair deficient (dMMR) is selected from colorectal, gastric, prostate, endometrial, adrenocortical, uterine, cervical, esophageal, breast, kidney and ovarian cancer

### DETAILED DESCRIPTION OF THE INVENTION

Given below are definitions of terms used in this application. Any term not defined herein takes the normal meaning as the skilled person would understand the term.

“Alkyl” refers to a saturated aliphatic hydrocarbon group including C₁-C₂₀ straight chain and branched chain groups. Preferably an alkyl group is an alkyl having 1 to 12, sometimes preferably 1 to 6, sometimes more preferably 1 to 4, carbon atoms. Representative examples include, but are not limited to methyl, ethyl, n-propyl, isopropyl, n-butyl, isobutyl, tert-butyl, sec-butyl, n-pentyl, 1,1-dimethyl propyl, 1,2-dimethyl propyl, 2,2-dimethyl propyl, 1-ethyl propyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and the isomers of branched chain thereof. More preferably an alkyl group is a lower alkyl having 1 to 6 carbon atoms. Representative examples include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, etc. The alkyl group can be substituted or unsubstituted. When substituted, the substituent group(s) can be substituted at any available connection point, preferably the substituent group(s) is one or more substituents independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Alkenyl” refers to an alkyl defined as above that has at least two carbon atoms and at least one carbon-carbon double bond, for example, vinyl, 1-propenyl, 2-propenyl, 1-, 2-, or 3-butenyl, etc., preferably C₂₋₂₀ alkenyl, more preferably C₂₋₁₂ alkenyl, and most preferably C₂₋₆ alkenyl. The alkenyl group can be substituted or unsubstituted. When substituted, the substituent group(s) is preferably one or more, sometimes preferably one to five, sometimes more preferably one to three, group(s) independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Alkynyl” refers to an alkyl defined as above that has at least two carbon atoms and at least one carbon-carbon triple bond, for example, ethynyl, 1-propynyl, 2-propynyl, 1-, 2-, or 3-butynyl etc., preferably C₂₋₂₀ alkynyl, more preferably C₂₋₁₂ alkynyl, and most preferably C₂₋₆ alkynyl. The alkynyl group can be substituted or unsubstituted. When substituted, the substituent group(s) is preferably one or more, sometimes preferably one to five, sometimes more preferably one to three, group(s) independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Alkylene” refers to a saturated linear or branched aliphatic hydrocarbon group, wherein having 2 residues derived by removing two hydrogen atoms from the same carbon atom of the parent alkane or two different carbon atoms. The straight or branched chain group containing 1 to 20 carbon atoms, preferably has 1 to 12 carbon atoms, more preferably 1 to 6 carbon atoms. Non-limiting examples of alkylene groups include, but are not limited to, methylene (—CH₂—), 1,1-ethylene (—CH(CH₃)—), 1,2-ethylene (—CH₂CH₂)—, 1,1-propylene (—CH(CH₂CH₃)—), 1,2-propylene (—CH₂CH(CH₃)—), 1,3-propylene (—CH₂CH₂CH₂—), 1,4-butylidene (—CH₂CH₂CH₂CH₂—) etc. The alkylene group can be substituted or unsubstituted. When substituted, the substituent group(s) is preferably one or more, sometimes preferably one to five, sometimes more preferably one to three, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Alkenylene” refers to an alkylene defined as above that has at least two carbon atoms and at least one carbon-carbon double bond, preferably C₂₋₂₀ alkenylene, more preferably C₂₋₁₂ alkenylene, and most preferably C₂₋₆ alkenylene. Non-limiting examples of alkenylene groups include, but are not limited to, —CH═CH—, —CH═CHCH₂—, —CH═CHCH₂CH₂—, —CH₂CH═CHCH₂— etc. The alkenylene group can be substituted or unsubstituted. When substituted, the substituent group(s) is preferably one or more, sometimes preferably one to five, sometimes more preferably one to three, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Alkynylene” refers to an alkynyl defined as above that has at least two carbon atoms and at least one carbon-carbon triple bond, preferably C₂₋₂₀ alkynylene, more preferably C₂₋₁₂ alkynylene, and most preferably C₂₋₆ alkynylene. Non-limiting examples of alkenylene groups include, but are not limited to, —CH≡CH—, —CH≡CHCH₂—, —CH≡CHCH₂CH₂—, —CH₂CH≡CHCH₂— etc. The alkynylene group can be substituted or unsubstituted. When substituted, the substituent group(s) is preferably one or more, sometimes preferably one to five, sometimes more preferably one to three, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Cycloalkyl” refers to a saturated and/or partially unsaturated monocyclic or polycyclic hydrocarbon group having 3 to 20 carbon atoms, preferably 3 to 12 carbon atoms, more preferably 3 to 10 carbon atoms, and most preferably 3 to 8 carbon atoms or 3 to 6 carbon atoms. Representative examples of monocyclic cycloalkyls include, but are not limited to, cyclopropyl, cyclobutyl, cyclopentyl, cyclopentenyl, cyclohexyl, cyclohexenyl, cyclohexadienyl, cycloheptyl, cycloheptatrienyl, cyclooctyl, etc. Polycyclic cycloalkyl includes a cycloalkyl having a spiro ring, fused ring or bridged ring.

“Spiro Cycloalkyl” refers to a 5 to 20 membered polycyclic group with rings connected through one common carbon atom (called a spiro atom), wherein one or more rings can contain one or more double bonds, but none of the rings has a completely conjugated pi-electron system. Preferably a spiro cycloalkyl is 6 to 14 membered, and more preferably 7 to 10 membered. According to the number of common spiro atoms, a spiro cycloalkyl is divided into mono-spiro cycloalkyl, di-spiro cycloalkyl, or poly-spiro cycloalkyl, and preferably refers to a mono-spiro cycloalkyl or di-spiro cycloalkyl, more preferably 4-membered/4-membered, 4-membered/5-membered, 4-membered/6-membered, 5-membered/5-membered, or 5-membered/6-membered mono-spiro cycloalkyl. Representative examples of spiro cycloalkyl include, but are not limited to the following substituents;

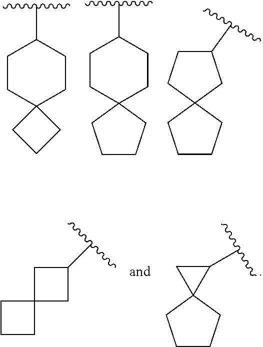

“Fused Cycloalkyl” refers to a 5 to 20 membered polycyclic hydrocarbon group, wherein each ring in the system shares an adjacent pair of carbon atoms with another ring, wherein one or more rings can contain one or more double bonds, but none of the rings has a completely conjugated pi-electron system. Preferably, a fused cycloalkyl group is 6 to 14 membered, more preferably 7 to 10 membered. According to the number of membered rings, fused cycloalkyl is divided into bicyclic, tricyclic, tetracyclic or polycyclic fused cycloalkyl, and preferably refers to a bicyclic or tricyclic fused cycloalkyl, more preferably 5-membered/5-membered, or 5-membered/6-membered bicyclic fused cycloalkyl. Representative examples of fused cycloalkyls include, but are not limited to, the following substituents:

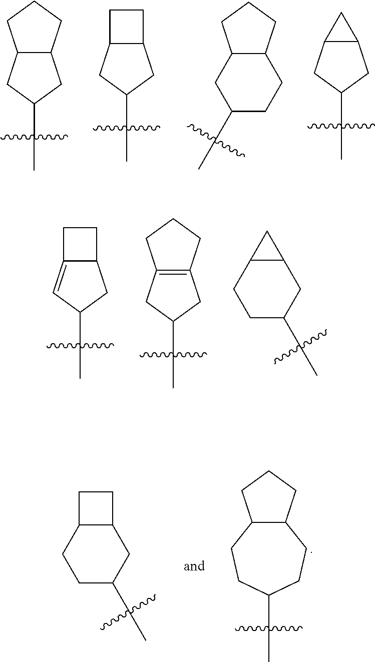

“Bridged Cycloalkyl” refers to a 5 to 20 membered polycyclic hydrocarbon group, wherein every two rings in the system share two disconnected carbon atoms. The rings can have one or more double bonds, but have no completely conjugated pi-electron system. Preferably, a bridged cycloalkyl is 6 to 14 membered, and more preferably 7 to 10 membered. According to the number of membered rings, bridged cycloalkyl is divided into bicyclic, tricyclic, tetracyclic or polycyclic bridged cycloalkyl, and preferably refers to a bicyclic, tricyclic or tetracyclic bridged cycloalkyl, more preferably a bicyclic or tricyclic bridged cycloalkyl. Representative examples of bridged cycloalkyls include, but are not limited to, the following substituents;

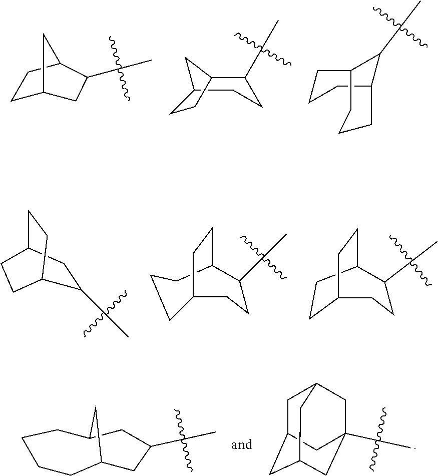

The cycloalkyl can be fused to the ring of an aryl, heteroaryl or heterocyclic alkyl, wherein the ring bound to the parent structure is cycloalkyl. Representative examples include, but are not limited to indanylacetic, tetrahydronaphthalene, benzocycloheptyl and so on. The cycloalkyl is optionally substituted or unsubstituted. When substituted, the substituent group(s) is preferably one or more, sometimes preferably one to five, sometimes more preferably one to three, substituents independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Heterocyclyl” refers to a 3 to 20 membered saturated and/or partially unsaturated monocyclic or polycyclic hydrocarbon group having one or more, sometimes preferably one to five, sometimes more preferably one to three, heteroatoms selected from the group consisting of N, O, and S(O)ₘ (wherein m is 0, 1, or 2) as ring atoms, but excluding —O—O—, —O—S— or —S—S— in the ring, the remaining ring atoms being C. Preferably, heterocyclyl is a 3 to 12 membered having 1 to 4 heteroatoms; more preferably a 3 to 10 membered having 1 to 3 heteroatoms; more preferably a 4 to 8 membered having 1 to 3 heteroatoms; most preferably a 5 to 6 membered having 1 to 2 heteroatoms. Representative examples of monocyclic heterocyclyls include, but are not limited to, oxetanyl, azabutyl, pyrrolidyl, piperidyl, piperazinyl, morpholinyl, sulfo-morpholinyl, homopiperazinyl, and so on. Polycyclic heterocyclyl includes the heterocyclyl having a spiro ring, fused ring or bridged ring.

“Spiro heterocyclyl” refers to a 5 to 20 membered polycyclic heterocyclyl with rings connected through one common carbon atom (called a spiro atom), wherein said rings have one or more, sometimes preferably one to five, sometimes more preferably one to three, heteroatoms selected from the group consisting of N, O, and S(O)ₘ (wherein m is 0.1 or 2) as ring atoms, the remaining ring atoms being C, wherein one or more rings can contain one or more double bonds, but none of the rings has a completely conjugated pi-electron system. Preferably a spiro heterocyclyl is 6 to 14 membered, and more preferably 7 to 10 membered. According to the number of common spiro atoms, spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, more preferably 4-membered/4-membered, 4-membered/5-membered, 4-membered/6-membered, 5-membered/5-membered, or 5-membered/6-membered mono-spiro heterocyclyl. Representative examples of spiro heterocyclyl include, but are not limited to the following substituents:

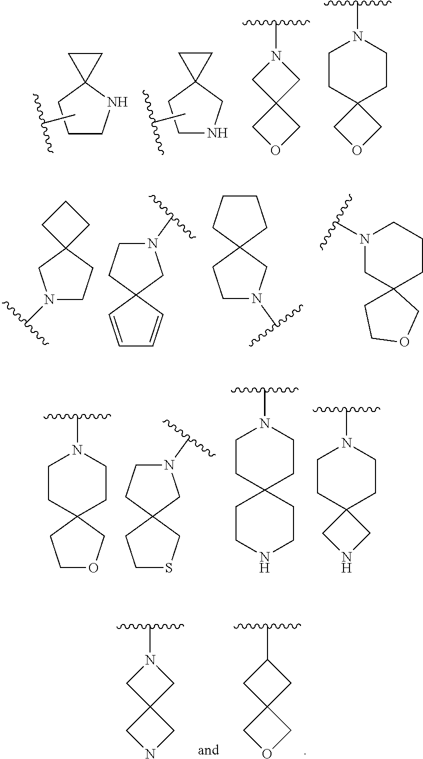

“Fused Heterocyclyl” refers to a 5 to 20 membered polycyclic heterocyclyl group, wherein each ring in the system shares an adjacent pair of carbon atoms with the other ring, wherein one or more rings can contain one or more double bonds, but none of the rings has a completely conjugated pi-electron system, and wherein said rings have one or more, sometimes preferably one to five, sometimes more preferably one to three, heteroatoms selected from the group consisting of N, O, and S(O)ₚ (wherein p is 0, 1, or 2) as ring atoms, the remaining ring atoms being C. Preferably a fused heterocyclyl is 6 to 14 membered, and more preferably 7 to 10 membered. According to the number of membered rings, fused heterocyclyl is divided into bicyclic, tricyclic, tetracyclic or polycyclic fused heterocyclyl, preferably refers to bicyclic or tricyclic fused heterocyclyl, more preferably 5-membered/5-membered, or 5-membered/6-membered bicyclic fused heterocyclyl. Representative examples of fused heterocyclyl include, but are not limited to, the following substituents:

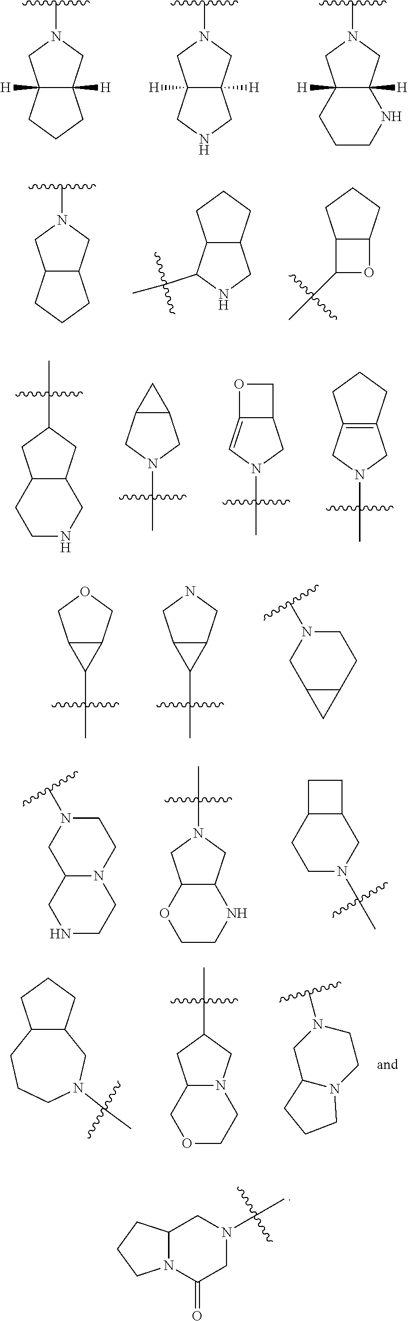

“Bridged Heterocyclyl” refers to a 5 to 14 membered polycyclic heterocyclic alkyl group, wherein every two rings in the system share two disconnected atoms, the rings can have one or more double bonds, but have no completely conjugated pi-electron system, and the rings have one or more heteroatoms selected from the group consisting of N, O, and S(O)ₘ (wherein m is 0, 1, or 2) as ring atoms, the remaining ring atoms being C. Preferably a bridged heterocyclyl is 6 to 14 membered, and more preferably 7 to 10 membered. According to the number of membered rings, bridged heterocyclyl is divided into bicyclic, tricyclic, tetracyclic or polycyclic bridged heterocyclyl, and preferably refers to bicyclic, tricyclic or tetracyclic bridged heterocyclyl, more preferably bicyclic or tricyclic bridged heterocyclyl. Representative examples of bridged heterocyclyl include, but are not limited to, the following substituents:

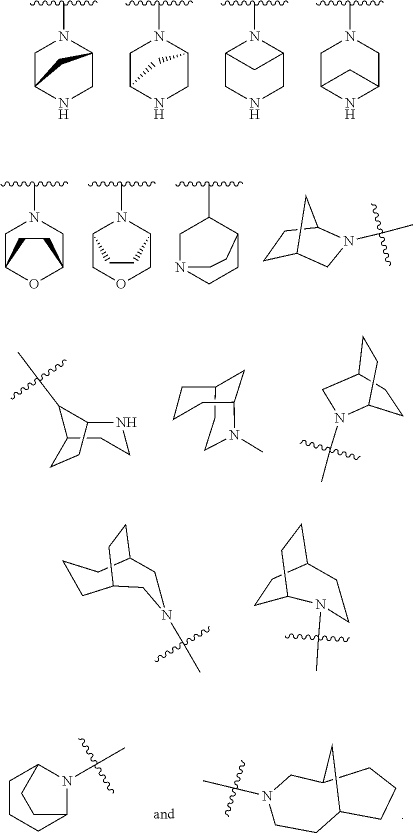

The ring of said heterocyclyl can be fused to the ring of an aryl, heteroaryl or cycloalkyl, wherein the ring bound to the parent structure is heterocyclyl. Representative examples include, but are not limited to the following substituents:

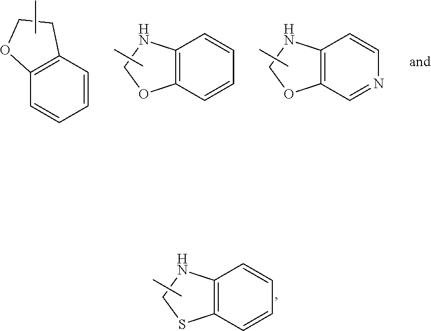

The heterocyclyl is optionally substituted or unsubstituted. When substituted, the substituent group(s) is preferably one or more, sometimes preferably one to five, sometimes more preferably one to three, group(s) independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio, heterocylic alkylthio.

“Aryl” refers to a 6 to 14 membered all-carbon monocyclic ring or a polycyclic fused ring (a “fused” ring system means that each ring in the system shares an adjacent pair of carbon atoms with another ring in the system) group, and has a completely conjugated pi-electron system. Preferably aryl is 6 to 10 membered, such as phenyl and naphthyl, most preferably phenyl. The aryl can be fused to the ring of heteroaryl, heterocyclyl or cycloalkyl, wherein the ring bound to parent structure is aryl. Representative examples include, but are not limited to, the following substituents:

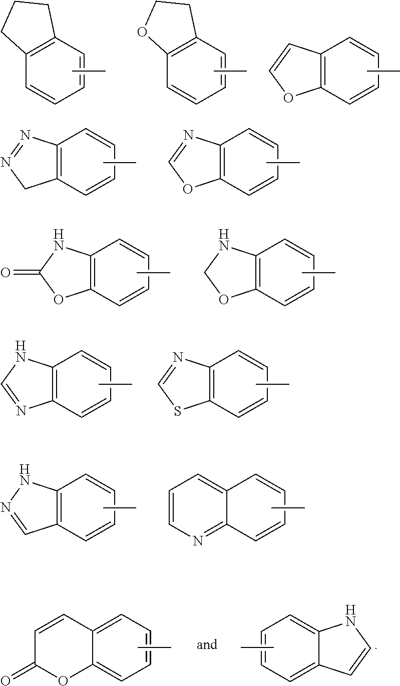

The aryl group can be substituted or unsubstituted. When substituted, the substituent group(s) is preferably one or more, sometimes preferably one to five, sometimes more preferably one to three, substituents independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Heteroaryl” refers to an aryl system having 1 to 4 heteroatoms selected from the group consisting of O, S and N as ring atoms and having 5 to 14 annular atoms. Preferably a heteroaryl is 5- to 10-membered, more preferably 5- or 6-membered, for example, thiadiazolyl, pyrazolyl, oxazolyl, oxadiazolyl, imidazolyl, triazolyl, thiazolyl, furyl, thienyl, pyridyl, pyrrolyl, N-alkyl pyrrolyl, pyrimidinyl, pyrazinyl, imidazolyl, tetrazolyl, and the like. The heteroaryl can be fused with the ring of an aryl, heterocyclyl or cycloalkyl, wherein the ring bound to parent structure is heteroaryl. Representative examples include, but are not limited to, the following substituents:

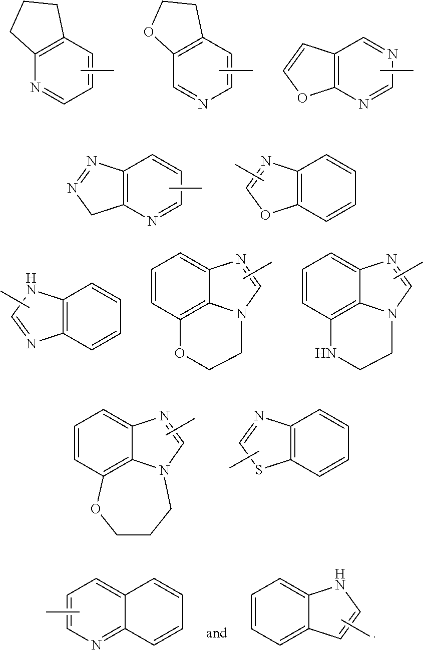

The heteroaryl group can be substituted or unsubstituted. When substituted, the substituent group(s) is preferably one or more, sometimes preferably one to five, sometimes more preferably one to three, substituents independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio, heterocylic alkylthio and —NR⁹R¹⁰.

“Alkoxy” refers to both an —O-(alkyl) and an —O-(unsubstituted cycloalkyl) group, wherein the alkyl is defined as above. Representative examples include, but are not limited to, methoxy, ethoxy, propoxy, butoxy, cyclopropyloxy, cyclobutyloxy, cyclopentyloxy, cyclohexyloxy, and the like. The alkoxyl can be substituted or unsubstituted. When substituted, the substituent is preferably one or more, sometimes preferably one to five, sometimes more preferably one to three, substituents independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Bond” refers to a covalent bond using a sign of “ [symbol] ”.

“Hydroxyalkyl” refers to an alkyl group substituted by a hydroxy group, wherein alkyl is as defined above.

“Hydroxy” refers to an —OH group.

“Halogen” refers to fluoro, chloro, bromo or iodo atoms.

“Amino” refers to a —NH₂ group.

“Cyano” refers to a —CN group.

“Nitro” refers to a —NO₂ group.

“Oxo group” refers to a ═O group.

“Carboxyl” refers to a —C(O)OH group.

“Alkoxycarbonyl” refers to a —C(O)O(alkyl) or (cycloalkyl) group, wherein the alkyl and cycloalkyl are defined as above.

“Optional” or “optionally” means that the event or circumstance described subsequently can, but need not, occur, and the description includes the instances in which the event or circumstance may or may not occur. For example, “the heterocyclic group optionally substituted by an alkyl” means that an alkyl group can be, but need not be, present, and the description includes the case of the heterocyclic group being substituted with an alkyl and the heterocyclic group being not substituted with an alkyl.

“Substituted” refers to one or more hydrogen atoms in the group, preferably up to 5, more preferably 1 to 3 hydrogen atoms, independently substituted with a corresponding number of substituents. It goes without saying that the substituents exist in their only possible chemical position. The person skilled in the art is able to determine if the substitution is possible or impossible without paying excessive efforts by experiment or theory. For example, the combination of amino or hydroxyl group having free hydrogen and carbon atoms having unsaturated bonds (such as olefinic) may be unstable.

A “pharmaceutical composition” refers to a mixture of one or more of the compounds described in the present invention or physiologically/pharmaceutically acceptable salts or prodrugs thereof and other chemical components such as physiologically/pharmaceutically acceptable carriers and excipients. The purpose of a pharmaceutical composition is to facilitate administration of a compound to an organism, which is conducive to the absorption of the active ingredient and thus displaying biological activity.

“Pharmaceutically acceptable salts” refer to salts of the compounds of the invention, such salts being safe and effective when used in a mammal and have corresponding biological activity.

Dosage Forms: the pharmaceutical composition or combination of the present invention may, for example, be in unit dosage of about 1-1000 mg of active ingredient(s) for a subject of about 50-70 kg.

In some embodiments, in above stated pharmaceutical composition, the amount of the compound, tautomer, cis- or trans-isomer, mesomer, racemate, enantiomer, diastereomer, or mixture thereof, or pharmaceutically acceptable salts thereof is about 0.1-95% by weight of free base; preferably, is about 5-70%, e. g. 70%, 65%, 60%, 55%, 50%, 45%, 40%, 35%, 30%, 25%, 20%, 15%, 10%, 5%.

In some embodiment, above stated pharmaceutical composition is formulated as a tablet, capsule, liquid form or injection form.

In some embodiment, in above stated pharmaceutical composition, the amount of the compound, tautomer, cis- or trans-isomer, mesomer, racemate, enantiomer, diastereomer, or mixture thereof, or pharmaceutically acceptable salts thereof is about 1-1000 mg, 0.1-95% by weight of free base; preferably, is about 1-500 mg, more preferably, is about 1 mg, 2 mg, 3 mg, 5 mg, 10 mg, 20 mg, 40 mg, 50 mg, 60 mg, 80 mg, 100 mg, 200 mg, 300 mg, 400 m or 500 mg.

In some embodiment, the compound, tautomer, cis- or trans-isomer, mesomer, racemate, enantiomer, diastereomer, or mixture thereof, or pharmaceutically acceptable salts thereof is can be administered by any suitable route of administration, e.g. oral, parenteral, buccal, sublingual, nasal, rectal, intrathecal or transdermal administration, and the pharmaceutical compositions adapted accordingly.

In some embodiment, the compound, tautomer, cis- or trans-isomer, mesomer, racemate, enantiomer, diastereomer, or mixture thereof, or pharmaceutically acceptable salts is formulated as a solid or liquid form, e.g. syrups, suspension, emulsion, tablets, capsules, powders, granules or lozenges.

In the compounds of Formulas above, the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure as described and claimed herein is meant to include all suitable isotopic variations of the compounds of Formulas above and embodiments thereof. For example, different isotopic forms of hydrogen (H) include protium (1H) and deuterium (2H, also denoted herein as D). Protium is the predominant hydrogen isotope found in nature. Enriching for deuterium may afford certain therapeutic advantages, such as increasing in vivo half-life or reducing dosage requirements or may provide a compound useful as a standard for characterization of biological samples. Isotopically-enriched compounds can be prepared without undue experimentation by conventional techniques well known to those skilled in the art or by processes analogous to those described in the Schemes and Examples herein using appropriate isotopically-enriched reagents and/or intermediates.

### EXAMPLES

### Synthesis Procedures

### Synthesis of Intermediates

### Synthesis of Ethyl 2-bromo-3-oxopentanoate Int 1

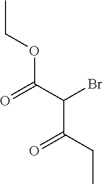

To a solution of ethyl 3-ocopentanoate (15 g, 104 mmol) in dichloromethane (150 mL) were added NBS (19.4 g, 109.2 mmol), followed by TsOH·H₂O (3.96 g, 20.8 mmol). The reaction mixture was stirred at room temperature for 2.5 h. The mixture was filtered, and the cake was washed with dichloromethane. The combined filtrate and washings were washed with water. The aqueous layer was back extracted with dichloromethane. The combined organic layers were dried over Na₂SO₄, filtered, and concentrated to give the title compound.

LCMS: m/z=221.1/223.1 [M+H]⁺

### Synthesis of Tert-butyl 6-(1-ethoxy-1,3-dioxopentan-2-yl)-2,6-diazaspiro[3.3]heptane-2-carboxylate Int 2

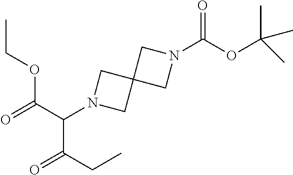 [SMILES: CCOC(=O)C(C(=O)CC)N1CC2(CN(C(=O)OC(C)(C)C)C2)C1]

Ethyl 2-bromo-3-oxopentanoate (1.1 g, 4.93 mmol), tert-Butyl 2,6-diazaspiro[3,3]heptane-2-carboxylate (977.5 mg, 27.1 mmol) and K₂CO₃ (4.1 g, 29.6 mmol) were mixed in acetonitrile (20 mL). The suspension was stirred at room temperature for 30 min. The reaction mixture was filtered. The combined filtrate and washings were concentrated under reduced pressure. The residue was purified by column chromatography (0-10% MeOH in dichloromethane) to provide the title compound.

LCMS (ESI): m/z=341.2 [M+H]⁺

### Synthesis of 2-Chloro-N-(2-methyl-4-(trifluoromethyl)phenyl)acetamide Int 3

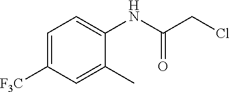 [SMILES: Cc1cc(C(F)(F)F)ccc1NC(=O)CCl]

To a solution of 2-methyl-4-(trifluoromethyl)aniline (6 g, 34.3 mmol) in dichloromethane (50 mL) at 0° C. was added 2-chloroacetyl chloride (2.87 mL, 36 mmol) and triethylamine (9.1 mL, 65 mmol). The reaction was stirred for 30 min, then was warmed to room temperature and stirred for another 1.5 h. The mixture was diluted with dichloromethane and washed with water. The organic layer was dried over Na₂SO₄, filtered, and concentrated. The residue was suspended in a small amount of dichloromethane and filtered. The cake was washed with dichloromethane and Et₂O, and dried under vacuum overnight to provide the title compound with acceptable purity. The combined filtrate and washings were concentrated, and the residue was purified by column chromatography (50-100% EtOAc in heptane) to provide the title compound which was combined with the cake.

LCMS: m/z=250.1/252.1 [M−H]⁻

### Synthesis of 2-Chloro-N-(2-chloro-4-(trifluoromethyl)phenyl)acetamide Int 4

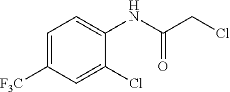 [SMILES: O=C(CCl)Nc1ccc(C(F)(F)F)cc1Cl]

To a solution of 2-chloro-4-(trifluoromethyl)aniline (5 g, 25.6 mmol) in dichloromethane (50 mL) at 0° C. was added 2-chloroacetyl chloride (2.14 mL, 26.9 mmol) and triethylamine (7.1 mL, 51 mmol). The reaction was stirred for 30 min, then was warmed to room temperature and stirred for another 1.5 h. The mixture was diluted with dichloromethane and washed with water. The organic layer was dried over Na₂SO₄, filtered, and concentrated. The residue was suspended in a small amount of dichloromethane and filtered. The cake was washed with dichloromethane and Et₂O, and dried under vacuum overnight to provide the title compound with acceptable purity. The combined filtrate and washings were concentrated, and the residue was purified by column chromatography (50-100% EtOAc in heptane) to provide the title compound which was combined with the cake.

### Synthesis of 4-Chloro-5-methoxy-6-methylpyrimidine Int 5

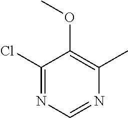

A mixture of 4,6-dichloro-5-methoxypyrimidine (25 g, 140 mmol), methylboronic acid (8.8 g, 147 mmol), K3PO4 (74 g, 350 mmol), and Pd(dppf)Cl₂·DCM (6.86 g, 8.4 mmol) in DME (100 mL) was stirred at 85° C. for 18 h. The reaction mixture was filtered through a celite plug and the filtrate was concentrated. The residue was dissolved in EtOAc and washed with aqueous saturated NaHCO₃. The organic layer was dried over Na₂SO₄, concentrated. The residue was purified by column chromatography (0-5% EtOAc in heptane) to provide the title compound.

¹H NMR (400 MHz, DMSO-d6) δ 8.66 (s, 1H), 3.86 (s, 3H), 2.50 (s, 3H)

### Synthesis of Methyl 5-methoxy-6-methylpyrimidine-4-carboxylate Int 6

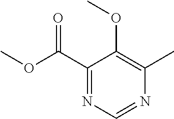

To a solution of 4-chloro-5-methoxy-6-methylpyrimidine (14 g, 88.2 mmol) in MeOH (70 mL) were added Pd(dppf)Cl₂·DCM (4.32 g, 5.3 mmol) and triethylamine (24.5 mL, 177 mmol). The resulting mixture was stirred at 60° C. under CO (0.4 MPa) for 16 h, then was allowed to cool to room temperature. The reaction mixture was filtered, and the collected filtrate was concentrated. The residue was dissolved in EtOAc and washed with water. The aqueous layer was back extracted with EtOAc. The combined organic layers were concentrated, and the residue was purified by column chromatography (10% EtOAc in heptane, isocratic) to provide the title compound.

¹H NMR (400 MHz, DMSO-d6) δ 8.85 (s, 1H), 3.93 (s, 3H), 3.84 (s, 3H), 1.17 (s, 3H).

### Synthesis of 5-Hydroxy-6-methylpyrimidine-4-carboxylic acid Int 7

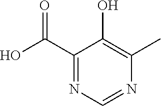

Methl 5-methocy-6-methylpyrifidine-4-carboxylate (5.5 g, 29 mmol) was dissolved in HBr (40% in water) (23.5 mL, 174 mmol). The resulting mixture was stirred at 40° C. for 10 h. Then HI (23 mL, 174 mmol) was added, and the mixture was stirred for another 6 h before adjusting its pH to 3 with 50% NaOH solution at 0° C. The yellow solid was separated by filtration and subsequently suspended in water (40 mL) and HCl (37% in water) (11 mL, 134 mmol). The resulting suspension was stirred at 60° C. for 2 h, then was filtered. The solid was collected and dried under vacuum to provide the title compound.

¹H NMR (400 MHz, DMSO-d6) δ 11.71 (s, 2H), 8.66 (s, 1H), 2.47 (s, 3H).

### Synthesis of tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate Int 8

### Step 1. tert-butyl 4-(1-ethoxy-1,3-dioxopentan-2-yl)piperazine-1-carboxylate

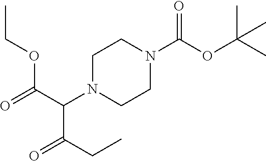 [SMILES: CCOC(=O)C(C(=O)CC)N1CCN(C(=O)OC(C)(C)C)CC1]

Ethyl 2-bromo-3-oxopentanoate (2 g, 8.96 mmol), tert-Butyl piperazine-1-carboxylate (977.5 mg, 27.1 mmol) and K₂CO₃ (9.18 g, 49.3 mmol) were mixed in acetonitrile (40 mL). The suspension was stirred at room temperature for 30 min. The reaction mixture was filtered. The combined filtrate and washings were concentrated under reduced pressure. The residue was purified by column chromatography (0-10% MeOH in dichloromethane) to provide the title compound.

### Step 2. tert-butyl 4-(2-bromo-5-ethyl-7-oxo-4,7-dihydro-[1,2,4]triazolo[1,5-a]pyrimidin-6-yl)piperazine-1-carboxylate

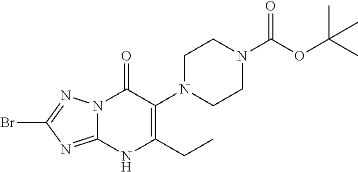 [SMILES: CCc1[nH]c2nc(Br)nn2c(=O)c1N1CCN(C(=O)OC(C)(C)C)CC1]

To a solution of tert-butyl 4-(1-ethoxy-1,3-dioxopentan-2-yl)piperazine-1-carboxylate (765.2 mg, 2.33 mmol) in EtOH (4 mL) were added 3-bromo-1H-1,2,4-trizaol-5-amine (400 mg, 2.33 mmol) and H₃PO₄ (282 mg, 2.95 mmol). The resulting mixture was stirred at 85° C. for 24 h, then cooled to room temperature. DIPEA (1.22 mL, 7 mmol) and Boc₂O (255 mg, 1.17 mmol) were added, and the mixture was stirred at room temperature for another 2 h before it was quenched with small amount of water. EtOH was removed under reduced pressure, and the residue was dissolved in EtOAc and washed with water, brine, dried (Na₂SO₄), and concentrated. The residue was purified by column chromatography (0-75% EtOAc in heptane) to provide the title compound.

### Step 3. tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate

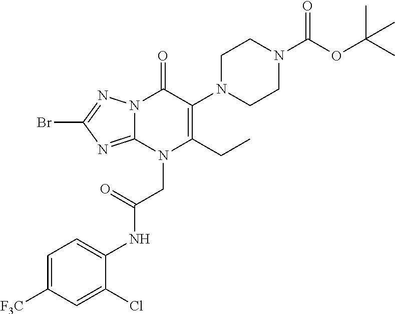 [SMILES: CCc1c(N2CCN(C(=O)OC(C)(C)C)CC2)c(=O)n2nc(Br)nc2n1CC(=O)Nc1ccc(C(F)(F)F)cc1Cl]

To a mixture of tert-butyl 4-(2-bromo-5-ethyl-7-oxo-4,7-dihydro-[1,2,4]triazolo[1,5-a]pyrimidin-6-yl)piperazine-1-carboxylate (125 mg, 0.29 mmol) and 2-chloro-N-(2-chloro-4-(trifluoromethyl)phenyl)acetamide (86.8 mg, 0.32 mmol) in 1,4-dioxane (4 mL) were added KI (48.1 mg, 0.29 mmol) and DIPEA (152 μL, 0.87 mmol). The resulting mixture was stirred at 80° C. for 3.5 h, then cooled to room temperature. The mixture was partitioned between EtOAc and water. The organic layer was washed with brine, dried (Na₂SO₄), filtered, and concentrated. The residue was purified by column chromatography (0-50% EtOAc in heptane) to provide the title compound.

### Synthesis of tert-butyl 4-(2-bromo-5-ethyl-4-(2-((2-methyl-4-(trifluoromethyl)phenyl)amino)-2-oxoethyl)-7-oxo-4,7-dihydro-[1,2,4]triazolo[1,5-a]pyrimidin-6-yl)piperazine-1-carboxylate Int 9

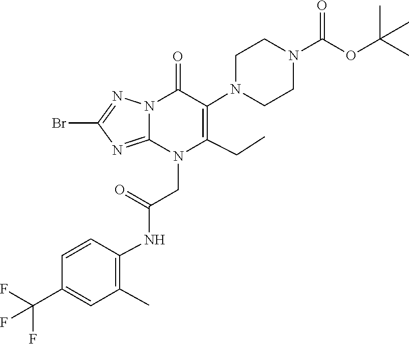 [SMILES: CCc1c(N2CCN(C(=O)OC(C)(C)C)CC2)c(=O)n2nc(Br)nc2n1CC(=O)Nc1ccc(C(F)(F)F)cc1C]

Int 9 could be made by following the same synthetic route of Int8 using 2-chloro-N-(2-methyl-4-(trifluoromethyl)phenyl)acetamide Int 3 as a synthetic intermediate.

### Synthesis of tert-butyl 3-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)-3,8-diazabicyclo[3.2.1]octane-8-carboxylate Int 10

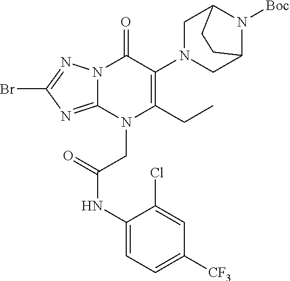 [SMILES: CCc1c(N2CC3CCC(C2)N3C(=O)OC(C)(C)C)c(=O)n2nc(Br)nc2n1CC(=O)Nc1ccc(C(F)(F)F)cc1Cl]

### Step 1. methyl 2-chloro-3-oxopentanoate

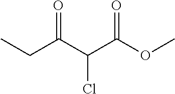

To a solution of methy3-oxopentanoate (10 g, 76.84 mmol) in DCM (100 ml) was added SO₂Cl₂ (13.48 g, 99.89 mmol) at 0° C. over 10 min. The reaction was allowed to warm to RT and stirred for 16 hours. The RM was concentrated under reduced pressure and the residue was dissolved in DCM (100 ml) and washed with water (100 ml), brine (100 ml), dried over Na2SO4 and filtered. The filtrate was concentrated under reduced pressure to give the title compound as light yellow liquid (12 g, 94% yield). Mass calc C6H9ClO3 for 164.0, found: 163.0 (M−H)− ESI.

1H NMR (400 MHz, DMSO-d6) δ: 5.62 (s, 1H), 3.76 (s, 3H), 2.50-2.48 (m, 2H), 0.99 (t, J=7.2 Hz, 3H).

### Step 2. tert-butyl 3-(1-methoxy-1,3-dioxopentan-2-yl)-3,8-diazabicyclo[3.2.1]octane-8-carboxylate

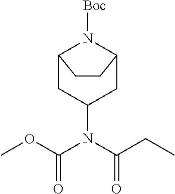 [SMILES: CCC(=O)N(C(=O)OC)C1CC2CCC(C1)N2C(=O)OC(C)(C)C]

To a solution of methy2-chloro-3-oxopentanoate (5.04 g, 30.62 mmol), in dry ACN (25 ml) was added TEA (7.15 g, 70.66 mmol) over 15 min, followed by dropwise addition of tert-butyl 3,8-diazabicyclo[3.2.1]octane-8-carboxylate (5 g, 23.55 mmol) in ACN over 30 min. The reaction was stirred at 60° C. for 16 hours. The RM was filtered and washed with EA. The filtrated was then concentrated under reduced pressure and the residue was dissolved in EA and washed with water, dried over Na₂SO₄ and filtered. The filtrate was concentrated under reduced pressure and the residue was purified by column chromatography eluent heptane:EA 100:0 to 90:10) to give the title compound (4 g, 50% yield). Mass calc C17H28N2O5 for 340.2, found: 339.1 (M−H)− ESI. 1H NMR (400 MHz, DMSO-d6) δ: 4.36 (s, 1H), 3.61 (s, 3H), 3.21-3.11 (m, 1H), 2.99-2.92 (m, 1H), 2.75-2.60 (m, 4H), 2.37-2.26 (m, 2H), 1.88-1.75 (m, 4H), 1.41-1.38 (s, 9H), 0.96 (t, J=7.3 Hz, 3H).

### Step 3. tert-butyl 3-(2-bromo-5-ethyl-7-oxo-4,7-dihydro-[1,2,4]triazolo[1,5-a]pyrimidin-6-yl)-3,8-diaza bicyclo[3.2.1]octane-8-carboxylate

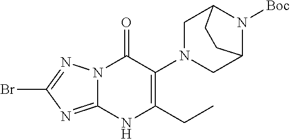 [SMILES: CCc1[nH]c2nc(Br)nn2c(=O)c1N1CC2CCC(C1)N2C(=O)OC(C)(C)C]

3-Bromo-1H-1,2,4-triazol-5-amine (843 mg, 5.17 mmol) and tert-butyl (1R,5S)-3-(1-methoxy-1,3-dioxopentan-2-yl)-3,8-diazabicyclo[3.2.1]octane-8-carboxylate (1.6 g, 4.70 mmol) were mixed in EtOH (3 mL). H3PO4 (461 mg, 4.70 mmol) was added. The mixture was stirred at 80° C. for 12 hours under nitrogen. The mixture was concentrated in vacuo to remove EtOH, then quenched by addition of aq sat NaHCO3, and extracted with DCM. The combined organic layers were washed with brine, dried over Na2SO4, filtered and concentrated under reduced pressure to give a residue. The residue was purified by column chromatography (Silica column, eluent DCM:MeOH 1:0 to 50:1) to give the title compound as a yellow solid (300 mg, 14% yield). Mass calc C18H25BrN6O3 for 452.1 454.1, found: 451.1 453.1 (M−H)− ESI. 1H NMR (400 MHz, DMSO-d6) δ: 13.25 (s, 1H), 4.16-4.02 (m, 2H), 3.56-3.50 (d, J=9.8 Hz, 2H), 2.81-2.71 (m, 2H), 2.46-2.38 (d, J=9.8 Hz, 2H), 1.93-1.80 (m, 4H), 1.44 (s, 9H), 1.21 (t, J=7.6 Hz, 3H).

### Step 4. tert-butyl 3-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)-3,8-diazabicyclo[3.2.1]octane-8-carboxylate

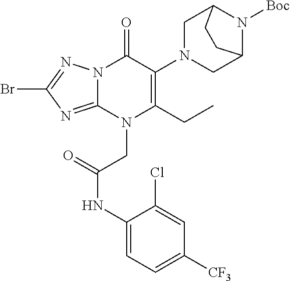 [SMILES: CCc1c(N2CC3CCC(C2)N3C(=O)OC(C)(C)C)c(=O)n2nc(Br)nc2n1CC(=O)Nc1ccc(C(F)(F)F)cc1Cl]

To a stirred solution of tert-butyl 3-(2-bromo-5-ethyl-7-oxo-4,7-dihydro-[1,2,4]triazolo[1,5-a]pyrimidin-6-yl)-3,8-diaza bicyclo[3.2.1]octane-8-carboxylate (140 mg, 0.31 mmol) and 2-chloro-N-(2-chloro-4-(trifluoromethyl)phenyl)acetamide (84 mg, 0.31 mmol) in 1,4-dioxane (2 mL) was added DIPEA (120 mg, 0.93 mmol) at RT and the RM was stirred at 80° C. for 72 hours. The RM was concentrated under reduced pressure. The crude product was diluted with EtOAc and water, extracted once with EtOAc and the organic layer was washed with brine, dried over Na₂SO₄ and concentrated under reduced pressure. The crude product was purified by column chromatography (eluent heptane:EtOAc 70:30 to 30:70) to give the title compound as a white solid (130 mg, 61% yield). Mass calc C27H30BrClF3N7O4 for 687.1 689.1, found: 686.1 688.1 (M−H)− ESI. 1H NMR (400 MHz, DMSO-d6) δ: 10.34 (s, 1H), 8.11-8.06 (d, J=8.6 Hz, 1H), 7.99-7.96 (d, J=1.7 Hz, 1H), 7.74-7.70 (dd, J=8.7, 1.7 Hz, 1H), 5.29 (s, 2H), 4.15-4.07 (m, 2H), 3.61-3.57 (m, 2H), 3.02-2.92 (m, 2H), 2.49-2.43 (m, 2H), 1.92-1.84 (m, 4H), 1.45 (s, 9H), 1.20 (t, J=7.4 Hz, 3H).

### Synthesis of tert-butyl 3-(2-bromo-5-ethyl-4-(2-((2-methyl-4-(trifluoromethyl)phenyl)amino)-2-oxoethyl)-7-oxo-4,7-dihydro-[1,2,4]triazolo[1,5-a]pyrimidin-6-yl)-3,8-diazabicyclo[3.2.1]octane-8-carboxylate Int 11

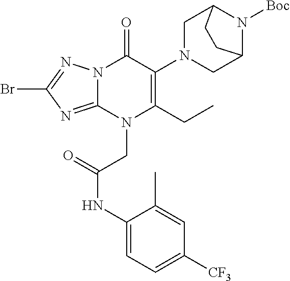 [SMILES: CCc1c(N2CC3CCC(C2)N3C(=O)OC(C)(C)C)c(=O)n2nc(Br)nc2n1CC(=O)Nc1ccc(C(F)(F)F)cc1C]

Int 11 could be made by following the same synthetic route of Int 10 using 2-chloro-N-(2-methyl-4-(trifluoromethyl)phenyl)acetamide Int 3 as a synthetic intermediate.

### Synthesis of tert-butyl 3-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-3,8-diazabicyclo[3.2.1]octane-8-carboxylate Int 12

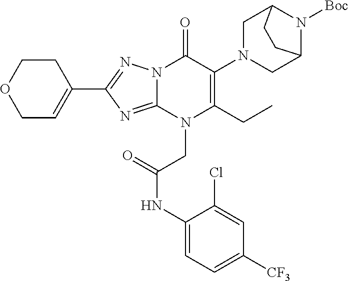 [SMILES: CCc1c(N2CC3CCC(C2)N3C(=O)OC(C)(C)C)c(=O)n2nc(C3=CCOCC3)nc2n1CC(=O)Nc1ccc(C(F)(F)F)cc1Cl]

Tert-butyl 3-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)-3,8-diazabicyclo[3.2.1]octane-8-carboxylate (186 mg, 0.27 mol) was suspended in 1,4-dioxane (0.5 mL). 2-(3,6-dihydro-2H-pyran-4-yl)-4,4,5,5-tetramethyl-1,3,2-dioxaborolane (113 mg, 0.54 mmol) and 0.6M aq Na2CO3 (0.25 mL) were added and the RM was degassed for 10 minutes with argon, PdCI2(PPh)2 (19 mg, 0.03 mmol) was added and the RM was stirred at 90° C. for 12 hours. The RM was concentrated under reduced pressure, and the crude product was purified by column chromatography (Silica gel column: Silica 12 g, eluent DCM:MeOH 100:0 to 97:3) to give the crude title compound (72 mg). Mass calc C32H37ClF3N705 for 691.2, found: 690.1 (M−H)− ESI.

### Synthesis of tert-butyl 3-(2-(3,6-dihydro-2H-pyran-4-yl)-5-ethyl-4-(2-((2-methyl-4-(trifluoromethyl)phenyl) amino)-2-oxoethyl)-7-oxo-4,7-dihydro-[1,2,4]triazolo[1,5-a]pyrimidin-6-yl)-3,8-diaza bicyclo[3.2.1]octane-8-carboxylate Int 13

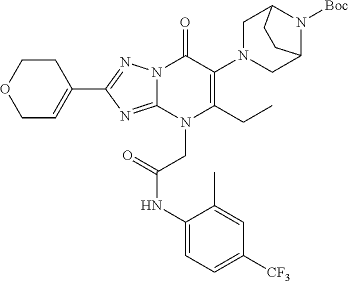 [SMILES: CCc1c(N2CC3CCC(C2)N3C(=O)OC(C)(C)C)c(=O)n2nc(C3=CCOCC3)nc2n1CC(=O)Nc1ccc(C(F)(F)F)cc1C]

Int 13 could be made by following the same method of Int 12 via the Suzuki coupling of Int 11 and 2-(3,6-dihydro-2H-pyran-4-yl)-4,4,5,5-tetramethyl-1,3,2-dioxaborolane.

### Synthesis of tert-butyl 3-(5-ethyl-2-(5-fluoro-3,6-dihydro-2H-pyran-4-yl)-4-(2-((2-methyl-4-(trifluoromethyl)phenyl)amino)-2-oxoethyl)-7-oxo-4,7-dihydro-[1,2,4]triazolo[1,5-a]pyrimidin-6-yl)-3,8-diazabicyclo[3.2.1]octane-8-carboxylate Int 14

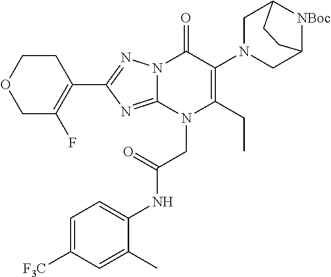 [SMILES: CCc1c(N2CC3CCC(C2)N3C(=O)OC(C)(C)C)c(=O)n2nc(C3=C(F)COCC3)nc2n1CC(=O)Nc1ccc(C(F)(F)F)cc1C]

Int 14 could be made by following the same method of Int 13 via the Suzuki coupling of Int 11 and 2-(5-fluoro-3,6-dihydro-2H-pyran-4-yl)-4,4,5,5-tetramethyl-1,3,2-dioxaborolane.

### Synthesis of tert-butyl 3-(5-ethyl-2-(4-fluoro-5,6-dihydro-2H-pyran-3-yl)-4-(2-((2-methyl-4-(trifluoromethyl)phenyl)amino)-2-oxoethyl)-7-oxo-4,7-dihydro-[1,2,4]triazolo[1,5-a]pyrimidin-6-yl)-3,8-diazabicyclo[3.2.1]octane-8-carboxylate Int 15

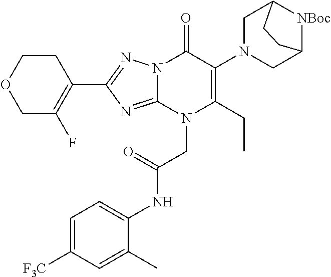 [SMILES: CCc1c(N2CC3CCC(C2)N3C(=O)OC(C)(C)C)c(=O)n2nc(C3=C(F)COCC3)nc2n1CC(=O)Nc1ccc(C(F)(F)F)cc1C]

Int 15 could be made by following the same method of Int 13 via the Suzuki coupling of Int 11 and 2-(4-fluoro-5,6-dihydro-2H-pyran-3-yl)-4,4,5,5-tetramethyl-1,3,2-dioxaborolane.

### Synthesis of tert-butyl 3-(2-(3,6-dihydro-2H-thiopyran-4-yl)-5-ethyl-4-(2-((2-methyl-4-(trifluoromethyl)phenyl)amino)-2-oxoethyl)-7-oxo-4,7-dihydro-[1,2,4]triazolo[1,5-a]pyrimidin-6-yl)-3,8-diazabicyclo[3.2.1]octane-8-carboxylate Int 16

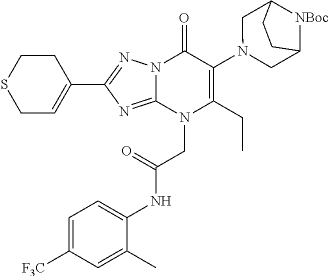 [SMILES: CCc1c(N2CC3CCC(C2)N3C(=O)OC(C)(C)C)c(=O)n2nc(C3=CCSCC3)nc2n1CC(=O)Nc1ccc(C(F)(F)F)cc1C]

Int 16 could be made by following the same method of Int 13 via the Suzuki coupling of Int 11 and 2-(3,6-dihydro-2H-thiopyran-4-yl)-4,4,5,5-tetramethyl-1,3,2-dioxaborolane.

### Synthesis of tert-butyl 3-(5-ethyl-2-(6-methyl-3,6-dihydro-2H-pyran-4-yl)-4-(2-((2-methyl-4-(trifluoromethyl)phenyl)amino)-2-oxoethyl)-7-oxo-4,7-dihydro-[1,2,4]triazolo[1,5-a]pyrimidin-6-yl)-3,8-diazabicyclo[3.2.1]octane-8-carboxylate Int 17

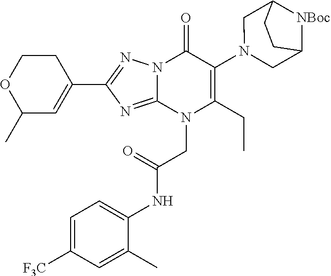 [SMILES: CCc1c(N2CC3CCC(C2)N3C(=O)OC(C)(C)C)c(=O)n2nc(C3=CC(C)OCC3)nc2n1CC(=O)Nc1ccc(C(F)(F)F)cc1C]

Int 17 could be made by following the same method of Int 13 via the Suzuki coupling of Int 11 and 4,4,5,5-tetramethyl-2-(6-methyl-3,6-dihydro-2H-pyran-4-yl)-1,3,2-dioxaborolane.

### Synthesis of tert-butyl 3-(5-ethyl-2-(2-methyl-3,6-dihydro-2H-pyran-4-yl)-4-(2-((2-methyl-4-(trifluoromethyl)phenyl)amino)-2-oxoethyl)-7-oxo-4,7-dihydro-[1,2,4]triazolo[1,5-a]pyrimidin-6-yl)-3,8-diazabicyclo[3.2.1]octane-8-carboxylate Int 18

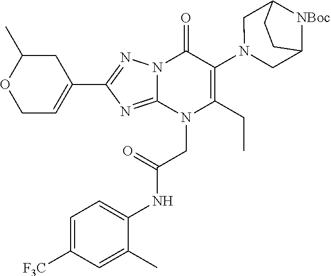 [SMILES: CCc1c(N2CC3CCC(C2)N3C(=O)OC(C)(C)C)c(=O)n2nc(C3=CCOC(C)C3)nc2n1CC(=O)Nc1ccc(C(F)(F)F)cc1C]

Int 18 could be made by following the same method of Int 13 via the Suzuki coupling of Int 11 and 4,4,5,5-tetramethyl-2-(2-methyl-3,6-dihydro-2H-pyran-4-yl)-1,3,2-dioxaborolane.

### Synthesis of tert-butyl 3-(5-ethyl-2-(4-methoxycyclopent-1-en-1-yl)-4-(2-((2-methyl-4-(trifluoromethyl)phen yl)amino)-2-oxoethyl)-7-oxo-4,7-dihydro-[1,2,4]triazolo[1,5-a]pyrimidin-6-yl)-3,8-di azabicyclo[3.2.1]octane-8-carboxylate Int 19

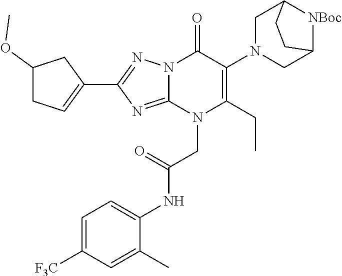 [SMILES: CCc1c(N2CC3CCC(C2)N3C(=O)OC(C)(C)C)c(=O)n2nc(C3=CCC(OC)C3)nc2n1CC(=O)Nc1ccc(C(F)(F)F)cc1C]

Int 19 could be made by following the same method of Int 13 via the Suzuki coupling of Int 11 and 2-(4-methoxycyclopent-1-en-1-yl)-4,4,5,5-tetramethyl-1,3,2-dioxaborolane.

### Synthesis of tert-butyl 3-(5-ethyl-2-(4-hydroxy-4-methylcyclopent-1-en-1-yl)-4-(2-((2-methyl-4-(trifluoromethyl)phenyl)amino)-2-oxoethyl)-7-oxo-4,7-dihydro-[1,2,4]triazolo[1,5-a]pyrimidin-6-yl)-3,8-diazabicyclo[3.2.1]octane-8-carboxylate Int 20

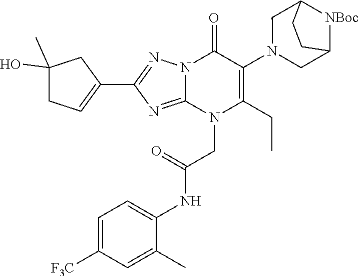 [SMILES: CCc1c(N2CC3CCC(C2)N3C(=O)OC(C)(C)C)c(=O)n2nc(C3=CCC(C)(O)C3)nc2n1CC(=O)Nc1ccc(C(F)(F)F)cc1C]

Int 20 could be made by following the same method of Int 13 via the Suzuki coupling of Int 11 and 1-methyl-3-(4,4,5,5-tetramethyl-1,3,2-dioxaborolan-2-yl)cyclopent-3-en-1-ol.

### Synthesis of tert-butyl 3-(5-ethyl-2-(3-methoxybicyclo[3.1.0]hexan-6-yl)-4-(2-((2-methyl-4-(trifluoromethyl)phenyl)amino)-2-oxoethyl)-7-oxo-4,7-dihydro-[1,2,4]triazolo[1,5-a]pyrimidin-6-yl)-3,8-diazabicyclo[3.2.1]octane-8-carboxylate Int 21

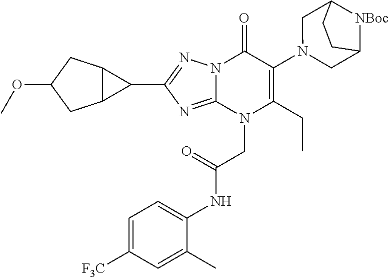 [SMILES: CCc1c(N2CC3CCC(C2)N3C(=O)OC(C)(C)C)c(=O)n2nc(C3C4CC(OC)CC43)nc2n1CC(=O)Nc1ccc(C(F)(F)F)cc1C]

Int 21 could be made by following the same method of Int 13 via the Suzuki coupling of Int 11 and 2-(3-methoxybicyclo[3.1.0]hexan-6-yl)-4,4,5,5-tetramethyl-1,3,2-dioxaborolane.

### Synthesis of tert-butyl 3-(5-ethyl-2-(3-(methoxycarbonyl)bicyclo[3.1.0]hexan-6-yl)-4-(2-((2-methyl-4-(trifluoromethyl)phenyl)amino)-2-oxoethyl)-7-oxo-4,7-dihydro-[1,2,4]triazolo[1,5-a]pyrimidin-6-yl)-3,8-diazabicyclo[3.2.1]octane-8-carboxylate Int 22

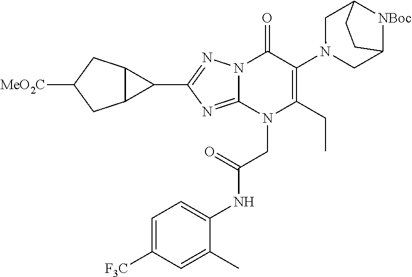 [SMILES: CCc1c(N2CC3CCC(C2)N3C(=O)OC(C)(C)C)c(=O)n2nc(C3C4CC(C(=O)OC)CC43)nc2n1CC(=O)Nc1ccc(C(F)(F)F)cc1C]

Int 22 could be made by following the same method of Int 13 via the Suzuki coupling of Int 11 and methyl 6-(4,4,5,5-tetramethyl-1,3,2-dioxaborolan-2-yl)bicyclo[3.1.0]hexane-3-carboxylate.

### Synthesis of tert-butyl 3-(2-(3-((tert-butyldimethylsilyl)oxy)bicyclo[3.1.0]hexan-6-yl)-5-ethyl-4-(2-((2-meth yl-4-(trifluoromethyl)phenyl)amino)-2-oxoethyl)-7-oxo-4,7-dihydro-[1,2,4]triazolo[1, 5-a]pyrimidin-6-yl)-3,8-diazabicyclo[3.2.1]octane-8-carboxylate Int 23

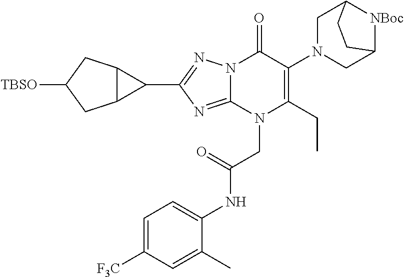 [SMILES: CCc1c(N2CC3CCC(C2)N3C(=O)OC(C)(C)C)c(=O)n2nc(C3C4CC(O[Si](C)(C)C(C)(C)C)CC43)nc2n1CC(=O)Nc1ccc(C(F)(F)F)cc1C]

Int 23 could be made by following the same method of Int 13 via the Suzuki coupling of Int 11 and tert-butyldimethyl((6-(4,4,5,5-tetramethyl-1,3,2-dioxaborolan-2-yl)bicyclo[3.1.0]hexan-3-yl)oxy)silane.

### Synthesis of tert-butyl 3-(2-(3-oxabicyclo[4.1.0]heptan-7-yl)-5-ethyl-4-(2-((2-methyl-4-(trifluoromethyl)phenyl)amino)-2-oxoethyl)-7-oxo-4,7-dihydro-[1,2,4]triazolo[1,5-a]pyrimidin-6-yl)-3,8-diazabicyclo[3.2.1]octane-8-carboxylate Int 24

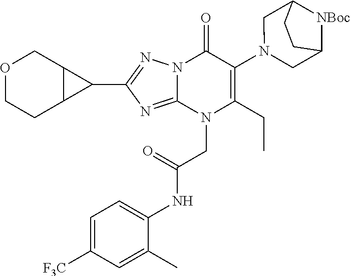 [SMILES: CCc1c(N2CC3CCC(C2)N3C(=O)OC(C)(C)C)c(=O)n2nc(C3C4CCOCC43)nc2n1CC(=O)Nc1ccc(C(F)(F)F)cc1C]

Int 24 could be made by following the same method of Int 13 via the Suzuki coupling of Int 11 and 2-(3-oxabicyclo[4.1.0]heptan-7-yl)-4,4,5,5-tetramethyl-1,3,2-dioxaborolane.

### Synthesis of tert-butyl 3-(5-ethyl-4-(2-((2-methyl-4-(trifluoromethyl)phenyl)amino)-2-oxoethyl)-7-oxo-2-(5-oxaspiro[2.4]heptan-1-yl)-4,7-dihydro-[1,2,4]triazolo[1,5-a]pyrimidin-6-yl)-3,8-diaza bicyclo[3.2.1]octane-8-carboxylate Int 25

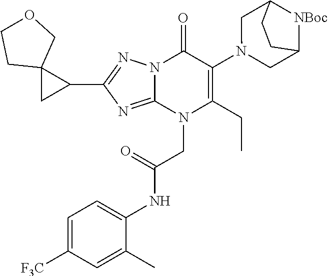 [SMILES: CCc1c(N2CC3CCC(C2)N3C(=O)OC(C)(C)C)c(=O)n2nc(C3CC34CCOC4)nc2n1CC(=O)Nc1ccc(C(F)(F)F)cc1C]

Int 25 could be made by following the same method of Int 13 via the Suzuki coupling of Int 11 and 4,4,5,5-tetramethyl-2-(5-oxaspiro[2.4]heptan-1-yl)-1,3,2-dioxaborolane.

### Synthesis of tert-butyl 3-(5-ethyl-4-(2-((2-methyl-4-(trifluoromethyl)phenyl)amino)-2-oxoethyl)-7-oxo-2-(6-oxaspiro[2.5]octan-1-yl)-4,7-dihydro-[1,2,4]triazolo[1,5-a]pyrimidin-6-yl)-3,8-diazabicyclo[3.2.1]octane-8-carboxylate Int 26

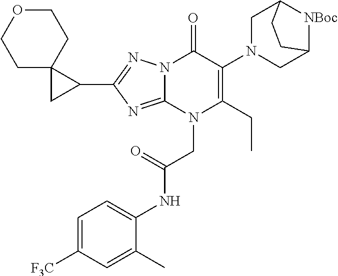 [SMILES: CCc1c(N2CC3CCC(C2)N3C(=O)OC(C)(C)C)c(=O)n2nc(C3CC34CCOCC4)nc2n1CC(=O)Nc1ccc(C(F)(F)F)cc1C]

Int 26 could be made by following the same method of Int 13 via the Suzuki coupling of Int 11 and 4,4,5,5-tetramethyl-2-(6-oxaspiro[2.5]octan-1-yl)-1,3,2-dioxaborolane.

### Synthesis of tert-butyl 3-(5-ethyl-4-(2-((2-methyl-4-(trifluoromethyl)phenyl)amino)-2-oxoethyl)-7-oxo-2-(5-oxaspiro[2.5]octan-1-yl)-4,7-dihydro-[1,2,4]triazolo[1,5-a]pyrimidin-6-yl)-3,8-diazabicyclo[3.2.1]octane-8-carboxylate Int 27

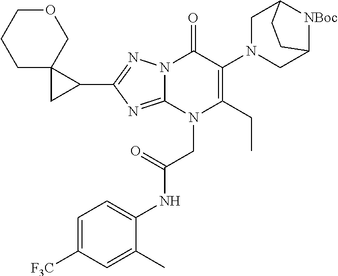 [SMILES: CCc1c(N2CC3CCC(C2)N3C(=O)OC(C)(C)C)c(=O)n2nc(C3CC34CCCOC4)nc2n1CC(=O)Nc1ccc(C(F)(F)F)cc1C]

Int 27 could be made by following the same method of Int 13 via the Suzuki coupling of Int 11 and 4,4,5,5-tetramethyl-2-(5-oxaspiro[2.5]octan-1-yl)-1,3,2-dioxaborolane.

### Synthesis of tert-butyl 3-(2-(cyclobut-1-en-1-yl)-5-ethyl-4-(2-((2-methyl-4-(trifluoromethyl)phenyl)amino)-2-oxoethyl)-7-oxo-4,7-dihydro-[1,2,4]triazolo[1,5-a]pyrimidin-6-yl)-3,8-diazabicyclo[3.2.1]octane-8-carboxylate Int 28

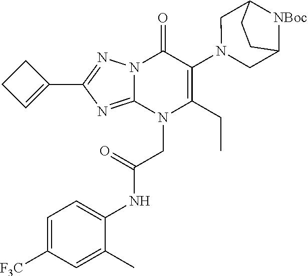 [SMILES: CCc1c(N2CC3CCC(C2)N3C(=O)OC(C)(C)C)c(=O)n2nc(C3=CCC3)nc2n1CC(=O)Nc1ccc(C(F)(F)F)cc1C]

Int 28 could be made by following the same method of Int 13 via the Suzuki coupling of Int 11 and 2-(cyclobut-1-en-1-yl)-4,4,5,5-tetramethyl-1,3,2-dioxaborolane.

### Synthesis of tert-butyl 3-(2-(3,3-difluorocyclobut-1-en-1-yl)-5-ethyl-4-(2-((2-methyl-4-(trifluoromethyl)phen yl)amino)-2-oxoethyl)-7-oxo-4,7-dihydro-[1,2,4]triazolo[1,5-a]pyrimidin-6-yl)-3,8-di azabicyclo[3.2.1]octane-8-carboxylate Int 29

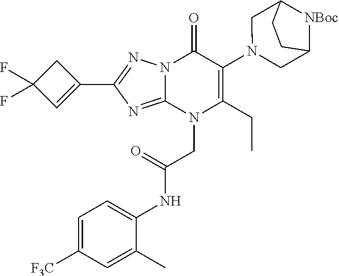 [SMILES: CCc1c(N2CC3CCC(C2)N3C(=O)OC(C)(C)C)c(=O)n2nc(C3=CC(F)(F)C3)nc2n1CC(=O)Nc1ccc(C(F)(F)F)cc1C]

Int 29 could be made by following the same method of Int 13 via the Suzuki coupling of Int 11 and 2-(3,3-difluorocyclobut-1-en-1-yl)-4,4,5,5-tetramethyl-1,3,2-dioxaborolane.

### Synthesis of tert-butyl 3-(2-(3-(benzyloxy)cyclobut-1-en-1-yl)-5-ethyl-4-(2-((2-methyl-4-(trifluoromethyl)phenyl)amino)-2-oxoethyl)-7-oxo-4,7-dihydro-[1,2,4]triazolo[1,5-a]pyrimidin-6-yl)-3,8-diazabicyclo[3.2.1]octane-8-carboxylate Int 30

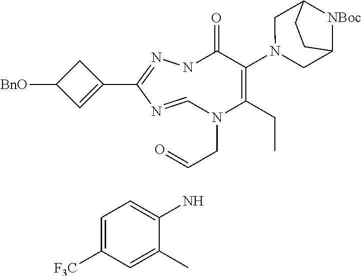

Int 30 could be made by following the same method of Int 13 via the Suzuki coupling of Int 11 and 2-(3-(benzyloxy)cyclobut-1-en-1-yl)-4,4,5,5-tetramethyl-1,3,2-dioxaborolane.

### Synthesis of tert-butyl 3-(5-ethyl-2-(3-(methoxycarbonyl)cyclobut-1-en-1-yl)-4-(2-((2-methyl-4-(trifluoromethyl)phenyl)amino)-2-oxoethyl)-7-oxo-4,7-dihydro-[1,2,4]triazolo[1,5-a]pyrimidin-6-yl)-3,8-diazabicyclo[3.2.1]octane-8-carboxylate Int 31

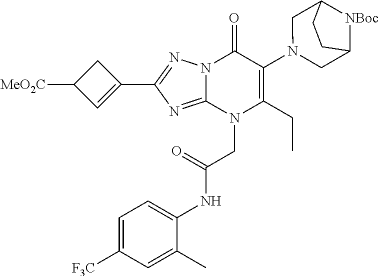 [SMILES: CCc1c(N2CC3CCC(C2)N3C(=O)OC(C)(C)C)c(=O)n2nc(C3=CC(C(=O)OC)C3)nc2n1CC(=O)Nc1ccc(C(F)(F)F)cc1C]

Int 31 could be made by following the same method of Int 13 via the Suzuki coupling of Int 11 and methyl 3-(4,4,5,5-tetramethyl-1,3,2-dioxaborolan-2-yl)cyclobut-2-ene-1-carboxylate.

### Synthesis of 1-(2,2-difluoroethyl)-4-(4,4,5,5-tetramethyl-1,3,2-dioxaborolan-2-yl)-1,2,3,6-tetrahydropyridine Int 32

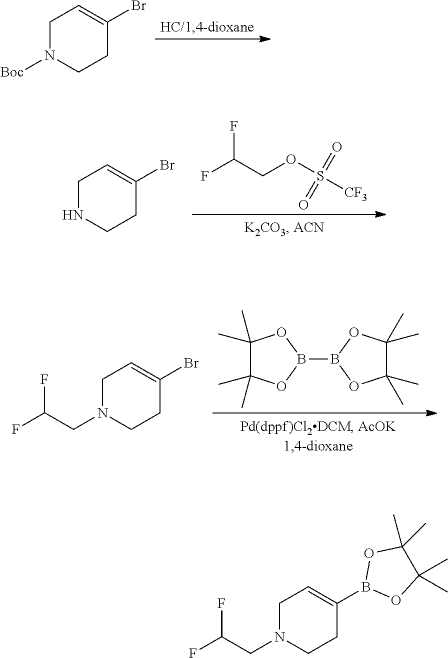

### Step 1: 4-bromo-1,2,3,6-tetrahydropyridine

Tert-butyl 4-bromo-3,6-dihydropyridine-1(2H)-carboxylate (4 g, 15.26 mmol) was suspended in hydrogen chloride in dioxane (30 mL) and the mixture was stirred at RT for 2 h. The reaction was concentrated under reduced pressure and used without purification. Mass calc C₅H₈BrN for 160.98, 162.98 found:162.2, 164.2 (M+H)⁺ ESI.

### Step 2: 4-bromo-1-(2,2-difluoroethyl)-1,2,3,6-tetrahydropyridine

4-bromo-1,2,3,6-tetrahydropyridine (1 g, 6.17 mmol) was suspended in MeCN (20 mL), 2,2-difluoroethyl trifluoromethanesulfonate (21.72 g, 8.02 mmol) and potassium carbonate (2.56 g, 18.52 mmol) were added, and the mixture was stirred at 80° C. for 16 h under N₂ atmosphere. The mixture was diluted with EA and water, extracted once with EA and the organic layer was washed with brine, dried over Na₂SO₄, filtered and concentrated under reduced pressure to give a residue. The crude product was purified by column chromatography to give the product (958 mg, 68.66% yield).

Mass calc C₇H₁₀BrF₂N for 225.00, 226.99 found: 226.1, 228.2 (M+H)⁺ ESI.

### Step 3: 1-(2,2-difluoroethyl)-4-(4,4,5,5-tetramethyl-1,3,2-dioxaborolan-2-yl)-1,2,3,6-tetrahydropyridine

4-bromo-1-(2,2-difluoroethyl)-1,2,3,6-tetrahydropyridine (958 mg, 4.24 mmol) was suspended in 1,4-dioxane (20 mL), 4,4,4′,4′,5,5,5′,5′-octamethyl-2,2′-bi(1,3,2-dioxaborolane) (1.18 g, 4.66 mmol) and potassium Acetate (1.25 g, 12.71 mmol) were added and the mixture was degassed for 10 minutes with argon, PdCl₂(dppf) (343.51 mg, 0.42 mmol) was added and the mixture was stirred at 90° C. overnight. The mixture was concentrated under reduced pressure and the crude product was purified by column chromatography to give the product (1.05 g, 90.72% yield). Mass calc C₁₃H₂₂BF₂NO₂ for 273.17 found: 274.3 (M+H)⁺ ESI.

### Synthesis of 4-(4,4,5,5-tetramethyl-1,3,2-dioxaborolan-2-yl)-1-(2,2,2-trifluoroethyl)-1,2,3,6-tetrahydropyridine Int 33

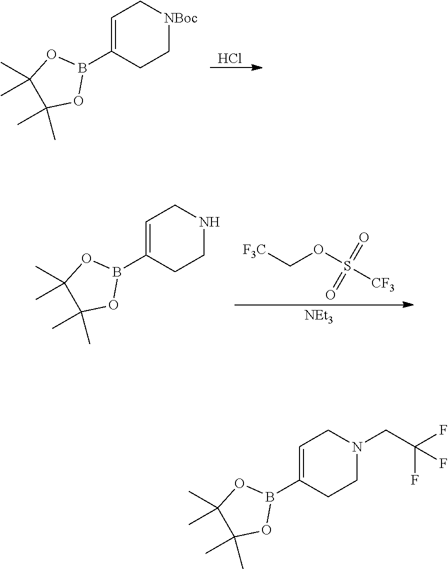 [SMILES: CC(C)(C)OC(=O)N1CC=C(B2OC(C)(C)C(C)(C)O2)CC1 ; Cl ; CC1(C)OB(C2=CCNCC2)OC1(C)C ; O=S(=O)(OCC(F)(F)F)C(F)(F)F ; CC1(C)OB(C2=CCN(CC(F)(F)F)CC2)OC1(C)C ; "NEt3"]

Int 33 is synthesized from tert-butyl 4-(4,4,5,5-tetramethyl-1,3,2-dioxaborolan-2-yl)-3,6-dihydropyridine-1(2H)-carboxylate according to the reported procedure (WO2018067512).

### Synthesis of 5-fluoro-4-(4,4,5,5-tetramethyl-1,3,2-dioxaborolan-2-yl)-1-(2,2,2-trifluoroethyl)-1,2,3,6-tetrahydropyridine Int 34

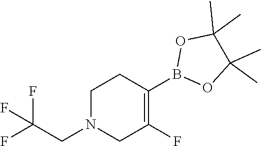 [SMILES: CC1(C)OB(C2=C(F)CN(CC(F)(F)F)CC2)OC1(C)C]

Int 34 could be made by following the same method of Int 33 from tert-butyl 5-fluoro-4-(4,4,5,5-tetramethyl-1,3,2-dioxaborolan-2-yl)-3,6-dihydropyridine-1(2H)-carboxylate

### Synthesis of 7-(4,4,5,5-tetramethyl-1,3,2-dioxaborolan-2-yl)-3-(2,2,2-trifluoroethyl)-3-azabicyclo[4.1.0]heptane Int 35

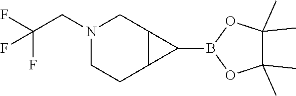 [SMILES: CC1(C)OB(C2C3CCN(CC(F)(F)F)CC32)OC1(C)C]

Int 35 could be made by following the same method of Int 33 from tert-butyl 7-(4,4,5,5-tetramethyl-1,3,2-dioxaborolan-2-yl)-3-azabicyclo[4.1.0]heptane-3-carboxy late.

### Synthesis of 6-(4,4,5,5-tetramethyl-1,3,2-dioxaborolan-2-yl)-N-(2,2,2-trifluoroethyl)bicyclo[3.1.0]hexan-3-amine Int 36

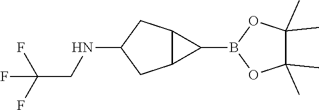 [SMILES: CC1(C)OB(C2C3CC(NCC(F)(F)F)CC32)OC1(C)C]

Int 36 could be made by following the same method of Int 33 from tert-butyl (6-(4,4,5,5-tetramethyl-1,3,2-dioxaborolan-2-yl)bicyclo[3.1.0]hexan-3-yl)carbamate.

### Synthesis of 1-(4,4,5,5-tetramethyl-1,3,2-dioxaborolan-2-yl)-6-(2,2,2-trifluoroethyl)-6-azaspiro[2.5]octane Int 37

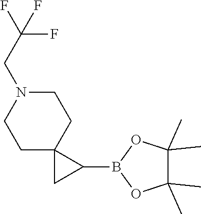 [SMILES: CC1(C)OB(C2CC23CCN(CC(F)(F)F)CC3)OC1(C)C]

Int 37 could be made by following the same method of Int 33 from tert-butyl 1-(4,4,5,5-tetramethyl-1,3,2-dioxaborolan-2-yl)-6-azaspiro[2.5]octane-6-carboxylate.

### Synthesis of 1-(4,4,5,5-tetramethyl-1,3,2-dioxaborolan-2-yl)-5-(2,2,2-trifluoroethyl)-5-azaspiro[2.4]heptane Int 38

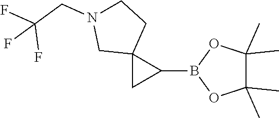 [SMILES: CC1(C)OB(C2CC23CCN(CC(F)(F)F)C3)OC1(C)C]

Int 38 could be made by following the same method of Int 33 from tert-butyl 1-(4,4,5,5-tetramethyl-1,3,2-dioxaborolan-2-yl)-5-azaspiro[2.4]heptane-5-carboxylate.

### Synthesis of 1-(4,4,5,5-tetramethyl-1,3,2-dioxaborolan-2-yl)-5-(2,2,2-trifluoroethyl)-5-azaspiro[2.3]hexane Int 39

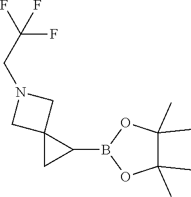 [SMILES: CC1(C)OB(C2CC23CN(CC(F)(F)F)C3)OC1(C)C]

Int 39 could be made by following the same method of Int 33 from tert-butyl 1-(4,4,5,5-tetramethyl-1,3,2-dioxaborolan-2-yl)-5-azaspiro[2.3]hexane-5-carboxylate.

### Synthesis of 3-(4,4,5,5-tetramethyl-1,3,2-dioxaborolan-2-yl)-N-(2,2,2-trifluoroethyl)cyclobut-2-en-1-amine Int 40

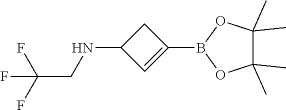 [SMILES: CC1(C)OB(C2=CC(NCC(F)(F)F)C2)OC1(C)C]

Int 40 could be made by following the same method of Int 33 from tert-butyl (3-(4,4,5,5-tetramethyl-1,3,2-dioxaborolan-2-yl)cyclobut-2-en-1-yl)carbamate.

### Preparation Example 1A

### Synthesis of Compound 86

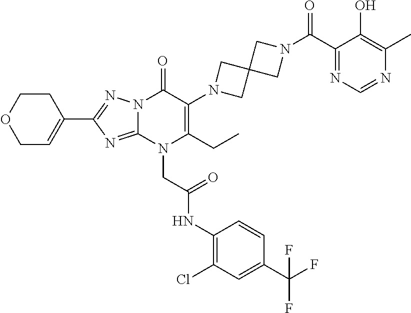 [SMILES: CCc1c(N2CC3(CN(C(=O)c4ncnc(C)c4O)C3)C2)c(=O)n2nc(C3=CCOCC3)nc2n1CC(=O)Nc1ccc(C(F)(F)F)cc1Cl]

### Step 1. tert-butyl 6-(2-bromo-5-ethyl-7-oxo-4,7-dihydro-[1,2,4]triazolo[1,5-a]pyrimidin-6-yl)-2,6-diaza spiro[3.3]heptane-2-carboxylate

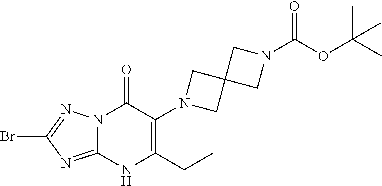 [SMILES: CCc1[nH]c2nc(Br)nn2c(=O)c1N1CC2(CN(C(=O)OC(C)(C)C)C2)C1]

To a solution of tert-butyl 6-(1-ethoxy-1,3-dioxopentan-2-yl)-2,6-diazaspiro[3.3]heptane-2-carboxylate (793.2 mg, 2.33 mmol) in EtOH (4 mL) were added 3-bromo-1H-1,2,4-trizaol-5-amine (400 mg, 2.33 mmol) and H₃PO₄ (282 mg, 2.95 mmol). The resulting mixture was stirred at 85° C. for 24 h, then cooled to room temperature. DIPEA (1.22 mL, 7 mmol) and Boc₂O (255 mg, 1.17 mmol) were added, and the mixture was stirred at room temperature for another 2 h before it was quenched with small amount of water. EtOH was removed under reduced pressure, and the residue was dissolved in EtOAc and washed with water, brine, dried (Na₂SO₄), and concentrated. The residue was purified by column chromatography (0-75% EtOAc in heptane) to provide the title compound.

### Step 2. tert-butyl 6-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)-2,6-diazaspiro[3.3]heptane-2-carboxylate

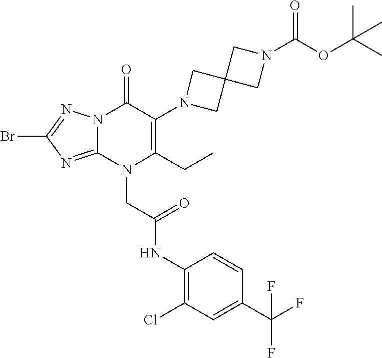 [SMILES: CCc1c(N2CC3(CN(C(=O)OC(C)(C)C)C3)C2)c(=O)n2nc(Br)nc2n1CC(=O)Nc1ccc(C(F)(F)F)cc1Cl]

To a mixture of tert-butyl 6-(2-bromo-5-ethyl-7-oxo-4,7-dihydro-[1,2,4]triazolo[1,5-a]pyrimidin-6-yl)-2,6-diaza spiro[3.3]heptane-2-carboxylate (110 mg, 0.25 mmol) and 2-chloro-N-(2-chloro-4-(trifluoromethyl)phenyl)acetamide (76.2 mg, 0.28 mmol) in 1,4-dioxane (3 mL) were added KI (41.5 mg, 0.25 mmol) and DIPEA (131 μL, 0.75 mmol). The resulting mixture was stirred at 80° C. for 3.5 h, then cooled to room temperature. The mixture was partitioned between EtOAc and water. The organic layer was washed with brine, dried (Na₂SO₄), filtered, and concentrated. The residue was purified by column chromatography (0-50% EtOAc in heptane) to provide the title compound.

### Step 3. tert-butyl 6-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-2,6-diazaspiro[3.3]heptane-2-carboxylat

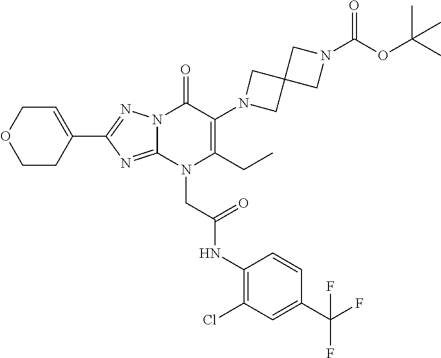 [SMILES: CCc1c(N2CC3(CN(C(=O)OC(C)(C)C)C3)C2)c(=O)n2nc(C3=CCOCC3)nc2n1CC(=O)Nc1ccc(C(F)(F)F)cc1Cl]

A mixture of tert-butyl 6-(2-bromo-5-ethyl-4-(2-((2-chloro-4-(trifluoromethyl)phenyl)amino)-2-oxoethyl)-7-oxo-4,7-dihydro-[1,2,4]triazolo[1,5-a]pyrimidin-6-yl)-2,6-diazaspiro[3.3]heptane-2-carboxylate (124.2 mg, 184 μmol), 2-(3,6-dihydro-2H-pyran-4-yl)-4,4,5,5-tetramethyl-1,3,2-dioxaborolane (58 mg, 276 μmol), K₃PO₄ (78.5 mg, 368 μmol), and Pd(dppf)Cl₂·DCM (15.1 mg, 18.4 μmol) in 1,4-dioxane (4 mL) and water (2 mL) was stirred at 80° C. for 8 h. The mixture was cooled to room temperature and diluted with EtOAc. The organic layer was washed with water and brine, and dried (Na₂SO₄) and concentrated. The residue was purified by column chromatography (0-100% EtOAc in heptane) to provide the title compound.

### Step 4. N-(2-chloro-4-(trifluoromethyl)phenyl)-2-(2-(3,6-dihydro-2H-pyran-4-yl)-5-ethyl-7-oxo-6-(2,6-diazaspiro[3.3]heptan-2-yl)-[1,2,4]triazolo[1,5-a]pyrimidin-4(7H)-yl)acetamide

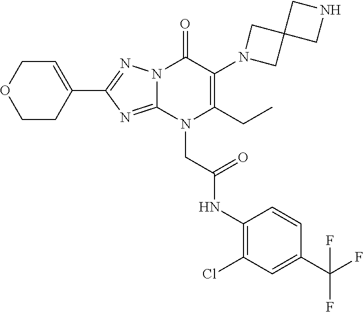 [SMILES: CCc1c(N2CC3(CNC3)C2)c(=O)n2nc(C3=CCOCC3)nc2n1CC(=O)Nc1ccc(C(F)(F)F)cc1Cl]

To a solution of tert-butyl 6-(2-(3,6-dihydro-2H-pyran-4-yl)-5-ethyl-4-(2-((2-methyl-4-(trifluoromethyl)phenyl) amino)-2-oxoethyl)-7-oxo-4,7-dihydro-[1,2,4]triazolo[1,5-a]pyrimidin-6-yl)-2,6-diaza spiro[3.3]heptane-2-carboxylate (51.5 mg, 76 μmol) in dichloromethane (1 mL) was added TFA (0.25 mL). The mixture was stirred at room temperature for 1 h, then diluted with dichloromethane and neutralized with aqueous saturated NaHCO₃. The organic layer was dried (Na₂SO₄), concentrated and dried under vacuum to provide the title compound.

### Step 5. N-(2-chloro-4-(trifluoromethyl)phenyl)-2-(2-(3,6-dihydro-2H-pyran-4-yl)-5-ethyl-6-(6-(5-hydroxy-6-methylpyrimidine-4-carbonyl)-2,6-diazaspiro[3.3]heptan-2-yl)-7-oxo-[1,2,4]triazolo[1,5-a]pyrimidin-4(7H)-yl)acetamide

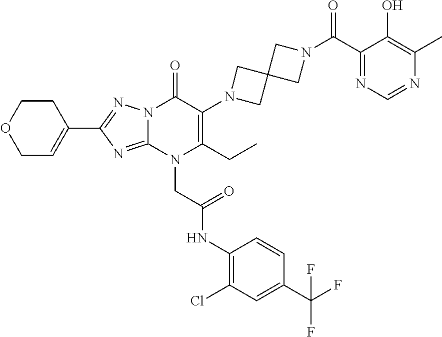 [SMILES: CCc1c(N2CC3(CN(C(=O)c4ncnc(C)c4O)C3)C2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-7-oxo-6-(2,6-diazaspiro[3.3]heptan-2-yl)-[1,2,4]triazolo[1,5-a]pyrimidin-4(7H)-yl)acetamide (40 mg, 69 μmol) and 5-hydroxy-6-methylpyrimidine-4-carboxylic acid (12.8 mg, 82.8 μmol) in DMF (1.5 mL) at room temperature were added HATU (40.5 mg, 103.5 μmol) and DIPEA (60.2 μL, 345 μmol). The mixture was stirred at room temperature for 2 h, then quenched with water. Aqueous saturated NaHCO₃ was added and the mixture was extracted with EtOAc. The organic layer was dried (Na₂SO₄), concentrated. The residue was purified by column chromatography (0-10% MeOH in dichloromethane) to provide the title compound.

### Preparation Example 1B

### Synthesis of compound 86 N-(2-chloro-4-(trifluoromethyl)phenyl)-2-(2-(3,6-dihydro-2H-pyran-4-yl)-5-ethyl-6-(6-(5-hydroxy-6-methylpyrimidine-4-carbonyl)-2,6-diazaspiro[3.3]heptan-2-yl)-7-oxo-[1,2,4]triazolo[1,5-a]pyrimidin-4(7H)-yl)acetamide

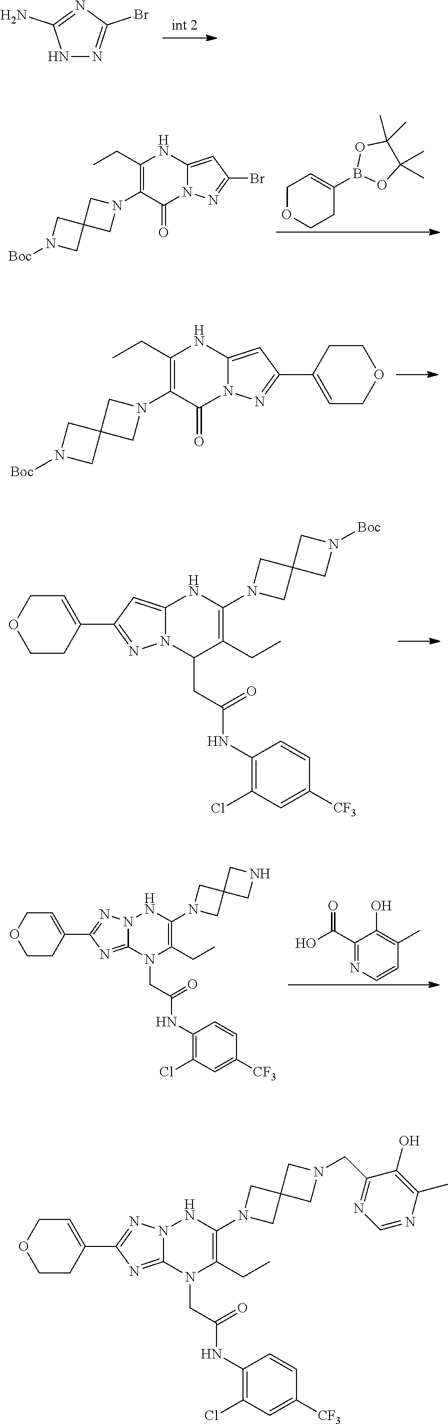

### Step 1. tert-butyl 6-(2-bromo-5-ethyl-7-oxo-4,7-dihydro-[1,2,4]triazolo[1,5-a]pyrimidin-6-yl)-2,6-diaza spiro[3.3]heptane-2-carboxylate

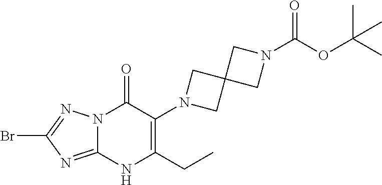 [SMILES: CCc1[nH]c2nc(Br)nn2c(=O)c1N1CC2(CN(C(=O)OC(C)(C)C)C2)C1]

To a solution of tert-butyl 6-(1-ethoxy-1,3-dioxopentan-2-yl)-2,6-diazaspiro[3.3]heptane-2-carboxylate (793.2 mg, 2.33 mmol) in EtOH (4 mL) were added 3-bromo-1H-1,2,4-trizaol-5-amine (400 mg, 2.33 mmol) and H₃PO₄ (282 mg, 2.95 mmol). The resulting mixture was stirred at 85° C. for 24 h, then cooled to room temperature. DIPEA (1.22 mL, 7 mmol) and Boc₂O (255 mg, 1.17 mmol) were added, and the mixture was stirred at room temperature for another 2 h before it was quenched with small amount of water. EtOH was removed under reduced pressure, and the residue was dissolved in EtOAc and washed with water, brine, dried (Na₂SO₄), and concentrated. The residue was purified by column chromatography (0-75% EtOAc in heptane) to provide the title compound.

### Step 2. tert-butyl 6-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)-2,6-diazaspiro[3.3]heptane-2-carboxylate

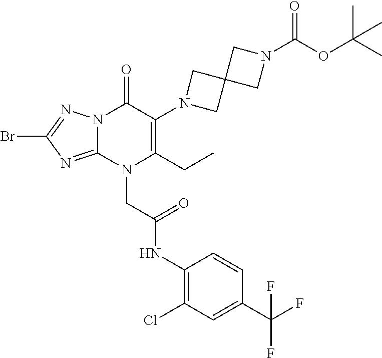 [SMILES: CCc1c(N2CC3(CN(C(=O)OC(C)(C)C)C3)C2)c(=O)n2nc(Br)nc2n1CC(=O)Nc1ccc(C(F)(F)F)cc1Cl]

To a mixture of tert-butyl 6-(2-bromo-5-ethyl-7-oxo-4,7-dihydro-[1,2,4]triazolo[1,5-a]pyrimidin-6-yl)-2,6-diaza spiro[3.3]heptane-2-carboxylate (110 mg, 0.25 mmol) and 2-chloro-N-(2-chloro-4-(trifluoromethyl)phenyl)acetamide (76.2 mg, 0.28 mmol) in 1,4-dioxane (3 mL) were added KI (41.5 mg, 0.25 mmol) and DIPEA (131 μL, 0.75 mmol). The resulting mixture was stirred at 80° C. for 3.5 h, then cooled to room temperature. The mixture was partitioned between EtOAc and water. The organic layer was washed with brine, dried (Na₂SO₄), filtered, and concentrated. The residue was purified by column chromatography (0-50% EtOAc in heptane) to provide the title compound.

### Step 3. tert-butyl 6-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-2,6-diazaspiro[3.3]heptane-2-carboxylat

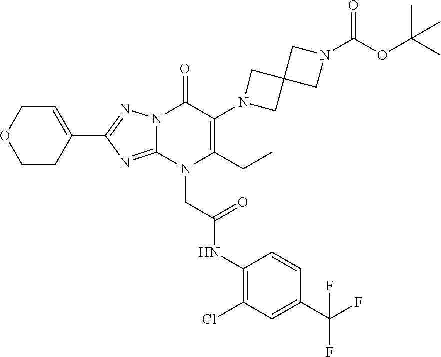 [SMILES: CCc1c(N2CC3(CN(C(=O)OC(C)(C)C)C3)C2)c(=O)n2nc(C3=CCOCC3)nc2n1CC(=O)Nc1ccc(C(F)(F)F)cc1Cl]

A mixture of tert-butyl 6-(2-bromo-5-ethyl-4-(2-((2-chloro-4-(trifluoromethyl)phenyl)amino)-2-oxoethyl)-7-oxo-4,7-dihydro-[1,2,4]triazolo[1,5-a]pyrimidin-6-yl)-2,6-diazaspiro[3.3]heptane-2-carboxylate (124.2 mg, 184 μmol), 2-(3,6-dihydro-2H-pyran-4-yl)-4,4,5,5-tetramethyl-1,3,2-dioxaborolane (58 mg, 276 μmol), K₃PO₄ (78.5 mg, 368 μmol), and Pd(dppf)Cl₂·DCM (15.1 mg, 18.4 μmol) in 1,4-dioxane (4 mL) and water (2 mL) was stirred at 80° C. for 8 h. The mixture was cooled to room temperature and diluted with EtOAc. The organic layer was washed with water and brine, and dried (Na₂SO₄) and concentrated. The residue was purified by column chromatography (0-100% EtOAc in heptane) to provide the title compound.

### Step 4. N-(2-chloro-4-(trifluoromethyl)phenyl)-2-(2-(3,6-dihydro-2H-pyran-4-yl)-5-ethyl-7-oxo-6-(2,6-diazaspiro[3.3]heptan-2-yl)-[1,2,4]triazolo[1,5-a]pyrimidin-4(7H)-yl)acetamide

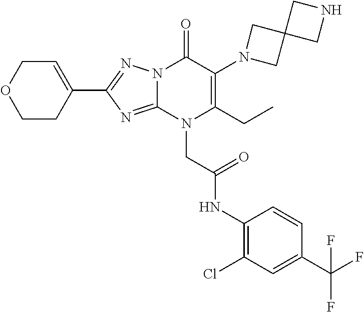 [SMILES: CCc1c(N2CC3(CNC3)C2)c(=O)n2nc(C3=CCOCC3)nc2n1CC(=O)Nc1ccc(C(F)(F)F)cc1Cl]

To a solution of tert-butyl 6-(2-(3,6-dihydro-2H-pyran-4-yl)-5-ethyl-4-(2-((2-methyl-4-(trifluoromethyl)phenyl) amino)-2-oxoethyl)-7-oxo-4,7-dihydro-[1,2,4]triazolo[1,5-a]pyrimidin-6-yl)-2,6-diaza spiro[3.3]heptane-2-carboxylate (51.5 mg, 76 μmol) in dichloromethane (1 mL) was added TFA (0.25 mL). The mixture was stirred at room temperature for 1 h, then diluted with dichloromethane and neutralized with aqueous saturated NaHCO₃. The organic layer was dried (Na₂SO₄), concentrated and dried under vacuum to provide the title compound.

### Step 5. N-(2-chloro-4-(trifluoromethyl)phenyl)-2-(2-(3,6-dihydro-2H-pyran-4-yl)-5-ethyl-6-(6-(5-hydroxy-6-methylpyrimidine-4-carbonyl)-2,6-diazaspiro[3.3]heptan-2-yl)-7-oxo-[1,2,4]triazolo[1,5-a]pyrimidin-4(7H)-yl)acetamide

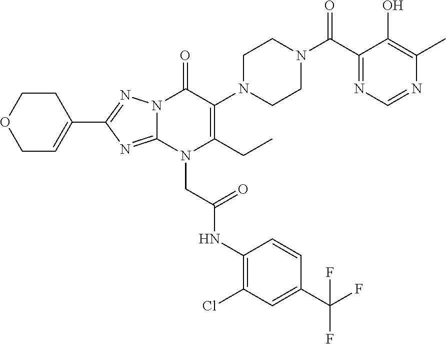 [SMILES: CCc1c(N2CCN(C(=O)c3ncnc(C)c3O)CC2)c(=O)n2nc(C3=CCOCC3)nc2n1CC(=O)Nc1ccc(C(F)(F)F)cc1Cl]

To a mixture of N-(2-chloro-4-(trifluoromethyl)phenyl)-2-(2-(3,6-dihydro-2H-pyran-4-yl)-5-ethyl-7-oxo-6-(2,6-diazaspiro[3.3]heptan-2-yl)-[1,2,4]triazolo[1,5-a]pyrimidin-4(7H)-yl)acetamide (40 mg, 69 mol) and 5-hydroxy-6-methylpyrimidine-4-carboxylic acid (12.8 mg, 82.8 μmol) in DMF (1.5 mL) at room temperature were added HATU (40.5 mg, 103.5 μmol) and DIPEA (60.2 μL, 345 μmol). The mixture was stirred at room temperature for 2 h, then quenched with water. Aqueous saturated NaHCO₃ was added and the mixture was extracted with EtOAc. The organic layer was dried (Na₂SO₄), concentrated. The residue was purified by column chromatography (0-10% MeOH in dichloromethane) to provide the title compound.

### Preparation Example 2

### Synthesis of Compound 163

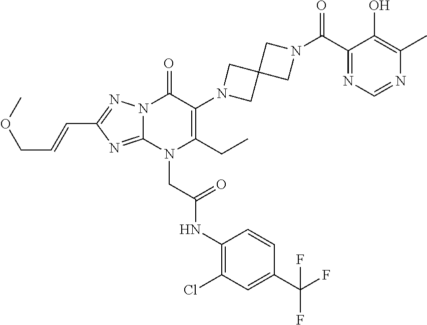 [SMILES: CCc1c(N2CC3(CN(C(=O)c4ncnc(C)c4O)C3)C2)c(=O)n2nc(/C=C/COC)nc2n1CC(=O)Nc1ccc(C(F)(F)F)cc1Cl]

### Step 1. tert-butyl (E)-6-(4-(2-((2-chloro-4-(trifluoromethyl)phenyl)amino)-2-oxoethyl)-5-ethyl-2-(3-methoxyprop-1-en-1-yl)-7-oxo-4,7-dihydro-[1,2,4]triazolo[1,5-a]pyrimidin-6-yl)-2,6-diazaspiro[3.3]heptane-2-carboxylate

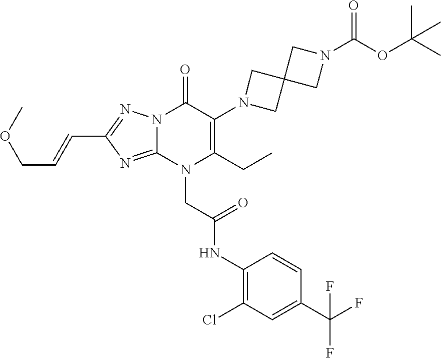 [SMILES: CCc1c(N2CC3(CN(C(=O)OC(C)(C)C)C3)C2)c(=O)n2nc(/C=C/COC)nc2n1CC(=O)Nc1ccc(C(F)(F)F)cc1Cl]

A mixture of tert-butyl 6-(2-bromo-5-ethyl-4-(2-((2-chloro-4-(trifluoromethyl)phenyl)amino)-2-oxoethyl)-7-oxo-4,7-dihydro-[1,2,4]triazolo[1,5-a]pyrimidin-6-yl)-2,6-diazaspiro[3.3]heptane-2-carboxylate (200 mg, 296 μmol), (E)-2-(3-Methoxyprop-1-enyl)-4,4,5,5-tetramnethyl-1,3,2-dioxaborolane (88 mg, 444 μmol), K₃PO₄ (126.3 mg, 592 μmol), and Pd(dppf)Cl₂·DCM (24.3 mg, 29.6 μmol) in 1,4-dioxane (6 mL) and water (3 mL) was stirred at 80° C. for 8 h. The mixture was cooled to room temperature and diluted with EtOAc. The organic layer was washed with water and brine, and dried (Na₂SO₄) and concentrated. The residue was purified by column chromatography (0-100% EtOAc in heptane) to provide the title compound.

### Step 2. (E)-N-(2-chloro-4-(trifluoromethyl)phenyl)-2-(5-ethyl-2-(3-methoxyprop-1-en-1-yl)-7-oxo-6-(2,6-diazaspiro[3.3]heptan-2-yl)-[1,2,4]triazolo[1,5-a]pyrimidin-4(7H)-yl)acetamide

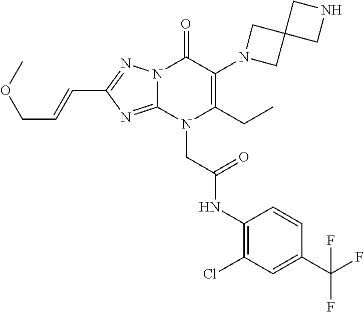 [SMILES: CCc1c(N2CC3(CNC3)C2)c(=O)n2nc(/C=C/COC)nc2n1CC(=O)Nc1ccc(C(F)(F)F)cc1Cl]

To a solution of tert-butyl (E)-6-(4-(2-((2-chloro-4-(trifluoromethyl)phenyl)amino)-2-oxoethyl)-5-ethyl-2-(3-methoxyprop-1-en-1-yl)-7-oxo-4,7-dihydro-[1,2,4]triazolo[1,5-a]pyrimidin-6-yl)-2,6-diazaspiro[3.3]heptane-2-carboxylate (70 mg, 105 μmol) in dichloromethane (1 mL) was added TFA (0.25 mL). The mixture was stirred at room temperature for 1 h, then diluted with dichloromethane and neutralized with aqueous saturated NaHCO₃. The organic layer was dried (Na₂SO₄), concentrated and dried under vacuum to provide the title compound.

### Step 3. (E)-N-(2-chloro-4-(trifluoromethyl)phenyl)-2-(5-ethyl-6-(6-(5-hydroxy-6-methylpyrimidine-4-carbonyl)-2,6-diazaspiro[3.3]heptan-2-yl)-2-(3-methoxyprop-1-en-1-yl)-7-oxo-[1,2,4]triazolo[1,5-a]pyrimidin-4(7H)-yl)acetamide

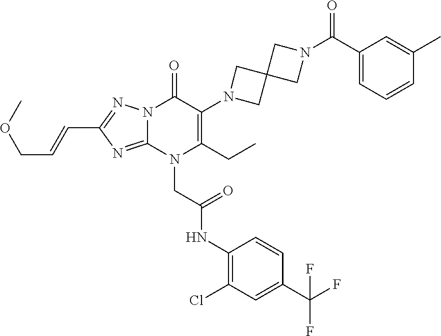 [SMILES: CCc1c(N2CC3(CN(C(=O)c4cccc(C)c4)C3)C2)c(=O)n2nc(/C=C/COC)nc2n1CC(=O)Nc1ccc(C(F)(F)F)cc1Cl]

To a mixture of (E)-N-(2-chloro-4-(trifluoromethyl)phenyl)-2-(5-ethyl-2-(3-methoxyprop-1-en-1-yl)-7-oxo-6-(2,6-diazaspiro[3.3]heptan-2-yl)-[1,2,4]triazolo[1,5-a]pyrimidin-4(7H)-yl)acetamide (45 mg, 79.5 μmol) and 5-hydroxy-6-methylpyrimidine-4-carboxylic acid (14.7 mg, 95.4 μmol) in DMF (2 mL) at room temperature were added HATU (46.7 mg, 119.3 μmol) and DIPEA (70 μL, 400 μmol). The mixture was stirred at room temperature for 2 h, then quenched with water. Aqueous saturated NaHCO₃ was added, and the mixture was extracted with EtOAc. The organic layer was dried (Na₂SO₄), concentrated. The residue was purified by column chromatography (0-10% MeOH in dichloromethane) to provide the title compound.

### Preparation Example 3

### Synthesis of Compound 71

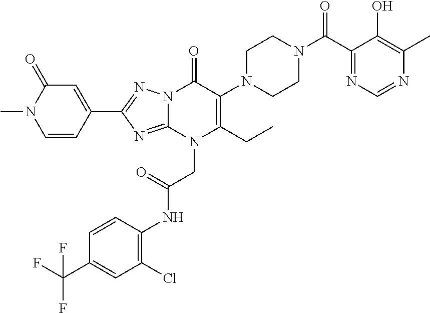

### Step 1. tert-butyl 4-(1-ethoxy-1,3-dioxopentan-2-yl)piperazine-1-carboxylate

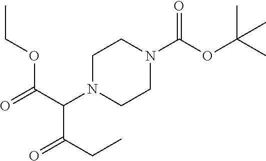 [SMILES: CCOC(=O)C(C(=O)CC)N1CCN(C(=O)OC(C)(C)C)CC1]

Ethyl 2-bromo-3-oxopentanoate (2 g, 8.96 mmol), tert-Butyl piperazine-1-carboxylate (977.5 mg, 27.1 mmol) and K₂CO₃ (9.18 g, 49.3 mmol) were mixed in acetonitrile (40 mL). The suspension was stirred at room temperature for 30 min. The reaction mixture was filtered. The combined filtrate and washings were concentrated under reduced pressure. The residue was purified by column chromatography (0-10% MeOH in dichloromethane) to provide the title compound.

### Step 2. tert-butyl 4-(2-bromo-5-ethyl-7-oxo-4,7-dihydro-[1,2,4]triazolo[1,5-a]pyrimidin-6-yl)piperazine-1-carboxylate

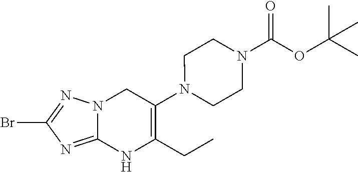 [SMILES: CCC1=C(N2CCN(C(=O)OC(C)(C)C)CC2)Cn2nc(Br)nc2N1]

To a solution of tert-butyl 4-(1-ethoxy-1,3-dioxopentan-2-yl)piperazine-1-carboxylate (765.2 mg, 2.33 mmol) in EtOH (4 mL) were added 3-bromo-1H-1,2,4-trizaol-5-amine (400 mg, 2.33 mmol) and H₃PO₄ (282 mg, 2.95 mmol). The resulting mixture was stirred at 85° C. for 24 h, then cooled to room temperature. DIPEA (1.22 mL, 7 mmol) and Boc₂O (255 mg, 1.17 mmol) were added, and the mixture was stirred at room temperature for another 2 h before it was quenched with small amount of water. EtOH was removed under reduced pressure, and the residue was dissolved in EtOAc and washed with water, brine, dried (Na₂SO₄), and concentrated. The residue was purified by column chromatography (0-75% EtOAc in heptane) to provide the title compound.

### Step 3. tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate

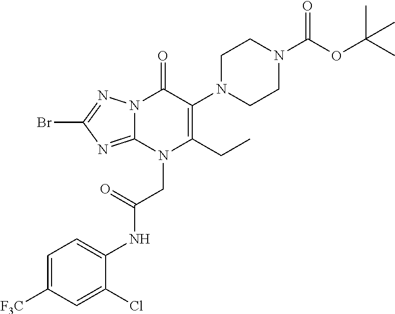 [SMILES: CCc1c(N2CCN(C(=O)OC(C)(C)C)CC2)c(=O)n2nc(Br)nc2n1CC(=O)Nc1ccc(C(F)(F)F)cc1Cl]

To a mixture of tert-butyl 4-(2-bromo-5-ethyl-7-oxo-4,7-dihydro-[1,2,4]triazolo[1,5-a]pyrimidin-6-yl)piperazine-1-carboxylate (125 mg, 0.29 mmol) and 2-chloro-N-(2-chloro-4-(trifluoromethyl)phenyl)acetamide (86.8 mg, 0.32 mmol) in 1,4-dioxane (4 mL) were added KI (48.1 mg, 0.29 mmol) and DIPEA (152 μL, 0.87 mmol). The resulting mixture was stirred at 80° C. for 3.5 h, then cooled to room temperature. The mixture was partitioned between EtOAc and water. The organic layer was washed with brine, dried (Na₂SO₄), filtered, and concentrated. The residue was purified by column chromatography (0-50% EtOAc in heptane) to provide the title compound.

### Step 4. tert-butyl 4-(4-(2-((2-chloro-4-(trifluoromethyl)phenyl)amino)-2-oxoethyl)-5-ethyl-2-(1-methyl-2-oxo-1,2-dihydropyridin-4-yl)-7-oxo-4,7-dihydro-[1,2,4]triazolo[1,5-a]pyrimidin-6-yl)piperazine-1-carboxylate

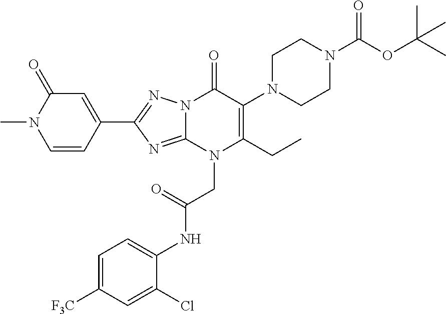 [SMILES: CCc1c(N2CCN(C(=O)OC(C)(C)C)CC2)c(=O)n2nc(-c3ccn(C)c(=O)c3)nc2n1CC(=O)Nc1ccc(C(F)(F)F)cc1Cl]

A mixture of tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate (200 mg, 302 μmol), 1-Methyl-2-oxo-1,2-dihydropyridin-4-ylboronic acid (69.3 mg, 453 μmol), K₃PO₄ (128.9 mg, 604 μmol), and Pd(dppf)Cl₂·DCM (24.8 mg, 30.2 μmol) in 1,4-dioxane (6 mL) and water (3 mL) was stirred at 80° C. for 8 h. The mixture was cooled to room temperature and diluted with EtOAc. The organic layer was washed with water and brine, and dried (Na₂SO₄) and concentrated. The residue was purified by column chromatography (0-100% EtOAc in heptane) to provide the title compound.

### Step 5. N-(2-chloro-4-(trifluoromethyl)phenyl)-2-(5-ethyl-2-(1-methyl-2-oxo-1,2-dihydropyridin-4-yl)-7-oxo-6-(piperazin-1-yl)-[1,2,4]triazolo[1,5-a]pyrimidin-4(7H)-yl)acetamide

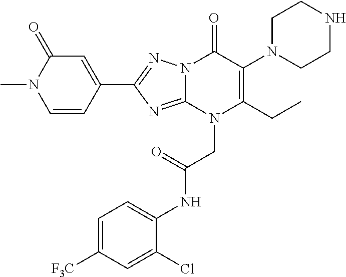 [SMILES: CCc1c(N2CCNCC2)c(=O)n2nc(-c3ccn(C)c(=O)c3)nc2n1CC(=O)Nc1ccc(C(F)(F)F)cc1Cl]

To a solution of tert-butyl 4-(4-(2-((2-chloro-4-(trifluoromethyl)phenyl)amino)-2-oxoethyl)-5-ethyl-2-(1-methyl-2-oxo-1,2-dihydropyridin-4-yl)-7-oxo-4,7-dihydro-[1,2,4]triazolo[1,5-a]pyrimidin-6-yl)piperazine-1-carboxylate (76 mg, 110 μmol) in dichloromethane (1 mL) was added TFA (0.25 mL). The mixture was stirred at room temperature for 1 h, then diluted with dichloromethane and neutralized with aqueous saturated NaHCO₃. The organic layer was dried (Na₂SO₄), concentrated and dried under vacuum to provide the title compound.

### Step 6. N-(2-chloro-4-(trifluoromethyl)phenyl)-2-(5-ethyl-6-(4-(5-hydroxy-6-methylpyrimidine-4-carbonyl)piperazin-1-yl)-2-(1-methyl-2-oxo-1,2-dihydropyridin-4-yl)-7-oxo-[1,2,4]triazolo[1,5-a]pyrimidin-4(7H)-yl)acetamide

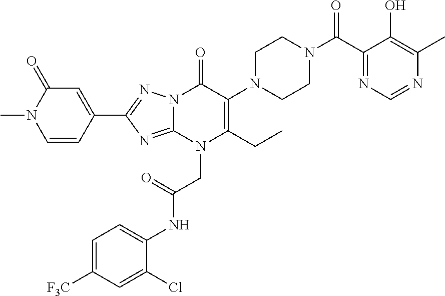

To a mixture of N-(2-chloro-4-(trifluoromethyl)phenyl)-2-(5-ethyl-2-(1-methyl-2-oxo-1,2-dihydropyridin-4-yl)-7-oxo-6-(piperazin-1-yl)-[1,2,4]triazolo[1,5-a]pyrimidin-4(7H)-yl)acetamide (55 mg, 93 μmol) and 5-hydroxy-6-methylpyrimidine-4-carboxylic acid (17.2 mg, 111.6 μmol) in DMF (2.5 mL) at room temperature were added HATU (54.6 mg, 139.5 μmol) and DIPEA (81 μL, 465 μmol). The mixture was stirred at room temperature for 2 h, then quenched with water. Aqueous saturated NaHCO₃ was added and the mixture was extracted with EtOAc. The organic layer was dried (Na₂SO₄), concentrated. The residue was purified by column chromatography (0-10% MeOH in dichloromethane) to provide the title compound.

### Preparation Example 4

### Synthesis of Compound 57

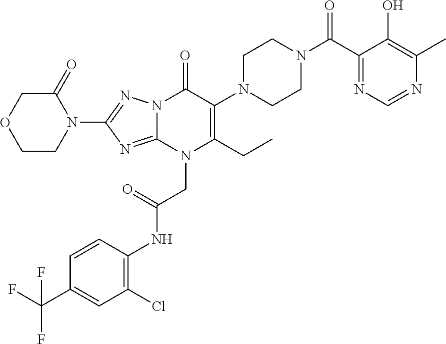 [SMILES: CCc1c(N2CCN(C(=O)c3ncnc(C)c3O)CC2)c(=O)n2nc(N3CCOCC3=O)nc2n1CC(=O)Nc1ccc(C(F)(F)F)cc1Cl]

### Step 1. tert-butyl 4-(2-bromo-5-ethyl-7-oxo-4-((2-(trimethylsilyl)ethoxy)methyl)-4,7-dihydro-[1,2,4]tri azolo[1,5-a]pyrimidin-6-yl)piperazine-1-carboxylate

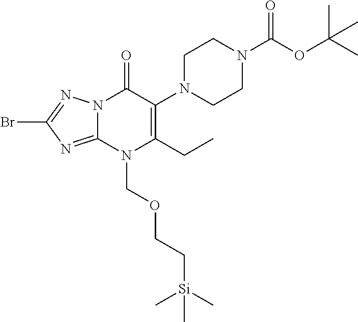 [SMILES: CCc1c(N2CCN(C(=O)OC(C)(C)C)CC2)c(=O)n2nc(Br)nc2n1COCC[Si](C)(C)C]

To a solution of tert-butyl 4-(2-bromo-5-ethyl-7-oxo-4,7-dihydro-[1,2,4]triazolo[1,5-a]pyrimidin-6-yl)piperazine-1-carboxylate (600 mg, 1.4 mmol) in THF (8 mL) and DMF (2 mL) at 0° C. was added NaH 60% in mineral oil (67.2 mg, 1.68 mmol). The mixture was stirred for 30 in, then (2-(chloromethoxy)ethyl)trimethylsilane (285 μL, 1.61 mmol) was added dropwise. The ice bath was removed, and the reaction mixture was allowed to warm to room temperature and stirred for 24 h. Aqueous saturated NaHCO₃ was added, and the mixture was extracted with dichloromethane. The combined organic layers were dried (Na₂SO₄) and concentrated to provide the title compound.

### Step 2. tert-butyl 4-(5-ethyl-7-oxo-2-(3-oxomorpholino)-4-((2-(trimethylsilyl)ethoxy)methyl)-4,7-dihydro-[1,2,4]triazolo[1,5-a]pyrimidin-6-yl)piperazine-1-carboxylate

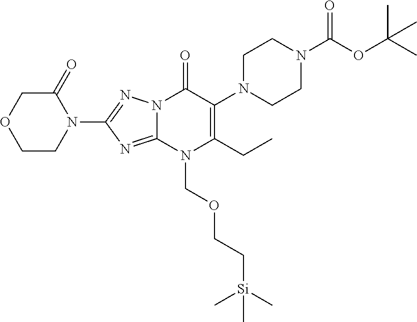 [SMILES: CCc1c(N2CCN(C(=O)OC(C)(C)C)CC2)c(=O)n2nc(N3CCOCC3=O)nc2n1COCC[Si](C)(C)C]

A mixture of tert-butyl 4-(2-bromo-5-ethyl-7-oxo-4-((2-(trimethylsilyl)ethoxy)methyl)-4,7-dihydro-[1,2,4]tri azolo[1,5-a]pyrimidin-6-yl)piperazine-1-carboxylate (100 mg, 179 μmol), morpholin-3-one (36.4 mg, 360 μmol), N,N′-dimethylethylene diamine (1.6 mg, 18 μmol), CuI (1.7 mg, 9 μmol), and K₂CO₃ (50 mg, 360 μmol) in toluene was stirred at 110° C. for 12 h. The mixture was cooled to room temperature and poured into water. The resulting mixture was extracted with EtOAc. The organic layer was dried (Na₂SO₄) and concentrated. The residue was purified by column chromatography (0-100% EtOAc in heptane) to provide the title compound.

### Step 3. tert-butyl 4-(5-ethyl-7-oxo-2-(3-oxomorpholino)-4,7-dihydro-[1,2,4]triazolo[1,5-a]pyrimidin-6-yl)piperazine-1-carboxylate

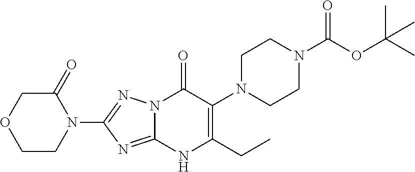 [SMILES: CCc1[nH]c2nc(N3CCOCC3=O)nn2c(=O)c1N1CCN(C(=O)OC(C)(C)C)CC1]

To a solution of tert-butyl 4-(5-ethyl-7-oxo-2-(3-oxomorpholino)-4-((2-(trimethylsilyl)ethoxy)methyl)-4,7-dihydro-[1,2,4]triazolo[1,5-a]pyrimidin-6-yl)piperazine-1-carboxylate (45 mg, 77.9 μmol) in dichloromethane (1 mL) was added TFA (0.5 mL). The mixture was stirred at 40° C. for 2 h and concentrated. The residue was suspended in dichloromethane (2 mL), and DIPEA (27 μL, 156 μmol) and Boc₂O (34 mg, 156 μmol) were added. The resulting mixture was stirred at room temperature for 1.5 h, then quenched with aqueous saturated NaHCO₃. The mixture was extracted with dichloromethane, and the organic layer was dried (Na₂SO₄) and concentrated to provide the title compound.

### Step 4. tert-butyl 4-(4-(2-((2-chloro-4-(trifluoromethyl)phenyl)amino)-2-oxoethyl)-5-ethyl-7-oxo-2-(3-oxomorpholino)-4,7-dihydro-[1,2,4]triazolo[1,5-a]pyrimidin-6-yl)piperazine-1-carboxylate

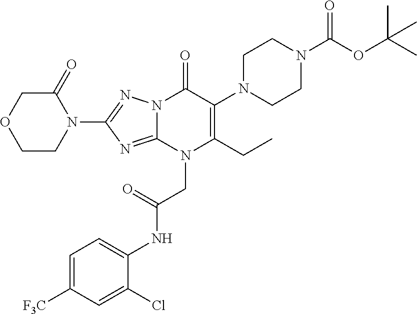 [SMILES: CCc1c(N2CCN(C(=O)OC(C)(C)C)CC2)c(=O)n2nc(N3CCOCC3=O)nc2n1CC(=O)Nc1ccc(C(F)(F)F)cc1Cl]

To a mixture of tert-butyl 4-(5-ethyl-7-oxo-2-(3-oxomorpholino)-4,7-dihydro-[1,2,4]triazolo[1,5-a]pyrimidin-6-yl)piperazine-1-carboxylate (92 mg, 206 μmol) in 1,4-dioxane (4 mL) were added KI (34.2 mg, 206 μmol) and DIPEA (108 μL, 618 μmol). The resulting mixture was stirred at 80° C. for 3.5 h, then cooled to room temperature. The mixture was partitioned between EtOAc and water. The organic layer was washed with brine, dried (Na₂SO₄), filtered, and concentrated. The residue was purified by column chromatography (0-50% EtOAc in heptane) to provide the title compound.

### Step 5. N-(2-chloro-4-(trifluoromethyl)phenyl)-2-(5-ethyl-7-oxo-2-(3-oxomorpholino)-6-(piperazin-1-yl)-[1,2,4]triazolo[1,5-a]pyrimidin-4(7H)-yl)acetamide

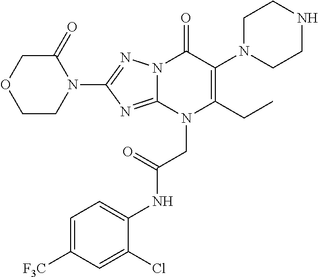 [SMILES: CCc1c(N2CCNCC2)c(=O)n2nc(N3CCOCC3=O)nc2n1CC(=O)Nc1ccc(C(F)(F)F)cc1Cl]

To a solution of tert-butyl 4-(4-(2-((2-chloro-4-(trifluoromethyl)phenyl)amino)-2-oxoethyl)-5-ethyl-7-oxo-2-(3-oxomorpholino)-4,7-dihydro-[1,2,4]triazolo[1,5-a]pyrimidin-6-yl)piperazine-1-carboxylate (40 mg, 58.6 μmol) in dichloromethane (1 mL) was added TFA (0.25 mL). The mixture was stirred at room temperature for 1 h, then diluted with dichloromethane and neutralized with aqueous saturated NaHCO₃. The organic layer was dried (Na₂SO₄), concentrated and dried under vacuum to provide the title compound.

### Step 6. N-(2-chloro-4-(trifluoromethyl)phenyl)-2-(5-ethyl-6-(4-(5-hydroxy-6-methylpyrimidine-4-carbonyl)piperazin-1-yl)-7-oxo-2-(3-oxomorpholino)-[1,2,4]triazolo[1,5-a]pyrimidin-4(7H)-yl)acetamide

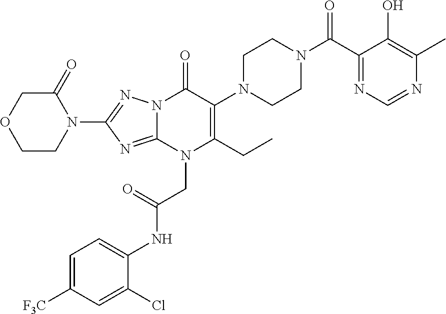 [SMILES: CCc1c(N2CCN(C(=O)c3ncnc(C)c3O)CC2)c(=O)n2nc(N3CCOCC3=O)nc2n1CC(=O)Nc1ccc(C(F)(F)F)cc1Cl]

To a mixture of N-(2-chloro-4-(trifluoromethyl)phenyl)-2-(5-ethyl-7-oxo-2-(3-oxomorpholino)-6-(piperazin-1-yl)-[1,2,4]triazolo[1,5-a]pyrimidin-4(7H)-yl)acetamide (35 mg, 60 μmol) and 5-hydroxy-6-methylpyrimidine-4-carboxylic acid (11.1 mg, 72 μmol) in DMF (1.5 mL) at room temperature were added HATU (35.2 mg, 90 μmol) and DIPEA (52 μL, 300 μmol). The mixture was stirred at room temperature for 2 h, then quenched with water. Aqueous saturated NaHCO₃ was added, and the mixture was extracted with EtOAc. The organic layer was dried (Na₂SO₄), concentrated. The residue was purified by column chromatography (0-10% MeOH in dichloromethane) to provide the title compound.

### Preparation Example 5

### Synthesis of Compound 44

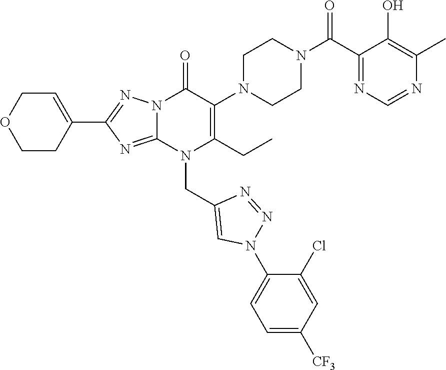 [SMILES: CCc1c(N2CCN(C(=O)c3ncnc(C)c3O)CC2)c(=O)n2nc(C3=CCOCC3)nc2n1Cc1cn(-c2ccc(C(F)(F)F)cc2Cl)nn1]

### Step 1. tert-butyl 4-(2-(3,6-dihydro-2H-pyran-4-yl)-5-ethyl-7-oxo-4,7-dihydro-[1,2,4]triazolo[1,5-a]pyrimidin-6-yl)piperazine-1-carboxylate

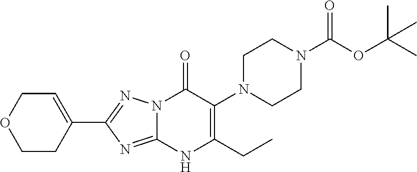 [SMILES: CCc1[nH]c2nc(C3=CCOCC3)nn2c(=O)c1N1CCN(C(=O)OC(C)(C)C)CC1]

A mixture of tert-butyl 4-(2-bromo-5-ethyl-7-oxo-4,7-dihydro-[1,2,4]triazolo[1,5-a]pyrimidin-6-yl)piperazine-1-carboxylate (120 mg), 2-(3,6-dihydro-2H-pyran-4-yl)-4,4,5,5-tetramethyl-1,3,2-dioxaborolane (50 mg), K₃PO₄ (78 mg), and Pd(dppf)Cl₂·DCM (15 mg) in 1,4-dioxane (4 mL) and water (2 mL) was stirred at 80° C. for 8 h. The mixture was cooled to room temperature and diluted with EtOAc. The organic layer was washed with water and brine, and dried (Na₂SO₄) and concentrated. The residue was purified by column chromatography (0-100% EtOAc in heptane) to provide the title compound.

### Step 2. tert-butyl 4-(2-(3,6-dihydro-2H-pyran-4-yl)-5-ethyl-7-oxo-4-(prop-2-yn-1-yl)-4,7-dihydro-[1,2, 4]triazolo[1,5-a]pyrimidin-6-yl)piperazine-1-carboxylate

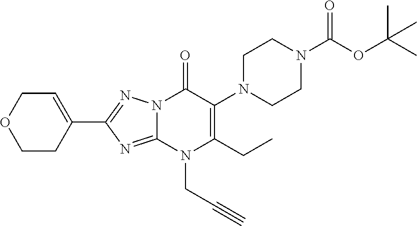 [SMILES: C#CCn1c(CC)c(N2CCN(C(=O)OC(C)(C)C)CC2)c(=O)n2nc(C3=CCOCC3)nc12]

To a DMF (5 mL) solution of tert-butyl 4-(2-(3,6-dihydro-2H-pyran-4-yl)-5-ethyl-7-oxo-4,7-dihydro-[1,2,4]triazolo[1,5-a]pyrimidin-6-yl)piperazine-1-carboxylate prepared according to above procedure (430 mg) stirred at 0° C. was added NaH (60 mg, 60% in mineral oil). After 30 mins, propargyl bromide (0.2 mL) was added. The reaction mixture was allowed to warm to r.t. gradually and stirred for 24 hrs. Saturated NH₄Cl solution was added, and the resulting solution was extracted with ethyl acetate (3×10 mL). The combined organic layers were dried over MgSO4, filtered and concentrated in vacuo. The residue was purified by column chromatography (0-10% MeOH in dichloromethane) to provide the tert-butyl 4-(2-(3,6-dihydro-2H-pyran-4-yl)-5-ethyl-7-oxo-4-(prop-2-yn-1-yl)-4,7-dihydro-[1,2,4]triazolo[1,5-a]pyrimidin-6-yl)piperazine-1-carboxylate (260 mg). LC-MS MS m/z [M+H-Boc]⁺413

### Step 3. tert-butyl 4-(4-((1-(2-chloro-4-(trifluoromethyl)phenyl)-1H-1,2,3-triazol-4-yl)methyl)-2-(3,6-di hydro-2H-pyran-4-yl)-5-ethyl-7-oxo-4,7-dihydro-[1,2,4]triazolo[1,5-a]pyrimidin-6-yl) piperazine-1-carboxylate

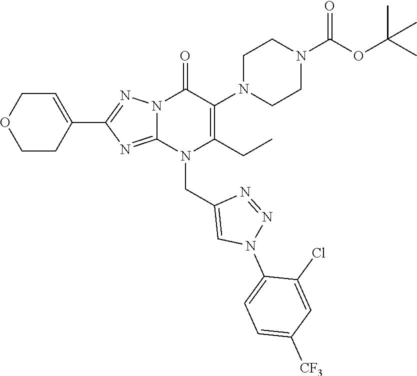 [SMILES: CCc1c(N2CCN(C(=O)OC(C)(C)C)CC2)c(=O)n2nc(C3=CCOCC3)nc2n1Cc1cn(-c2ccc(C(F)(F)F)cc2Cl)nn1]

To a DMF solution (3 mL) of tert-butyl 4-(2-(3,6-dihydro-2H-pyran-4-yl)-5-ethyl-7-oxo-4-(prop-2-yn-1-yl)-4,7-dihydro-[1,2, 4]triazolo[1,5-a]pyrimidin-6-yl)piperazine-1-carboxylate (250 mg) and 1-Azido-2-chloro-4-(trifluoromethyl)benzene (110 mg) was added CuI (10 mol %), DIPEA (0.07 mL) at 0° C. After 5 minutes, ice cold water was added to quench the reaction. The resulting solution was extracted with ethyl acetate (3×10 mL). The combined organic layers were dried over MgSO4, filtered and concentrated in vacuo. The residue was purified by column chromatography (0-10% MeOH in dichloromethane) to provide tert-butyl 4-(4-((1-(2-chloro-4-(trifluoromethyl)phenyl)-1H-1,2,3-triazol-4-yl)methyl)-2-(3,6-di hydro-2H-pyran-4-yl)-5-ethyl-7-oxo-4,7-dihydro-[1,2,4]triazolo[1,5-a]pyrimidin-6-yl) piperazine-1-carboxylate (200 mg). LC-MS MS m/z [M+H-Boc]⁺634

### Step 4. 4-((1-(2-chloro-4-(trifluoromethyl)phenyl)-1H-1,2,3-triazol-4-yl)methyl)-2-(3,6-dihydro-2H-pyran-4-yl)-5-ethyl-6-(piperazin-1-yl)-[1,2,4]triazolo[1,5-a]pyrimidin-7(4H)-one

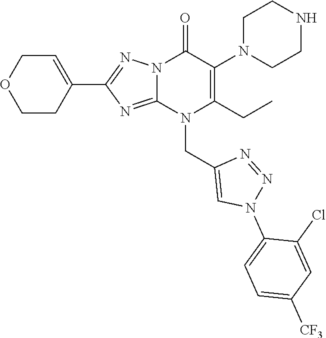 [SMILES: CCc1c(N2CCNCC2)c(=O)n2nc(C3=CCOCC3)nc2n1Cc1cn(-c2ccc(C(F)(F)F)cc2Cl)nn1]

To tert-butyl 4-(4-((1-(2-chloro-4-(trifluoromethyl)phenyl)-1H-1,2,3-triazol-4-yl)methyl)-2-(3,6-di hydro-2H-pyran-4-yl)-5-ethyl-7-oxo-4,7-dihydro-[1,2,4]triazolo[1,5-a]pyrimidin-6-yl) piperazine-1-carboxylate (190 mg) was added HCl in dioxane (4M, 2 mL). After stirring ar r.t. for 16 hrs, it was concentrated in vacuo to give a crude 4-((1-(2-chloro-4-(trifluoromethyl)phenyl)-1H-1,2,3-triazol-4-yl)methyl)-2-(3,6-dihydro-2H-pyran-4-yl)-5-ethyl-6-(piperazin-1-yl)-[1,2,4]triazolo[1,5-a]pyrimidin-7(4H)-one. LC-MS MS m/z [M+1]⁺ 590

### Step 5

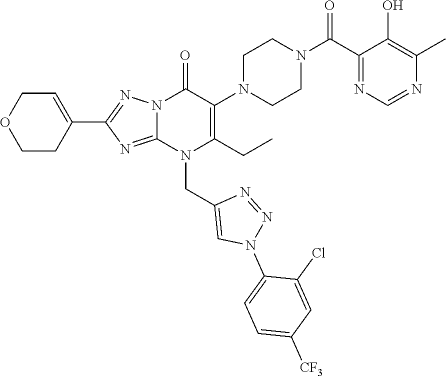 [SMILES: CCc1c(N2CCN(C(=O)c3ncnc(C)c3O)CC2)c(=O)n2nc(C3=CCOCC3)nc2n1Cc1cn(-c2ccc(C(F)(F)F)cc2Cl)nn1]

To a stirred DMF (2 mL) solution of the above crude 4-((1-(2-chloro-4-(trifluoromethyl)phenyl)-1H-1,2,3-triazol-4-yl)methyl)-2-(3,6-dihydro-2H-pyran-4-yl)-5-ethyl-6-(piperazin-1-yl)-[1,2,4]triazolo[1,5-a]pyrimidin-7(4H)-one from step 3 above was added DIPEA (0.1 mL), N, N, N′, N′-tetramethylchloroformamidinium hexafluorophosphate (140 mg) and 5-hydroxy-6-methylpyrimidine-4-carboxylic acid (130 mg). After r.t. for 24 hrs, it was concentrated in vacuo. The residue was purified on a HPLC prep column to yield

4-((1-(2-chloro-4-(trifluoromethyl)phenyl)-1H-1,2,3-triazol-4-yl)methyl)-2-(3,6-dihydro-2H-pyran-4-yl)-5-ethyl-6-(4-(5-hydroxy-6-methylpyrimidine-4-carbonyl)piperazin-1-yl)-[1,2,4]triazolo[1,5-a]pyrimidin-7(4H)-one (20 mg). LC-MS MS m/z [M+1]⁺ 726

### Preparation Example 6

### Synthesis of Compound 97

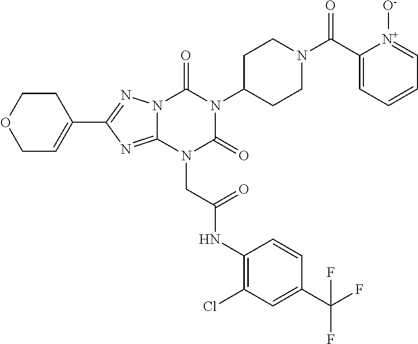 [SMILES: O=C(Cn1c(=O)n(C2CCN(C(=O)c3cccc[n+]3[O-])CC2)c(=O)n2nc(C3=CCOCC3)nc12)Nc1ccc(C(F)(F)F)cc1Cl]

### Synthesis Scheme:

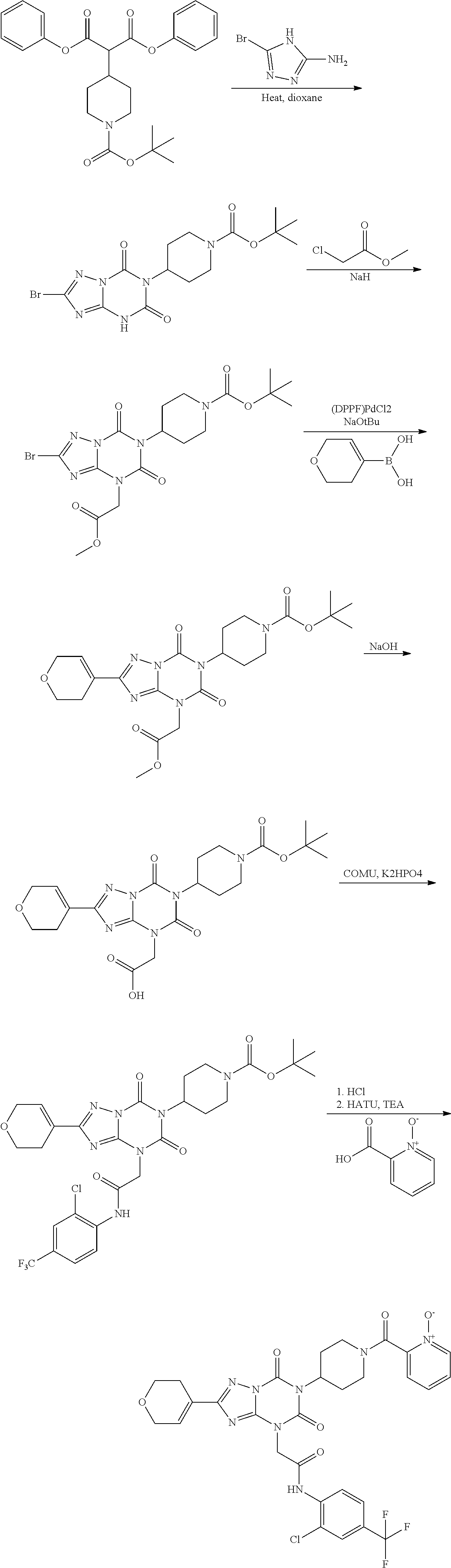

### Preparation Example 7

### Synthesis of Compound 187

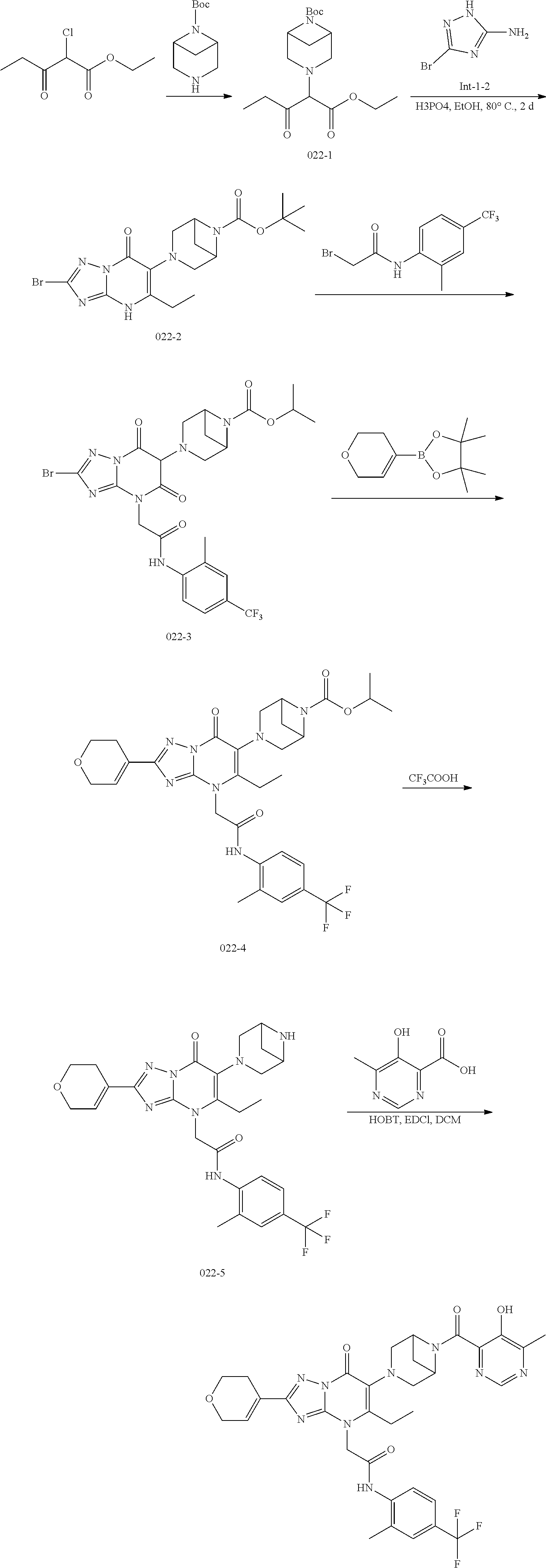

### Step 1: tert-butyl 3-(1-ethoxy-1,3-dioxopentan-2-yl)-3,6-diazabicyclo[3.1.1]heptane-6-carboxylate

To a stirred solution of tert-butyl 3,6-diazabicyclo[3.1.1]heptane-6-carboxylate (5 g, 25.22 mmol) and ethyl 2-chloro-3-oxopentanoate (4.95 g, 27.74 mmol) in ACN (50 mL) was added TEA (7.66 g, 75.66 mmol) at RT and stirred at 55° C. overnight. The mixture was concentrated under reduced pressure. The crude product was diluted with EA and water, extracted once with EA and the organic layer was washed with brine, dried over Na₂SO₄ and concentrated under reduced pressure. The crude product was purified by column chromatography (PE:EA 10:1 to 5:1) to give compound 022-1 (4.5 g, 52% yield).

Mass calc C₁₇H₂₈N₂O₅ for 340.2 found: 341.2 (M+H)⁺ ESI.

### Step 2: tert-butyl 3-(2-bromo-5-ethyl-7-oxo-4,7-dihydro-[1,2,4]triazolo[1,5-a]pyrimidin-6-yl)-3,6-diazabicyclo[3.1.1]heptane-6-carboxylate, 022-2

Int-1-2 (1.9 g, 11.66 mmol) and tert-butyl 3-(1-ethoxy-1,3-dioxopentan-2-yl)-3,6-diazabicyclo[3.1.1]heptane-6-carboxylate (4.5 g, 13.22 mmol) were mixed in EtOH (50 mL), H₃PO₄ (1.14 g, 11.66 mmol) was added. The mixture was stirred at 80° C. for 2 days under nitrogen. The mixture was concentrated in vacuo to remove EtOH, then quenched by addition of sat NaHCO₃ solution, and extracted with EA. The combined organic layers were washed with brine, dried over Na₂SO₄, filtered and concentrated under reduced pressure to give a residue. The residue was purified by column chromatography (Silica column, eluent DCM:MeOH 20:1 to 10:1) to give compound 022-2 as a yellow oil (650 mg, 12% yield).

Mass calc C₁₇H₂₃BrN₆O₃ for 438.10, 440.10 found: 339.10, 341.10 (M-Boc+H) ESI.

### Step 3: tert-butyl 3-(2-bromo-5-ethyl-4-(2-((2-methyl-4-(trifluoromethyl) phenyl)amino)-2-oxoethyl)-7-oxo-4,7-dihydro-[1,2,4]triazolo[1,5-a]pyrimidin-6-yl)-3,6-diazabicyclo[3.1.1]heptane-6-carboxylate, 022-3

To a stirred solution of 002-2 (600 mg, 1.37 mmol) and 2-bromo-N-(2-methyl-4-(trifluoromethyl)phenyl)acetamide (44.83 mg, 1.50 mmol) in 1,4-dioxane (15 mL) was added DIPEA (529.55 mg, 4.10 mmol) at RT and stirred at 80° C. for 6 hours. The mixture was concentrated under reduced pressure. The crude product was diluted with EA and water, extracted once with EA and the organic layer was washed with brine, dried over Na₂SO₄ and concentrated under reduced pressure. The crude product was purified by column chromatography (DCM:MeOH 25:1 to 10:1) to give compound 022-3 (710 mg, 79% yield).

Mass calc C₂₇H₃₁BrF₃N₇O₄ for 653.16, 655.16 found: 554.06, 556.04 (M-Boc+H)⁺ ESI.

### Step 4: tert-butyl 3-(2-(3,6-dihydro-2H-pyran-4-yl)-5-ethyl-4-(2-((2-methyl-4-(trifluoromethyl)phenyl)amino)-2-oxoethyl)-7-oxo-4,7-dihydro-[1,2,4]triazolo

[1,5-a]pyrimidin-6-yl)-3,6-diazabicyclo[3.1.1]heptane-6-carboxylate, 022-4 022-3 (750 mg, 1.15 mmol) was suspended in 1,4-dioxane (7 mL), H₂O (3.5 mL), 2-(3,6-dihydro-2H-pyran-4-yl)-4,4,5,5-tetramethyl-1,3,2-dioxaborolane (264.81 mg, 1.26 mmol) and K₃PO₄ (729.75 mg, 3.44 mmol) were added and the mixture was degassed for 10 minutes with argon, PdCl₂(dppf) (46.44 mg, 0.06 mmol) was added and the mixture was stirred at 80° C. for 2 hours. The mixture was concentrated under reduced pressure. The crude product was diluted with EA and water, extracted once with EA and the organic layer was washed with brine, dried over Na₂SO₄ and concentrated under reduced pressure. The crude product was purified by column chromatography to give the compound 022-4 (580 mg, 76.9% yield).

Mass calc C₃₂H₃₈F₃N₇O₅ for 657.29, 659.30 found: 558.23, 559.25 (M-Boc+H)⁺ ESI.

### Step 5: 2-(6-(3,6-diazabicyclo[3.1.1]heptan-3-yl)-2-(3,6-dihydro-2H-pyran-4-yl)-5-ethyl-7-oxo-[1,2,4]triazolo[1,5-a]pyrimidin-4(7H)-yl)-N-(2-methyl-4-(trifluoromethyl)phenyl)acetamide, 022-5

022-4 (100 mg, 0.15 mmol) was suspended in DCM (2 mL), TFA (1 mL) was added and the RM was stirred at RT for 2 h. The RM was concentrated under reduce pressure and used without purification.

Mass calc C₂₇H₃₀F₃N₇O₃ for 557.24, found: 558.2, (M+H)⁺ ESI.

### Step 6: 2-(2-(3,6-dihydro-2H-pyran-4-yl)-5-ethyl-6-(6-(5-hydroxy-6-methyl pyrimidine-4-carbonyl)-3,6-diazabicyclo[3.1.1]heptan-3-yl)-7-oxo-[1,2,4]triazolo [1,5-a]pyrimidin-4(7H)-yl)-N-(2-methyl-4-(trifluoromethyl)phenyl)acetamide

5-hydroxy-6-methylpyrimidine-4-carboxylic acid (46.44 mg, 0.30 mmol) was dissolved in DCM (5 mL), the solution was added HOBT (40.71 mg, 0.30 mmol), EDCI (57.70 mg, 0.30 mmol), Pyridine (35.75 mg, 0.45 mmol) and 022-5 (84 mg, 0.15 mmol) was added, then stirring at R.T for 2 h, the RM was purified by reverse phase column (C18 column) to provide the product (60 mg, 57% yield).

Mass calc C₃₃H₃₄F₃N₉O₅ for 693.26 found: 694.25 (M+H)⁺ ESI.

### Preparation Example 8

### Synthesis of Compound 189

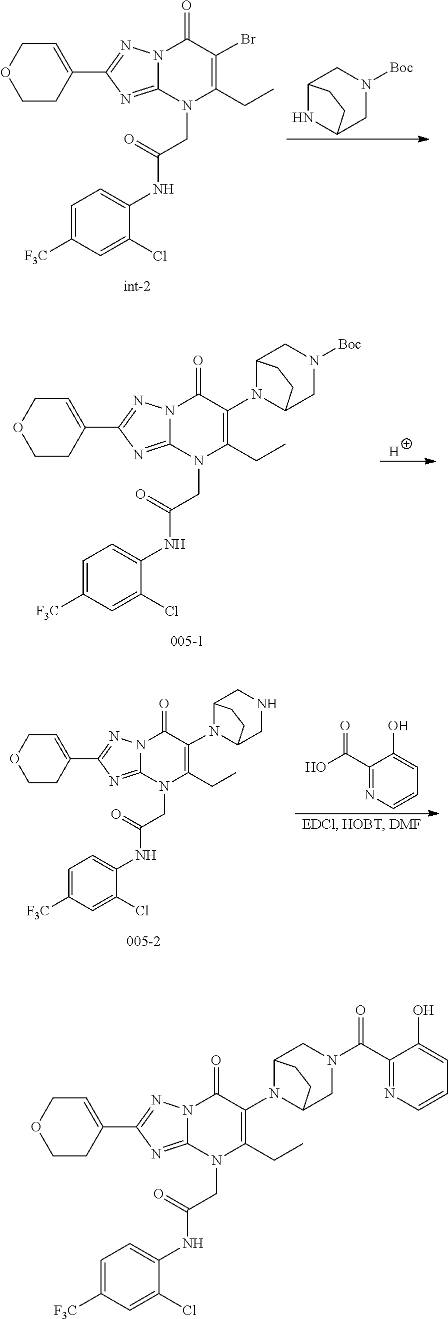

### Step 1: tert-butyl 8-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-3,8-diazabicyclo[3.2.1]octane-3-carboxylate, 005-1

Int-2 (300 mg, 0.54 mmol) was suspended in DMF (10 mL), AgBF₄ (104.15 mg, 0.54 mmol) and tert-butyl 3,8-diazabicyclo[3.2.1]octane-3-carboxylate (1.14 g, 5.4 mmol) was added and then stirred at 140° C. for 2 h under N₂ atmosphere. The mixture was filtered and diluted with 10 mL H₂O and 10 mL EA, the organic phase was washed by brine and concentrated under vacuum to give the crude. The crude was purified by reverse phase column (C18 column) to provide the product 005-1 (104 mg, 28% yield) as a yellow solid.

Mass calc C₃₂H₃₇ClF₃N₇O₅ for 691.25, 693.25 found: 636.2, 638.2 (M-tBu+H)⁺ ESI.

### Step 2: 2-(6-(3,8-diazabicyclo[3.2.1]octan-8-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoro methyl)phenyl)acetamide, 005-2

005-1 (100 mg, 0.14 mmol), hydrogen chloride in dioxane (2 mL) was added and the RM was stirred at RT for 2 h. The RM was concentrated under reduce pressure and used without purification.

Mass calc C₂₇H₂₉ClF₃N₇O₃ for 591.20 found: 592.2 (M+H)⁺ ESI.

### Step 3: N-(2-chloro-4-(trifluoromethyl)phenyl)-2-(2-(3,6-dihydro-2H-pyran-4-yl)-5-ethyl-6-(3-(3-hydroxypicolinoyl)-3,8-diazabicyclo[3.2.1]octan-8-yl)-7-oxo-[1,2,4]triazolo[1,5-a]pyrimidin-4(7H)-yl)acetamide

3-hydroxypicolinic acid (14.10 mg, 0.10 mmol) was dissolved in DMF (1 mL), the solution was added HOBT (13.69 mg, 0.10 mmol), EDCI (19.43 mg, 0.10 mmol), Pyridine (12.03 mg, 0.10 mmol) and 005-2 (30 mg, 0.051 mmol) was added, then stirring at R.T for 2 h, the RM was purified by reverse phase column (C18 column) to provide the product (11 mg, 30% yield) as a white solid.

Mass calc C₃₃H₃₂ClF₃N₈O₅ for 712.21, 714.21 found: 713.2, 715.4 (M+H)⁺ ESI.

¹H NMR (400 MHz, DMSO-d₆) δ: 10.24 (s, 1H), 8.08-8.06 (m, 2H), 7.97 (s, 1H), 7.73-7.71 (d, J=8.0 Hz, 1H), 7.28 (s, 2H), 6.82 (s, 1H), 5.32 (s, 2H), 4.31-4.25 (m, 3H), 3.80 (s, 2H), 3.63 (s, 1H), 3.44-3.41 (m, 4H), 3.15-3.10 (m, 4H), 2.33-2.22 (m, 2H), 1.86 (s, 1H), 1.70 (s, 1H), 2.37 (s, 3H), 1.27-1.23 (m, 3H).

### Preparation Example 9

### Synthesis of Compound 4

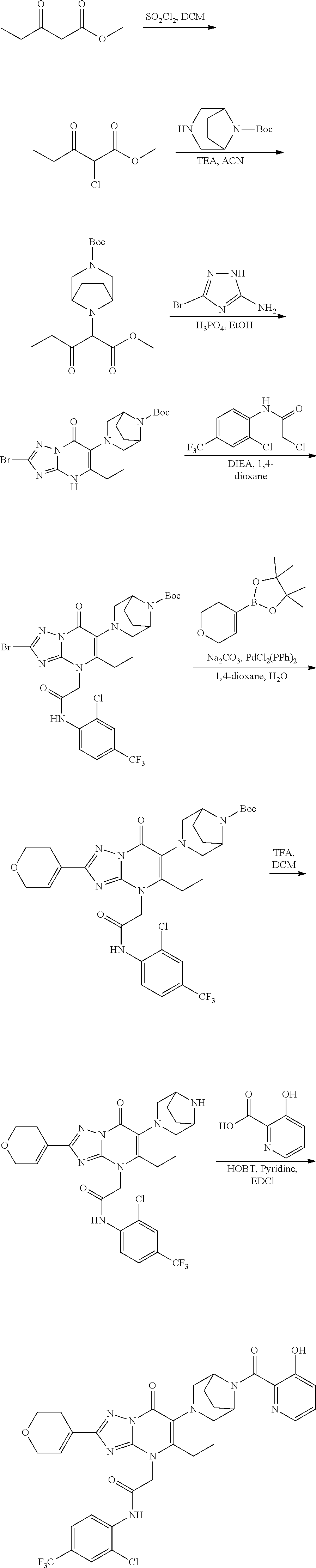

### Step 1: methy2-chloro-3-oxopentanoate

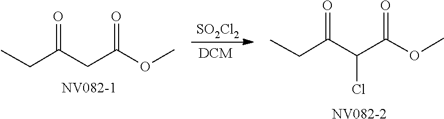

To a solution of methy3-oxopentanoate (10 g, 76.84 mmol) in DCM (100 ml) was added SO₂Cl₂ (13.48 g, 99.89 mmol) at 0° C. over 10 min. The reaction was allowed to warm to RT and stirred for 16 hours. The RM was concentrated under reduced pressure and the residue was dissolved in DCM (100 ml) and washed with water (100 ml), brine (100 ml), dried over Na₂SO₄ and filtered. The filtrate was concentrated under reduced pressure to give the title compound as light yellow liquid (12 g, 94% yield).

Mass calc C₆H₉ClO₃ for 164.0, found: 163.0 (M−H)− ESI.

¹H NMR (400 MHz, DMSO-d₆) δ: 5.62 (s, 1H), 3.76 (s, 3H), 2.50-2.48 (m, 2H), 0.99 (t, J=7.2 Hz, 3H).

### Step 2: tert-butyl (1R,5S)-3-(1-methoxy-1,3-dioxopentan-2-yl)-3,8-diazabicyclo[3.2.1]octane-8-carboxylate

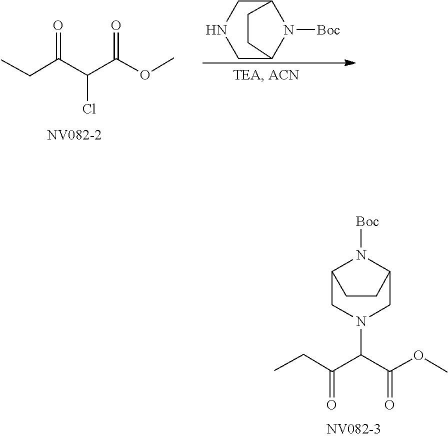

To a solution of methy2-chloro-3-oxopentanoate (5.04 g, 30.62 mmol), in dry ACN (25 ml) was added TEA (7.15 g, 70.66 mmol) over 15 min, followed by dropwise addition of tert-butyl 3,8-diazabicyclo[3.2.1]octane-8-carboxylate (5 g, 23.55 mmol) in ACN over 30 min. The reaction was stirred at 60° C. for 16 hours. The RM was filtered and washed with EA. The filtrated was then concentrated under reduced pressure and the residue was dissolved in EA and washed with water, dried over Na₂SO₄ and filtered. The filtrate was concentrated under reduced pressure and the residue was purified by column chromatography eluent heptane:EA 100:0 to 90:10) to give the title compound (4 g, 50% yield).

Mass calc C₁₇H₂₈N₂O₅ for 340.2, found: 339.1 (M−H)− ESI.

¹H NMR (400 MHz, DMSO-d₆) δ: 4.36 (s, 1H), 3.61 (s, 3H), 3.21-3.11 (m, 1H), 2.99-2.92 (m, 1H), 2.75-2.60 (m, 4H), 2.37-2.26 (m, 2H), 1.88-1.75 (m, 4H), 1.41-1.38 (s, 9H), 0.96 (t, J=7.3 Hz, 3H).

### Step 3: tert-butyl 3-(2-bromo-5-ethyl-7-oxo-4,7-dihydro-[1,2,4]triazolo[1,5-a]pyrimidin-6-yl)-3,8-diazabicyclo[3.2.1]octane-8-carboxylate

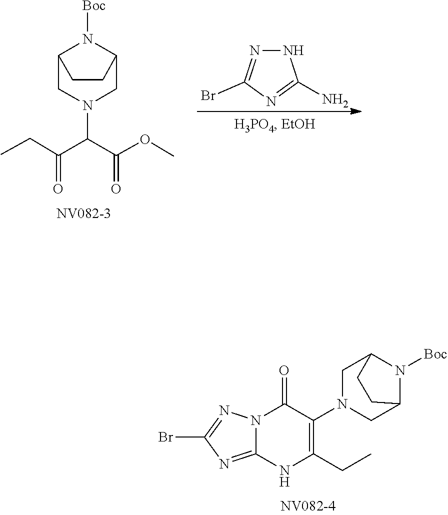 [SMILES: CCC(=O)C(C(=O)OC)N1CC2CCC(C1)N2C(=O)OC(C)(C)C ; Nc1nc(Br)n[nH]1 ; CCc1[nH]c2nc(Br)nn2c(=O)c1N1CC2CCC(C1)N2C(=O)OC(C)(C)C]

3-Bromo-1H-1,2,4-triazol-5-amine (843 mg, 5.17 mmol) and tert-butyl (1R,5S)-3-(1-methoxy-1,3-dioxopentan-2-yl)-3,8-diazabicyclo[3.2.1]octane-8-carboxylate (1.6 g, 4.70 mmol) were mixed in EtOH (3 mL). H₃PO₄ (461 mg, 4.70 mmol) was added. The mixture was stirred at 80° C. for 12 hours under nitrogen. The mixture was concentrated in vacuo to remove EtOH, then quenched by addition of aq sat NaHCO₃, and extracted with DCM. The combined organic layers were washed with brine, dried over Na₂SO₄, filtered and concentrated under reduced pressure to give a residue. The residue was purified by column chromatography (Silica column, eluent DCM:MeOH 1:0 to 50:1) to give the title compound as a yellow solid (300 mg, 14% yield).

Mass calc C₁₈H₂₅BrN₆O₃ for 452.1 454.1, found: 451.1 453.1 (M−H)− ESI.

¹H NMR (400 MHz, DMSO-d6) δ: 13.25 (s, 1H), 4.16-4.02 (m, 2H), 3.56-3.50 (d, J=9.8 Hz, 2H), 2.81-2.71 (m, 2H), 2.46-2.38 (d, J=9.8 Hz, 2H), 1.93-1.80 (m, 4H), 1.44 (s, 9H), 1.21 (t, J=7.6 Hz, 3H).

### Step 4: tert-butyl 3-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)-3,8-diazabicyclo[3.2.1]octane-8-carboxylate

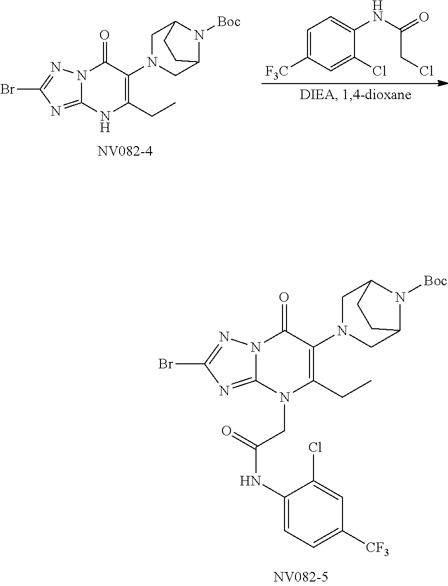 [SMILES: CCc1[nH]c2nc(Br)nn2c(=O)c1N1CC2CCC(C1)N2C(=O)OC(C)(C)C ; O=C(CCl)Nc1ccc(C(F)(F)F)cc1Cl ; CCc1c(N2CC3CCC(C2)N3C(=O)OC(C)(C)C)c(=O)n2nc(Br)nc2n1CC(=O)Nc1ccc(C(F)(F)F)cc1Cl]

To a stirred solution of tert-butyl 3-(2-bromo-5-ethyl-7-oxo-4,7-dihydro-[1,2,4]triazolo[1,5-a]pyrimidin-6-yl)-3,8-diaza bicyclo[3.2.1]octane-8-carboxylate (140 mg, 0.31 mmol) and 2-chloro-N-(2-chloro-4-(trifluoromethyl)phenyl)acetamide (84 mg, 0.31 mmol) in 1,4-dioxane (2 mL) was added DIPEA (120 mg, 0.93 mmol) at RT and the RM was stirred at 80° C. for 72 hours. The RM was concentrated under reduced pressure. The crude product was diluted with EtOAc and water, extracted once with EtOAc and the organic layer was washed with brine, dried over Na₂SO₄ and concentrated under reduced pressure. The crude product was purified by column chromatography (eluent heptane:EtOAc 70:30 to 30:70) to give the title compound as a white solid (130 mg, 610% yield).

Mass calc C₂₇H₃₀BrClF₃N₇O₄ for 687.1 689.1, found: 686.1 688.1 (M−H)− ESI.

¹H NMR (400 MHz, DMSO-d₆) δ: 10.34 (s, 1H), 8.11-8.06 (d, J=8.6 Hz, 1H), 7.99-7.96 (d, J=1.7 Hz, 1H), 7.74-7.70 (dd, J=8.7, 1.7 Hz, 1H), 5.29 (s, 2H), 4.15-4.07 (m, 2H), 3.61-3.57 (m, 2H), 3.02-2.92 (m, 2H), 2.49-2.43 (m, 2H), 1.92-1.84 (m, 4H), 1.45 (s, 9H), 1.20 (t, J=7.4 Hz, 3H).

### Step 5: tert-butyl 3-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-3,8-diazabicyclo[3.2.1]octane-8-carboxylate

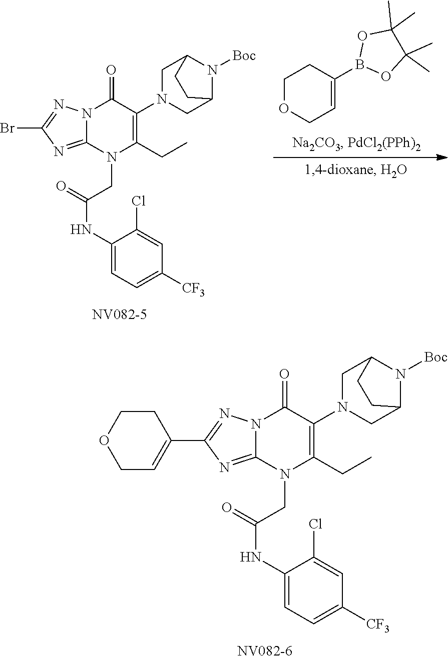 [SMILES: CCc1c(N2CC3CCC(C2)N3C(=O)OC(C)(C)C)c(=O)n2nc(Br)nc2n1CC(=O)Nc1ccc(C(F)(F)F)cc1Cl ; CC1(C)OB(C2=CCOCC2)OC1(C)C ; CCc1c(N2CC3CCC(C2)N3C(=O)OC(C)(C)C)c(=O)n2nc(C3=CCOCC3)nc2n1CC(=O)Nc1ccc(C(F)(F)F)cc1Cl]

Tert-butyl 3-(2-bromo-4-(2-((2-chloro-4-(trifluoromethyl)phenyl)amino)-2-oxoethyl)-5-ethyl-7-oxo-4,7-dihydro-[1,2,4]triazolo [1,5-a]pyrimidin-6-yl)-3,8-diazabicyclo[3.2.1]octane-8-carboxylate (186 mg, 0.27 mol) was suspended in 1,4-dioxane (0.5 mL). 2-(3,6-dihydro-2H-pyran-4-yl)-4,4,5,5-tetramethyl-1,3,2-dioxaborolane (113 mg, 0.54 mmol) and 0.6M aq Na₂CO₃ (0.25 mL) were added and the RM was degassed for 10 minutes with argon, PdCI₂(PPh)₂ (19 mg, 0.03 mmol) was added and the RM was stirred at 90° C. for 12 hours. The RM was concentrated under reduced pressure, and the crude product was purified by column chromatography (Silica gel column: Silica 12 g, eluent DCM:MeOH 100:0 to 97:3) to give the crude title compound (72 mg).

Mass calc C₃₂H₃₇ClF₃N₇O₅ for 691.2, found: 690.1 (M−H)− ESI.

### Step 6: 2-(6-(3,8-diazabicyclo[3.2.1]octan-3-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide

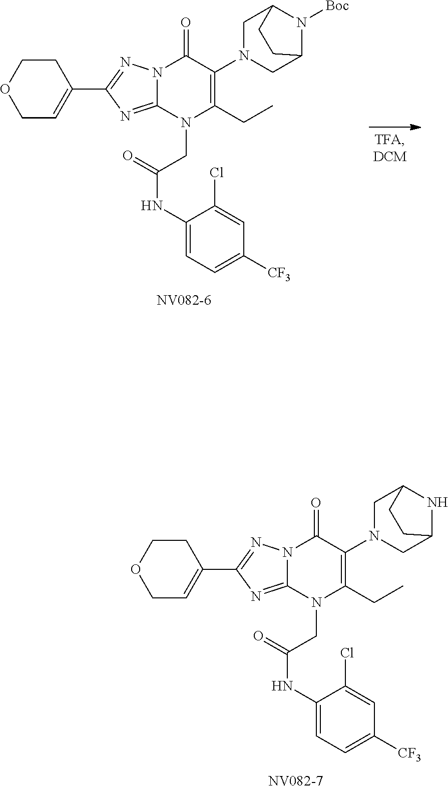 [SMILES: CCc1c(N2CC3CCC(C2)N3C(=O)OC(C)(C)C)c(=O)n2nc(C3=CCOCC3)nc2n1CC(=O)Nc1ccc(C(F)(F)F)cc1Cl ; CCc1c(N2CC3CCC(C2)N3)c(=O)n2nc(C3=CCOCC3)nc2n1CC(=O)Nc1ccc(C(F)(F)F)cc1Cl]

Tert-butyl 3-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-3,8-diazabicyclo[3.2.1]octane-8-carboxylate (40 mg, 0.06 mmol) was suspended in DCM (2 ml), TFA (1 ml) was added and the RM was stirred at RT for 4 h. The RM was concentrated under reduce pressure and used without purification.

Mass calc C₂₇H₂₉ClF₃N₇O₃ for 591.2, found: 592.1 (M+H)+ ESI.

### Step 7: N-(2-chloro-4-(trifluoromethyl)phenyl)-2-(2-(3,6-dihydro-2H-pyran-4-yl)-5-ethyl-6-(8-(3-hydroxy-4-methylpicolinoyl)-3,8-diazabicyclo[3.2.1]octan-3-yl)-7-oxo-[1,2,4]triazolo[1,5-a]pyrimidin-4(7H)-yl)acetamide

3-hydroxypicolinic acid (9 mg, 0.07 mmol) was dissolved in DCM (2 ml), to this solution were added Pyridine (12 mg, 0.16 mmol), HOBT (14.18 mg, 0.10 mmol), EDCI (20.1 mg, 0.10 mmol), 2-(6-(3,8-diazabicyclo[3.2.1]octan-3-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide (30 mg, 0.05 mmol). After stirring for 3 hours, the RM was purified by reverse phase column (C18 column) to provide the product (12 mg, 33% yield).

Mass calc C₃₄H₃₄ClF₃N₈O₅ for 712.2, found: 713.1 (M+H)+ ESI.

¹H NMR (400 MHz, DMSO-d₆) δ: 11.84 (s, 1H), 10.37 (s, 1H), 8.15-8.10 (m, 1H), 8.07-8.04 (d, J=8.5 Hz, 1H), 7.99-7.95 (d, J=1.6 Hz, 1H), 7.74-7.68 (m, 1H), 7.44-7.35 (m, 2H), 6.82 (s, 2H), 5.31 (s, 2H), 5.08 (s, 1H), 4.83 (s, 1H), 4.27-4.20 (d, J=2.5 Hz, 1H) 3.09-2.96 (m, 2H), 2.71-2.64 (d, J=8.8 Hz, 1H), 2.63-2.57 (d, J=8.7 Hz, 1H), 2.49-2.45 (m, 2H), 2.08-1.91 (m, 4H), 1.25-1.20 (m, 3H),

### Preparation Example 10

### Synthesis of Compound 190 and 196

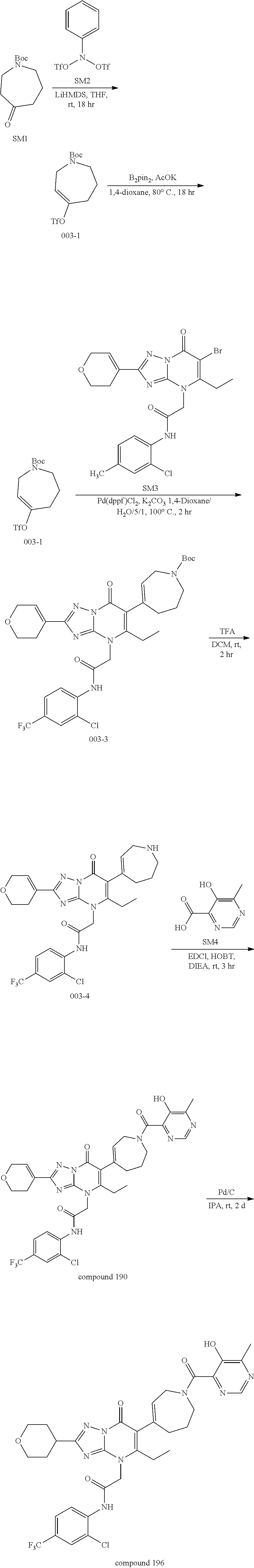

### Step 1: tert-butyl 5-(((trifluoromethyl)sulfonyl)oxy)-2,3,4,7-tetrahydro-1H-azepine-1-carboxylate (003-1)

A mixture of SM1 (2 g, 9.38 mmol) in THF (30 mL) was stirred at −78° C., LiHMDS (1.13 mL, 11.25 mmol) was added, the mixture was stirred at −78° C. for 1.0 hr, SM2 (4.02 g, 11.25 mmol) was added, the mixture was stirred at 25° C. for 16 hr. TLC (PE:EA=5:1) showed new point was detected and the starting material was consumed completely. The reaction was quenched with water (20 mL) and extracted with EA (20 mL×3), the organic layers were dried over anhydrous Na₂SO₄ and concentrated under reduced pressure. The crude residue obtained was purified by silical gel column chromatography using PE/EA (5/1) to give product 003-1 (1.6 g, 49.41% yield) as yellow oil.

¹H NMR (400 MHz, Chloroform-d) δ 5.88 (q, J=5.5 Hz, 1H), 3.95 (dd, J=32.5, 5.1 Hz, 2H), 3.56 (dt, J=21.7, 6.2 Hz, 2H), 2.68-2.49 (m, 2H), 1.94 (dt, J=13.5, 6.5 Hz, 2H), 1.46 (s, 9H).

### Step 2: tert-butyl 5-(4,4,5,5-tetramethyl-1,3,2-dioxaborolan-2-yl)-2,3,4,7-tetrahydro-1H-azepine-1-carboxylate (003-2)

To a solution of 003-1 (500 mg, 1.45 mmol) and B₂pin₂ (0.38 mL, 1.45 mmol) in 1,4-dioxane (10 mL) was added Pd(dppf)Cl₂ (105.94 mg, 0.14 mmol) and AcOK (426.29 mg, 4.34 mmol), the mixture was stirred at 90° C. for 3 hr under N₂. TLC (PE:EA=5:1) showed new point was detected and the starting material was consumed completely. The solvent was removed. The mixture was diluted with water (10 mL) and extracted with EA (10 mL×3), and combined organic layers were dried over anhydrous Na₂SO₄ and concentrated under reduced pressure. The crude residue obtained was purified by silical gel column chromatography using PE/EA (5/1) to obtain 003-2 (216 mg, 46.15% yield) as yellow oil which checked with 2D NMR.

¹H NMR (400 MHz, Chloroform-d) δ 4.20 (dd, J=14.7, 5.3 Hz, 1H), 4.00 (d, J=13.4 Hz, 1H), 2.64 (s, 1H), 2.52-2.35 (m, 2H), 1.60 (ddt, J=21.1, 10.4, 4.2 Hz, 4H), 1.47 (s, 9H), 1.18 (d, J=1.9 Hz, 12H), 0.96 (dd, J=8.0, 3.9 Hz, 1H), 0.51 (dd, J=5.3, 3.9 Hz, 1H)

### Step 3: tert-butyl 5-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-2,3,4,7-tetrahydro-1H-azepine-1-carboxylate (003-3)

To a solution of 003-2 (100 mg, 0.31 mmol) and SM3 (118.40 mg, 0.22 mmol) in 1,4-dioxane (5 mL) and H₂O (0.5 mL) was added K₂CO₃ (128.26 mg, 0.93 mmol) and Pd(dppf)Cl₂ (25.26 mg, 0.03 mmol), the mixture was stirred at 70° C. for 3 hr under N₂. LCMS (XT221416-309-6563-Z-1) showed desired product was detected and the starting material was consumed completely. The solvent was removed. The mixture was diluted with water (20 mL) and extracted with EA (20 mL×3), and combined organic layers were dried over anhydrous Na₂SO₄ and concentrated under reduced pressure. The residue was purified by silical gel column chromatography using PE/EA (1/1) to give product 003-3 (63 mg, 28.56% yield) as white solid. MS: m/z=621.3 (M+1, ESI+) at 1.65 min.

### Step 4: N-(2-chloro-4-(trifluoromethyl)phenyl)-2-(2-(3,6-dihydro-2H-pyran-4-yl)-5-ethyl-7-oxo-6-(2,5,6,7-tetrahydro-1H-azepin-4-yl)-[1,2,4]triazolo[1,5-a]pyrimidin-4(7H)-yl)acetamide (003-4)

A mixture of 003-3 (63 mg, 0.01 mmol) and TFA (0.2 mL, 1.34 mmol) in DCM (2 mL) was stirred at 25° C. for 1 hr. LCMS (XT221416-322-21052-LCMS06) indicated desired MS peak was found. The solvent was removed to give product 003-4 (69 mg, crude) as yellow oil. MS: m/z=577.2 (M+1, ESI+) at 1.70 min.

### Step 5: N-(2-chloro-4-(trifluoromethyl)phenyl)-2-(2-(3,6-dihydro-2H-pyran-4-yl)-5-ethyl-6-(1-(5-hydroxy-6-methylpyrimidine-4-carbonyl)-2,5,6,7-tetrahydro-1H-azepin-4-yl)-7-oxo-[1,2,4]triazolo[1,5-a]pyrimidin-4(7H)-yl)acetamide (Compound 190)

To a solution of 003-4 (60 mg, 0.10 mmol) and SM4 (32.05 mg, 0.21 mmol) in DMF (3 mL) was added EDC (39.87 mg, 0.21 mmol), HOBt (28.10 mg, 0.21 mmol) and DIEA (0.09 mL, 0.52 mmol), the mixture was stirred at 25° C. for 3 hr. LCMS (XT221416-337-P1-25473-LCMS02) showed desired product was detected and the starting material was consumed completely. The reaction was diluted with water (20 mL) and extracted with EA (30 mL×3). The organic layer was separated, washed with water (30 mL×3). The organic layers were dried over anhydrous Na₂SO₄ and concentrated under reduced pressure. The residue was purified by perp-HPLC to give product Compound 190 (15.2 mg, 20.5% yield) as white solid. MS: m/z=713.4 (M+1, ESI+) at 1.49 min.

¹H NMR (400 MHz, Methanol-d4) δ 8.53 (d, J=9.0 Hz, 1H), 8.15 (t, J=7.6 Hz, 1H), 7.81 (d, J=2.1 Hz, 1H), 7.61 (d, J=8.7 Hz, 1H), 7.03-6.90 (m, 1H), 6.06-5.76 (m, 1H), 5.48-5.28 (m, 2H), 4.60 (ddd, J=30.3, 16.5, 6.1 Hz, 1H), 4.31 (q, J=2.7 Hz, 3H), 3.94-3.60 (m, 4H), 2.96-2.73 (m, 2H), 2.57 (d, J=49.0 Hz, 7H), 2.40-2.23 (m, 1H), 2.14-1.91 (m, 1H), 1.33-1.18 (m, 3H).

### Step 6: N-(2-chloro-4-(trifluoromethyl)phenyl)-2-(5-ethyl-6-(1-(5-hydroxy-6-meth ylpyrimidine-4-carbonyl)-2,5,6,7-tetrahydro-1H-azepin-4-yl)-7-oxo-2-(tetrahydro-2H-pyran-4-yl)-[1,2,4]triazolo[1,5-a]pyrimidin-4(7H)-yl)acetamide (Compound 196)

A solution of 003 (9 mg, 0.01 mmol) and Pd/C (0.00 mL, 0.01 mmol) in IPA (5 mL) was stirred at 25° C. for 24 hr under H₂. LCMS (XT221416-361-P1-27380-LCMS02) showed desired product was detected and the starting material was consumed completely. The solution was filtered and concentrated. The residue was purified by perp-HPLC. to give product Compound 196 (9.87 mg, 20.14% yield) as white solid. MS: m/z=715.4 (M+1, ESI+) at 1.46 min.

¹H NMR (400 MHz, Methanol-d4) δ 8.50 (s, 1H), 8.14 (t, J=7.4 Hz, 1H), 7.81 (s, 1H), 7.62 (d, J=8.7 Hz, 1H), 5.88 (d, J=69.5 Hz, 1H), 5.34 (dd, J=33.2, 17.0 Hz, 2H), 4.60 (d, J=15.4 Hz, 2H), 4.42-4.22 (m, 1H), 4.04 (dd, J=38.0, 13.7 Hz, 3H), 3.77 (d, J=16.1 Hz, 1H), 3.55 (t, J=11.0 Hz, 2H), 3.10 (d, J=20.2 Hz, 1H), 2.97-2.75 (m, 2H), 2.50 (s, 4H), 1.95 (d, J=6.1 Hz, 4H), 1.25 (dt, J=45.8, 7.8 Hz, 5H).

### Preparation Example 11

### Synthesis of Compound 191

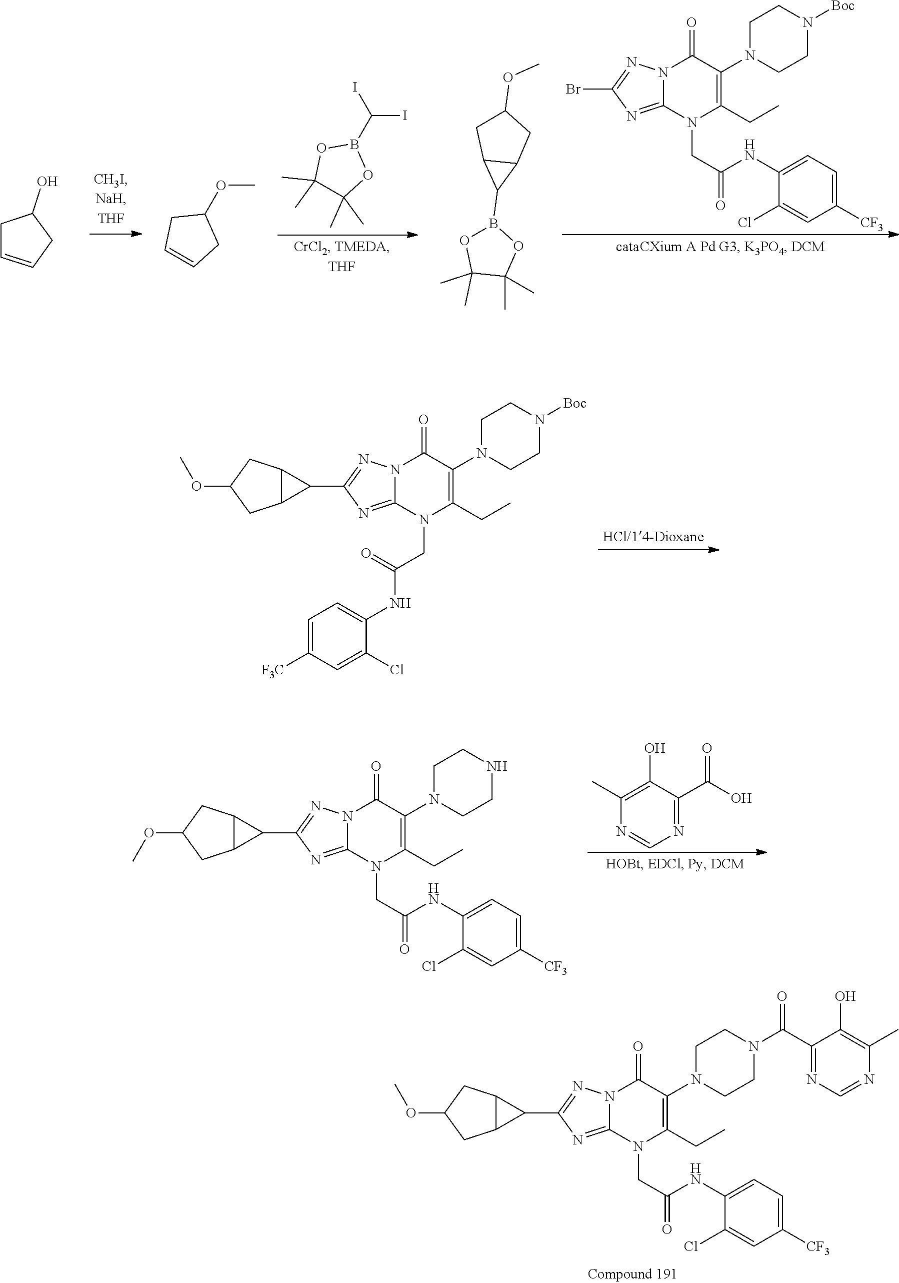

### Step 1: 4-methoxycyclopent-1-ene

To a solution of cyclopent-3-en-1-ol (5.0 g, 59.44 mmol) and methy iodide (9.28 g, 65.39 mmol) in THF (50 mL), 60% sodium hydride (1.71 g, 71.33 mmol) was added in small portions under ice cooling, and the thus obtained mixture was stirred at room temperature overnight. The reaction mixture was partitioned by adding water and diethyl ether, and the organic layer was dried over anhydrous magnesium sulfate. The solvent was distilled away under reduced pressure under ice cooling, to thereby give crude 4-methoxycyclopent-1-ene 1.3 g, 22.28% yield.

Mass calc. C6H10O for: 98.1; found:not show MS.

¹H NMR (400 MHz, CD₂Cl₂) δ 5.669 (s, 1H), 5.468 (s, 1H), 4.061-4.027 (m, 1H), 3.184 (s, 3H), 2.473-2.454 (d, J=7.6 Hz, 1H), 2.276-2.269 (d, J=2.8 Hz, 1H), 2.237-2.230 (d, J=2.8 Hz, 1H), 2.157-2.111 d, J=2.8 Hz, 1H).

### Step 2: 2-(3-methoxybicyclo[3.1.0]hexan-6-yl)-4,4,5,5-tetramethyl-1,3,2-dioxaborolane

Under nitrogen, a solution of CrCl₂ (9.77 g, 79.48 mmol) and TMEDA (9.24 g, 79.48 mmol) in THF (80 mL) was stirred for 1 h at room temperature. The 2-(diiodomethyl)-4,4,5,5-tetramethyl-1,3,2-dioxaborolane (7.82 g, 19.87 mmol) was added. The mixture was stirred at room temperature for 1 h. 4-methoxycyclopent-1-ene (1.3 g, 13.25 mmol) was added and the mixture was stirred at 50° C. for 20 h. The reaction was quenched with water and extracted with EA. The organic layers were combined, dried over Na₂SO₄, filtered and concentrated under vacuum. The residue was purified by column chromatography eluting with 20/1 PE/EA to afford 2-(3-methoxybicyclo[3.1.0]hexan-6-yl)-4,4,5,5-tetramethyl-1,3,2-dioxaborolane 200 mg, 6.3% yield.

Mass calc. C11H19O3B for: 238.1; found:not show MS.

¹H NMR (400 MHz, CD₂Cl₂) δ 3.796-3.760 (m, 1H), 3.103 (s, 3H), 2.077-2.062 (m, 2H), 1.921-1.895 (m, 1H), 1.703-1.664 (m, 2H), 1.351-1.342 (m, 1H), 1.152-1.147 (m, 1H), 1.187 (s, 12H).

### Step 3: tert-butyl 4-(2-(3-oxabicyclo[3.1.0]hexan-6-yl)-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl) piperazine-1-carboxylate

Under nitrogen, a mixture of tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate (500 mg, 0.75 mmol), 2-(3-methoxybicyclo[3.1.0]hexan-6-yl)-4,4,5,5-tetramethyl-1,3,2-dioxaborolane (269 mg, 1.13 mmol), cataCXium A Pd G3 (110 mg, 0.15 mmol) and K₃PO₄ (480 mg, 2.26 mmol) in DMF (10 mL) and H₂O(1 mL) was stirred at 100° C. for 2 h. The reaction was quenched with water and extracted with EA. The organic layers were combined, dried over Na₂SO₄, filtered and concentrated under vacuum. The residue was purified by column chromatography eluting with 1/1 PE/EA to afford tert-butyl 4-(2-(3-oxabicyclo[3.1.0]hexan-6-yl)-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate 70 mg, 4.77% yield.

Mass calc. C32H39O5N7F3Cl for: 693.1.0; found: 692.1 [M−H]⁺, ESI.

### Step 4: N-(2-chloro-4-(trifluoromethyl)phenyl)-2-(5-ethyl-2-(3-methoxybicyclo[3.1.0]hexan-6-yl)-7-oxo-6-(piperazin-1-yl)-[1,2,4]triazolo[1,5-a]pyrimidin-4(7H)-yl)acetamide

tert-butyl 4-(2-(3-oxabicyclo[3.1.0]hexan-6-yl)-4-(2-((2-chloro-4-(trifluoromethyl) phenyl)amino)-2-oxoethyl)-5-ethyl-7-oxo-4,7-dihydro-[1,2,4]triazolo[1,5-a]pyrimidin-6-yl)piperazine-1-carboxylate (60 mg, 0.08 mmol) in HCl/1′4-Dioxane (5 mL) was stirred overnight at room temperature. LC-MS showed full conversion. The mixture was concentrated in vacuo. The compound was directly used for the next step without further purification.

Mass calc. C27H31O3N7F3Cl for: 593.0; found: 592.0 [M−H]⁺, ESI.

### Step 5: N-(2-chloro-4-(trifluoromethyl)phenyl)-2-(5-ethyl-6-(4-(5-hydroxy-6-methylpyrimidine-4-carbonyl)piperazin-1-yl)-2-(3-methoxybicyclo[3.1.0]hexan-6-yl)-7-oxo-[1,2,4]triazolo[1,5-a]pyrimidin-4(7H)-yl)acetamide, Compound 191

5-hydroxy-6-methylpyrimidine-4-carboxylic acid (26.24 mg, 0.17 mmol), Py (20.51 mg, 0.26 mmol), HOBT (23.36 mg, 0.17 mmol), EDCI (33.14 mg, 0.17 mmol) in DCM (2 mL) was stirred at room temperature for 0.5 h, N-(2-chloro-4-(trifluoromethyl)phenyl)-2-(5-ethyl-2-(3-methoxybicyclo[3.1.0]hexan-6-yl)-7-oxo-6-(piperazin-1-yl)-[1,2,4]triazolo[1,5-a]pyrimidin-4(7H)-yl)acetamide (60 mg, 0.08 mmol) was added, and stirred at r.t. for 3 h. LC-MS showed full conversion. The reaction mixture was diluted by additional DCM (10 mL), and washed by water (10 mL), concentrated and purified by prep-HPLC (C18 column, ACN/water) to afford N-(2-chloro-4-(trifluoromethyl)phenyl)-2-(5-ethyl-6-(4-(5-hydroxy-6-methylpyrimidine-4-carbonyl)piperazin-1-yl)-2-(3-methoxybicyclo[3.1.0]hexan-6-yl)-7-oxo-[1,2,4]tri azolo[1,5-a]pyrimidin-4(7H)-yl)acetamide (Compound 191) 10 mg, 15.85% yield.

Mass calc. C33H35O5N9F3Cl for: 730.0; found: 728.0 [M−H]⁺, ESI.

¹H NMR (400 MHz, CD₂Cl₂) δ 10.322 (s, 1H), 8.573 (s, 1H), 8.065-8.044 (d, J=8.4 Hz, 1H), 7.974 (s, 1H), 7.740-7.714 (m, 1H), 5.252 (s, 2H), 4.530-4.500 (d, J=12 Hz, 1H), 3.717-3.681 (m, 2H), 3.501-3.445 (m, 2H), 3.173 (s, 3H), 2.973-2.946 (m, 4H), 2.803-2.777 (d, J=10.4 Hz, 1H), 2.624-2.599 (d, J=10 Hz, 1H), 2.445 (s, 3H), 2.274-2.225 (m, 2H), 1.795 (s, 3H), 1.719-1.673 (m, 2H), 1.183-1.146 (m, 3H).

### Preparation Example 12

### Synthesis of Compound 192

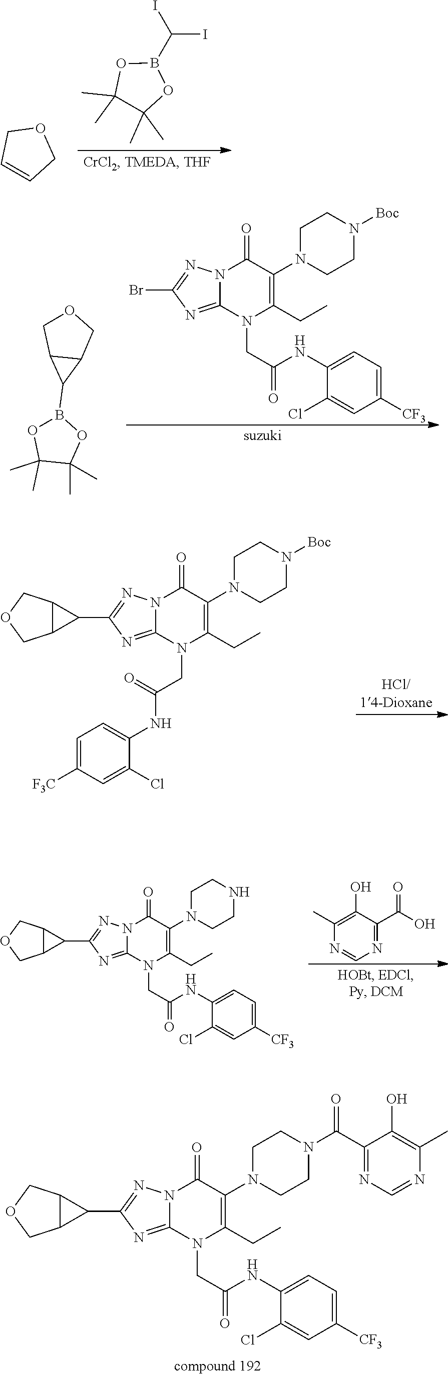

### Step 1: 2-(3-oxabicyclo[3.1.0]hexan-6-yl)-4,4,5,5-tetramethyl-1,3,2-dioxaborolane

Under nitrogen, a solution of CrCl₂ (10.52 g, 85.6 mmol, 6.0 equiv) and TMEDA (9.95 g, 85.6 mmol, 6.0 equiv) in THF (50 mL) was stirred for 1 h at room temperature. The 2-(diiodomethyl)-4,4,5,5-tetramethyl-1,3,2-dioxaborolane (8.43 g, 21.4 mmol, 1.5 equiv) was added. The mixture was stirred at room temperature for 1 h. 2,5-dihydrofuran (1.0 g, 14.27 mmol, 1 equiv) was added and the mixture was stirred at 50° C. for 20 h. The reaction was quenched with water and extracted with EA. The organic layers were combined, dried over Na₂SO₄, filtered and concentrated under vacuum. The residue was purified by column chromatography eluting with 20/1 PE/EA to afford 2-(3-oxabicyclo[3.1.0]hexan-6-yl)-4,4,5,5-tetramethyl-1,3,2-dioxaborolane 1.8 g, 60.85% yield.

Mass calc. C11H19O3B for: 210.0; found:not show MS.

¹H NMR (400 MHz, CD₂Cl₂) δ 3.693-3.673 (d, J=8 Hz, 2H), 3.574-3.553 (d, J=8.4 Hz, 2H), 1.664-1.655 (d, J=3.6 Hz, 2H), 1.163 (s, 12H).

### Step 2: tert-butyl 4-(2-(3-oxabicyclo[3.1.0]hexan-6-yl)-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl) piperazine-1-carboxylate

Under nitrogen, a mixture of tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate (500 mg, 0.75 mmol), 2-(3-oxabicyclo[3.1.0]hexan-6-yl)-4,4,5,5-tetramethyl-1,3,2-dioxaborolane (206 mg, 0.98 mmol), cataCXium A Pd G3 (110 mg, 0.15 mmol) and K₃PO₄ (480 mg, 2.26 mmol) in DMF (10 mL) and H₂O(1 mL) was stirred at 100° C. for 2 h. The reaction was quenched with water and extracted with EA. The organic layers were combined, dried over Na₂SO₄, filtered and concentrated under vacuum. The residue was purified by column chromatography eluting with 98/2 DCM/MeOH to afford tert-butyl 4-(2-(3-oxabicyclo[3.1.0]hexan-6-yl)-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate 26 mg, 5.17% yield.

Mass calc. C30H35O5N7F3Cl for: 665.0; found: 664.0 [M−H]⁺, ESI.

¹H NMR (400 MHz, CD₂Cl₂) δ 10.342 (s, J=1H), 8.078-8.057 (d, J=8.4 Hz, 1H), 7.975-7.971 (d, J=1.6 Hz, 1H), 7.739-7.713 (m, 1H), 5.279 (s, 2H), 3.906-3.885 (m, 4H), 3.692-3.671 (d, J=8.4 Hz, 2H), 3.406-3.353 (m, 2H), 2.955-2.937 (m, 4H), 2.649-2.622 (d, J=10.8 Hz, 2H), 2.146-2.139 (d, J=2.8 Hz, 2H), 1.854-1.837 (m, 1H), 1.433 (s, 9H), 1.178-1.141 (m, 3H).

### Step 3: 2-(2-(3-oxabicyclo[3.1.0]hexan-6-yl)-5-ethyl-7-oxo-6-(piperazin-1-yl)-[1,2,4]triazolo[1,5-a]pyrimidin-4(7H)-yl)-N-(2-chloro-4-(trifluoromethyl)phenyl)acetamide

tert-butyl 4-(2-(3-oxabicyclo[3.1.0]hexan-6-yl)-4-(2-((2-chloro-4-(trifluoromethyl) phenyl)amino)-2-oxoethyl)-5-ethyl-7-oxo-4,7-dihydro-[1,2,4]triazolo [1,5-a]pyrimidin-6-yl)piperazine-1-carboxylate (40 mg, 0.06 mmol) in HCl/1′4-Dioxane (5 mL) was stirred overnight at room temperature. LC-MS showed full conversion. The mixture was concentrated in vacuo. The compound was directly used for the next step without further purification.

Mass calc. C25H27O3N7F3Cl for: 565.9; found: 564.9 [M−H]⁺, ESI.

### Step 4: 2-(2-(3-oxabicyclo[3.1.0]hexan-6-yl)-5-ethyl-6-(4-(5-hydroxy-6-methylpyrimidine-4-carbonyl)piperazin-1-yl)-7-oxo-[1,2,4]triazolo[1,5-a]pyrimidin-4(7H)-yl)-N-(2-chloro-4-(trifluoromethyl)phenyl)acetamide, compound 192

5-hydroxy-6-methylpyrimidine-4-carboxylic acid (21.79 mg, 0.14 mmol), Py (16.77 mg, 0.21 mmol), HOBT (19.10 mg, 0.14 mmol), EDCI (21.70 mg, 0.14 mmol) in DCM (2 mL) was stirred at room temperature for 0.5 h, 2-(2-(3-oxabicyclo[3.1.0]hexan-6-yl)-5-ethyl-7-oxo-6-(piperazin-1-yl)-[1,2,4]triazolo[1,5-a]pyrimidin-4(7H)-yl)-N-(2-chloro-4-(trifluoromethyl)phenyl)acetamide (40 mg, 0.07 mmol) was added, and stirred at r.t. overnight. LC-MS showed full conversion. The reaction mixture was diluted by additional DCM (10 mL), and washed by water (10 mL), concentrated and purified by prep-HPLC (C18 column, ACN/water) to afford 2-(2-(3-oxabicyclo[3.1.0]hexan-6-yl)-5-ethyl-6-(4-(5-hydroxy-6-methylpyrimidine-4-carbonyl)piperazin-1-yl)-7-oxo-[1,2,4]triazolo[1,5-a]pyrimidin-4(7H)-yl)-N-(2-chloro-4-(trifluoromethyl)phenyl)acetamide (compound 192) 5.6 mg, 11.29% yield.

Mass calc. C31H31O5N9F3Cl for: 702.0; found: 700.0 [M−H]⁺, ESI.

¹H NMR (400 MHz, CD₂Cl₂) δ 10.342 (s, 1H), 8.503 (s, 1H), 8.071-8.050 (d, J=8.4 Hz, 1H), 7.973 (s, 1H), 7.735-7.713 (d, J=8.8 Hz, 1H), 5.279 (s, 2H), 3.904-3.883 (d, J=8.4 Hz, 2H), 3.731-3.670 (m, 4H), 3.598 (s, 2H), 2.973-2.960 (m, 3H), 2.802-2.775 (d, J=10.8 Hz, 1H), 2.264-2.259 (d, J=2 Hz, 1H), 2.423 (s, 2H), 2.142 (s, 2H), 1.854-1.846 (m, 1H), 1.189-1.153 (m, 3H).

### Preparation Example 13

### Synthesis of Compound 195

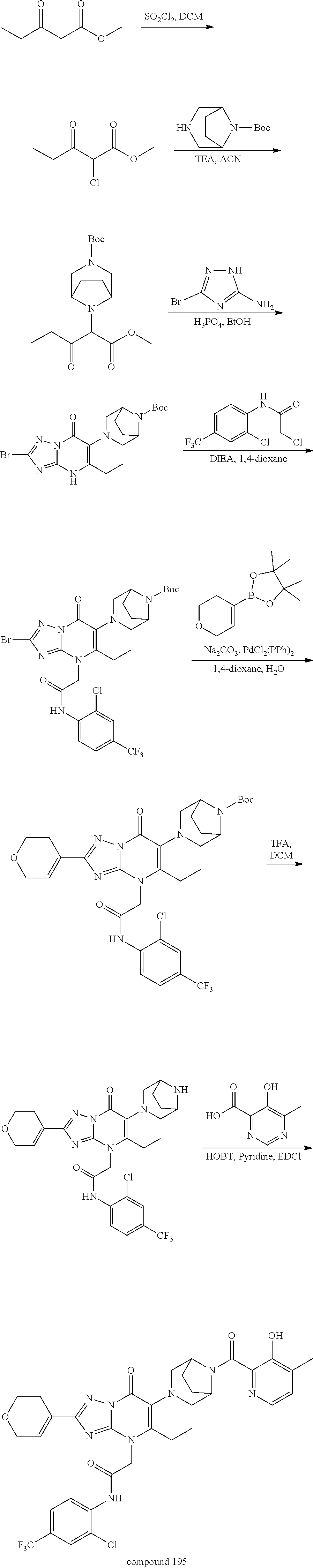

### Step 1: methy2-chloro-3-oxopentanoate

To a solution of methy3-oxopentanoate (10 g, 76.84 mmol) in DCM (100 ml) was added SO₂Cl₂ (13.48 g, 99.89 mmol) at 0° C. over 10 min. The reaction was allowed to warm to RT and stirred for 16 hours. The RM was concentrated under reduced pressure and the residue was dissolved in DCM (100 ml) and washed with water (100 ml), brine (100 ml), dried over Na₂SO₄ and filtered. The filtrate was concentrated under reduced pressure to give the title compound as light yellow liquid (12 g, 94% yield).

Mass calc C₆H₉ClO₃ for 164.0, found: 163.0 (M−H)− ESI.

¹H NMR (400 MHz, DMSO-d₆) δ: 5.62 (s, 1H), 3.76 (s, 3H), 2.50-2.48 (m, 2H), 0.99 (t, J=7.2 Hz, 3H).

### Step 2: tert-butyl (1R,5S)-3-(1-methoxy-1,3-dioxopentan-2-yl)-3,8-diazabicyclo[3.2.1]octane-8-carboxylate

To a solution of methy2-chloro-3-oxopentanoate (5.04 g, 30.62 mmol), in dry ACN (25 ml) was added TEA (7.15 g, 70.66 mmol) over 15 min, followed by dropwise addition of tert-butyl 3,8-diazabicyclo[3.2.1]octane-8-carboxylate (5 g, 23.55 mmol) in ACN over 30 min. The reaction was stirred at 60° C. for 16 hours. The RM was filtered and washed with EA. The filtrated was then concentrated under reduced pressure and the residue was dissolved in EA and washed with water, dried over Na₂SO₄ and filtered. The filtrate was concentrated under reduced pressure and the residue was purified by column chromatography eluent heptane:EA 100:0 to 90:10) to give the title compound (4 g, 50% yield).

Mass calc C₁₇H₂₈N₂O₅ for 340.2, found: 339.1 (M−H)− ESI.

¹H NMR (400 MHz, DMSO-d₆) δ: 4.36 (s, 1H), 3.61 (s, 3H), 3.21-3.11 (m, 1H), 2.99-2.92 (m, 1H), 2.75-2.60 (m, 4H), 2.37-2.26 (m, 2H), 1.88-1.75 (m, 4H), 1.41-1.38 (s, 9H), 0.96 (t, J=7.3 Hz, 3H).

### Step 3: tert-butyl 3-(2-bromo-5-ethyl-7-oxo-4,7-dihydro-[1,2,4]triazolo[1,5-a]pyrimidin-6-yl)-3,8-diaza bicyclo[3.2.1]octane-8-carboxylate

3-Bromo-1H-1,2,4-triazol-5-amine (843 mg, 5.17 mmol) and tert-butyl (1R,5S)-3-(1-methoxy-1,3-dioxopentan-2-yl)-3,8-diazabicyclo[3.2.1]octane-8-carboxylate (1.6 g, 4.70 mmol) were mixed in EtOH (3 mL). H₃PO₄ (461 mg, 4.70 mmol) was added. The mixture was stirred at 80° C. for 12 hours under nitrogen. The mixture was concentrated in vacuo to remove EtOH, then quenched by addition of aq sat NaHCO₃, and extracted with DCM. The combined organic layers were washed with brine, dried over Na₂SO₄, filtered and concentrated under reduced pressure to give a residue. The residue was purified by column chromatography (Silica column, eluent DCM:MeOH 1:0 to 50:1) to give the title compound as a yellow solid (300 mg, 14% yield).

Mass calc C₁₈H₂₅BrN₆O₃ for 452.1 454.1, found: 451.1 453.1 (M−H)− ESI.

¹H NMR (400 MHz, DMSO-d6) δ: 13.25 (s, 1H), 4.16-4.02 (m, 2H), 3.56-3.50 (d, J=9.8 Hz, 2H), 2.81-2.71 (m, 2H), 2.46-2.38 (d, J=9.8 Hz, 2H), 1.93-1.80 (m, 4H), 1.44 (s, 9H), 1.21 (t, J=7.6 Hz, 3H).

### Step 4: tert-butyl 3-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)-3,8-diazabicyclo[3.2.1]octane-8-carboxylate

To a stirred solution of tert-butyl 3-(2-bromo-5-ethyl-7-oxo-4,7-dihydro-[1,2,4]triazolo[1,5-a]pyrimidin-6-yl)-3,8-diaza bicyclo[3.2.1]octane-8-carboxylate (140 mg, 0.31 mmol) and 2-chloro-N-(2-chloro-4-(trifluoromethyl)phenyl)acetamide (84 mg, 0.31 mmol) in 1,4-dioxane (2 mL) was added DIPEA (120 mg, 0.93 mmol) at RT and the RM was stirred at 80° C. for 72 hours. The RM was concentrated under reduced pressure. The crude product was diluted with EtOAc and water, extracted once with EtOAc and the organic layer was washed with brine, dried over Na₂SO₄ and concentrated under reduced pressure. The crude product was purified by column chromatography (eluent heptane:EtOAc 70:30 to 30:70) to give the title compound as a white solid (130 mg, 610% yield).

Mass calc C₂₇H₃₀BrClF₃N₇O₄ for 687.1 689.1, found: 686.1 688.1 (M−H)− ESI.

¹H NMR (400 MHz, DMSO-d₆) δ: 10.34 (s, 1H), 8.11-8.06 (d, J=8.6 Hz, 1H), 7.99-7.96 (d, J=1.7 Hz, 1H), 7.74-7.70 (dd, J=8.7, 1.7 Hz, 1H), 5.29 (s, 2H), 4.15-4.07 (m, 2H), 3.61-3.57 (m, 2H), 3.02-2.92 (m, 2H), 2.49-2.43 (m, 2H), 1.92-1.84 (m, 4H), 1.45 (s, 9H), 1.20 (t, J=7.4 Hz, 3H).

### Step 5: tert-butyl 3-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-3,8-diazabicyclo[3.2.1]octane-8-carboxylate

Tert-butyl

3-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)-3,8-diazabicyclo[3.2.1]octane-8-carboxylate (186 mg, 0.27 mol) was suspended in 1,4-dioxane (0.5 mL). 2-(3,6-dihydro-2H-pyran-4-yl)-4,4,5,5-tetramethyl-1,3,2-dioxaborolane (113 mg, 0.54 mmol) and 0.6M aq Na₂CO₃ (0.25 mL) were added and the RM was degassed for 10 minutes with argon, PdCI₂(PPh)₂ (19 mg, 0.03 mmol) was added and the RM was stirred at 90° C. for 12 hours. The RM was concentrated under reduced pressure, and the crude product was purified by column chromatography (Silica gel column: Silica 12 g, eluent DCM:MeOH 100:0 to 97:3) to give the crude title compound (72 mg).

Mass calc C₃₂H₃₇ClF₃N₇O₅ for 691.2, found: 690.1 (M−H)− ESI.

### Step 6: 2-(6-(3,8-diazabicyclo[3.2.1]octan-3-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide

Tert-butyl

3-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)-3,8-diazabicyclo[3.2.1]octane-8-carboxylate (40 mg, 0.06 mmol) was suspended in DCM (2 ml), TFA (1 ml) was added and the RM was stirred at RT for 4 h. The RM was concentrated under reduce pressure and used without purification.

Mass calc C₂₇H₂₉ClF₃N₇O₃ for 591.2, found: 592.1 (M+H)+ ESI.

### Step 7: N-(2-chloro-4-(trifluoromethyl)phenyl)-2-(2-(3,6-dihydro-2H-pyran-4-yl)-5-ethyl-6-(8-(3-hydroxy-4-methylpicolinoyl)-3,8-diazabicyclo[3.2.1]octan-3-yl)-7-oxo-[1,2,4]tri azolo[1,5-a]pyrimidin-4(7H)-yl)acetamide

5-hydroxy-6-methyl-pyrimidine-4-carboxylic acid (18 mg, 0.12 mmol) was dissolved in DCM (4 ml), to this solution were added Pyridine (14 mg, 0.18 mmol), HOBT (16 mg, 0.11 mmol), EDCI (23 mg, 0.11 mmol),

2-(6-(3,8-diazabicyclo[3.2.1]octan-3-yl)-2-(3,6-dihydro-2H-pyran-4-yl)-5-ethyl-7-oxo-[1,2,4]triazolo[1,5-a]pyrimidin-4(7H)-yl)-N-(2-chloro-4-(trifluoromethyl)phenyl)acetamide (35 mg, 0.06 mmol). After stirring for 3 hours, the RM was purified by reverse phase column (C18 column) to provide the product compound 195 (15 mg, 34% yield).

Mass calc C₃₄H₃₄ClF₃N₈O₅ for 726.2, found: 727.1 (M+H)+ ESI.

¹H NMR (400 MHz, DMSO-d₆) δ: 11.75 (s, 1H), 10.39 (s, 1H), 8.62 (s, 1H), 8.08-8.04 (d, J=8.5 Hz, 1H), 7.99-7.97 (s, 1H), 7.74-7.70 (d, J=8.5 Hz, 1H), 6.82 (m, 1H), 5.32 (s, 2H), 5.15 (s, 1H), 4.85-4.81 (d, J=5.7 Hz, 1H), 4.26-4.23 (m, 2H), 3.79 (t, J=5.3 Hz, 2H), 3.72 (t, J=10.6 Hz, 2H), 3.07-3.98 (m, 2H), 2.73-2.61 (m, 2H), 2.46 (s, 4H), 2.06-1.92 (m, 4H), 1.28-1.21 (m, 5H).

### Preparation Example 14

### Synthesis of Compound 202

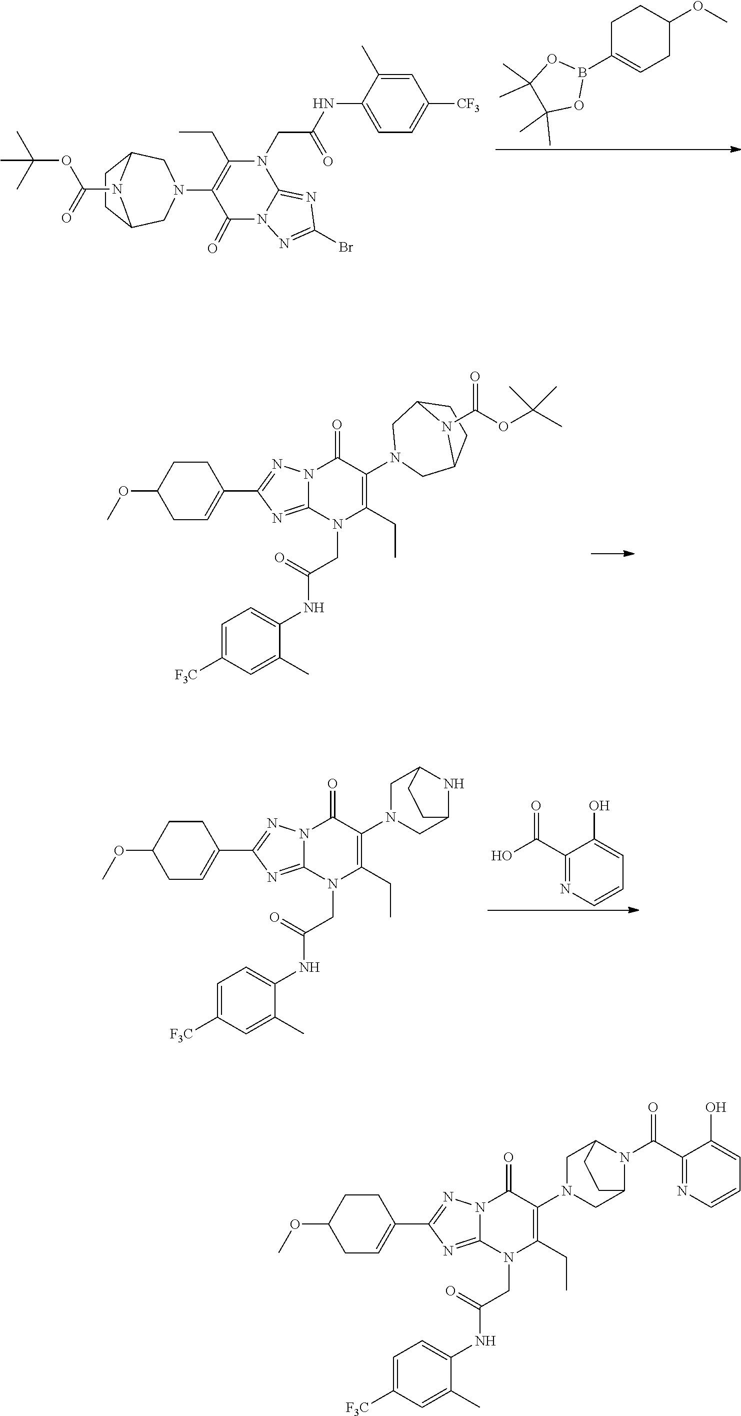

### Step 1: tert-butyl 3-(5-ethyl-2-(4-methoxycyclohex-1-en-1-yl)-4-(2-((2-methyl-4-(trifluoromethyl)phenyl)amino)-2-oxoethyl)-7-oxo-4,7-dihydro-[1,2,4]triazolo

[1,5-a]pyrimidin-6-yl)-3,8-diazabicyclo[3.2.1]octane-8-carboxylate, 139-4 139-3 (100 mg, 0.22 mmol) was suspended in DMF (5 mL), H₂O (0.5 mL), 2-(4-methoxycyclohexen-1-yl)-4,4,5,5-tetramethyl-1,3,2-dioxaborolane (105.06 mg, 0.44 mmol) and Na₂CO₃ (70.14 mg, 0.66 mmol) were added and the mixture was degassed for 10 minutes with argon, PdCl₂(PPh₃)₂ (15.48 mg, 22.06 μmol) was added and the mixture was stirred at 90° C. overnight. The mixture was concentrated under reduced pressure, and the crude was diluted with DCM and water, extracted once with DCM and the organic layer was washed with brine, dried over Na₂SO₄ and concentrated under reduced pressure. The product was purified by column chromatography to give the compound (120 mg, 77.74% yield).

Mass calc C₃₅H₄₄F₃N₇O₅ for 699.34 found:644.3 (M-tBu+H)⁺ ESI.

### Step 2: 2-(6-(3,8-diazabicyclo[3.2.1]octan-3-yl)-5-ethyl-2-(4-methoxycyclohex-1-en-1-yl)-7-oxo-[1,2,4]triazolo[1,5-a]pyrimidin-4(7H)-yl)-N-(2-methyl-4-(trifluoromethyl)phenyl)acetamide, 139-5

139-4 (100 mg, 0.22 mmol) was suspended in DCM (5 mL), hydrogen chloride in dioxane (2 mL) was added and the mixture was stirred at RT for 2 h. The RM was concentrated under reduce pressure and used without purification.

Mass calc C₃₀H₃₆F₃N₇O₃ for 599.28 found: 566.2, 568.2 (M+H)⁺ ESI.

Step 3: 2-(5-ethyl-6-(8-(3-hydroxypicolinoyl)-3,8-diazabicyclo[3.2.1]octan-3-yl)-2-(4-methoxycyclohex-1-en-1-yl)-7-oxo-[1,2,4]triazolo[1,5-a]pyrimidin-4(7H)-yl)-N-(2-methyl-4-(trifluoromethyl)phenyl)acetamide. 139-5 (100 mg, 0.17 mmol) was dissolved in DCM (5 mL), the solution was added 3-hydroxypyridine-2-carboxylic acid (23.20 mg, 0.17 mmol), EDCI (63.94 mg, 0.33 mmol), HOBT (45.07 mg, 0.33 mmol). After stirring at RT for 2 h, the RM was purified by reverse phase column (C18 column) to provide the product HBP-109-LY-NV-139 (38 mg, 31.62% yield). Mass calc C₃₆H₃₉F₃N₈O₅ for 720.30 found:719.4 (M−H)-ESI. ¹H NMR (400 MHz, DMSO-d₆) δ: 11.88 (br, 1H), 10.01 (s, 1H), 8.13-8.12 (m, 1H), 7.62 (s, 1H), 7.72 (d, J=8.4 Hz, 1H), 7.53 (d, J=8.4 Hz, 1H), 7.43-7.36 (m, 2H), 6.73 (s, 1H), 5.23 (s, 2H), 5.11-5.10 (m, 1H), 4.83-4.82 (m, 1H), 3.73-3.70 (m, 2H), 3.52-3.50 (m, 1H), 3.27 (s, 3H), 3.04-3.02 (m, 2H), 2.68-2.59 (m, 5H), 2.36 (s, 3H), 2.03-2.02 (m, 1H), 1.96-1.93 (m, 5H), 1.70-1.66 (m, 1H), 1.27-1.23 (m, 3H).

### Preparation Example 15

### Synthesis of Compound 203

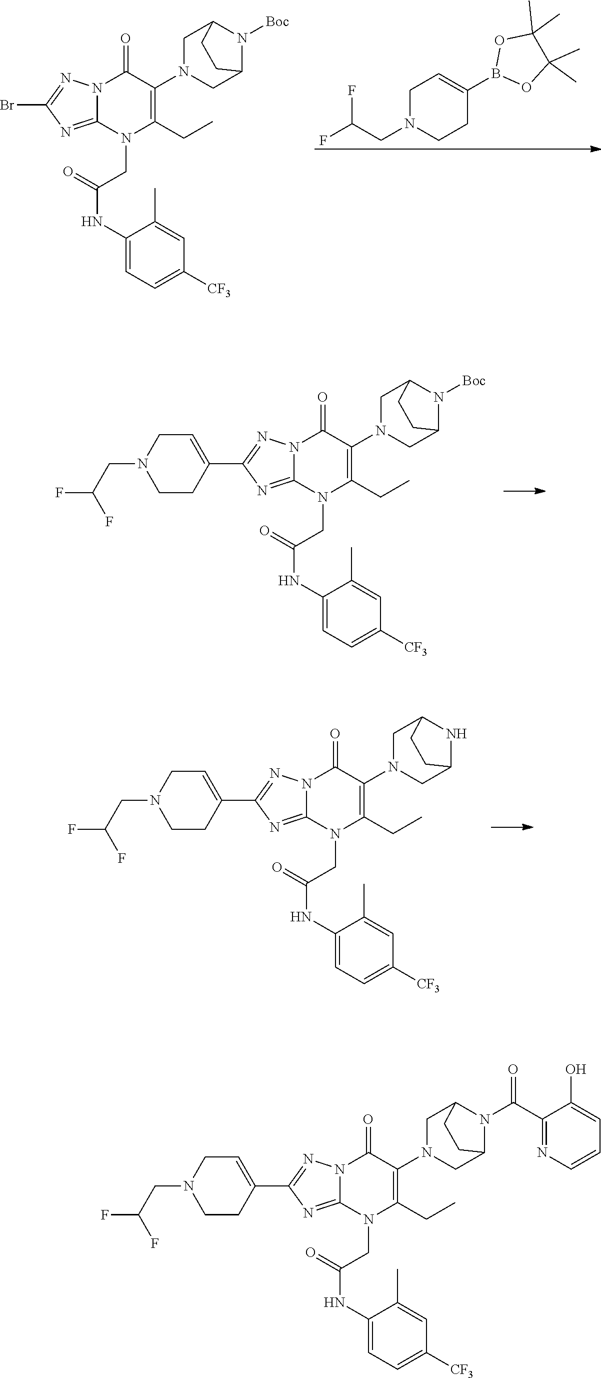

### Step 1: tert-butyl 3-(2-(1-(2,2-difluoroethyl)-1,2,3,6-tetrahydropyridin-4-yl)-5-ethyl-4-(2-((2-methyl-4-(trifluoromethyl)phenyl)amino)-2-oxoethyl)-7-oxo-4,7-dihydro-[1,2,4]triazolo[1,5-a]pyrimidin-6-yl)-3,8-diazabicyclo[3.2.1]octane-8-carboxylate, 138-1

3 (390 mg, 0.58 mmol) was suspended in DMF (10 mL), water (1 mL), 1-(2,2-difluoroethyl)-4-(4,4,5,5-tetramethyl-1,3,2-dioxaborolan-2-yl)-1,2,3,6-tetrahydropyridine (239.01 mg, 0.88 mmol) and Na₂CO₃ (185.50 mg, 1.75 mmol) were added and the mixture was degassed for 10 minutes with argon, PdCl₂(PPh₃)₂ (40.95 mg, 0.06 mmol) was added and the mixture was stirred at 90° C. for 8 h. The mixture was concentrated under reduced pressure and the crude product was purified by reverse phase column (C18 column) to give 138-1 (178 mg, 41.53% yield). Mass calc C₃₅H₄₃F₅N₈O₄ for 734.33 found: 733.51 (M−H)⁻ ESI.

### Step 2: 2-(6-(3,8-diazabicyclo[3.2.1]octan-3-yl)-2-(1-(2,2-difluoroethyl)-1,2,3,6-tetrahydro pyridin-4-yl)-5-ethyl-7-oxo-[1,2,4]triazolo[1,5-a]pyrimidin-4(7H)-yl)-N-(2-methyl-4-(trifluoromethyl)phenyl)acetamide, 138-2

138-1 (55 mg, 0.07 mmol) was suspended in hydrogen chloride in dioxane (5 mL) and the mixture was stirred at RT for 2 h. The RM was concentrated under reduce pressure and used without purification. Mass calc C₃₀H₃₅F₅N₈O₂ for 634.28 found: 635.36 (M+H)⁺ ESI.

### Step 3: 2-(2-(1-(2,2-difluoroethyl)-1,2,3,6-tetrahydropyridin-4-yl)-5-ethyl-6-(8-(3-hydroxypicolinoyl)-3,8-diazabicyclo[3.2.1]octan-3-yl)-7-oxo-[1,2,4]triazolo[1,5-a]pyrimidin-4(7H)-yl)-N-(2-methyl-4-(trifluoromethyl)phenyl)acetamide, HBP-109-LY-NV-138

138-2 (47 mg, 0.07 mmol), 3-hydroxypyridine-2-carboxylic acid (20.60 mg, 0.15 mmol), EDCI (28.39 mg, 0.15 mmol), HOBT (20.01 mg, 0.15 mmol) and Pyridine (17.57 mg, 0.22 mmol) were dissolved in DCM (3 mL), then stirring at RT for 2 h. The RM was purified by reverse phase column (C18 column) to provide the product HBP-109-LY-NV-138 (18 mg, 32.16% yield). Mass calc C₃₆H₃₈F₅N₉O₄ for 755.30 found: 756.41 (M+H)+ ESI. 1H NMR (400 MHz, DMSO-d6) δ: 11.83 (br, 1H), 10.00 (s, 1H), 8.13-8.11 (m, 1H), 7.71 (d, J=8.4 Hz, 1H), 7.62 (s, 1H), 7.53 (d, J=8.4 Hz, 1H), 7.40-7.38 (m, 2H), 6.75 (s, 1H), 6.30-6.01 (m, 1H), 5.23 (s, 2H), 5.09-5.08 (m, 1H), 4.83-4.82 (m, 1H), 3.73-3.70 (m, 2H), 3.29-3.28 (m, 2H), 3.05-3.02 (m, 2H), 2.86-2.82 (m, 2H), 2.78-2.75 (m, 2H), 2.66-2.59 (m, 2H), 2.35 (s, 3H), 2.02-1.99 (m, 3H), 1.27-1.23 (m, 4H).

The compounds in the table below were synthesized according to Examples described above, or with a similar route as Examples described above.

TABLE 1

| | | LCMS: |
|---|---|---|
| Cmpd. | | m/z |
| # | Structure | [M + H]⁺ |
| 1 | 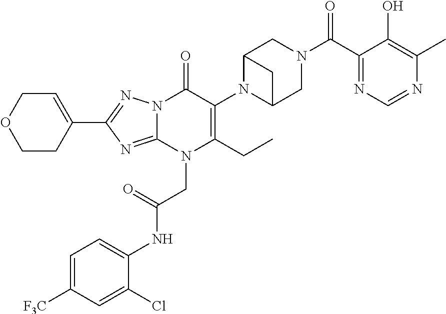 [SMILES: CCc1c(N2C3CC2CN(C(=O)c2ncnc(C)c2O)C3)c(=O)n2nc(C3=CCOCC3)nc2n1CC(=O)Nc1ccc(C(F)(F)F)cc1Cl] | 714.2 |
| 2 | 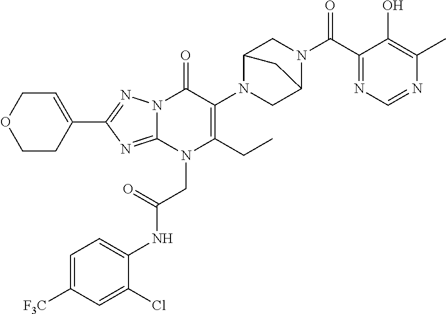 [SMILES: CCc1c(N2CC3CC2CN3C(=O)c2ncnc(C)c2O)c(=O)n2nc(C3=CCOCC3)nc2n1CC(=O)Nc1ccc(C(F)(F)F)cc1Cl] | 714.2 |
| 3 | 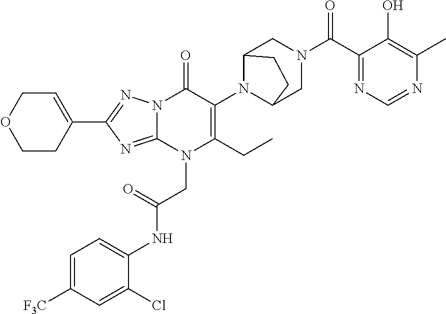 [SMILES: CCc1c(N2C3CCC2CN(C(=O)c2ncnc(C)c2O)C3)c(=O)n2nc(C3=CCOCC3)nc2n1CC(=O)Nc1ccc(C(F)(F)F)cc1Cl] | 728.2 |
| 4 | 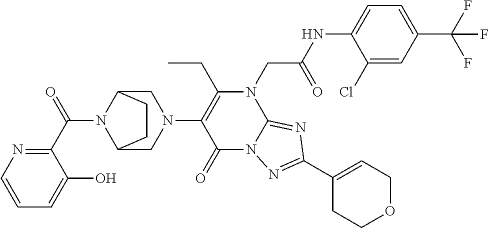 [SMILES: CCc1c(N2CC3CCC(C2)N3C(=O)c2ncccc2O)c(=O)n2nc(C3=CCOCC3)nc2n1CC(=O)Nc1ccc(C(F)(F)F)cc1Cl] | 713.2 |
| 5 | 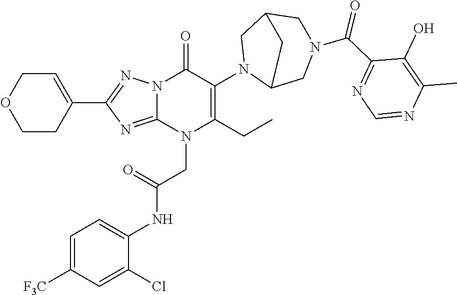 [SMILES: CCc1c(N2CC3CC2CN(C(=O)c2ncnc(C)c2O)C3)c(=O)n2nc(C3=CCOCC3)nc2n1CC(=O)Nc1ccc(C(F)(F)F)cc1Cl] | 728.2 |
| 6 | 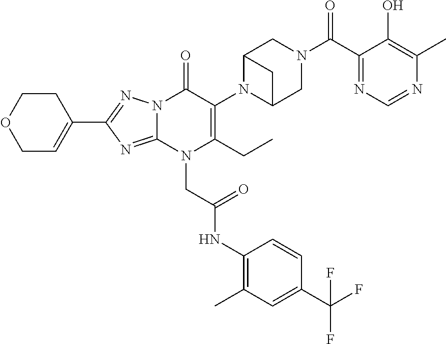 [SMILES: CCc1c(N2C3CC2CN(C(=O)c2ncnc(C)c2O)C3)c(=O)n2nc(C3=CCOCC3)nc2n1CC(=O)Nc1ccc(C(F)(F)F)cc1C] | 694.3 |
| 7 | 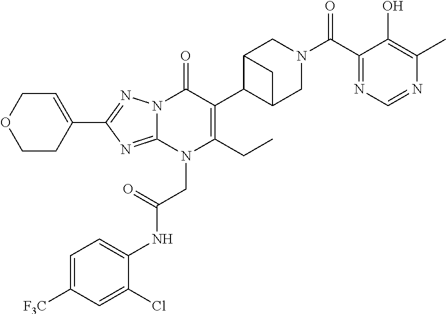 [SMILES: CCc1c(C2C3CC2CN(C(=O)c2ncnc(C)c2O)C3)c(=O)n2nc(C3=CCOCC3)nc2n1CC(=O)Nc1ccc(C(F)(F)F)cc1Cl] | 713.2 |
| 8 | 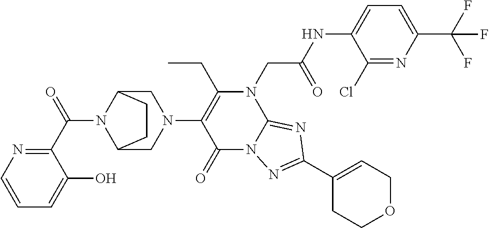 [SMILES: CCc1c(N2CC3CCC(C2)N3C(=O)c2ncccc2O)c(=O)n2nc(C3=CCOCC3)nc2n1CC(=O)Nc1ccc(C(F)(F)F)nc1Cl] | 714.2 |
| 9 | 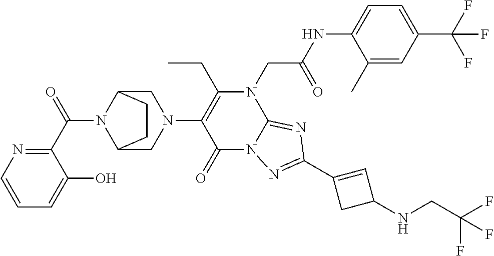 [SMILES: CCc1c(N2CC3CCC(C2)N3C(=O)c2ncccc2O)c(=O)n2nc(C3=CC(NCC(F)(F)F)C3)nc2n1CC(=O)Nc1ccc(C(F)(F)F)cc1C] | 760.3 |
| 10 | 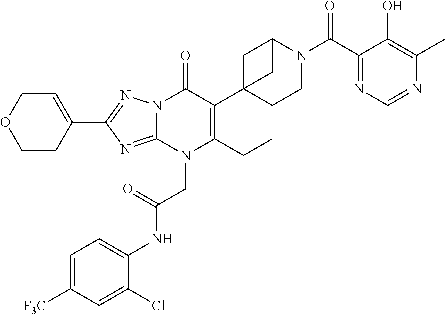 [SMILES: CCc1c(C23CCN(C(=O)c4ncnc(C)c4O)C(C2)C3)c(=O)n2nc(C3=CCOCC3)nc2n1CC(=O)Nc1ccc(C(F)(F)F)cc1Cl] | 713.2 |
| 11 | 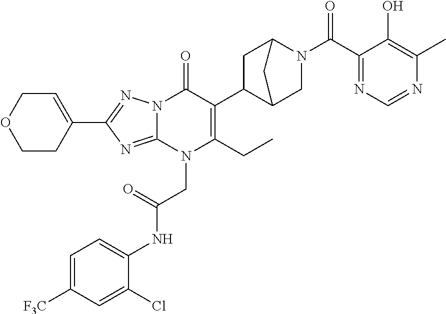 [SMILES: CCc1c(C2CC3CC2CN3C(=O)c2ncnc(C)c2O)c(=O)n2nc(C3=CCOCC3)nc2n1CC(=O)Nc1ccc(C(F)(F)F)cc1Cl] | 713.2 |
| 12 | 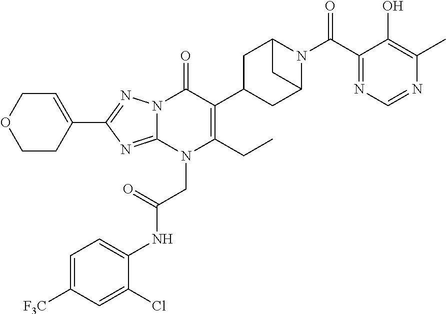 [SMILES: CCc1c(C2CC3CC(C2)N3C(=O)c2ncnc(C)c2O)c(=O)n2nc(C3=CCOCC3)nc2n1CC(=O)Nc1ccc(C(F)(F)F)cc1Cl] | 713.2 |
| 13 | 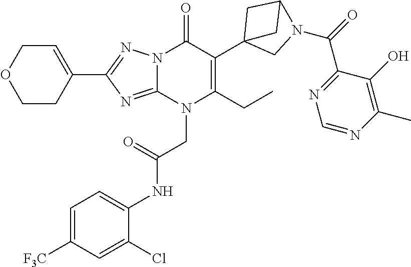 [SMILES: CCc1c(C23CC(C2)N(C(=O)c2ncnc(C)c2O)C3)c(=O)n2nc(C3=CCOCC3)nc2n1CC(=O)Nc1ccc(C(F)(F)F)cc1Cl] | 699.2 |
| 14 | 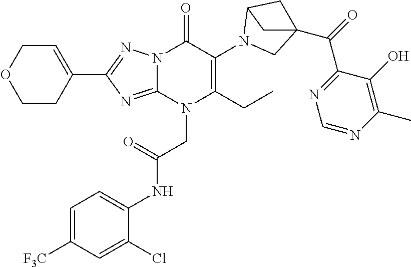 [SMILES: CCc1c(N2CC3(C(=O)c4ncnc(C)c4O)CC2C3)c(=O)n2nc(C3=CCOCC3)nc2n1CC(=O)Nc1ccc(C(F)(F)F)cc1Cl] | 699.2 |
| 15 | 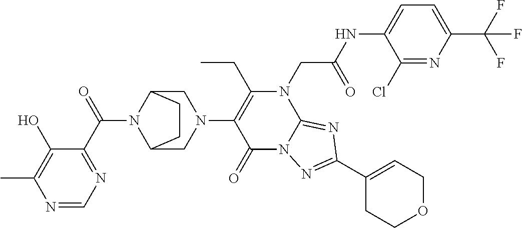 [SMILES: CCc1c(N2CC3CCC(C2)N3C(=O)c2ncnc(C)c2O)c(=O)n2nc(C3=CCOCC3)nc2n1CC(=O)Nc1ccc(C(F)(F)F)nc1Cl] | 729.2 |
| 16 | 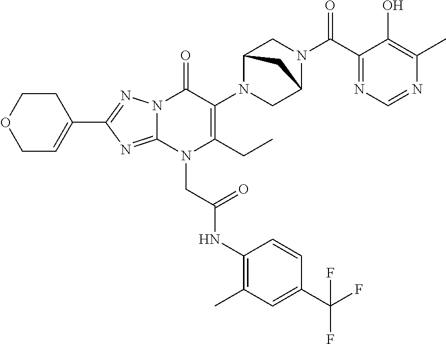 [SMILES: CCc1c(N2C[C@@H]3C[C@H]2CN3C(=O)c2ncnc(C)c2O)c(=O)n2nc(C3=CCOCC3)nc2n1CC(=O)Nc1ccc(C(F)(F)F)cc1C] | 694.3 |
| 17 | 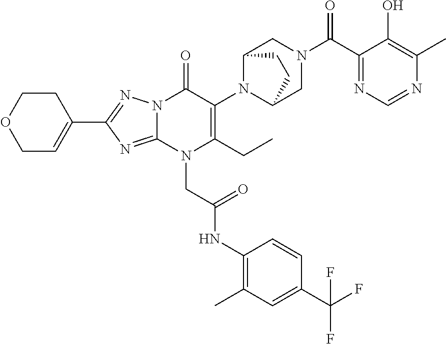 [SMILES: CCc1c(N2[C@@H]3CC[C@H]2CN(C(=O)c2ncnc(C)c2O)C3)c(=O)n2nc(C3=CCOCC3)nc2n1CC(=O)Nc1ccc(C(F)(F)F)cc1C] | 708.3 |
| 18 | 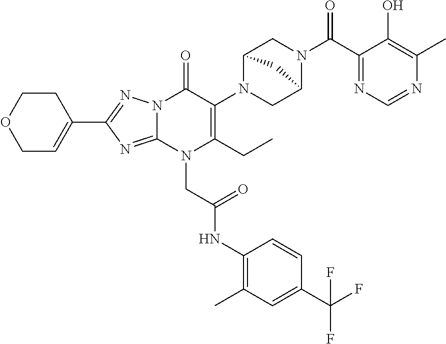 [SMILES: CCc1c(N2C[C@H]3C[C@@H]2CN3C(=O)c2ncnc(C)c2O)c(=O)n2nc(C3=CCOCC3)nc2n1CC(=O)Nc1ccc(C(F)(F)F)cc1C] | 694.3 |
| 19 | 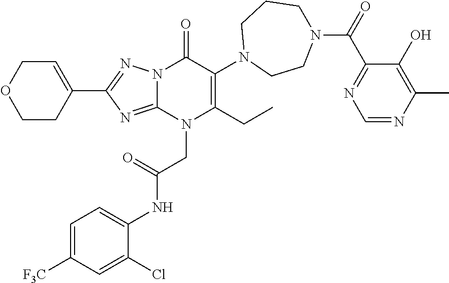 [SMILES: CCc1c(N2CCCN(C(=O)c3ncnc(C)c3O)CC2)c(=O)n2nc(C3=CCOCC3)nc2n1CC(=O)Nc1ccc(C(F)(F)F)cc1Cl] | 716.2 |
| 20 | 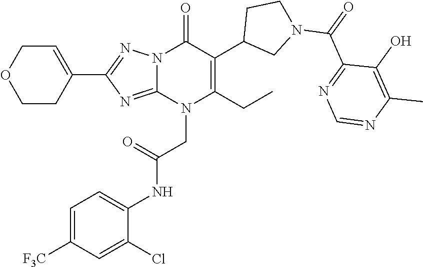 [SMILES: CCc1c(C2CCN(C(=O)c3ncnc(C)c3O)C2)c(=O)n2nc(C3=CCOCC3)nc2n1CC(=O)Nc1ccc(C(F)(F)F)cc1Cl] | 687.2 |
| 21 | 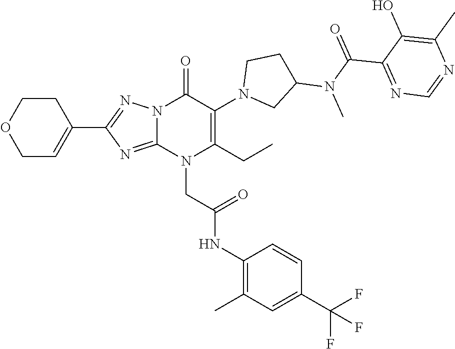 [SMILES: CCc1c(N2CCC(N(C)C(=O)c3ncnc(C)c3O)C2)c(=O)n2nc(C3=CCOCC3)nc2n1CC(=O)Nc1ccc(C(F)(F)F)cc1C] | 696.3 |
| 22 | 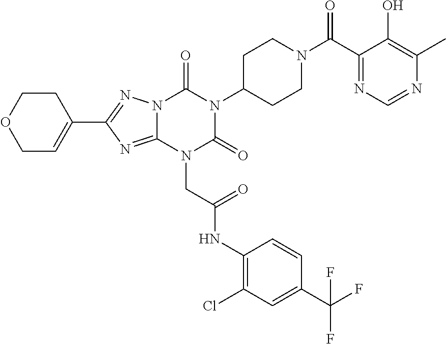 [SMILES: Cc1ncnc(C(=O)N2CCC(n3c(=O)n(CC(=O)Nc4ccc(C(F)(F)F)cc4Cl)c4nc(C5=CCOCC5)nn4c3=O)CC2)c1O] | 690.3 |
| 23 | 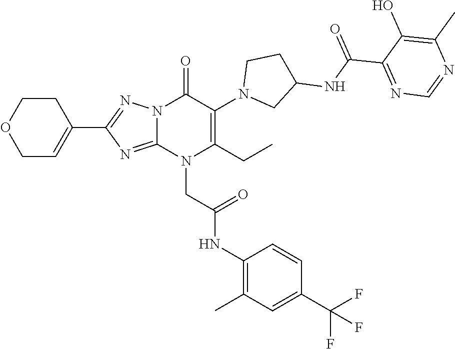 [SMILES: CCc1c(N2CCC(NC(=O)c3ncnc(C)c3O)C2)c(=O)n2nc(C3=CCOCC3)nc2n1CC(=O)Nc1ccc(C(F)(F)F)cc1C] | 682.3 |
| 24 | 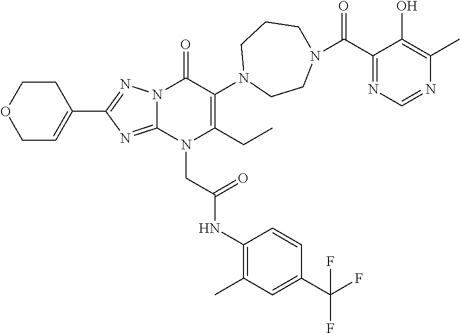 [SMILES: CCc1c(N2CCCN(C(=O)c3ncnc(C)c3O)CC2)c(=O)n2nc(C3=CCOCC3)nc2n1CC(=O)Nc1ccc(C(F)(F)F)cc1C] | 696.3 |
| 25 | 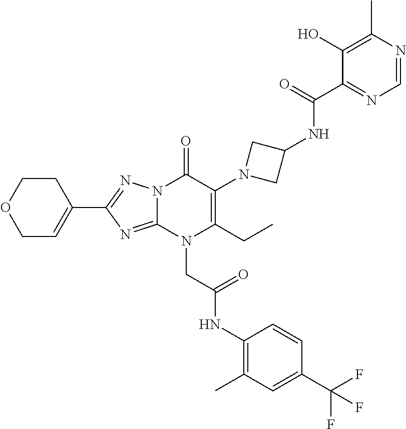 [SMILES: CCc1c(N2CC(NC(=O)c3ncnc(C)c3O)C2)c(=O)n2nc(C3=CCOCC3)nc2n1CC(=O)Nc1ccc(C(F)(F)F)cc1C] | 668.2 |
| 26 | 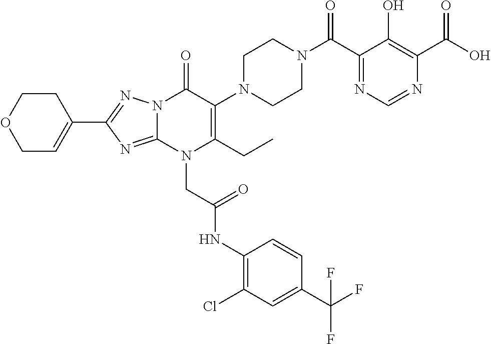 [SMILES: CCc1c(N2CCN(C(=O)c3ncnc(C(=O)O)c3O)CC2)c(=O)n2nc(C3=CCOCC3)nc2n1CC(=O)Nc1ccc(C(F)(F)F)cc1Cl] | 732.2 |
| 27 | 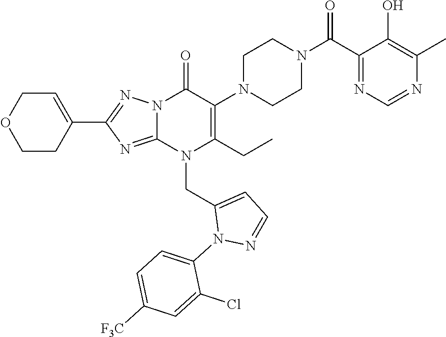 [SMILES: CCc1c(N2CCN(C(=O)c3ncnc(C)c3O)CC2)c(=O)n2nc(C3=CCOCC3)nc2n1Cc1ccnn1-c1ccc(C(F)(F)F)cc1Cl] | 725.2 |
| 28 | 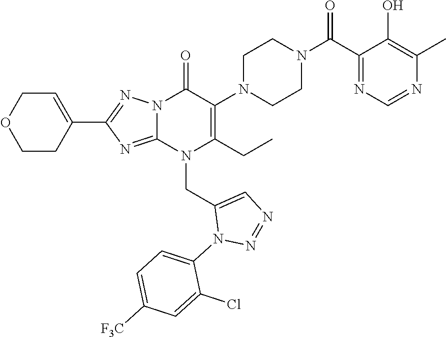 [SMILES: CCc1c(N2CCN(C(=O)c3ncnc(C)c3O)CC2)c(=O)n2nc(C3=CCOCC3)nc2n1Cc1cnnn1-c1ccc(C(F)(F)F)cc1Cl] | 726.2 |
| 29 | 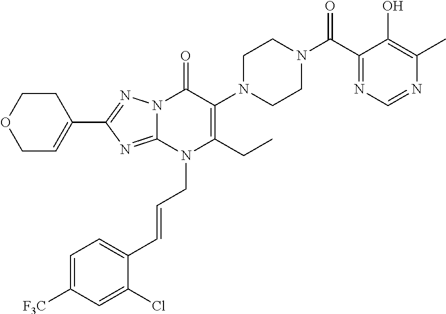 [SMILES: CCc1c(N2CCN(C(=O)c3ncnc(C)c3O)CC2)c(=O)n2nc(C3=CCOCC3)nc2n1C/C=C/c1ccc(C(F)(F)F)cc1Cl] | 685.2 |
| 30 | 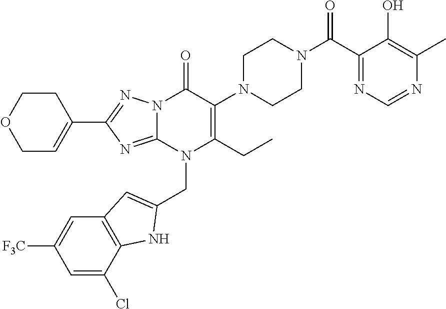 [SMILES: CCc1c(N2CCN(C(=O)c3ncnc(C)c3O)CC2)c(=O)n2nc(C3=CCOCC3)nc2n1Cc1cc2cc(C(F)(F)F)cc(Cl)c2[nH]1] | 698.2 |
| 31 | 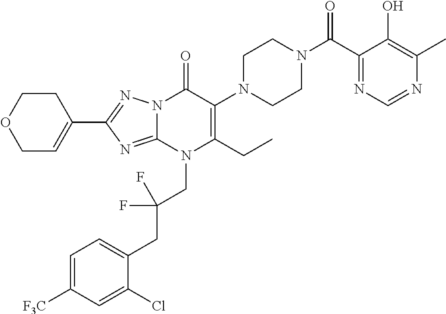 [SMILES: CCc1c(N2CCN(C(=O)c3ncnc(C)c3O)CC2)c(=O)n2nc(C3=CCOCC3)nc2n1CC(F)(F)Cc1ccc(C(F)(F)F)cc1Cl] | 723.2 |
| 32 | 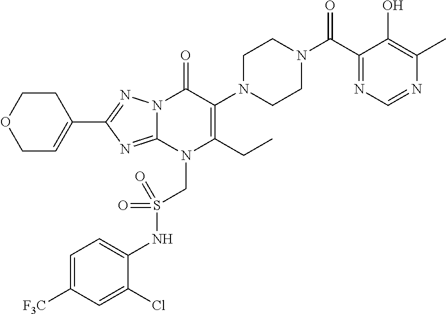 [SMILES: CCc1c(N2CCN(C(=O)c3ncnc(C)c3O)CC2)c(=O)n2nc(C3=CCOCC3)nc2n1CS(=O)(=O)Nc1ccc(C(F)(F)F)cc1Cl] | 738.2 |
| 33 | 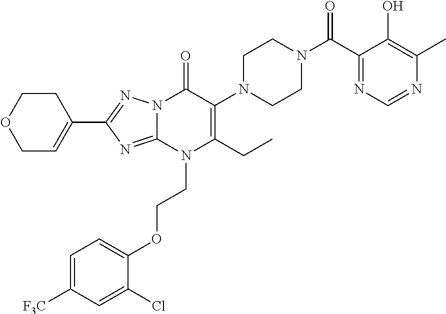 [SMILES: CCc1c(N2CCN(C(=O)c3ncnc(C)c3O)CC2)c(=O)n2nc(C3=CCOCC3)nc2n1CCOc1ccc(C(F)(F)F)cc1Cl] | 689. |
| 34 | 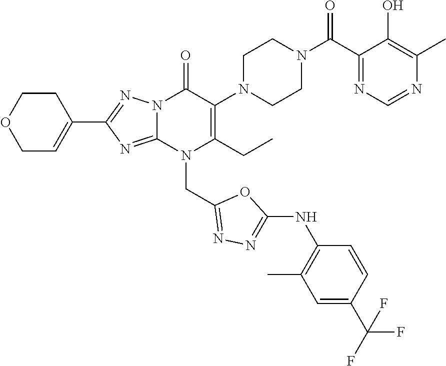 [SMILES: CCc1c(N2CCN(C(=O)c3ncnc(C)c3O)CC2)c(=O)n2nc(C3=CCOCC3)nc2n1Cc1nnc(Nc2ccc(C(F)(F)F)cc2C)o1] | 722.3 |
| 35 | 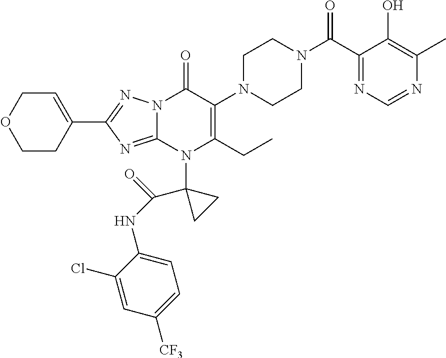 [SMILES: CCc1c(N2CCN(C(=O)c3ncnc(C)c3O)CC2)c(=O)n2nc(C3=CCOCC3)nc2n1C1(C(=O)Nc2ccc(C(F)(F)F)cc2Cl)CC1] | 728.2 |
| 36 | 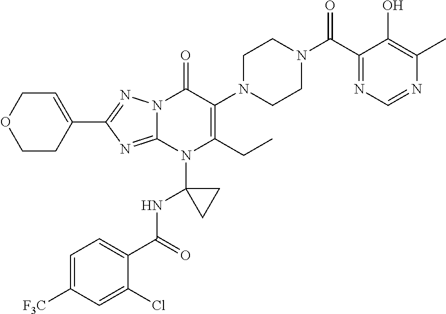 [SMILES: CCc1c(N2CCN(C(=O)c3ncnc(C)c3O)CC2)c(=O)n2nc(C3=CCOCC3)nc2n1C1(NC(=O)c2ccc(C(F)(F)F)cc2Cl)CC1] | 728.2 |
| 37 | 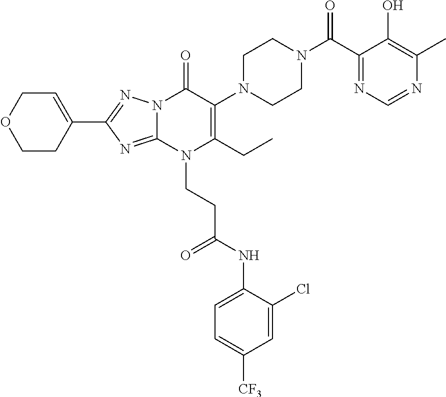 [SMILES: CCc1c(N2CCN(C(=O)c3ncnc(C)c3O)CC2)c(=O)n2nc(C3=CCOCC3)nc2n1CCC(=O)Nc1ccc(C(F)(F)F)cc1Cl] | 716.2 |
| 38 | 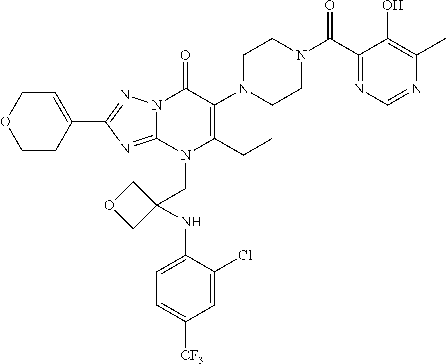 [SMILES: CCc1c(N2CCN(C(=O)c3ncnc(C)c3O)CC2)c(=O)n2nc(C3=CCOCC3)nc2n1CC1(Nc2ccc(C(F)(F)F)cc2Cl)COC1] | 730.2 |
| 39 | 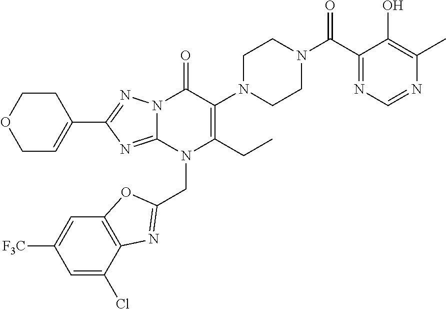 [SMILES: CCc1c(N2CCN(C(=O)c3ncnc(C)c3O)CC2)c(=O)n2nc(C3=CCOCC3)nc2n1Cc1nc2c(Cl)cc(C(F)(F)F)cc2o1] | 670.2 |
| 40 | 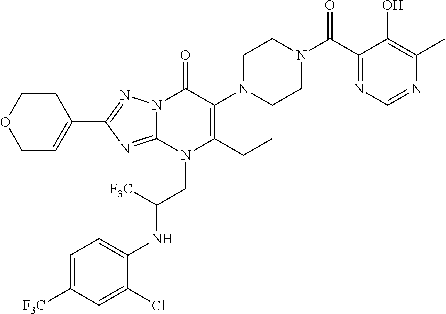 [SMILES: CCc1c(N2CCN(C(=O)c3ncnc(C)c3O)CC2)c(=O)n2nc(C3=CCOCC3)nc2n1CC(Nc1ccc(C(F)(F)F)cc1Cl)C(F)(F)F] | 756.2 |
| 41 | 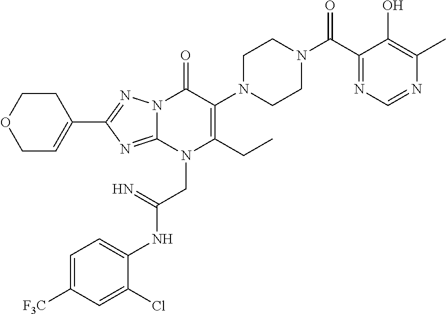 [SMILES: CCc1c(N2CCN(C(=O)c3ncnc(C)c3O)CC2)c(=O)n2nc(C3=CCOCC3)nc2n1CC(=N)Nc1ccc(C(F)(F)F)cc1Cl] | 701.2 |
| 42 | 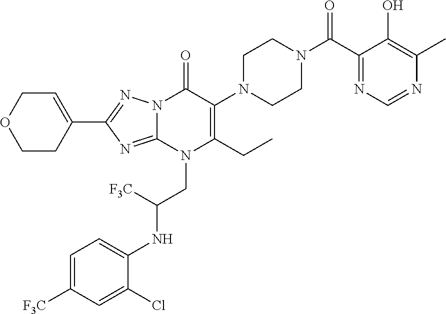 [SMILES: CCc1c(N2CCN(C(=O)c3ncnc(C)c3O)CC2)c(=O)n2nc(C3=CCOCC3)nc2n1CC(Nc1ccc(C(F)(F)F)cc1Cl)C(F)(F)F] | 756.2 |
| 43 | 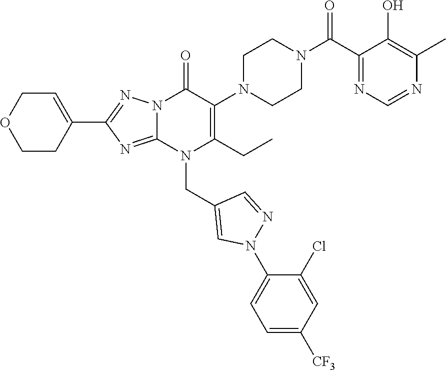 [SMILES: CCc1c(N2CCN(C(=O)c3ncnc(C)c3O)CC2)c(=O)n2nc(C3=CCOCC3)nc2n1Cc1cnn(-c2ccc(C(F)(F)F)cc2Cl)c1] | 725.2 |
| 44 | 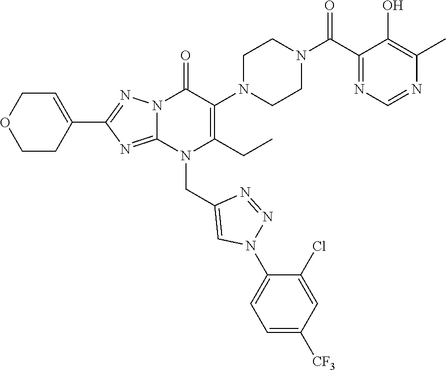 [SMILES: CCc1c(N2CCN(C(=O)c3ncnc(C)c3O)CC2)c(=O)n2nc(C3=CCOCC3)nc2n1Cc1cn(-c2ccc(C(F)(F)F)cc2Cl)nn1] | 726.2 |
| 45 | 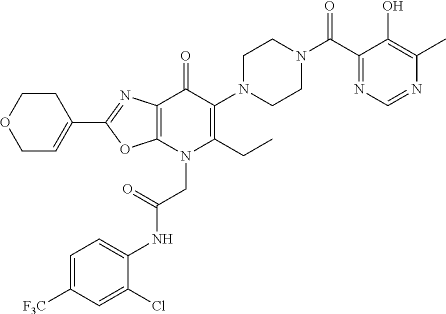 [SMILES: CCc1c(N2CCN(C(=O)c3ncnc(C)c3O)CC2)c(=O)c2nc(C3=CCOCC3)oc2n1CC(=O)Nc1ccc(C(F)(F)F)cc1Cl] | 702.2 |
| 46 | 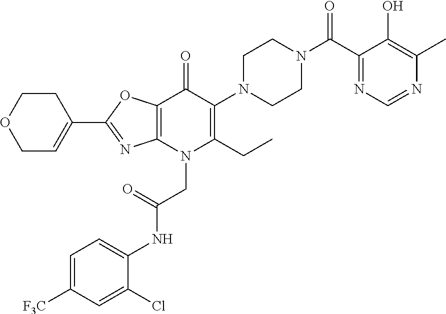 [SMILES: CCc1c(N2CCN(C(=O)c3ncnc(C)c3O)CC2)c(=O)c2oc(C3=CCOCC3)nc2n1CC(=O)Nc1ccc(C(F)(F)F)cc1Cl] | 702.2 |
| 47 | 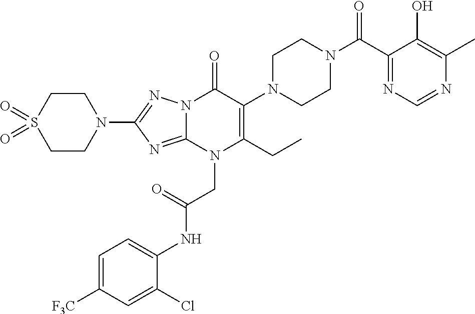 [SMILES: CCc1c(N2CCN(C(=O)c3ncnc(C)c3O)CC2)c(=O)n2nc(N3CCS(=O)(=O)CC3)nc2n1CC(=O)Nc1ccc(C(F)(F)F)cc1Cl] | 753.2 |
| 48 | 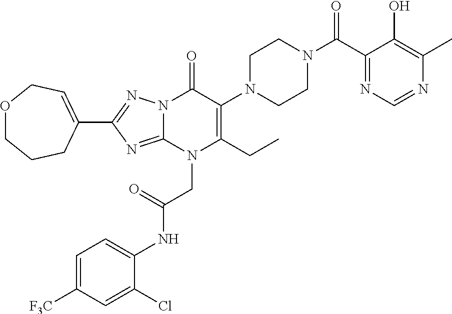 [SMILES: CCc1c(N2CCN(C(=O)c3ncnc(C)c3O)CC2)c(=O)n2nc(C3=CCOCCC3)nc2n1CC(=O)Nc1ccc(C(F)(F)F)cc1Cl] | 716.2 |
| 49 | 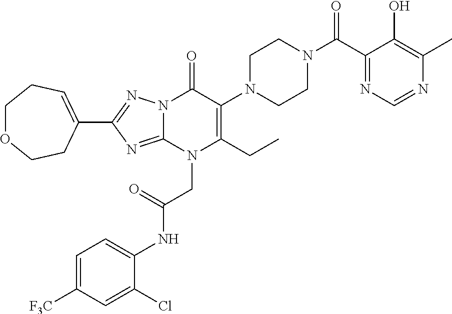 [SMILES: CCc1c(N2CCN(C(=O)c3ncnc(C)c3O)CC2)c(=O)n2nc(C3=CCCOCC3)nc2n1CC(=O)Nc1ccc(C(F)(F)F)cc1Cl] | 716.2 |
| 50 | 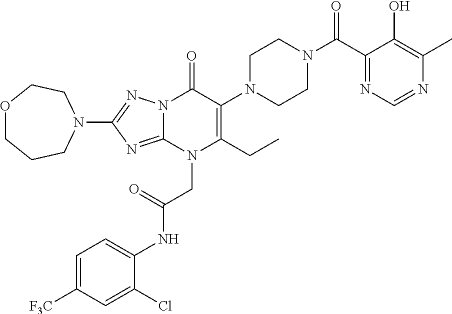 [SMILES: CCc1c(N2CCN(C(=O)c3ncnc(C)c3O)CC2)c(=O)n2nc(N3CCCOCC3)nc2n1CC(=O)Nc1ccc(C(F)(F)F)cc1Cl] | 719.2 |
| 51 | 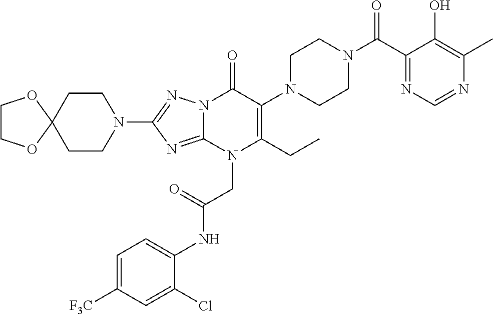 [SMILES: CCc1c(N2CCN(C(=O)c3ncnc(C)c3O)CC2)c(=O)n2nc(N3CCC4(CC3)OCCO4)nc2n1CC(=O)Nc1ccc(C(F)(F)F)cc1Cl] | 761.2 |
| 52 | 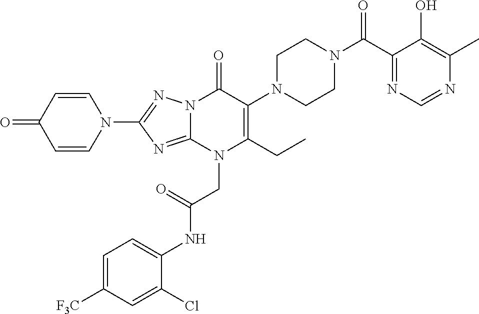 [SMILES: CCc1c(N2CCN(C(=O)c3ncnc(C)c3O)CC2)c(=O)n2nc(-n3ccc(=O)cc3)nc2n1CC(=O)Nc1ccc(C(F)(F)F)cc1Cl] | 713.2 |
| 53 | 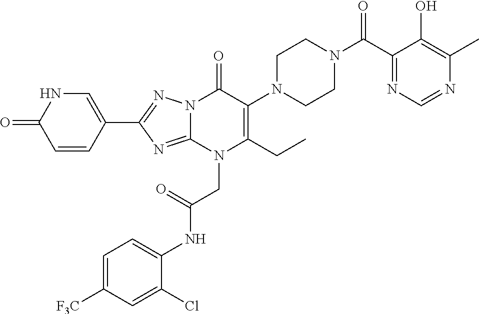 [SMILES: CCc1c(N2CCN(C(=O)c3ncnc(C)c3O)CC2)c(=O)n2nc(-c3ccc(=O)[nH]c3)nc2n1CC(=O)Nc1ccc(C(F)(F)F)cc1Cl] | 713.2 |
| 54 | 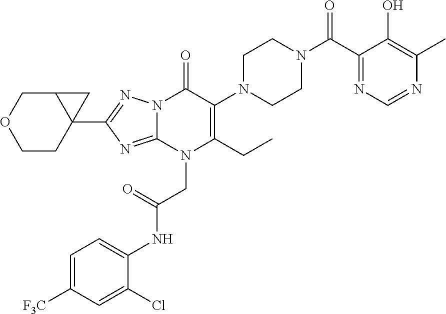 [SMILES: CCc1c(N2CCN(C(=O)c3ncnc(C)c3O)CC2)c(=O)n2nc(C34CCOCC3C4)nc2n1CC(=O)Nc1ccc(C(F)(F)F)cc1Cl] | 716.2 |
| 55 | 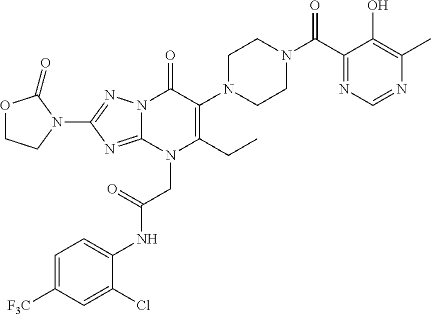 [SMILES: CCc1c(N2CCN(C(=O)c3ncnc(C)c3O)CC2)c(=O)n2nc(N3CCOC3=O)nc2n1CC(=O)Nc1ccc(C(F)(F)F)cc1Cl] | 705.2 |
| 56 | 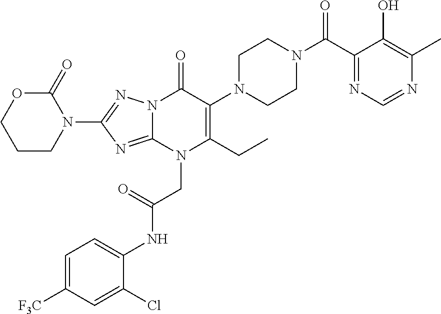 [SMILES: CCc1c(N2CCN(C(=O)c3ncnc(C)c3O)CC2)c(=O)n2nc(N3CCCOC3=O)nc2n1CC(=O)Nc1ccc(C(F)(F)F)cc1Cl] | 719.2 |
| 57 | 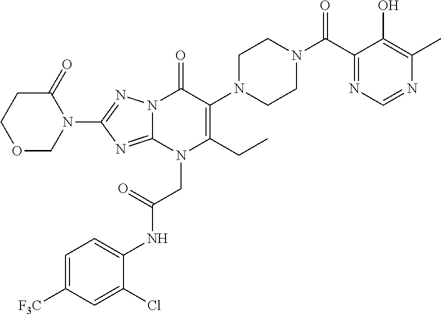 [SMILES: CCc1c(N2CCN(C(=O)c3ncnc(C)c3O)CC2)c(=O)n2nc(N3COCCC3=O)nc2n1CC(=O)Nc1ccc(C(F)(F)F)cc1Cl] | 719.2 |
| 58 | 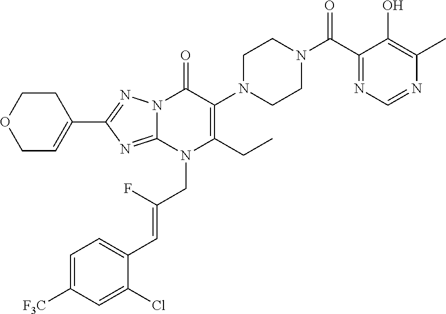 [SMILES: CCc1c(N2CCN(C(=O)c3ncnc(C)c3O)CC2)c(=O)n2nc(C3=CCOCC3)nc2n1C/C(F)=C/c1ccc(C(F)(F)F)cc1Cl] | 703.2 |
| 59 | 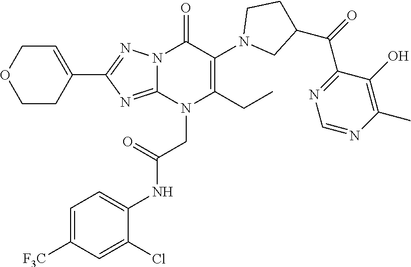 [SMILES: CCc1c(N2CCC(C(=O)c3ncnc(C)c3O)C2)c(=O)n2nc(C3=CCOCC3)nc2n1CC(=O)Nc1ccc(C(F)(F)F)cc1Cl] | 687.2 |
| 60 | 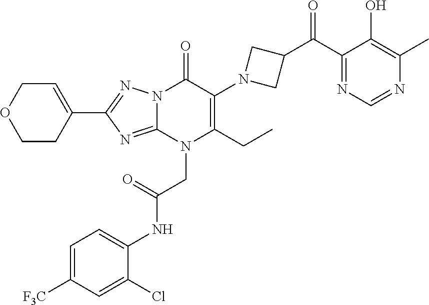 [SMILES: CCc1c(N2CC(C(=O)c3ncnc(C)c3O)C2)c(=O)n2nc(C3=CCOCC3)nc2n1CC(=O)Nc1ccc(C(F)(F)F)cc1Cl] | 673.2 |
| 61 | 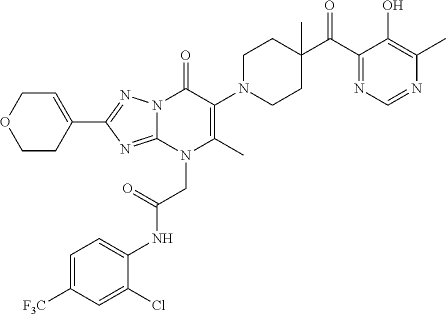 [SMILES: Cc1ncnc(C(=O)C2(C)CCN(c3c(C)n(CC(=O)Nc4ccc(C(F)(F)F)cc4Cl)c4nc(C5=CCOCC5)nn4c3=O)CC2)c1O] | 701.2 |
| 62 | 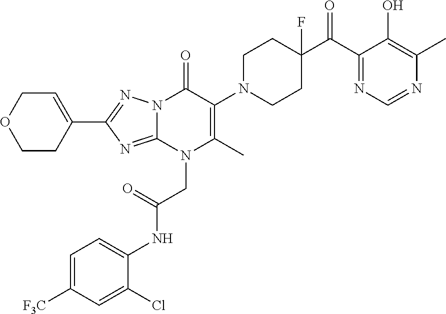 [SMILES: Cc1ncnc(C(=O)C2(F)CCN(c3c(C)n(CC(=O)Nc4ccc(C(F)(F)F)cc4Cl)c4nc(C5=CCOCC5)nn4c3=O)CC2)c1O] | 705.2 |
| 63 | 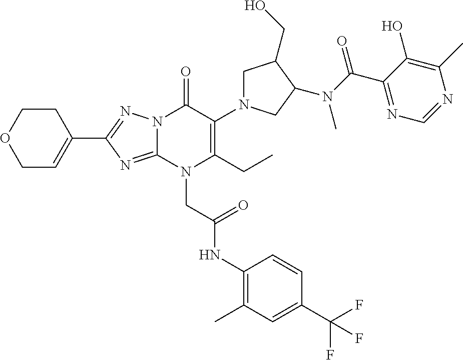 [SMILES: CCc1c(N2CC(CO)C(N(C)C(=O)c3ncnc(C)c3O)C2)c(=O)n2nc(C3=CCOCC3)nc2n1CC(=O)Nc1ccc(C(F)(F)F)cc1C] | 726.3 |
| 64 | 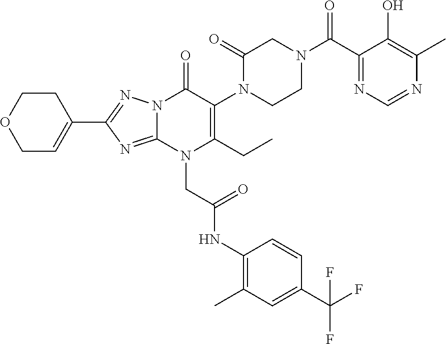 [SMILES: CCc1c(N2CCN(C(=O)c3ncnc(C)c3O)CC2=O)c(=O)n2nc(C3=CCOCC3)nc2n1CC(=O)Nc1ccc(C(F)(F)F)cc1C] | 796.2 |
| 65 | 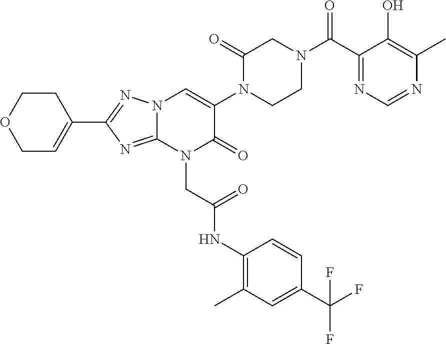 [SMILES: Cc1cc(C(F)(F)F)ccc1NC(=O)Cn1c(=O)c(N2CCN(C(=O)c3ncnc(C)c3O)CC2=O)cn2nc(C3=CCOCC3)nc12] | 668.2 |
| 66 | 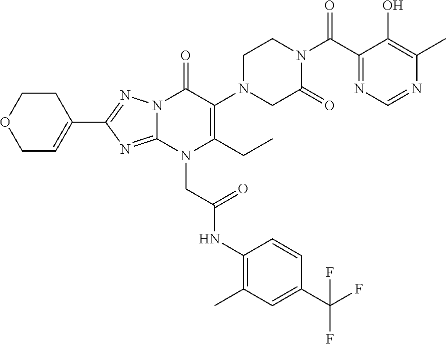 [SMILES: CCc1c(N2CCN(C(=O)c3ncnc(C)c3O)C(=O)C2)c(=O)n2nc(C3=CCOCC3)nc2n1CC(=O)Nc1ccc(C(F)(F)F)cc1C] | 682.3 |
| 67 | 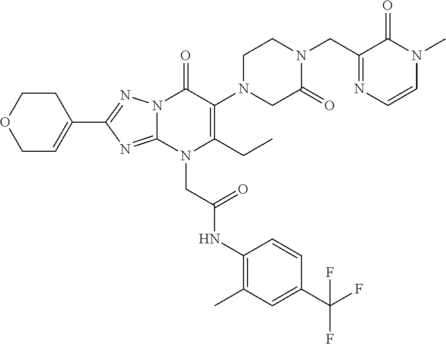 [SMILES: CCc1c(N2CCN(Cc3nccn(C)c3=O)C(=O)C2)c(=O)n2nc(C3=CCOCC3)nc2n1CC(=O)Nc1ccc(C(F)(F)F)cc1C] | 682.3 |
| 68 | 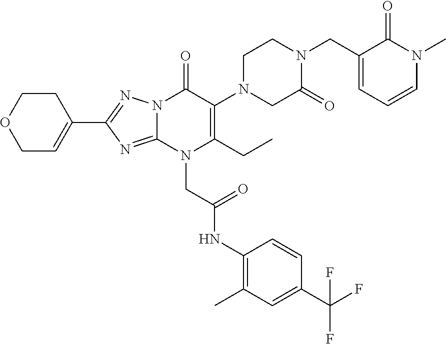 [SMILES: CCc1c(N2CCN(Cc3cccn(C)c3=O)C(=O)C2)c(=O)n2nc(C3=CCOCC3)nc2n1CC(=O)Nc1ccc(C(F)(F)F)cc1C] | 681.3 |
| 69 | 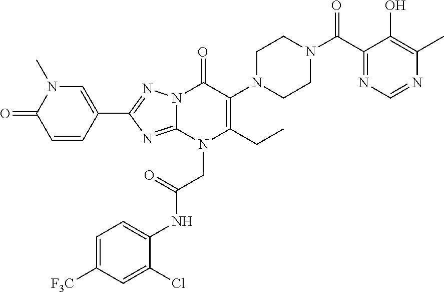 [SMILES: CCc1c(N2CCN(C(=O)c3ncnc(C)c3O)CC2)c(=O)n2nc(-c3ccc(=O)n(C)c3)nc2n1CC(=O)Nc1ccc(C(F)(F)F)cc1Cl] | 727.2 |
| 70 | 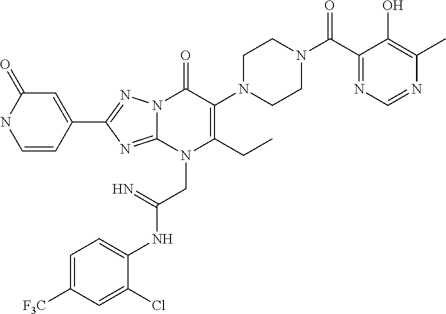 [SMILES: CCc1c(N2CCN(C(=O)c3ncnc(C)c3O)CC2)c(=O)n2nc(-c3cc[nH]c(=O)c3)nc2n1CC(=N)Nc1ccc(C(F)(F)F)cc1Cl] | 712.2 |
| 71 | 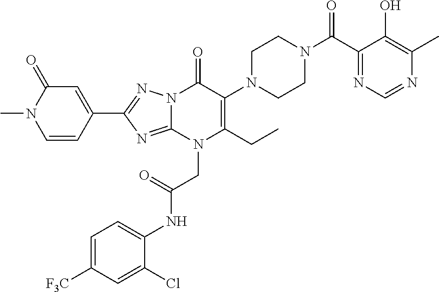 | 727.2 |
| 72 | 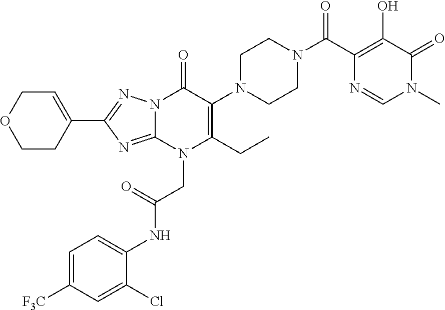 [SMILES: CCc1c(N2CCN(C(=O)c3ncn(C)c(=O)c3O)CC2)c(=O)n2nc(C3=CCOCC3)nc2n1CC(=O)Nc1ccc(C(F)(F)F)cc1Cl] | 718.2 |
| 73 | 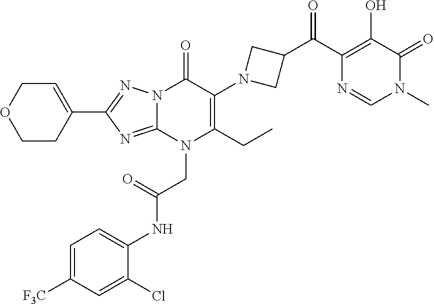 [SMILES: CCc1c(N2CC(C(=O)c3ncn(C)c(=O)c3O)C2)c(=O)n2nc(C3=CCOCC3)nc2n1CC(=O)Nc1ccc(C(F)(F)F)cc1Cl] | 689.2 |
| 74 | 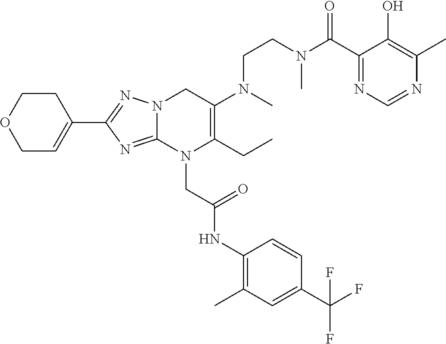 [SMILES: CCC1=C(N(C)CCN(C)C(=O)c2ncnc(C)c2O)Cn2nc(C3=CCOCC3)nc2N1CC(=O)Nc1ccc(C(F)(F)F)cc1C] | 684.3 |
| 75 | 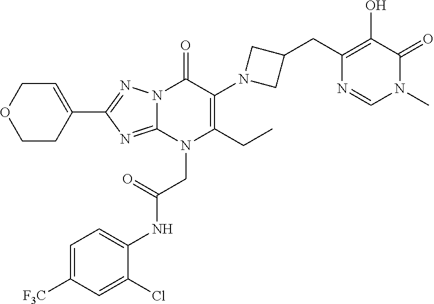 [SMILES: CCc1c(N2CC(Cc3ncn(C)c(=O)c3O)C2)c(=O)n2nc(C3=CCOCC3)nc2n1CC(=O)Nc1ccc(C(F)(F)F)cc1Cl] | 675.2 |
| 76 | 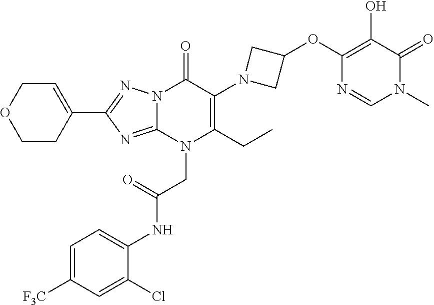 [SMILES: CCc1c(N2CC(Oc3ncn(C)c(=O)c3O)C2)c(=O)n2nc(C3=CCOCC3)nc2n1CC(=O)Nc1ccc(C(F)(F)F)cc1Cl] | 677.2 |
| 77 | 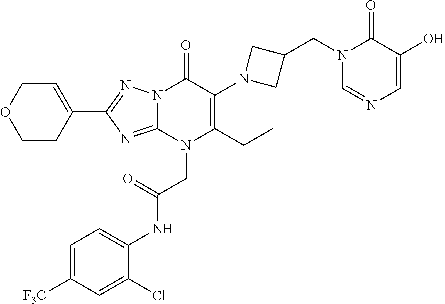 [SMILES: CCc1c(N2CC(Cn3cncc(O)c3=O)C2)c(=O)n2nc(C3=CCOCC3)nc2n1CC(=O)Nc1ccc(C(F)(F)F)cc1Cl] | 661.2 |
| 78 | 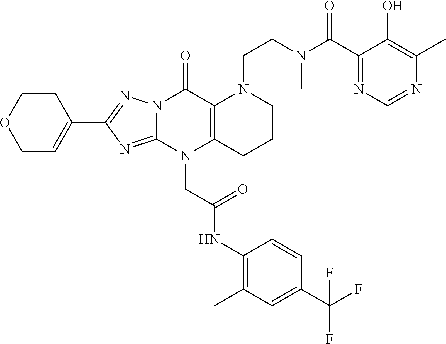 [SMILES: Cc1cc(C(F)(F)F)ccc1NC(=O)Cn1c2c(c(=O)n3nc(C4=CCOCC4)nc13)N(CCN(C)C(=O)c1ncnc(C)c1O)CCC2] | 682.3 |
| 79 | 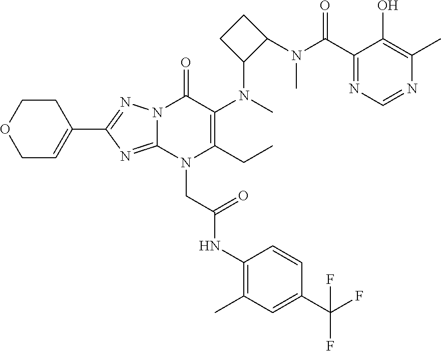 [SMILES: CCc1c(N(C)C2CCC2N(C)C(=O)c2ncnc(C)c2O)c(=O)n2nc(C3=CCOCC3)nc2n1CC(=O)Nc1ccc(C(F)(F)F)cc1C] | 710.3 |
| 80 | 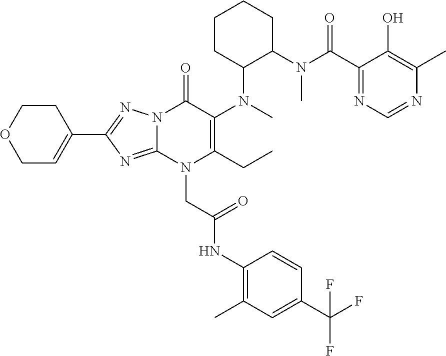 [SMILES: CCc1c(N(C)C2CCCCC2N(C)C(=O)c2ncnc(C)c2O)c(=O)n2nc(C3=CCOCC3)nc2n1CC(=O)Nc1ccc(C(F)(F)F)cc1C] | 738.3 |
| 81 | 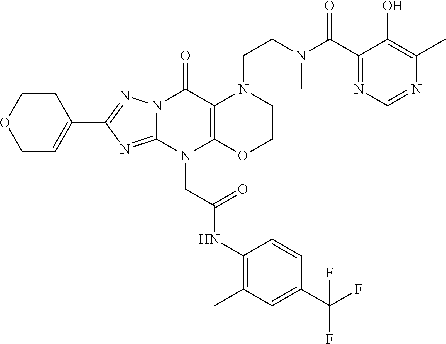 [SMILES: Cc1cc(C(F)(F)F)ccc1NC(=O)Cn1c2c(c(=O)n3nc(C4=CCOCC4)nc13)N(CCN(C)C(=O)c1ncnc(C)c1O)CCO2] | 684.3 |
| 82 | 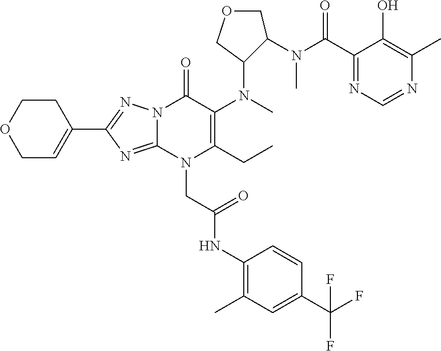 [SMILES: CCc1c(N(C)C2COCC2N(C)C(=O)c2ncnc(C)c2O)c(=O)n2nc(C3=CCOCC3)nc2n1CC(=O)Nc1ccc(C(F)(F)F)cc1C] | 726.3 |
| 83 | 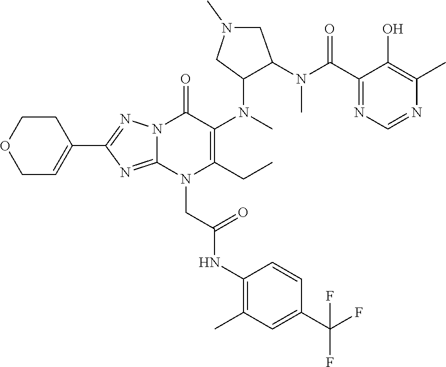 [SMILES: CCc1c(N(C)C2CN(C)CC2N(C)C(=O)c2ncnc(C)c2O)c(=O)n2nc(C3=CCOCC3)nc2n1CC(=O)Nc1ccc(C(F)(F)F)cc1C] | 739.3 |
| 84 | 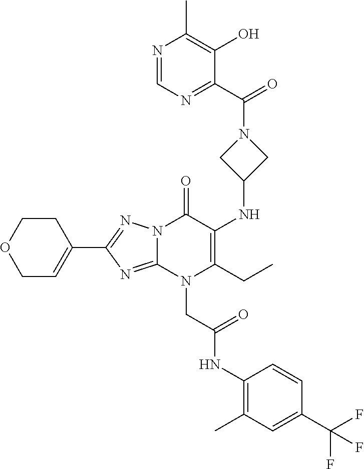 [SMILES: CCc1c(NC2CN(C(=O)c3ncnc(C)c3O)C2)c(=O)n2nc(C3=CCOCC3)nc2n1CC(=O)Nc1ccc(C(F)(F)F)cc1C] | 668.2 |
| 85 | 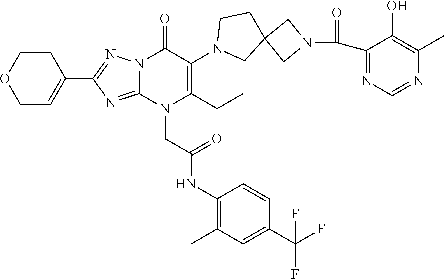 [SMILES: CCc1c(N2CCC3(CN(C(=O)c4ncnc(C)c4O)C3)C2)c(=O)n2nc(C3=CCOCC3)nc2n1CC(=O)Nc1ccc(C(F)(F)F)cc1C] | 708.3 |
| 86 | 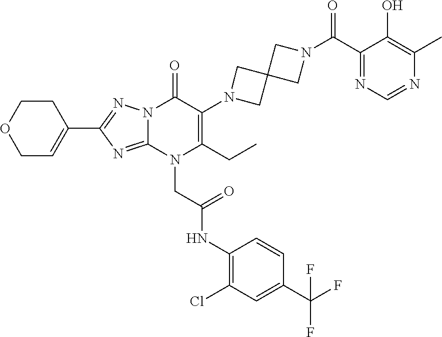 [SMILES: CCc1c(N2CC3(CN(C(=O)c4ncnc(C)c4O)C3)C2)c(=O)n2nc(C3=CCOCC3)nc2n1CC(=O)Nc1ccc(C(F)(F)F)cc1Cl] | 714.2 |
| 87 | 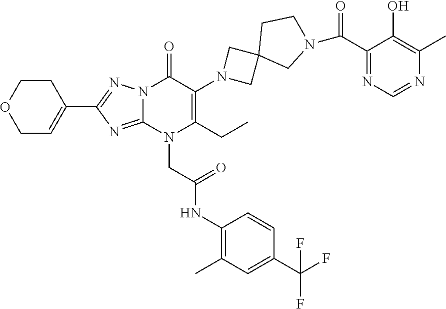 [SMILES: CCc1c(N2CC3(CCN(C(=O)c4ncnc(C)c4O)C3)C2)c(=O)n2nc(C3=CCOCC3)nc2n1CC(=O)Nc1ccc(C(F)(F)F)cc1C] | 708.3 |
| 88 | 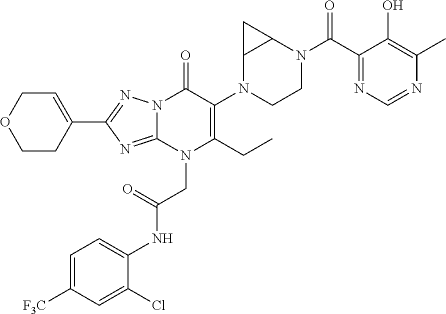 [SMILES: CCc1c(N2CCN(C(=O)c3ncnc(C)c3O)C3CC32)c(=O)n2nc(C3=CCOCC3)nc2n1CC(=O)Nc1ccc(C(F)(F)F)cc1Cl] | 714.2 |
| 89 | 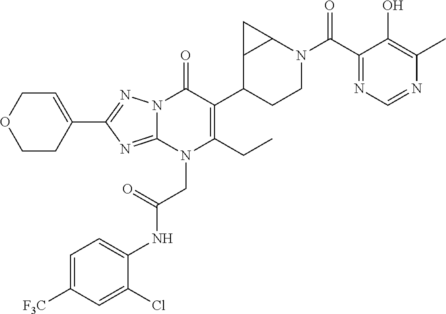 [SMILES: CCc1c(C2CCN(C(=O)c3ncnc(C)c3O)C3CC23)c(=O)n2nc(C3=CCOCC3)nc2n1CC(=O)Nc1ccc(C(F)(F)F)cc1Cl] | 713.2 |
| 90 | 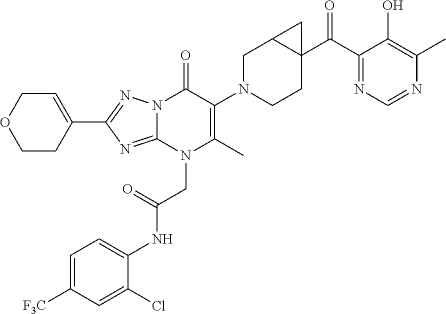 [SMILES: Cc1ncnc(C(=O)C23CCN(c4c(C)n(CC(=O)Nc5ccc(C(F)(F)F)cc5Cl)c5nc(C6=CCOCC6)nn5c4=O)CC2C3)c1O] | 699.2 |
| 91 | 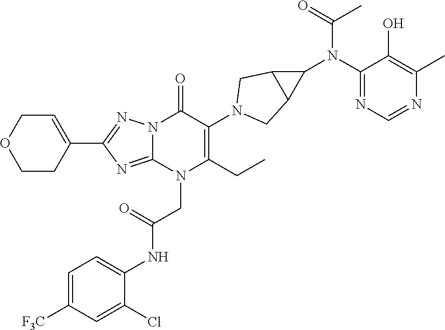 [SMILES: CCc1c(N2CC3C(C2)C3N(C(C)=O)c2ncnc(C)c2O)c(=O)n2nc(C3=CCOCC3)nc2n1CC(=O)Nc1ccc(C(F)(F)F)cc1Cl] | 728.2 |
| 92 | 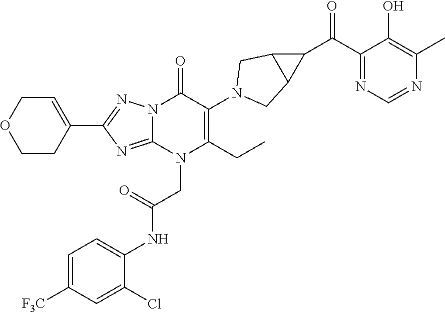 [SMILES: CCc1c(N2CC3C(C2)C3C(=O)c2ncnc(C)c2O)c(=O)n2nc(C3=CCOCC3)nc2n1CC(=O)Nc1ccc(C(F)(F)F)cc1Cl] | 699.2 |
| 93 | 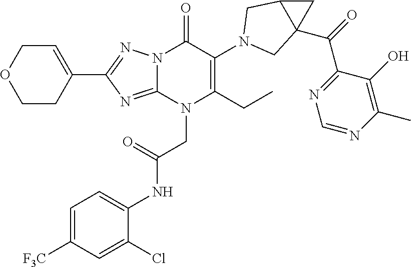 [SMILES: CCc1c(N2CC3CC3(C(=O)c3ncnc(C)c3O)C2)c(=O)n2nc(C3=CCOCC3)nc2n1CC(=O)Nc1ccc(C(F)(F)F)cc1Cl] | 699.2 |
| 94 | 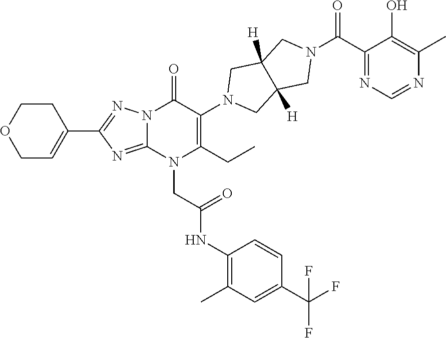 [SMILES: CCc1c(N2C[C@H]3CN(C(=O)c4ncnc(C)c4O)C[C@H]3C2)c(=O)n2nc(C3=CCOCC3)nc2n1CC(=O)Nc1ccc(C(F)(F)F)cc1C] | 708.3 |
| 95 | 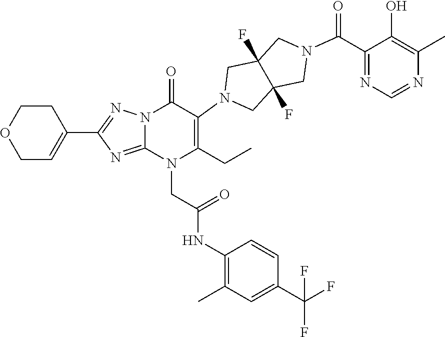 [SMILES: CCc1c(N2C[C@]3(F)CN(C(=O)c4ncnc(C)c4O)C[C@]3(F)C2)c(=O)n2nc(C3=CCOCC3)nc2n1CC(=O)Nc1ccc(C(F)(F)F)cc1C] | 744.3 |
| 96 | 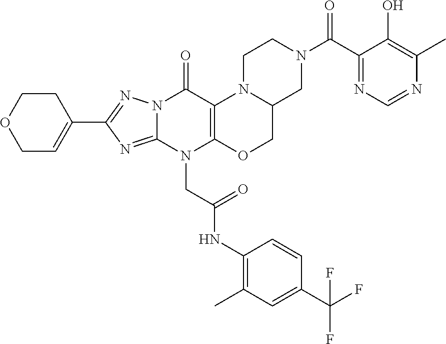 [SMILES: Cc1cc(C(F)(F)F)ccc1NC(=O)Cn1c2c(c(=O)n3nc(C4=CCOCC4)nc13)N1CCN(C(=O)c3ncnc(C)c3O)CC1CO2] | 682.2 |
| 97 | 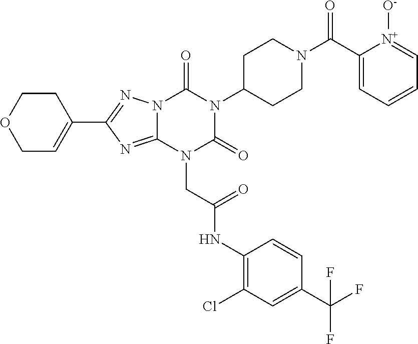 [SMILES: O=C(Cn1c(=O)n(C2CCN(C(=O)c3cccc[n+]3[O-])CC2)c(=O)n2nc(C3=CCOCC3)nc12)Nc1ccc(C(F)(F)F)cc1Cl] | 675.2 |
| 98 | 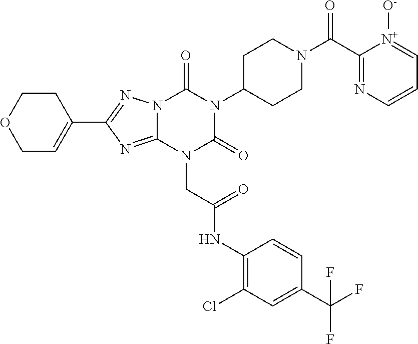 [SMILES: O=C(Cn1c(=O)n(C2CCN(C(=O)c3nccc[n+]3[O-])CC2)c(=O)n2nc(C3=CCOCC3)nc12)Nc1ccc(C(F)(F)F)cc1Cl] | 676.2 |
| 99 | 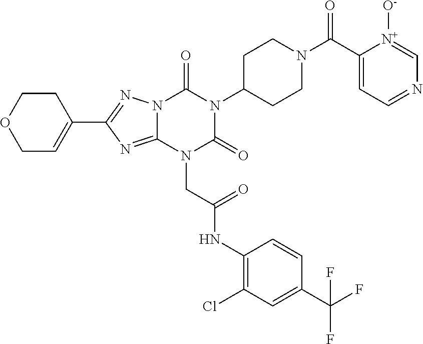 [SMILES: O=C(Cn1c(=O)n(C2CCN(C(=O)c3ccnc[n+]3[O-])CC2)c(=O)n2nc(C3=CCOCC3)nc12)Nc1ccc(C(F)(F)F)cc1Cl] | 676.2 |
| 100 | 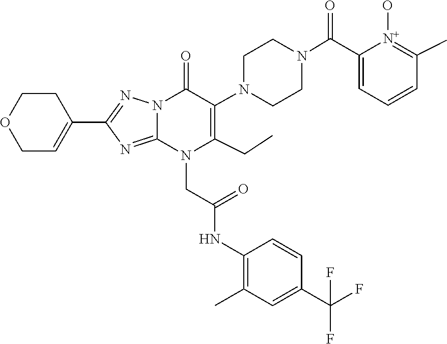 [SMILES: CCc1c(N2CCN(C(=O)c3cccc(C)[n+]3O)CC2)c(=O)n2nc(C3=CCOCC3)nc2n1CC(=O)Nc1ccc(C(F)(F)F)cc1C] | 681.3 |
| 101 | 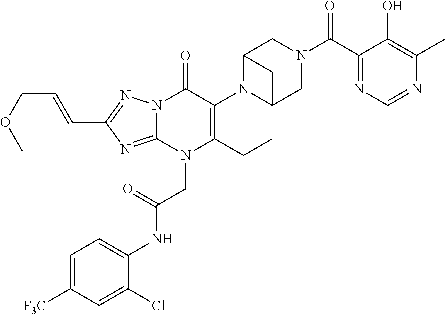 [SMILES: CCc1c(N2C3CC2CN(C(=O)c2ncnc(C)c2O)C3)c(=O)n2nc(/C=C/COC)nc2n1CC(=O)Nc1ccc(C(F)(F)F)cc1Cl] | 702.2 |
| 102 | 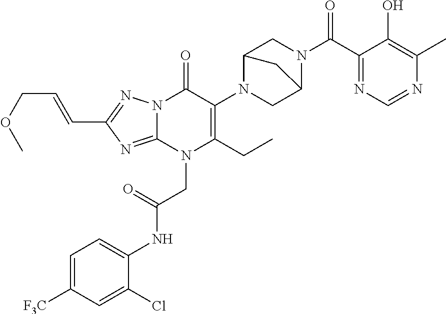 [SMILES: CCc1c(N2CC3CC2CN3C(=O)c2ncnc(C)c2O)c(=O)n2nc(/C=C/COC)nc2n1CC(=O)Nc1ccc(C(F)(F)F)cc1Cl] | 702.2 |
| 103 | 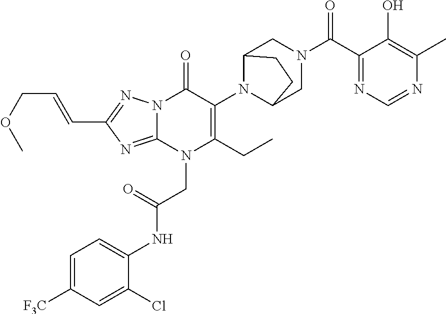 [SMILES: CCc1c(N2C3CCC2CN(C(=O)c2ncnc(C)c2O)C3)c(=O)n2nc(/C=C/COC)nc2n1CC(=O)Nc1ccc(C(F)(F)F)cc1Cl] | 716.2 |
| 104 | 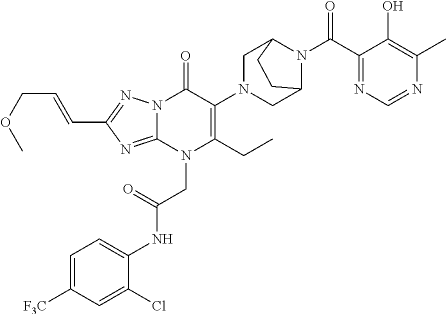 [SMILES: CCc1c(N2CC3CCC(C2)N3C(=O)c2ncnc(C)c2O)c(=O)n2nc(/C=C/COC)nc2n1CC(=O)Nc1ccc(C(F)(F)F)cc1Cl] | 716.2 |
| 105 | 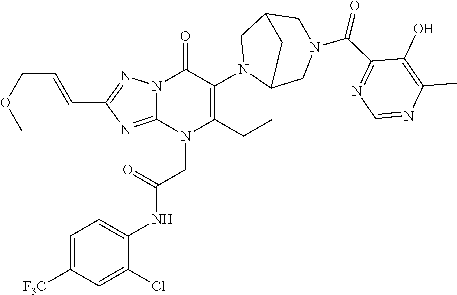 [SMILES: CCc1c(N2CC3CC2CN(C(=O)c2ncnc(C)c2O)C3)c(=O)n2nc(/C=C/COC)nc2n1CC(=O)Nc1ccc(C(F)(F)F)cc1Cl] | 716.2 |
| 106 | 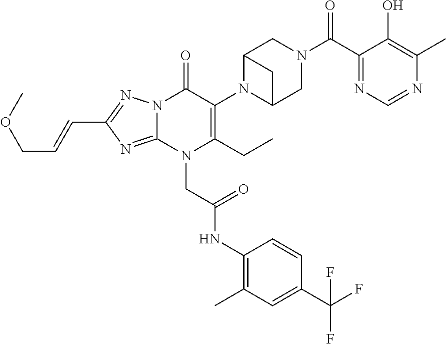 [SMILES: CCc1c(N2C3CC2CN(C(=O)c2ncnc(C)c2O)C3)c(=O)n2nc(/C=C/COC)nc2n1CC(=O)Nc1ccc(C(F)(F)F)cc1C] | 682.3 |
| 107 | 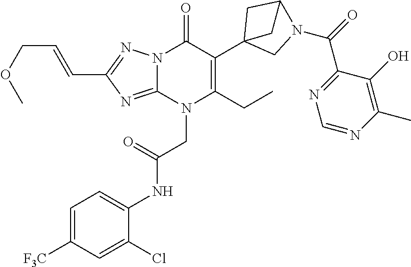 [SMILES: CCc1c(C23CC(C2)N(C(=O)c2ncnc(C)c2O)C3)c(=O)n2nc(/C=C/COC)nc2n1CC(=O)Nc1ccc(C(F)(F)F)cc1Cl] | 687.2 |
| 108 | 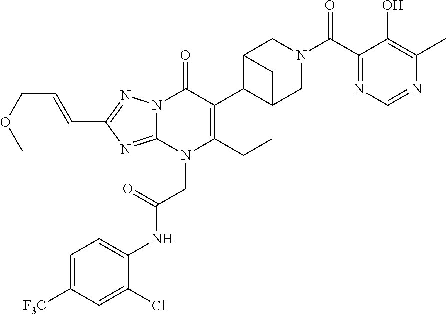 [SMILES: CCc1c(C2C3CC2CN(C(=O)c2ncnc(C)c2O)C3)c(=O)n2nc(/C=C/COC)nc2n1CC(=O)Nc1ccc(C(F)(F)F)cc1Cl] | 701.2 |
| 109 | 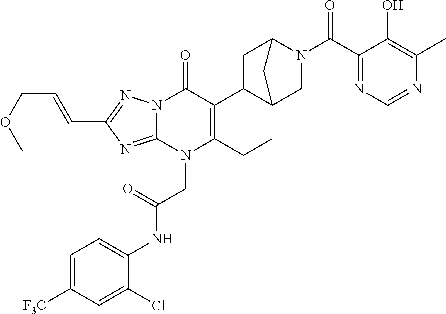 [SMILES: CCc1c(C2CC3CC2CN3C(=O)c2ncnc(C)c2O)c(=O)n2nc(/C=C/COC)nc2n1CC(=O)Nc1ccc(C(F)(F)F)cc1Cl] | 701.2 |
| 110 | 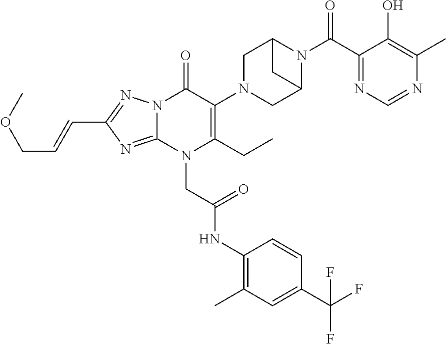 [SMILES: CCc1c(N2CC3CC(C2)N3C(=O)c2ncnc(C)c2O)c(=O)n2nc(/C=C/COC)nc2n1CC(=O)Nc1ccc(C(F)(F)F)cc1C] | 682.3 |
| 111 | 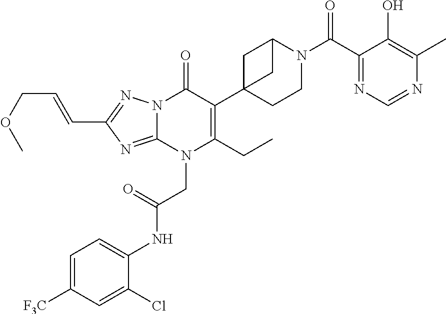 [SMILES: CCc1c(C23CCN(C(=O)c4ncnc(C)c4O)C(C2)C3)c(=O)n2nc(/C=C/COC)nc2n1CC(=O)Nc1ccc(C(F)(F)F)cc1Cl] | 701.2 |
| 112 | 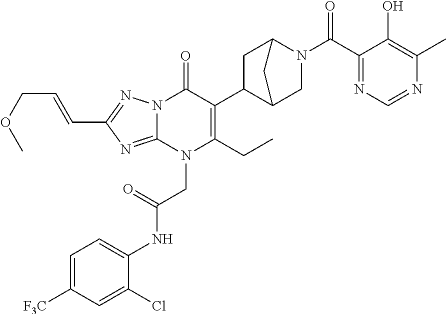 [SMILES: CCc1c(C2CC3CC2CN3C(=O)c2ncnc(C)c2O)c(=O)n2nc(/C=C/COC)nc2n1CC(=O)Nc1ccc(C(F)(F)F)cc1Cl] | 701.2 |
| 113 | 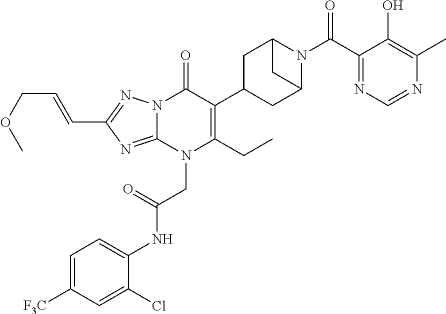 [SMILES: CCc1c(C2CC3CC(C2)N3C(=O)c2ncnc(C)c2O)c(=O)n2nc(/C=C/COC)nc2n1CC(=O)Nc1ccc(C(F)(F)F)cc1Cl] | 701.2 |
| 114 | 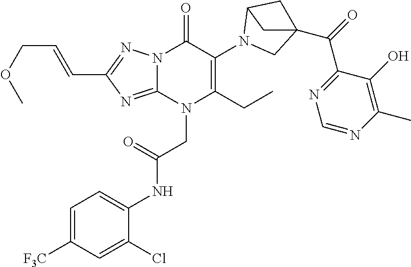 [SMILES: CCc1c(N2CC3(C(=O)c4ncnc(C)c4O)CC2C3)c(=O)n2nc(/C=C/COC)nc2n1CC(=O)Nc1ccc(C(F)(F)F)cc1Cl] | 687.2 |
| 115 | 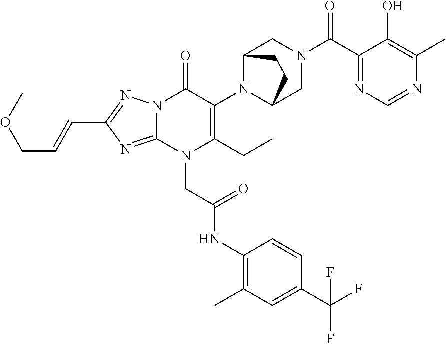 [SMILES: CCc1c(N2[C@@H]3CC[C@H]2CN(C(=O)c2ncnc(C)c2O)C3)c(=O)n2nc(/C=C/COC)nc2n1CC(=O)Nc1ccc(C(F)(F)F)cc1C] | 696.3 |
| 116 | 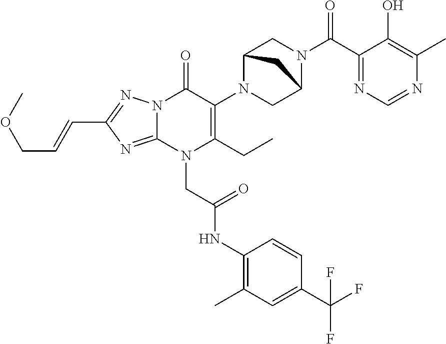 [SMILES: CCc1c(N2C[C@@H]3C[C@H]2CN3C(=O)c2ncnc(C)c2O)c(=O)n2nc(/C=C/COC)nc2n1CC(=O)Nc1ccc(C(F)(F)F)cc1C] | 682.3 |
| 117 | 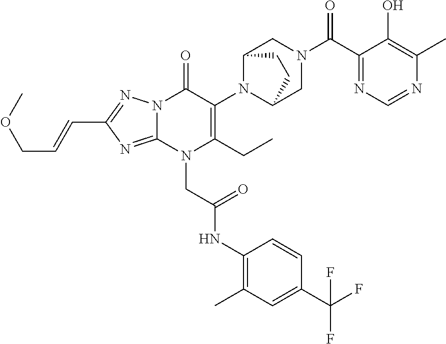 [SMILES: CCc1c(N2[C@@H]3CC[C@H]2CN(C(=O)c2ncnc(C)c2O)C3)c(=O)n2nc(/C=C/COC)nc2n1CC(=O)Nc1ccc(C(F)(F)F)cc1C] | 696.3 |
| 118 | 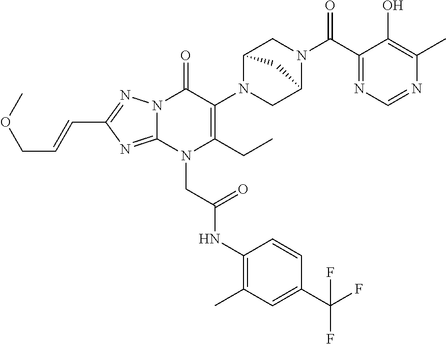 [SMILES: CCc1c(N2C[C@H]3C[C@@H]2CN3C(=O)c2ncnc(C)c2O)c(=O)n2nc(/C=C/COC)nc2n1CC(=O)Nc1ccc(C(F)(F)F)cc1C] | 682.3 |
| 119 | 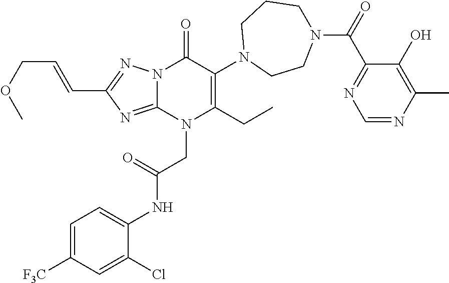 [SMILES: CCc1c(N2CCCN(C(=O)c3ncnc(C)c3O)CC2)c(=O)n2nc(/C=C/COC)nc2n1CC(=O)Nc1ccc(C(F)(F)F)cc1Cl] | 704.2 |
| 120 | 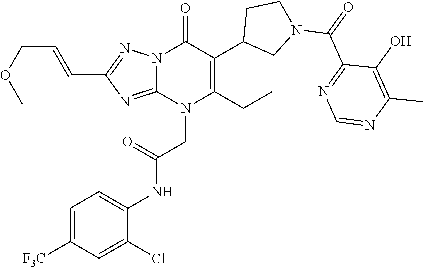 [SMILES: CCc1c(C2CCN(C(=O)c3ncnc(C)c3O)C2)c(=O)n2nc(/C=C/COC)nc2n1CC(=O)Nc1ccc(C(F)(F)F)cc1Cl] | 675.2 |
| 121 | 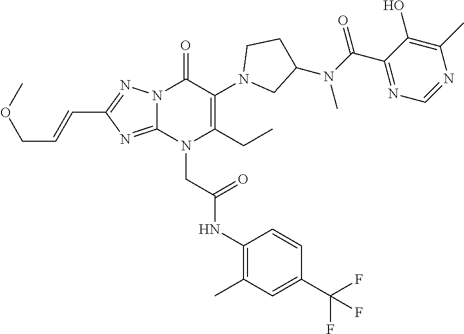 [SMILES: CCc1c(N2CCC(N(C)C(=O)c3ncnc(C)c3O)C2)c(=O)n2nc(/C=C/COC)nc2n1CC(=O)Nc1ccc(C(F)(F)F)cc1C] | 684.3 |
| 122 | 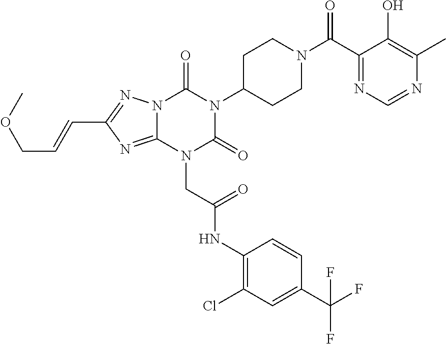 [SMILES: COC/C=C/c1nc2n(CC(=O)Nc3ccc(C(F)(F)F)cc3Cl)c(=O)n(C3CCN(C(=O)c4ncnc(C)c4O)CC3)c(=O)n2n1] | 678.2 |
| 123 | 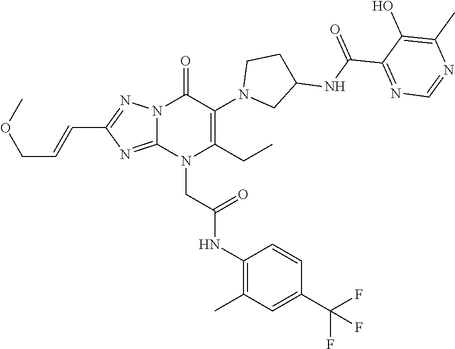 [SMILES: CCc1c(N2CCC(NC(=O)c3ncnc(C)c3O)C2)c(=O)n2nc(/C=C/COC)nc2n1CC(=O)Nc1ccc(C(F)(F)F)cc1C] | 670.3 |
| 124 | 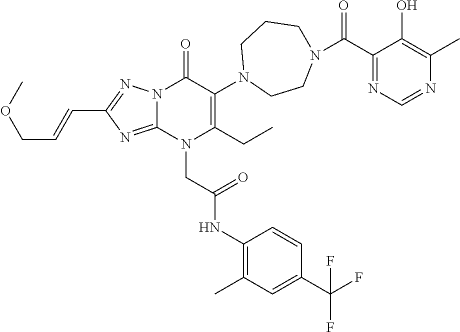 [SMILES: CCc1c(N2CCCN(C(=O)c3ncnc(C)c3O)CC2)c(=O)n2nc(/C=C/COC)nc2n1CC(=O)Nc1ccc(C(F)(F)F)cc1C] | 684.3 |
| 125 | 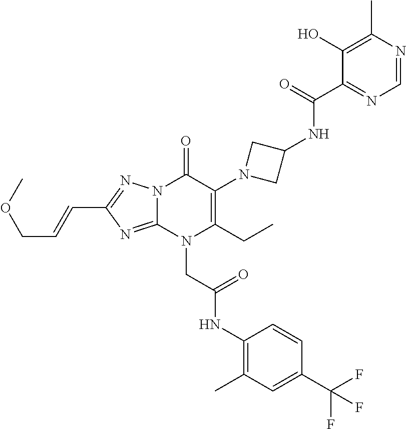 [SMILES: CCc1c(N2CC(NC(=O)c3ncnc(C)c3O)C2)c(=O)n2nc(/C=C/COC)nc2n1CC(=O)Nc1ccc(C(F)(F)F)cc1C] | 656.2 |
| 126 | 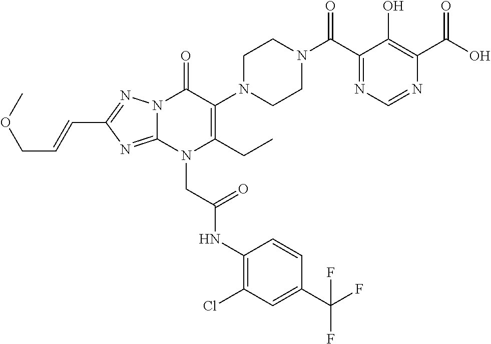 [SMILES: CCc1c(N2CCN(C(=O)c3ncnc(C(=O)O)c3O)CC2)c(=O)n2nc(/C=C/COC)nc2n1CC(=O)Nc1ccc(C(F)(F)F)cc1Cl] | 720.2 |
| 127 | 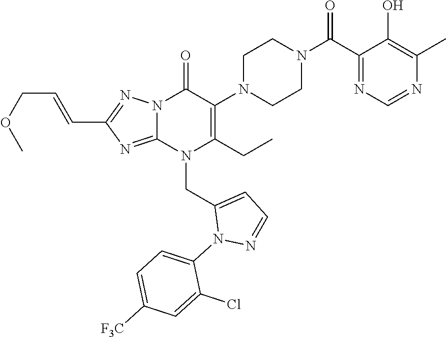 [SMILES: CCc1c(N2CCN(C(=O)c3ncnc(C)c3O)CC2)c(=O)n2nc(/C=C/COC)nc2n1Cc1ccnn1-c1ccc(C(F)(F)F)cc1Cl] | 713.2 |
| 128 | 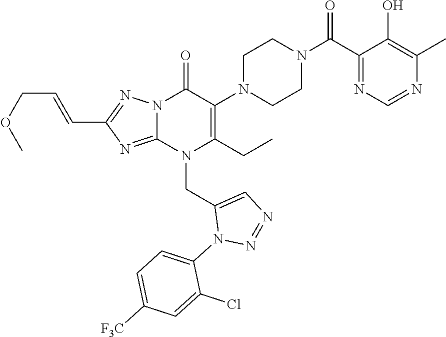 [SMILES: CCc1c(N2CCN(C(=O)c3ncnc(C)c3O)CC2)c(=O)n2nc(/C=C/COC)nc2n1Cc1cnnn1-c1ccc(C(F)(F)F)cc1Cl] | 714.2 |
| 129 | 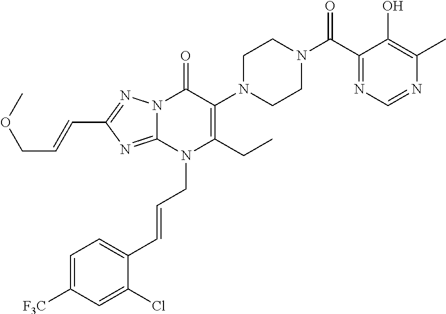 [SMILES: CCc1c(N2CCN(C(=O)c3ncnc(C)c3O)CC2)c(=O)n2nc(/C=C/COC)nc2n1C/C=C/c1ccc(C(F)(F)F)cc1Cl] | 673.2 |
| 130 | 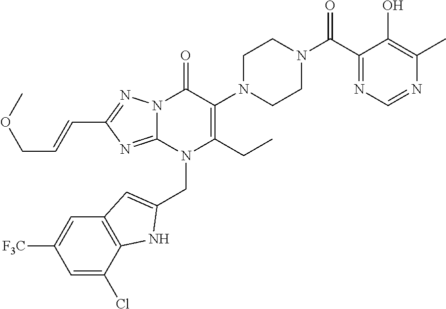 [SMILES: CCc1c(N2CCN(C(=O)c3ncnc(C)c3O)CC2)c(=O)n2nc(/C=C/COC)nc2n1Cc1cc2cc(C(F)(F)F)cc(Cl)c2[nH]1] | 686.2 |
| 131 | 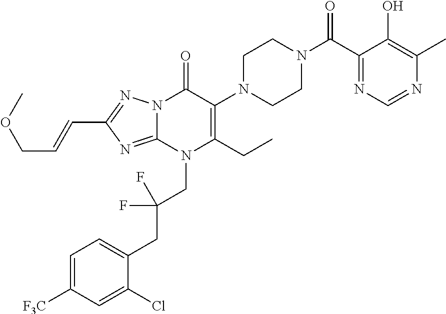 [SMILES: CCc1c(N2CCN(C(=O)c3ncnc(C)c3O)CC2)c(=O)n2nc(/C=C/COC)nc2n1CC(F)(F)Cc1ccc(C(F)(F)F)cc1Cl] | 711.2 |
| 132 | 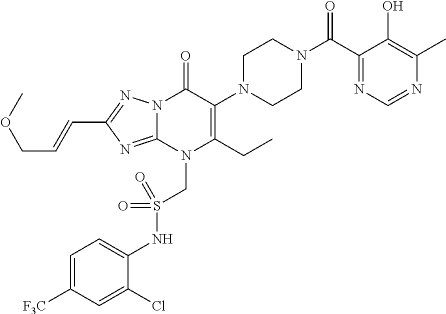 [SMILES: CCc1c(N2CCN(C(=O)c3ncnc(C)c3O)CC2)c(=O)n2nc(/C=C/COC)nc2n1CS(=O)(=O)Nc1ccc(C(F)(F)F)cc1Cl] | 726.2 |
| 133 | 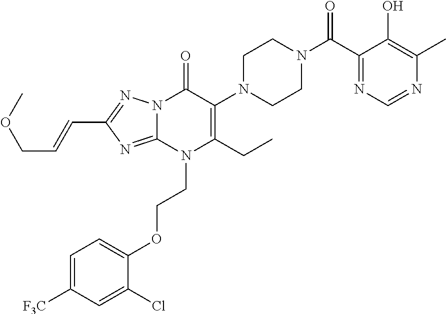 [SMILES: CCc1c(N2CCN(C(=O)c3ncnc(C)c3O)CC2)c(=O)n2nc(/C=C/COC)nc2n1CCOc1ccc(C(F)(F)F)cc1Cl] | 677.2 |
| 134 | 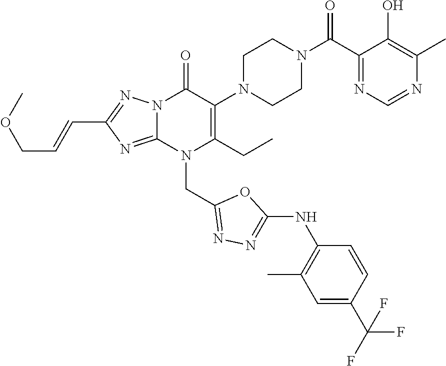 [SMILES: CCc1c(N2CCN(C(=O)c3ncnc(C)c3O)CC2)c(=O)n2nc(/C=C/COC)nc2n1Cc1nnc(Nc2ccc(C(F)(F)F)cc2C)o1] | 710.3 |
| 135 | 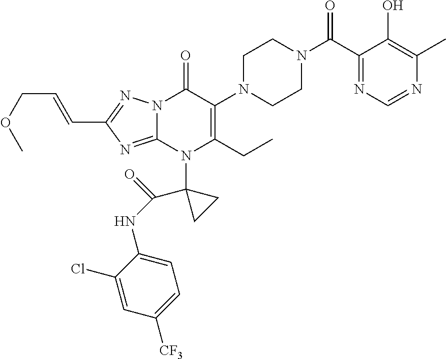 [SMILES: CCc1c(N2CCN(C(=O)c3ncnc(C)c3O)CC2)c(=O)n2nc(/C=C/COC)nc2n1C1(C(=O)Nc2ccc(C(F)(F)F)cc2Cl)CC1] | 716.2 |
| 136 | 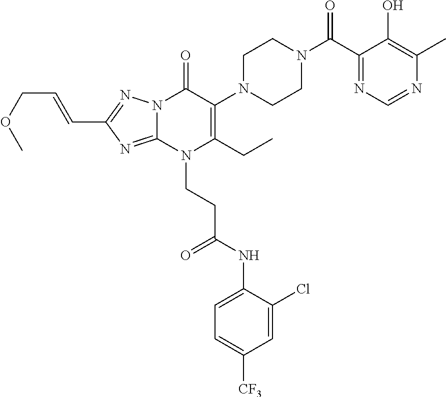 [SMILES: CCc1c(N2CCN(C(=O)c3ncnc(C)c3O)CC2)c(=O)n2nc(/C=C/COC)nc2n1CCC(=O)Nc1ccc(C(F)(F)F)cc1Cl] | 704.3 |
| 137 | 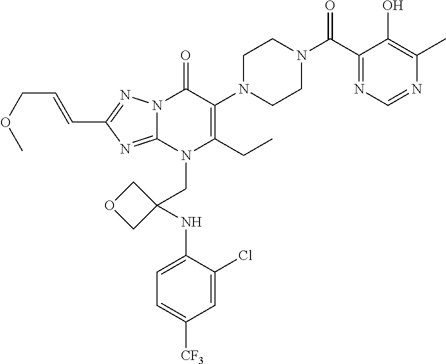 [SMILES: CCc1c(N2CCN(C(=O)c3ncnc(C)c3O)CC2)c(=O)n2nc(/C=C/COC)nc2n1CC1(Nc2ccc(C(F)(F)F)cc2Cl)COC1] | 718.2 |
| 138 | 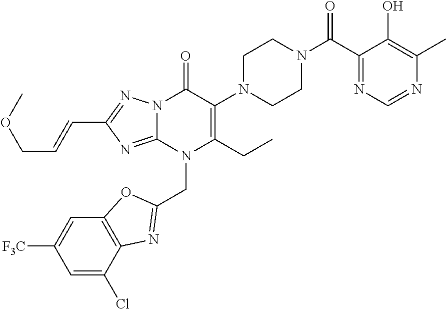 [SMILES: CCc1c(N2CCN(C(=O)c3ncnc(C)c3O)CC2)c(=O)n2nc(/C=C/COC)nc2n1Cc1nc2c(Cl)cc(C(F)(F)F)cc2o1] | 688.2 |
| 139 | 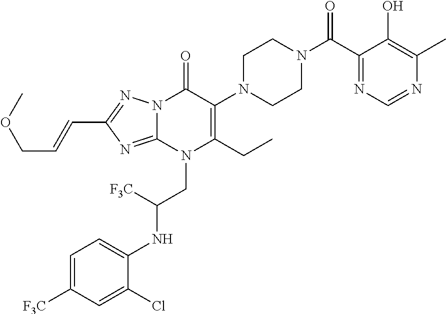 [SMILES: CCc1c(N2CCN(C(=O)c3ncnc(C)c3O)CC2)c(=O)n2nc(/C=C/COC)nc2n1CC(Nc1ccc(C(F)(F)F)cc1Cl)C(F)(F)F] | 744.2 |
| 140 | 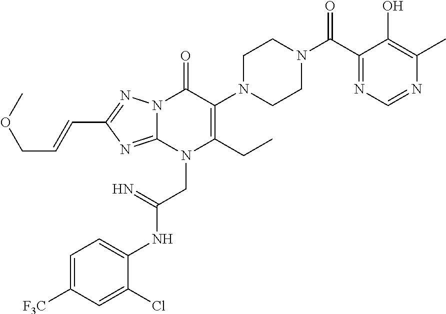 [SMILES: CCc1c(N2CCN(C(=O)c3ncnc(C)c3O)CC2)c(=O)n2nc(/C=C/COC)nc2n1CC(=N)Nc1ccc(C(F)(F)F)cc1Cl] | 689.2 |
| 141 | 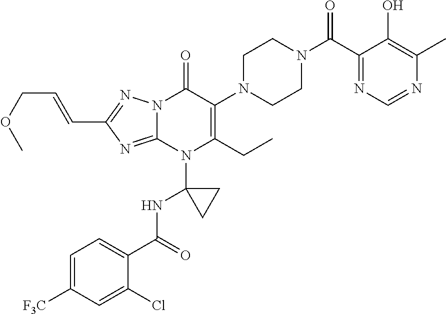 [SMILES: CCc1c(N2CCN(C(=O)c3ncnc(C)c3O)CC2)c(=O)n2nc(/C=C/COC)nc2n1C1(NC(=O)c2ccc(C(F)(F)F)cc2Cl)CC1] | 716.2 |
| 142 | 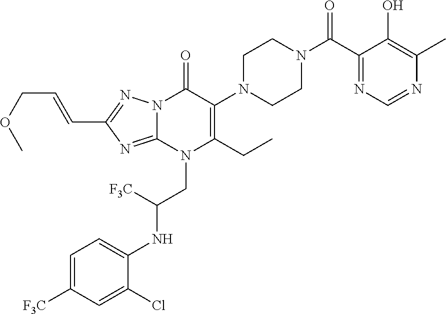 [SMILES: CCc1c(N2CCN(C(=O)c3ncnc(C)c3O)CC2)c(=O)n2nc(/C=C/COC)nc2n1CC(Nc1ccc(C(F)(F)F)cc1Cl)C(F)(F)F] | 744.2 |
| 143 | 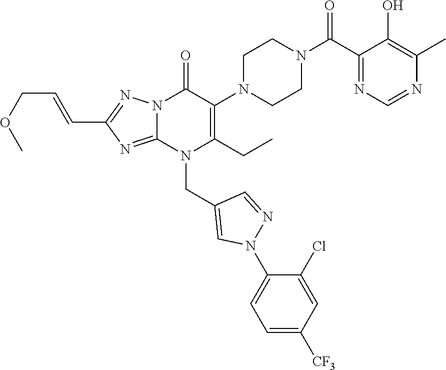 [SMILES: CCc1c(N2CCN(C(=O)c3ncnc(C)c3O)CC2)c(=O)n2nc(/C=C/COC)nc2n1Cc1cnn(-c2ccc(C(F)(F)F)cc2Cl)c1] | 713.2 |
| 144 | 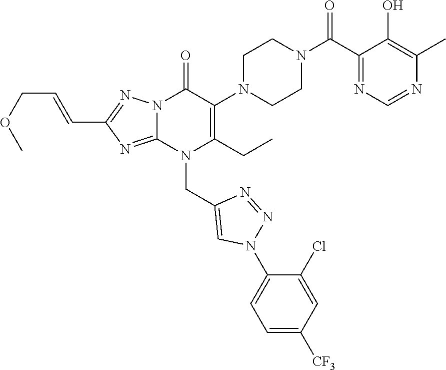 [SMILES: CCc1c(N2CCN(C(=O)c3ncnc(C)c3O)CC2)c(=O)n2nc(/C=C/COC)nc2n1Cc1cn(-c2ccc(C(F)(F)F)cc2Cl)nn1] | 714.2 |
| 145 | 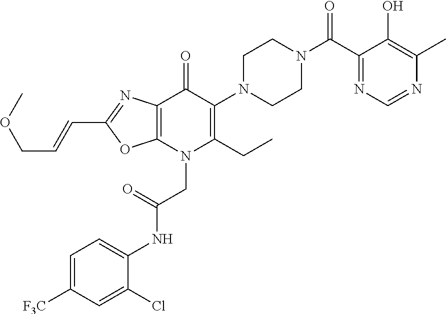 [SMILES: CCc1c(N2CCN(C(=O)c3ncnc(C)c3O)CC2)c(=O)c2nc(/C=C/COC)oc2n1CC(=O)Nc1ccc(C(F)(F)F)cc1Cl] | 790.2 |
| 146 | 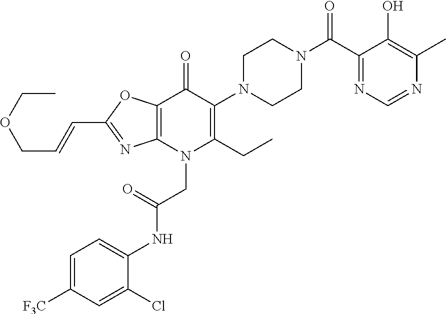 [SMILES: CCOC/C=C/c1nc2c(o1)c(=O)c(N1CCN(C(=O)c3ncnc(C)c3O)CC1)c(CC)n2CC(=O)Nc1ccc(C(F)(F)F)cc1Cl] | 704.2 |
| 147 | 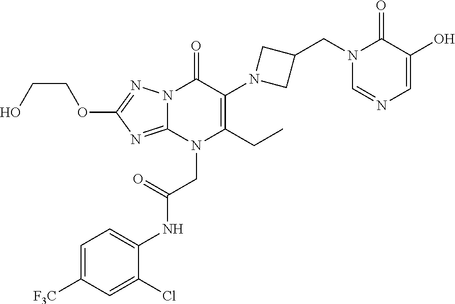 [SMILES: CCc1c(N2CC(Cn3cncc(O)c3=O)C2)c(=O)n2nc(OCCO)nc2n1CC(=O)Nc1ccc(C(F)(F)F)cc1Cl] | 639.2 |
| 148 | 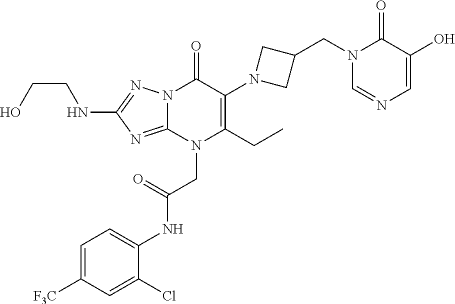 [SMILES: CCc1c(N2CC(Cn3cncc(O)c3=O)C2)c(=O)n2nc(NCCO)nc2n1CC(=O)Nc1ccc(C(F)(F)F)cc1Cl] | 638.2 |
| 149 | 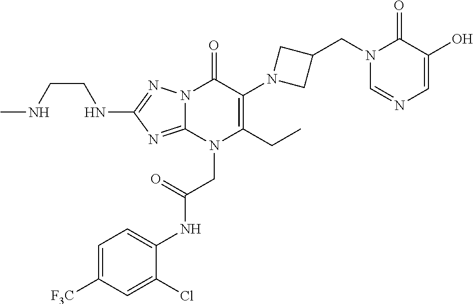 [SMILES: CCc1c(N2CC(Cn3cncc(O)c3=O)C2)c(=O)n2nc(NCCNC)nc2n1CC(=O)Nc1ccc(C(F)(F)F)cc1Cl] | 651.2 |
| 150 | 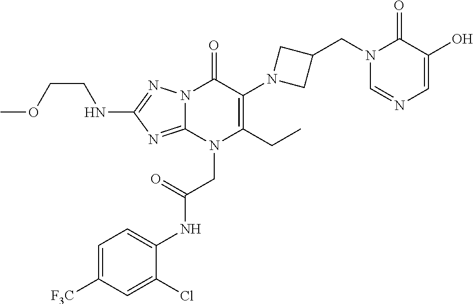 [SMILES: CCc1c(N2CC(Cn3cncc(O)c3=O)C2)c(=O)n2nc(NCCOC)nc2n1CC(=O)Nc1ccc(C(F)(F)F)cc1Cl] | 652.2 |
| 151 | 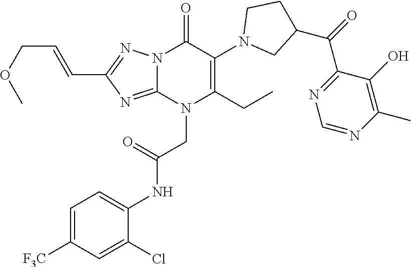 [SMILES: CCc1c(N2CCC(C(=O)c3ncnc(C)c3O)C2)c(=O)n2nc(/C=C/COC)nc2n1CC(=O)Nc1ccc(C(F)(F)F)cc1Cl] | 675.2 |
| 152 | 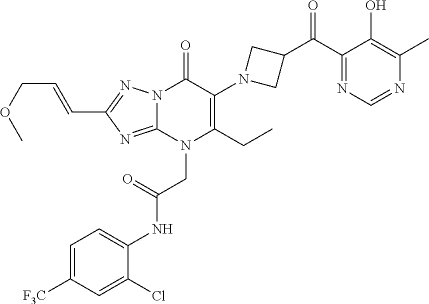 [SMILES: CCc1c(N2CC(C(=O)c3ncnc(C)c3O)C2)c(=O)n2nc(/C=C/COC)nc2n1CC(=O)Nc1ccc(C(F)(F)F)cc1Cl] | 661.2 |
| 153 | 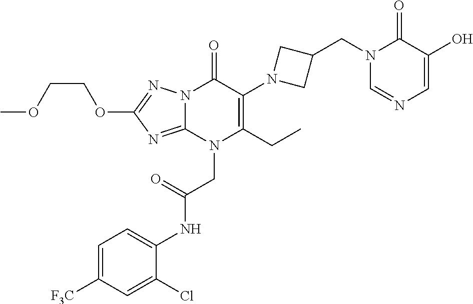 [SMILES: CCc1c(N2CC(Cn3cncc(O)c3=O)C2)c(=O)n2nc(OCCOC)nc2n1CC(=O)Nc1ccc(C(F)(F)F)cc1Cl] | 653.2 |
| 154 | 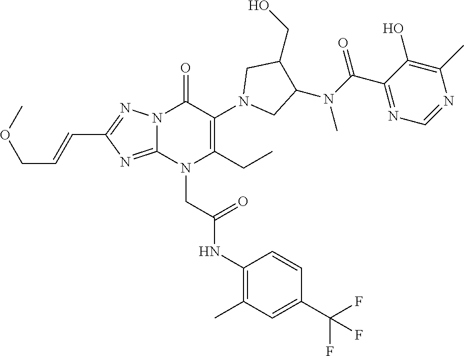 [SMILES: CCc1c(N2CC(CO)C(N(C)C(=O)c3ncnc(C)c3O)C2)c(=O)n2nc(/C=C/COC)nc2n1CC(=O)Nc1ccc(C(F)(F)F)cc1C] | 714.3 |
| 155 | 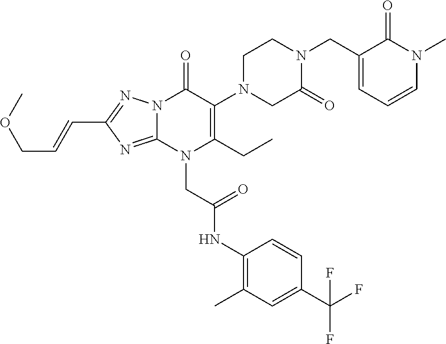 [SMILES: CCc1c(N2CCN(Cc3cccn(C)c3=O)C(=O)C2)c(=O)n2nc(/C=C/COC)nc2n1CC(=O)Nc1ccc(C(F)(F)F)cc1C] | 669.3 |
| 156 | 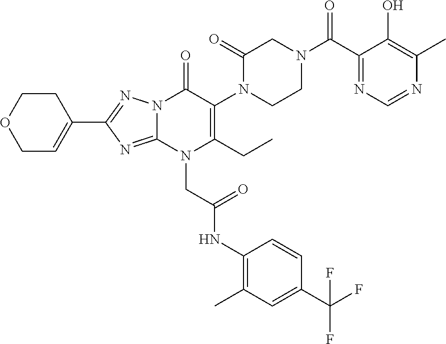 [SMILES: CCc1c(N2CCN(C(=O)c3ncnc(C)c3O)CC2=O)c(=O)n2nc(C3=CCOCC3)nc2n1CC(=O)Nc1ccc(C(F)(F)F)cc1C] | 696.2 |
| 157 | 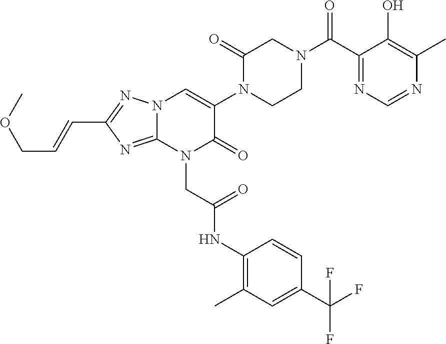 [SMILES: COC/C=C/c1nc2n(CC(=O)Nc3ccc(C(F)(F)F)cc3C)c(=O)c(N3CCN(C(=O)c4ncnc(C)c4O)CC3=O)cn2n1] | 656.2 |
| 158 | 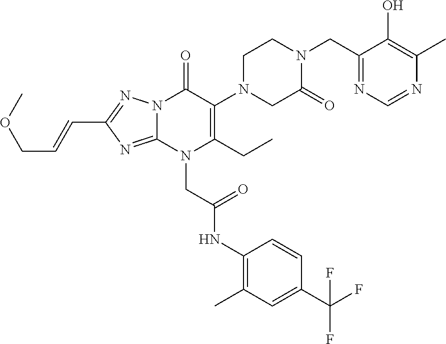 [SMILES: CCc1c(N2CCN(Cc3ncnc(C)c3O)C(=O)C2)c(=O)n2nc(/C=C/COC)nc2n1CC(=O)Nc1ccc(C(F)(F)F)cc1C] | 670.3 |
| 159 | 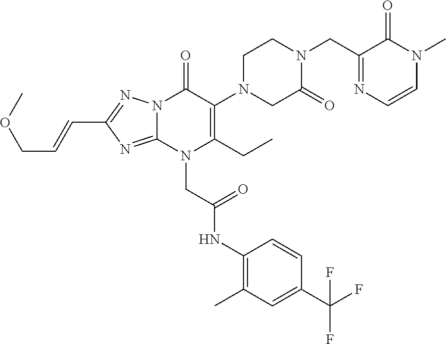 [SMILES: CCc1c(N2CCN(Cc3nccn(C)c3=O)C(=O)C2)c(=O)n2nc(/C=C/COC)nc2n1CC(=O)Nc1ccc(C(F)(F)F)cc1C] | 670.3 |
| 160 | 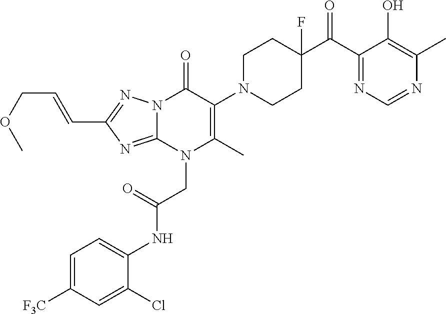 [SMILES: COC/C=C/c1nc2n(CC(=O)Nc3ccc(C(F)(F)F)cc3Cl)c(C)c(N3CCC(F)(C(=O)c4ncnc(C)c4O)CC3)c(=O)n2n1] | 693.2 |
| 161 | 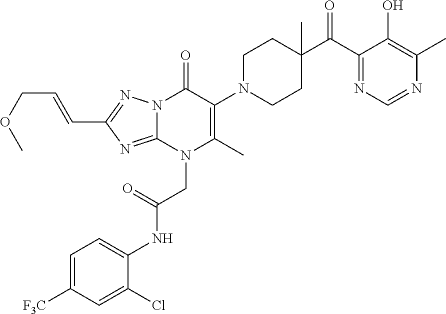 [SMILES: COC/C=C/c1nc2n(CC(=O)Nc3ccc(C(F)(F)F)cc3Cl)c(C)c(N3CCC(C)(C(=O)c4ncnc(C)c4O)CC3)c(=O)n2n1] | 689.2 |
| 162 | 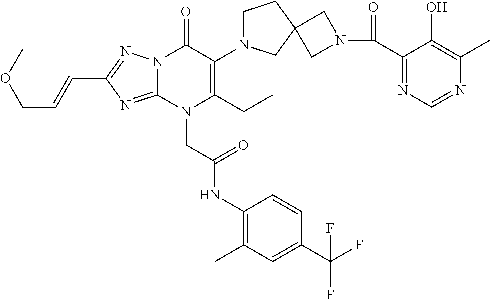 [SMILES: CCc1c(N2CCC3(CN(C(=O)c4ncnc(C)c4O)C3)C2)c(=O)n2nc(/C=C/COC)nc2n1CC(=O)Nc1ccc(C(F)(F)F)cc1C] | 696.3 |
| 163 | 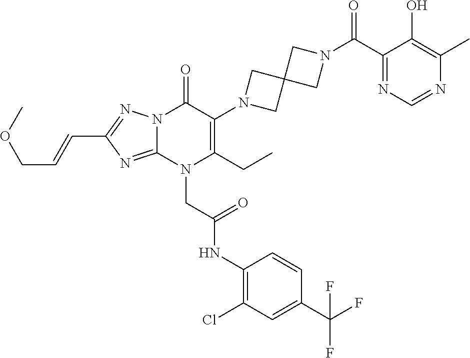 [SMILES: CCc1c(N2CC3(CN(C(=O)c4ncnc(C)c4O)C3)C2)c(=O)n2nc(/C=C/COC)nc2n1CC(=O)Nc1ccc(C(F)(F)F)cc1Cl] | 702.2 |
| 164 | 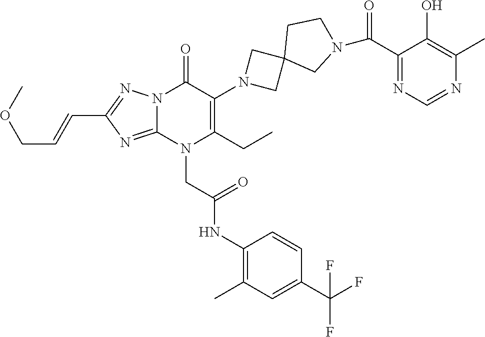 [SMILES: CCc1c(N2CC3(CCN(C(=O)c4ncnc(C)c4O)C3)C2)c(=O)n2nc(/C=C/COC)nc2n1CC(=O)Nc1ccc(C(F)(F)F)cc1C] | 696.3 |
| 165 | 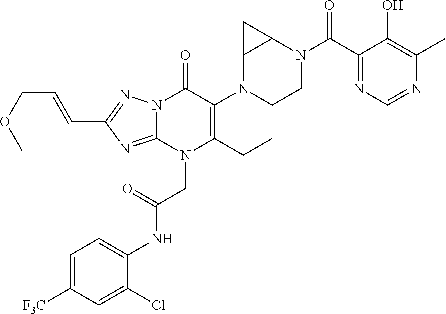 [SMILES: CCc1c(N2CCN(C(=O)c3ncnc(C)c3O)C3CC32)c(=O)n2nc(/C=C/COC)nc2n1CC(=O)Nc1ccc(C(F)(F)F)cc1Cl] | 702.2 |
| 166 | 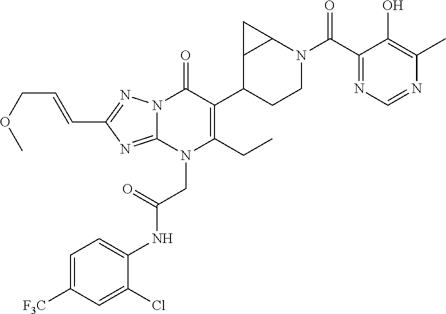 [SMILES: CCc1c(C2CCN(C(=O)c3ncnc(C)c3O)C3CC23)c(=O)n2nc(/C=C/COC)nc2n1CC(=O)Nc1ccc(C(F)(F)F)cc1Cl] | 701.2 |
| 167 | 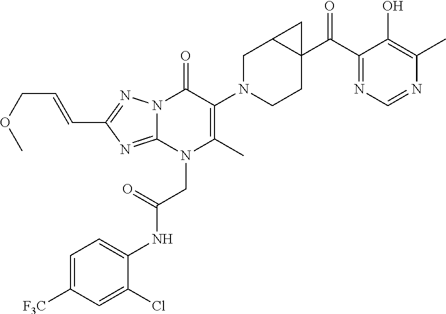 [SMILES: COC/C=C/c1nc2n(CC(=O)Nc3ccc(C(F)(F)F)cc3Cl)c(C)c(N3CCC4(C(=O)c5ncnc(C)c5O)CC4C3)c(=O)n2n1] | 687.2 |
| 168 | 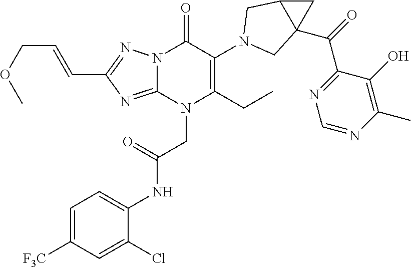 [SMILES: CCc1c(N2CC3CC3(C(=O)c3ncnc(C)c3O)C2)c(=O)n2nc(/C=C/COC)nc2n1CC(=O)Nc1ccc(C(F)(F)F)cc1Cl] | 687.2 |
| 169 | 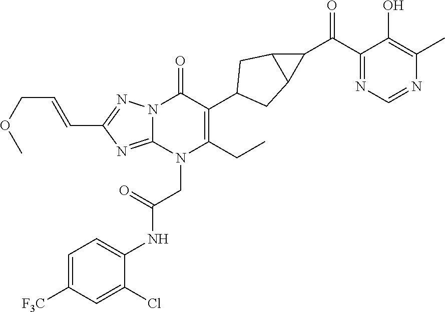 [SMILES: CCc1c(C2CC3C(C2)C3C(=O)c2ncnc(C)c2O)c(=O)n2nc(/C=C/COC)nc2n1CC(=O)Nc1ccc(C(F)(F)F)cc1Cl] | 687.2 |
| 170 | 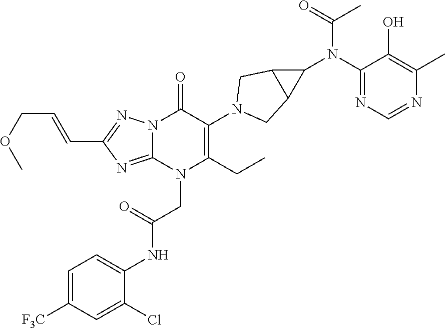 [SMILES: CCc1c(N2CC3C(C2)C3N(C(C)=O)c2ncnc(C)c2O)c(=O)n2nc(/C=C/COC)nc2n1CC(=O)Nc1ccc(C(F)(F)F)cc1Cl] | 716.2 |
| 171 | 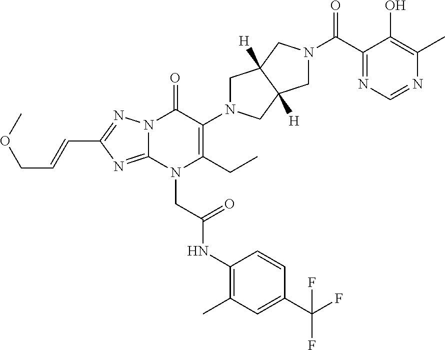 [SMILES: CCc1c(N2C[C@H]3CN(C(=O)c4ncnc(C)c4O)C[C@H]3C2)c(=O)n2nc(/C=C/COC)nc2n1CC(=O)Nc1ccc(C(F)(F)F)cc1C] | 696.3 |
| 172 | 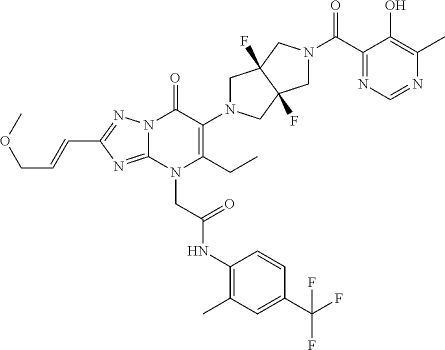 [SMILES: CCc1c(N2C[C@]3(F)CN(C(=O)c4ncnc(C)c4O)C[C@]3(F)C2)c(=O)n2nc(/C=C/COC)nc2n1CC(=O)Nc1ccc(C(F)(F)F)cc1C] | 732.2 |
| 173 | 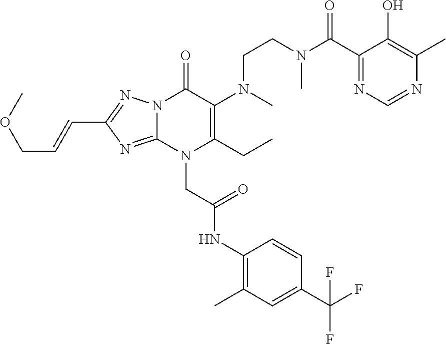 [SMILES: CCc1c(N(C)CCN(C)C(=O)c2ncnc(C)c2O)c(=O)n2nc(/C=C/COC)nc2n1CC(=O)Nc1ccc(C(F)(F)F)cc1C] | 672.3 |
| 174 | 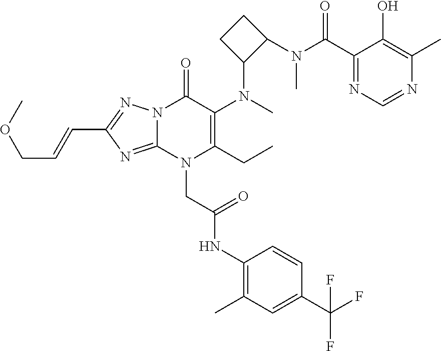 [SMILES: CCc1c(N(C)C2CCC2N(C)C(=O)c2ncnc(C)c2O)c(=O)n2nc(/C=C/COC)nc2n1CC(=O)Nc1ccc(C(F)(F)F)cc1C] | 698.3 |
| 175 | 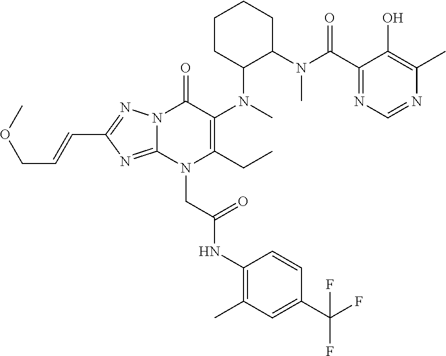 [SMILES: CCc1c(N(C)C2CCCCC2N(C)C(=O)c2ncnc(C)c2O)c(=O)n2nc(/C=C/COC)nc2n1CC(=O)Nc1ccc(C(F)(F)F)cc1C] | 726.3 |
| 176 | 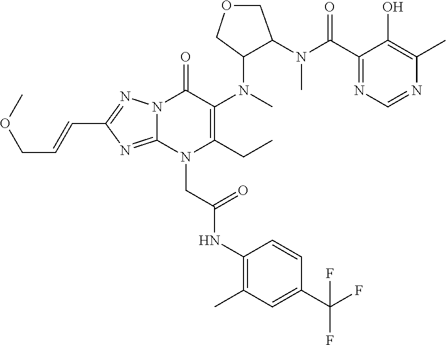 [SMILES: CCc1c(N(C)C2COCC2N(C)C(=O)c2ncnc(C)c2O)c(=O)n2nc(/C=C/COC)nc2n1CC(=O)Nc1ccc(C(F)(F)F)cc1C] | 714.3 |
| 177 | 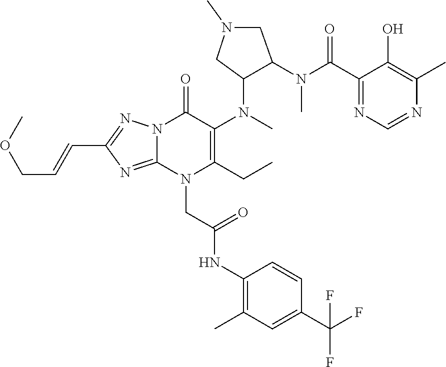 [SMILES: CCc1c(N(C)C2CN(C)CC2N(C)C(=O)c2ncnc(C)c2O)c(=O)n2nc(/C=C/COC)nc2n1CC(=O)Nc1ccc(C(F)(F)F)cc1C] | 727.3 |
| 178 | 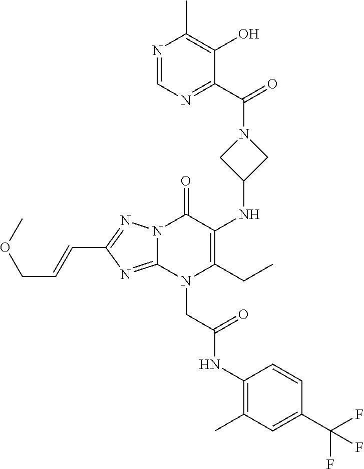 [SMILES: CCc1c(NC2CN(C(=O)c3ncnc(C)c3O)C2)c(=O)n2nc(/C=C/COC)nc2n1CC(=O)Nc1ccc(C(F)(F)F)cc1C] | 655.2 |
| 179 | 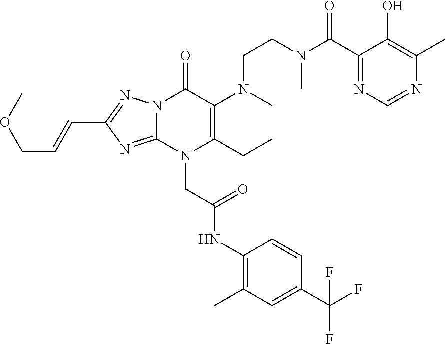 [SMILES: CCc1c(N(C)CCN(C)C(=O)c2ncnc(C)c2O)c(=O)n2nc(/C=C/COC)nc2n1CC(=O)Nc1ccc(C(F)(F)F)cc1C] | 670.3 |
| 180 | 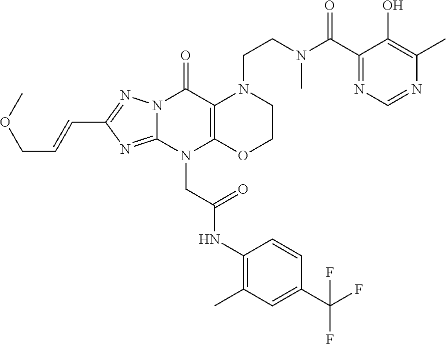 [SMILES: COC/C=C/c1nc2n(CC(=O)Nc3ccc(C(F)(F)F)cc3C)c3c(c(=O)n2n1)N(CCN(C)C(=O)c1ncnc(C)c1O)CCO3] | 672.2 |
| 181 | 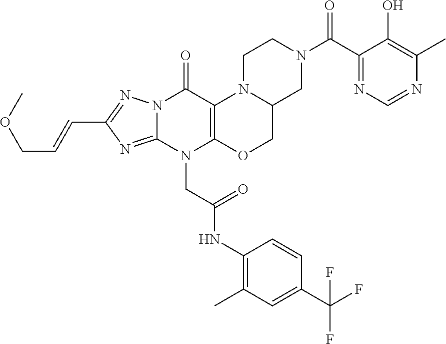 [SMILES: COC/C=C/c1nc2n(CC(=O)Nc3ccc(C(F)(F)F)cc3C)c3c(c(=O)n2n1)N1CCN(C(=O)c2ncnc(C)c2O)CC1CO3] | 670.2 |
| 182 | 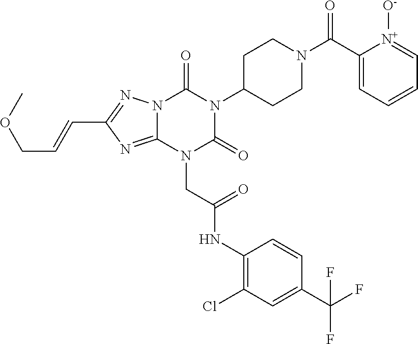 [SMILES: COC/C=C/c1nc2n(CC(=O)Nc3ccc(C(F)(F)F)cc3Cl)c(=O)n(C3CCN(C(=O)c4cccc[n+]4[O-])CC3)c(=O)n2n1] | 663.2 |
| 183 | 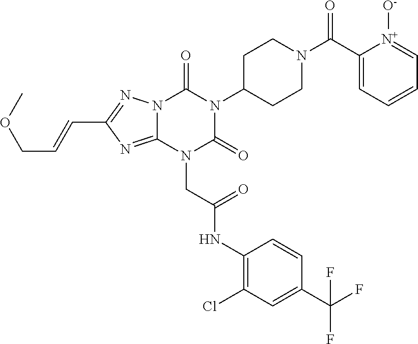 [SMILES: COC/C=C/c1nc2n(CC(=O)Nc3ccc(C(F)(F)F)cc3Cl)c(=O)n(C3CCN(C(=O)c4cccc[n+]4[O-])CC3)c(=O)n2n1] | 664.2 |
| 184 | 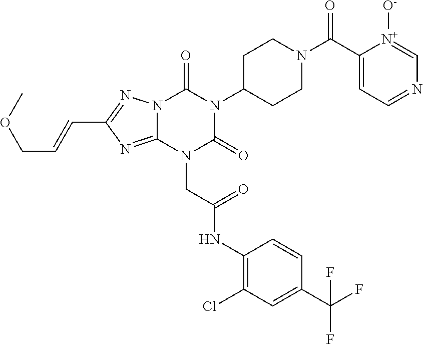 [SMILES: COC/C=C/c1nc2n(CC(=O)Nc3ccc(C(F)(F)F)cc3Cl)c(=O)n(C3CCN(C(=O)c4ccnc[n+]4[O-])CC3)c(=O)n2n1] | 664.2 |
| 185 | 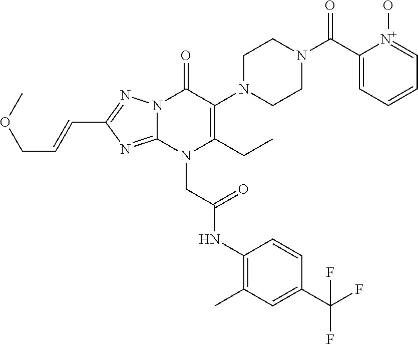 [SMILES: CCc1c(N2CCN(C(=O)c3cccc[n+]3O)CC2)c(=O)n2nc(/C=C/COC)nc2n1CC(=O)Nc1ccc(C(F)(F)F)cc1C] | 669.3 |
| 186 | 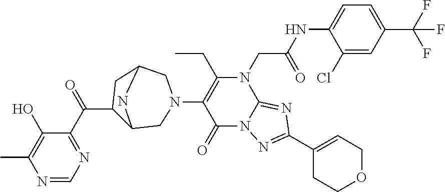 [SMILES: CCc1c(N2CC3CC(C(=O)c4ncnc(C)c4O)C(C2)N3)c(=O)n2nc(C3=CCOCC3)nc2n1CC(=O)Nc1ccc(C(F)(F)F)cc1Cl] | 728.2 |
| 187 | 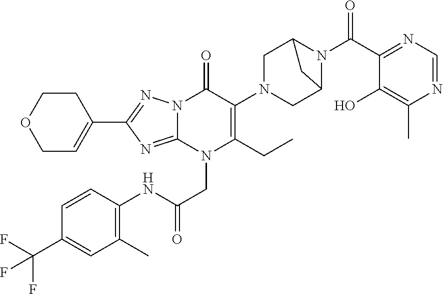 [SMILES: CCc1c(N2CC3CC(C2)N3C(=O)c2ncnc(C)c2O)c(=O)n2nc(C3=CCOCC3)nc2n1CC(=O)Nc1ccc(C(F)(F)F)cc1C] | 694.3 |
| 188 | 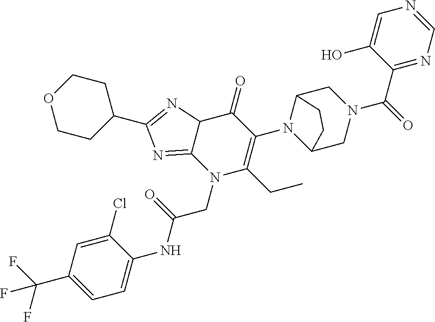 [SMILES: CCC1=C(N2C3CCC2CN(C(=O)c2ncncc2O)C3)C(=O)C2N=C(C3CCOCC3)N=C2N1CC(=O)Nc1ccc(C(F)(F)F)cc1Cl] | 714.2 |
| 189 | 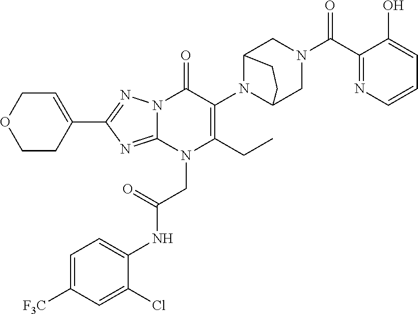 [SMILES: CCc1c(N2C3CCC2CN(C(=O)c2ncccc2O)C3)c(=O)n2nc(C3=CCOCC3)nc2n1CC(=O)Nc1ccc(C(F)(F)F)cc1Cl] | 713.2 |
| 190 | 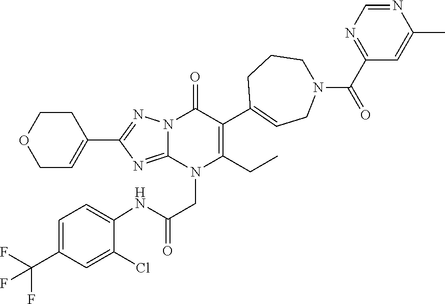 [SMILES: CCc1c(C2=CCN(C(=O)c3cc(C)ncn3)CCC2)c(=O)n2nc(C3=CCOCC3)nc2n1CC(=O)Nc1ccc(C(F)(F)F)cc1Cl] | 713.2 |
| 191 | 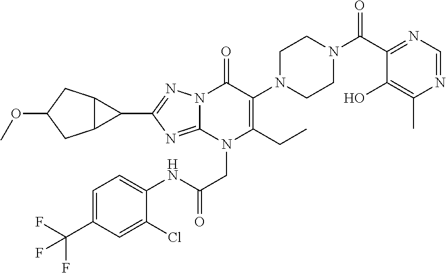 [SMILES: CCc1c(N2CCN(C(=O)c3ncnc(C)c3O)CC2)c(=O)n2nc(C3C4CC(OC)CC43)nc2n1CC(=O)Nc1ccc(C(F)(F)F)cc1Cl] | 730.2 |
| 192 | 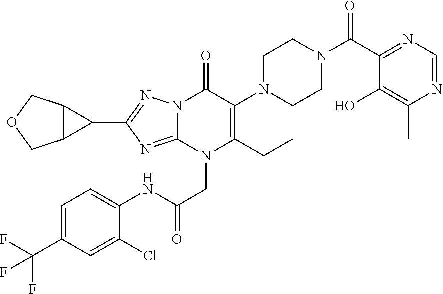 [SMILES: CCc1c(N2CCN(C(=O)c3ncnc(C)c3O)CC2)c(=O)n2nc(C3C4COCC43)nc2n1CC(=O)Nc1ccc(C(F)(F)F)cc1Cl] | 702.2 |
| 193 | 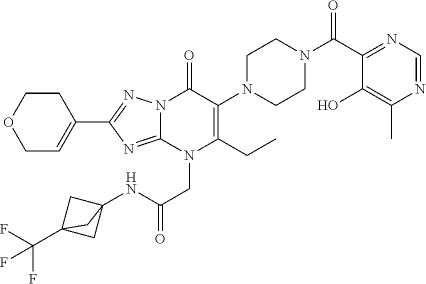 [SMILES: CCc1c(N2CCN(C(=O)c3ncnc(C)c3O)CC2)c(=O)n2nc(C3=CCOCC3)nc2n1CC(=O)NC12CC(C(F)(F)F)(C1)C2] | 658.3 |
| 194 | 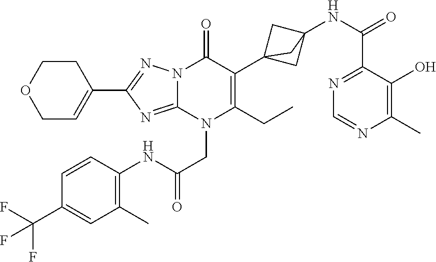 [SMILES: CCc1c(C23CC(NC(=O)c4ncnc(C)c4O)(C2)C3)c(=O)n2nc(C3=CCOCC3)nc2n1CC(=O)Nc1ccc(C(F)(F)F)cc1C] | 679.3 |
| 195 | 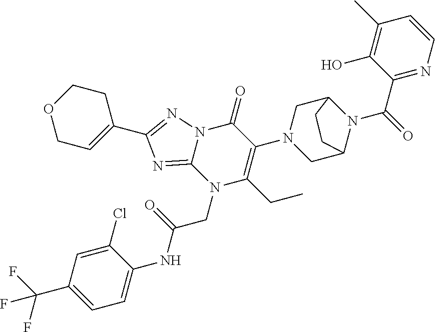 [SMILES: CCc1c(N2CC3CCC(C2)N3C(=O)c2nccc(C)c2O)c(=O)n2nc(C3=CCOCC3)nc2n1CC(=O)Nc1ccc(C(F)(F)F)cc1Cl] | 727.2 |
| 196 | 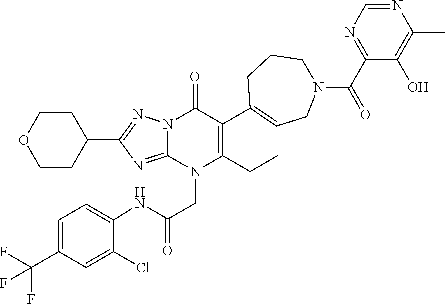 [SMILES: CCc1c(C2=CCN(C(=O)c3ncnc(C)c3O)CCC2)c(=O)n2nc(C3CCOCC3)nc2n1CC(=O)Nc1ccc(C(F)(F)F)cc1Cl] | 715.2 |
| 197 | 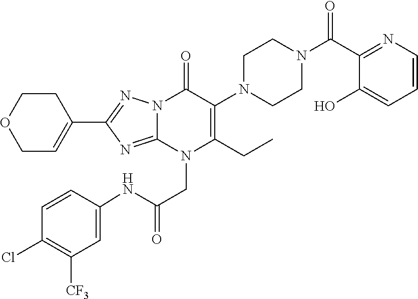 [SMILES: CCc1c(N2CCN(C(=O)c3ncccc3O)CC2)c(=O)n2nc(C3=CCOCC3)nc2n1CC(=O)Nc1ccc(Cl)c(C(F)(F)F)c1] | 687.2 |
| 198 | 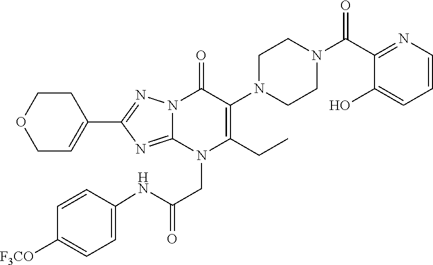 [SMILES: CCc1c(N2CCN(C(=O)c3ncccc3O)CC2)c(=O)n2nc(C3=CCOCC3)nc2n1CC(=O)Nc1ccc(OC(F)(F)F)cc1] | 669.2 |
| 199 | 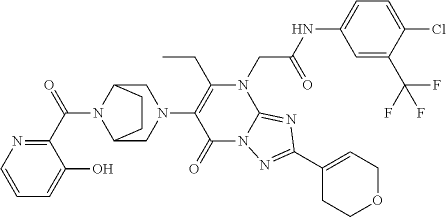 [SMILES: CCc1c(N2CC3CCC(C2)N3C(=O)c2ncccc2O)c(=O)n2nc(C3=CCOCC3)nc2n1CC(=O)Nc1ccc(Cl)c(C(F)(F)F)c1] | 713.2 |
| 200 | 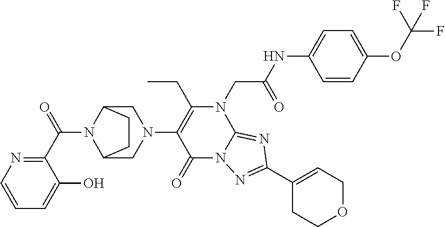 [SMILES: CCc1c(N2CC3CCC(C2)N3C(=O)c2ncccc2O)c(=O)n2nc(C3=CCOCC3)nc2n1CC(=O)Nc1ccc(OC(F)(F)F)cc1] | 695.3 |
| 201 | 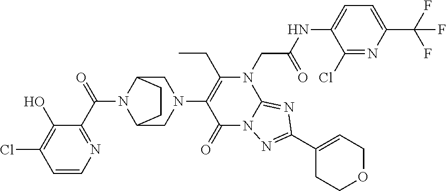 [SMILES: CCc1c(N2CC3CCC(C2)N3C(=O)c2nccc(Cl)c2O)c(=O)n2nc(C3=CCOCC3)nc2n1CC(=O)Nc1ccc(C(F)(F)F)nc1Cl] | 748.2 |
| 202 | 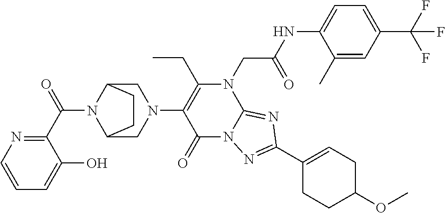 [SMILES: CCc1c(N2CC3CCC(C2)N3C(=O)c2ncccc2O)c(=O)n2nc(C3=CCC(OC)CC3)nc2n1CC(=O)Nc1ccc(C(F)(F)F)cc1C] | 721.3 |
| 203 | 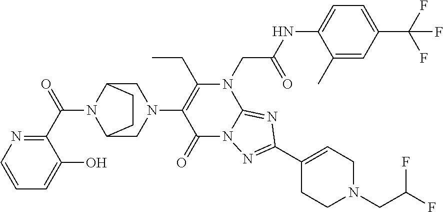 [SMILES: CCc1c(N2CC3CCC(C2)N3C(=O)c2ncccc2O)c(=O)n2nc(C3=CCN(CC(F)F)CC3)nc2n1CC(=O)Nc1ccc(C(F)(F)F)cc1C] | 756.3 |
| 204 | 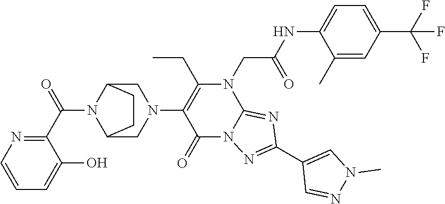 [SMILES: CCc1c(N2CC3CCC(C2)N3C(=O)c2ncccc2O)c(=O)n2nc(-c3cnn(C)c3)nc2n1CC(=O)Nc1ccc(C(F)(F)F)cc1C] | 691.3 |
| 205 | 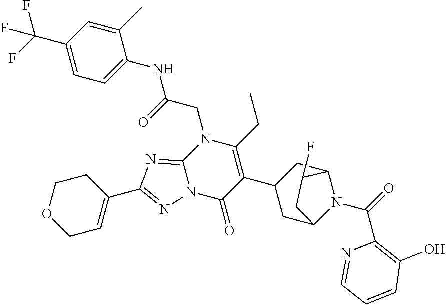 [SMILES: CCc1c(C2CC3CC(F)C(C2)N3C(=O)c2ncccc2O)c(=O)n2nc(C3=CCOCC3)nc2n1CC(=O)Nc1ccc(C(F)(F)F)cc1C] | 711.3 |
| 206 | 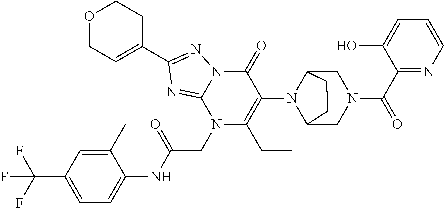 [SMILES: CCc1c(N2C3CCC2CN(C(=O)c2ncccc2O)C3)c(=O)n2nc(C3=CCOCC3)nc2n1CC(=O)Nc1ccc(C(F)(F)F)cc1C] | 693.3 |
| 207 | 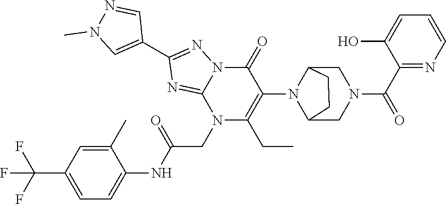 [SMILES: CCc1c(N2C3CCC2CN(C(=O)c2ncccc2O)C3)c(=O)n2nc(-c3cnn(C)c3)nc2n1CC(=O)Nc1ccc(C(F)(F)F)cc1C] | 691.3 |
| 208 | 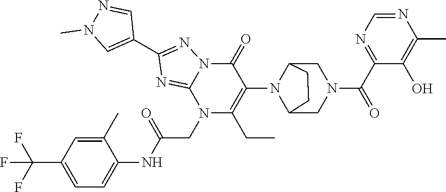 [SMILES: CCc1c(N2C3CCC2CN(C(=O)c2ncnc(C)c2O)C3)c(=O)n2nc(-c3cnn(C)c3)nc2n1CC(=O)Nc1ccc(C(F)(F)F)cc1C] | 706.3 |
| 209 | 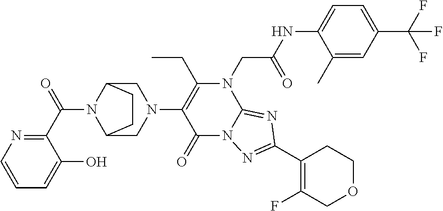 [SMILES: CCc1c(N2CC3CCC(C2)N3C(=O)c2ncccc2O)c(=O)n2nc(C3=C(F)COCC3)nc2n1CC(=O)Nc1ccc(C(F)(F)F)cc1C] | 711.3 |
| 210 | 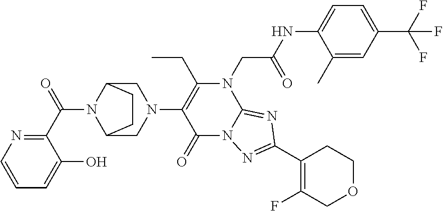 [SMILES: CCc1c(N2CC3CCC(C2)N3C(=O)c2ncccc2O)c(=O)n2nc(C3=C(F)COCC3)nc2n1CC(=O)Nc1ccc(C(F)(F)F)cc1C] | 711.3 |
| 211 | 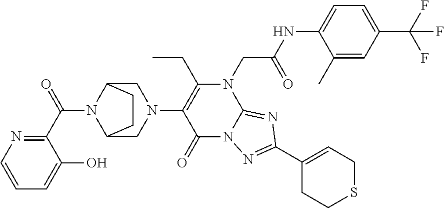 [SMILES: CCc1c(N2CC3CCC(C2)N3C(=O)c2ncccc2O)c(=O)n2nc(C3=CCSCC3)nc2n1CC(=O)Nc1ccc(C(F)(F)F)cc1C] | 709.3 |
| 212 | 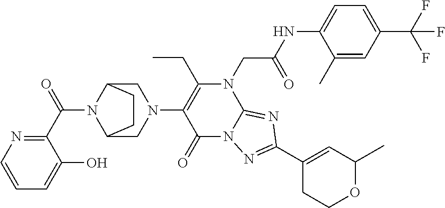 [SMILES: CCc1c(N2CC3CCC(C2)N3C(=O)c2ncccc2O)c(=O)n2nc(C3=CC(C)OCC3)nc2n1CC(=O)Nc1ccc(C(F)(F)F)cc1C] | 707.3 |
| 213 | 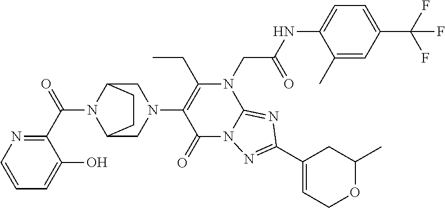 [SMILES: CCc1c(N2CC3CCC(C2)N3C(=O)c2ncccc2O)c(=O)n2nc(C3=CCOC(C)C3)nc2n1CC(=O)Nc1ccc(C(F)(F)F)cc1C] | 707.3 |
| 214 | 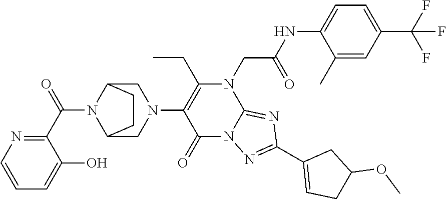 [SMILES: CCc1c(N2CC3CCC(C2)N3C(=O)c2ncccc2O)c(=O)n2nc(C3=CCC(OC)C3)nc2n1CC(=O)Nc1ccc(C(F)(F)F)cc1C] | 707.3 |
| 215 | 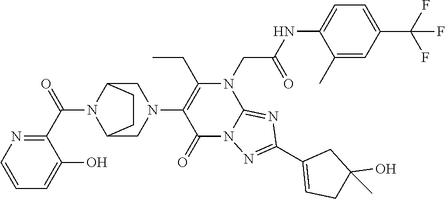 [SMILES: CCc1c(N2CC3CCC(C2)N3C(=O)c2ncccc2O)c(=O)n2nc(C3=CCC(C)(O)C3)nc2n1CC(=O)Nc1ccc(C(F)(F)F)cc1C] | 707.3 |
| 216 | 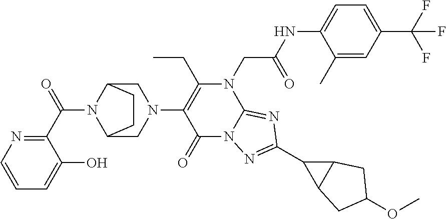 [SMILES: CCc1c(N2CC3CCC(C2)N3C(=O)c2ncccc2O)c(=O)n2nc(C3C4CC(OC)CC43)nc2n1CC(=O)Nc1ccc(C(F)(F)F)cc1C] | 721.3 |
| 217 | 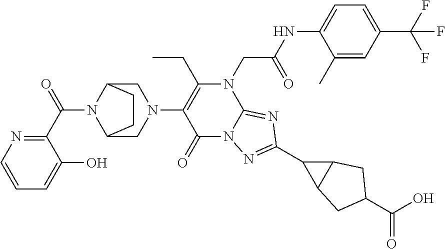 [SMILES: CCc1c(N2CC3CCC(C2)N3C(=O)c2ncccc2O)c(=O)n2nc(C3C4CC(C(=O)O)CC43)nc2n1CC(=O)Nc1ccc(C(F)(F)F)cc1C] | 735.3 |
| 218 | 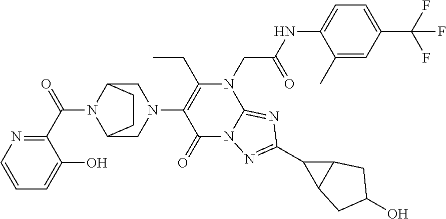 [SMILES: CCc1c(N2CC3CCC(C2)N3C(=O)c2ncccc2O)c(=O)n2nc(C3C4CC(O)CC43)nc2n1CC(=O)Nc1ccc(C(F)(F)F)cc1C] | 707.3 |
| 219 | 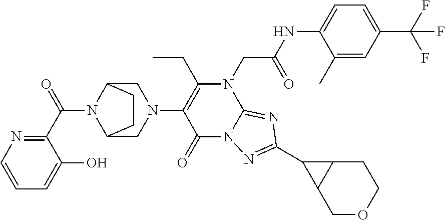 [SMILES: CCc1c(N2CC3CCC(C2)N3C(=O)c2ncccc2O)c(=O)n2nc(C3C4CCOCC43)nc2n1CC(=O)Nc1ccc(C(F)(F)F)cc1C] | 707.3 |
| 220 | 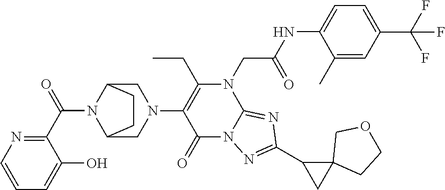 [SMILES: CCc1c(N2CC3CCC(C2)N3C(=O)c2ncccc2O)c(=O)n2nc(C3CC34CCOC4)nc2n1CC(=O)Nc1ccc(C(F)(F)F)cc1C] | 707.3 |
| 221 | 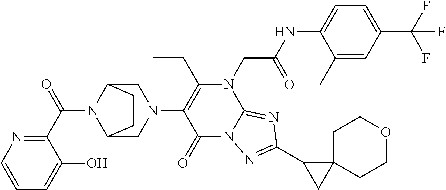 [SMILES: CCc1c(N2CC3CCC(C2)N3C(=O)c2ncccc2O)c(=O)n2nc(C3CC34CCOCC4)nc2n1CC(=O)Nc1ccc(C(F)(F)F)cc1C] | 721.3 |
| 222 | 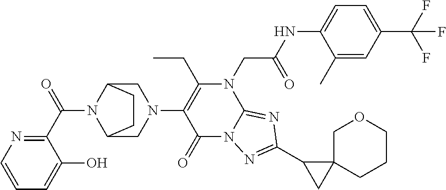 [SMILES: CCc1c(N2CC3CCC(C2)N3C(=O)c2ncccc2O)c(=O)n2nc(C3CC34CCCOC4)nc2n1CC(=O)Nc1ccc(C(F)(F)F)cc1C] | 721.3 |
| 223 | 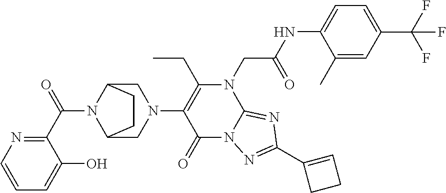 [SMILES: CCc1c(N2CC3CCC(C2)N3C(=O)c2ncccc2O)c(=O)n2nc(C3=CCC3)nc2n1CC(=O)Nc1ccc(C(F)(F)F)cc1C] | 663.3 |
| 224 | 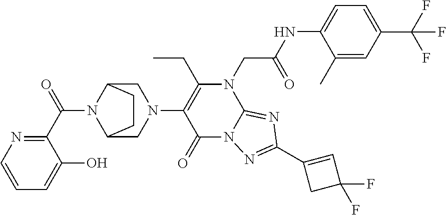 [SMILES: CCc1c(N2CC3CCC(C2)N3C(=O)c2ncccc2O)c(=O)n2nc(C3=CC(F)(F)C3)nc2n1CC(=O)Nc1ccc(C(F)(F)F)cc1C] | 699.2 |
| 225 | 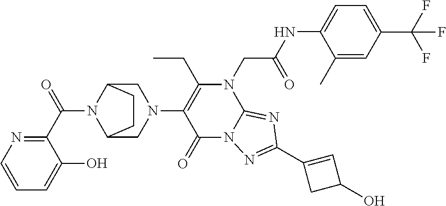 [SMILES: CCc1c(N2CC3CCC(C2)N3C(=O)c2ncccc2O)c(=O)n2nc(C3=CC(O)C3)nc2n1CC(=O)Nc1ccc(C(F)(F)F)cc1C] | 679.3 |
| 226 | 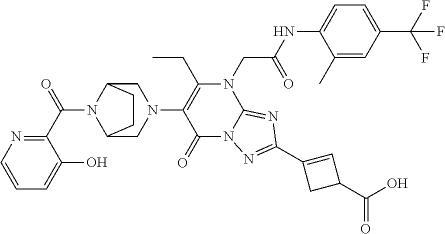 [SMILES: CCc1c(N2CC3CCC(C2)N3C(=O)c2ncccc2O)c(=O)n2nc(C3=CC(C(=O)O)C3)nc2n1CC(=O)Nc1ccc(C(F)(F)F)cc1C] | 707.3 |
| 227 | 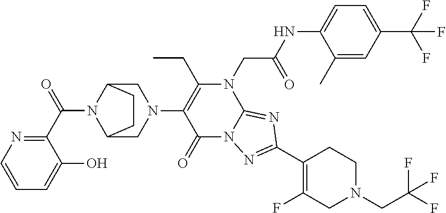 [SMILES: CCc1c(N2CC3CCC(C2)N3C(=O)c2ncccc2O)c(=O)n2nc(C3=C(F)CN(CC(F)(F)F)CC3)nc2n1CC(=O)Nc1ccc(C(F)(F)F)cc1C] | 792.3 |
| 228 | 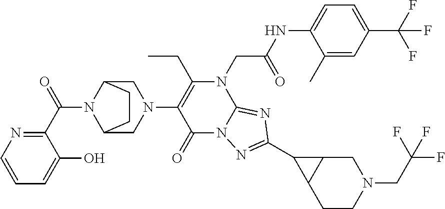 [SMILES: CCc1c(N2CC3CCC(C2)N3C(=O)c2ncccc2O)c(=O)n2nc(C3C4CCN(CC(F)(F)F)CC43)nc2n1CC(=O)Nc1ccc(C(F)(F)F)cc1C] | 788.3 |
| 229 | 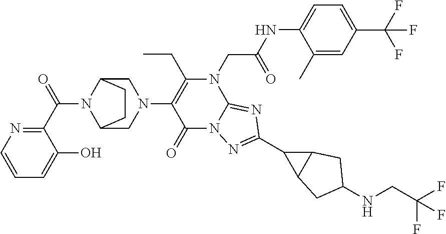 [SMILES: CCc1c(N2CC3CCC(C2)N3C(=O)c2ncccc2O)c(=O)n2nc(C3C4CC(NCC(F)(F)F)CC43)nc2n1CC(=O)Nc1ccc(C(F)(F)F)cc1C] | 788.3 |
| 230 | 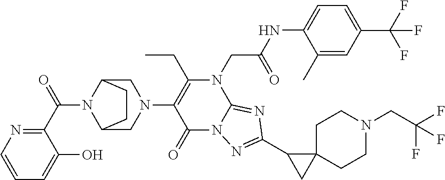 [SMILES: CCc1c(N2CC3CCC(C2)N3C(=O)c2ncccc2O)c(=O)n2nc(C3CC34CCN(CC(F)(F)F)CC4)nc2n1CC(=O)Nc1ccc(C(F)(F)F)cc1C] | 802.3 |
| 231 | 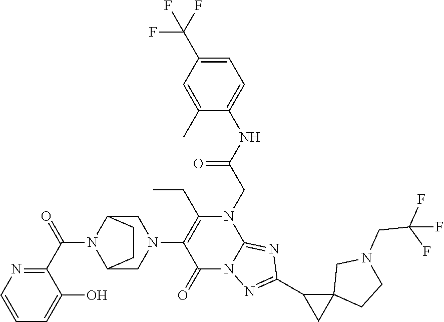 [SMILES: CCc1c(N2CC3CCC(C2)N3C(=O)c2ncccc2O)c(=O)n2nc(C3CC34CCN(CC(F)(F)F)C4)nc2n1CC(=O)Nc1ccc(C(F)(F)F)cc1C] | 788.3 |
| 232 | 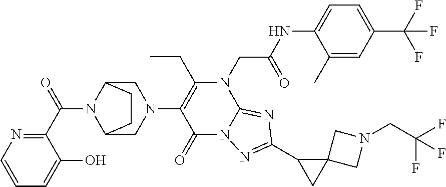 [SMILES: CCc1c(N2CC3CCC(C2)N3C(=O)c2ncccc2O)c(=O)n2nc(C3CC34CN(CC(F)(F)F)C4)nc2n1CC(=O)Nc1ccc(C(F)(F)F)cc1C] | 774.3 |

### Biological Assays

### WRN Helicase Assay

To assess the inhibition properties of the invented compounds on WRN helicase activity, a fluorescent assay using forked DNA was set up. In the helicase assay, in-house produced His-WRN₅₂₇₋₀₇₂ was used. The fluorescent forked DNA, prepared by annealing of OLIGOA-BHQ2 (TTTTTTTTTTTTTTTTTTTTTTTTTTTTTTCGTACCCGATGTGTTCGTTC-BHQ 2) and OLIGOB-TAMRA (TAMRA-GAACGAACACATCGGGTACGTTTTTTTTTTTTTTTTTTTTTTTTTTT TTT), was used as the substrate. 2.5-fold serial dilution of test compounds were prepared for 7 gradient points with DMSO, and then 1.5 μl of each concentration were added to 48.5 μl of assay buffer (25 mM Tris-HCl (pH 8.0), 50 mM NaCl, 2 mM MgCl₂, 1 mM DTT, 0.0500 Tween-20, and 2.5 μg/ml BSA) for further dilution. 5 μl of 45 nM WRN protein and 5 μl of each diluted compound solution were transferred to 384-well assay plate, and pre-incubated at room temperature for 30 minutes. Each concentration of the compounds was added as triplicate in the plate. Controls were included in the test to validate the assay. Instead of compound solution, assay buffer containing 3% DMSO were added to the positive control (no inhibition), and no proteins were added to the negative control (maximal inhibition). Forked DNA and ATP were diluted into 300 nM and 6 mM, respectively, with assay buffer. To initiate the reaction, 5 μl of the dsDNA and ATP solution were added to the assay plate. After 60 minutes room temperature incubation, the plate was transferred to Victor Nivo multimode plate reader (Perkin Elmer, Waltham, MA) and the fluorescence outputs were monitored. GraphPad Prism 9 (GraphPad Software, San Diego, CA) was used to carry out the dose-response curves, deriving IC₅₀s.

### WRN ATPase Assay

To evaluate the inhibition properties of the invented compounds on DNA dependent WRN ATPase activity, an ATPase assay using ADP-Glo assay kit (Promega, Madison, WI) was established. In the ATPase assay, the same WRN protein and DNA substrate as described in helicase assay were used. The serial dilution of the compounds in DMSO was same as helicase assay. In further dilution, 2 μl of each concentration were transferred to 48 μl of assay buffer. 5 μl of 100 nM WRN proteins and 2.5 μl of each diluted compound solution were pre-incubated at room temperature for 30 minutes in the 384-well assay plate. All the test wells were set as triplicate. Afterwards, 2.5 μl of assay buffer containing 400 nM DNA substrate and 4 mM ATP were added to each well and incubated for 60 minutes. The protein-free control (maximal inhibition) and no-compound control (no inhibition) were included in the test. To stop the reaction and deplete the excessive ATP, 10 μl of the ADP-Glo™ Reagent from the assay kit were added and incubated for 40 minutes. Then, 20 μl of the ATP detection reagent was added. After 30 minutes incubation, the luminescence was measured using Victor Nivo multimode plate reader (Perkin Elmer, Waltham, MA). Dose-response curves, spanning IC₅₀s, were generated by GraphPad Prism 9 (GraphPad Software, San Diego, CA). Table TA shows data for selected compounds in above test.

TABLE 1A

| Cmpd. No. | IC50(nM) |
|---|---|
| 186 | 13 |

TABLE 2

| Cmpd. No. | Helicase (IC50, uM) | SW48 (IC50, uM) |
|---|---|---|
| 1 | 48.05 | N/A |
| 2 | 0.0064 | 5.744 |
| 4 | 0.0045 | 1.35 |
| 8 | 0.0059 | 1.626 |
| 15 | 0.0092 | 3.487 |
| 17 | 0.0035 | 0.183 |
| 19 | 0.0051 | 0.818 |
| 23 | 0.0063 | 1.874 |
| 68 | 5 | N/A |
| 74 | 15.29 | N/A |
| 84 | 100 | N/A |
| 86 | 25.63 | N/A |
| 87 | 100 | N/A |
| 94 | 100 | N/A |
| 186 | 0.0027 | 0.716 |
| 187 | 0.0086 | 4.497 |
| 188 | 0.0022 | 1.797 |
| 189 | 0.0046 | 1.107 |
| 190 | 0.0052 | 2.241 |
| 191 | 0.0037 | 1.165 |
| 192 | 0.0084 | 4.185 |
| 196 | 1.28 | N/A |
| 197 | 0.0038 | 0.028 |
| 198 | 0.0023 | 0.129 |
| 199 | 0.0093 | 3.353 |
| 200 | 0.0044 | 7.236 |
| 201 | 0.0073 | 0.802 |
| 202 | 0.007 | 1.045 |
| 203 | 0.0087 | 0.967 |
| 204 | 0.0071 | 1.794 |
| 205 | 0.0109 | 2.37 |
| 206 | 0.0036 | 1.142 |
| 207 | 0.0051 | 1.135 |
| 208 | 0.0062 | 0.44 |

1. A compound of formulas (I)-(II), 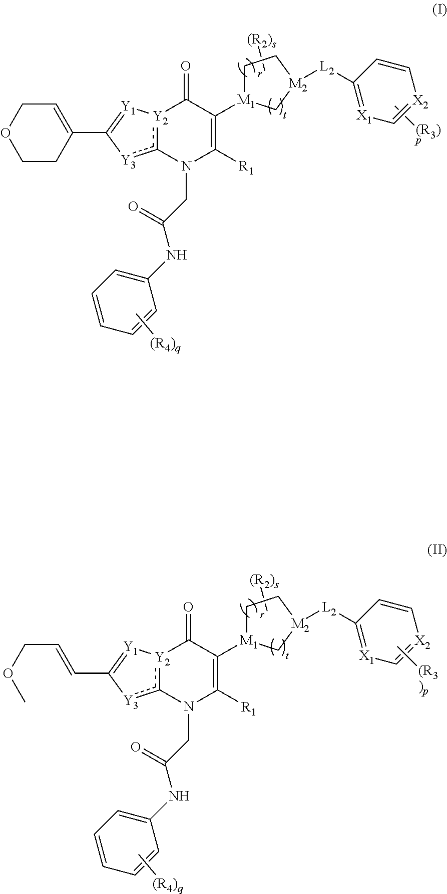
or a tautomer, cis- or trans-isomer, mesomer, racemate, enantiomer, diastereomer, or mixture thereof, or a pharmaceutically acceptable salt, solvate, or prodrug thereof, wherein,
Y₁ is CH, NH, N, O or S;
Y₂ is CH, NH, N, O or S;
Y₃ is CH, NH, N, O or S;
X₁ and X₂ are each independently CH or N;
M₁, M₂ are each independently CH, NH, N, O or S; [symbol]
is single bond or double bond, with the proviso that [symbol] are not double bond simultaneously;
R₁, R₃ and R₄ are each independently selected from the group consisting of hydrogen, deuterium, halogen, amino, cyano, oxo, hydroxy, alkyl, alkoxy, haloalkyl and hydroxyalkyl;
two R₂ together with atom to which they are bound with form a C₃-C₁₀ cycloalkyl or C₃-C₁₀ heterocyclyl,
L₂ is bond, O, —C(O), —CONH—, C₁-C₆ alkyl, C₂-C₆ alkenyl, —C₁-C₆ alkyl-NH—, —C₂-C₆ alkenyl-NH—, C₂-C₆ alkynyl, C₁-C₆ alkoxy, C₁-C₆ alkylthio, C₁-C₆ haloalkyl and —CON(R_{c})—;
R_{c} is selected from the group consisting of hydrogen, deuterium, halogen, amino, cyano, oxo, hydroxy, alkyl, alkoxy, haloalkyl and hydroxyalkyl;
p is 0, 1, 2 or 3;
q is 0, 1, 2 or 3;
r is 0, 1, 2 or 3;
s is 2;
t is 0, 1, 2 or 3.

2-23. (canceled)

24. The compound of claim 1, or a tautomer, cis- or trans-isomer, mesomer, racemate, enantiomer, diastereomer, or mixture thereof, or a pharmaceutically acceptable salt, solvate, or prodrug thereof, wherein the compound is of one of formulas (III)-(XI) 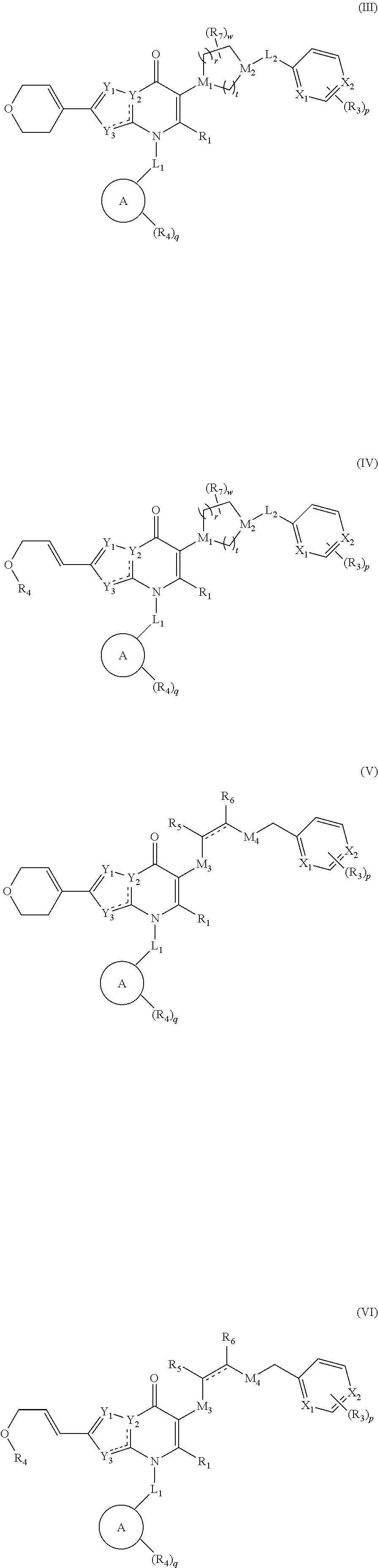 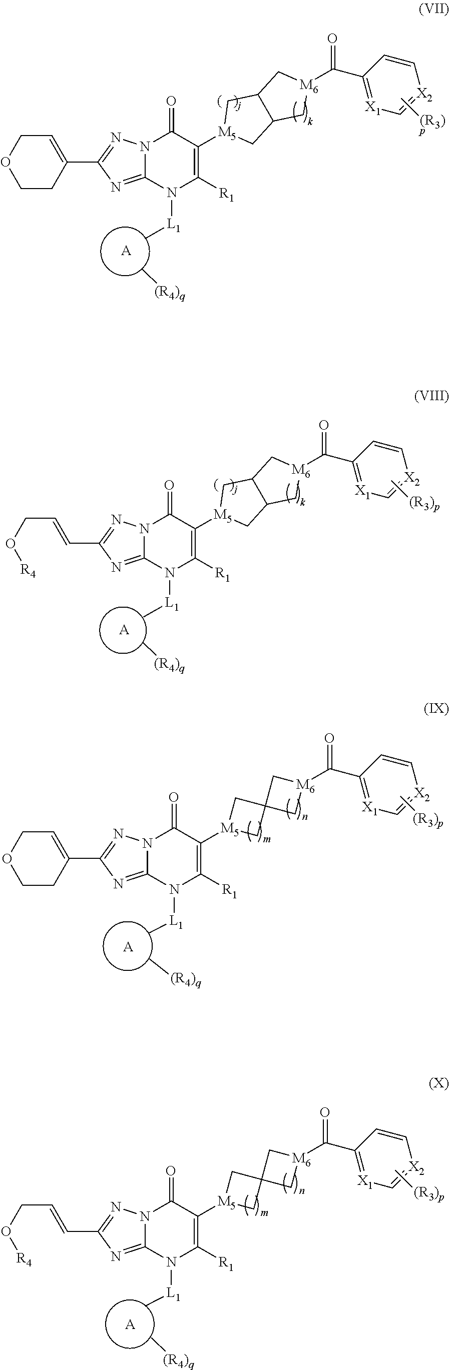 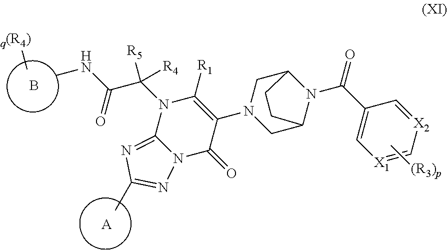
wherein,
ring A and ring B are each independently selected from aryl or heteroaryl;
Y₁ is CH, NH, N, O or S;
Y₂ is CH, NH, N, O or S;
Y₃ is CH, NH, N, O or S;
X₁ and X₂ are each independently CH or N;
M₁, M₂, M₅, M₆, are each independently CH, NH, N, O or S;
M₃ and M₄ are each independently —NRₐ, —CRₐR_{b};
Rₐ and R_{b} are each independently selected from the group consisting of hydrogen, deuterium, halogen, amino, cyano, oxo, hydroxy, alkyl, alkoxy, haloalkyl and hydroxyalkyl; [symbol]
is single bond or double bond, with the proviso that [symbol] are not double bond simultaneously;
R₁, R₃ and R₄ are each independently selected from the group consisting of hydrogen, deuterium, halogen, amino, cyano, oxo, hydroxy, alkyl, alkoxy, haloalkyl and hydroxyalkyl;
R₅ and R₆ are each independently selected from the group consisting of hydrogen, deuterium, halogen, amino, cyano, oxo, hydroxy, alkyl, alkoxy, haloalkyl and hydroxyalkyl;
or, R₅ and R₆ together with atom to which they are bound with form a C₃-C₁₀ cycloalkyl, C₃-C₁₀ heterocyclyl, C₆-C₁₄ aryl or C₅-C₁₂ heteroaryl containing 1 to 4 heteroatoms selected from the group consisting of O, S and N;
R₇ is selected from the group consisting of hydrogen, deuterium, halogen, amino, cyano, oxo, hydroxy, alkyl, alkoxy, haloalkyl and hydroxyalkyl;
or, two of R₇ together with atom to which they are bound with form a C₃-C₁₀ cycloalkyl or C₃-C₁₀ heterocyclyl,
L₁ is bond, O, C₁-C₆ alkyl, C₂-C₆ alkenyl, —C₁-C₆ alkyl-NH—, —C₂-C₆ alkenyl-NH—, C₂-C₆ alkynyl, C₁-C₆ alkoxy, C₁-C₆ alkylthio, C₁-C₆ haloalkyl, —C₃-C₁₀ cycloalkyl-CONH—, —C₁-C₆ alkyl-C₃-C₁₀ cycloalkyl-CONH, —C₁-C₆ haloalkyl-NH—, —C₃-C₁₀-heterocyclic-NH-alkyl, —C₃-C₁₀ cycloalkyl-NH—, —C₁-C₆ alkyl-C(NH)—NH, —C₆-C₁₀ heteroaryl-C₁-C₆ alkyl, —S(O)₂—NH— and —C₁-C₆ alkyl-S(O)₂—NH—;
L₂ is bond, O, —C(O), —CONH—, C₁-C₆ alkyl, C₂-C₆ alkenyl, —C₁-C₆ alkyl-NH—, —C₂-C₆ alkenyl-NH—, C₂-C₆ alkynyl, C₁-C₆ alkoxy, C₁-C₆ alkylthio, C₁-C₆ haloalkyl and —CON(R_{c})—;
R_{c} is selected from the group consisting of hydrogen, deuterium, halogen, amino, cyano, oxo, hydroxy, alkyl, alkoxy, haloalkyl and hydroxyalkyl;
m is 0, 1, 2, 3 or 4;
p is 0, 1, 2 or 3;
q is 0, 1, 2 or 3;
r is 0, 1, 2 or 3;
s is 2 or 3;
t is 0, 1, 2 or 3;
w is 0, 1, 2 or 3; and
n is 0, 1, 2 or 3.

25. The compound of claim 1, or a tautomer, cis- or trans-isomer, mesomer, racemate, enantiomer, diastereomer, or mixture thereof, or a pharmaceutically acceptable salt, solvate, or prodrug thereof, the compound is of formulas (XII)-(XIII), (XIX)-(XXI) 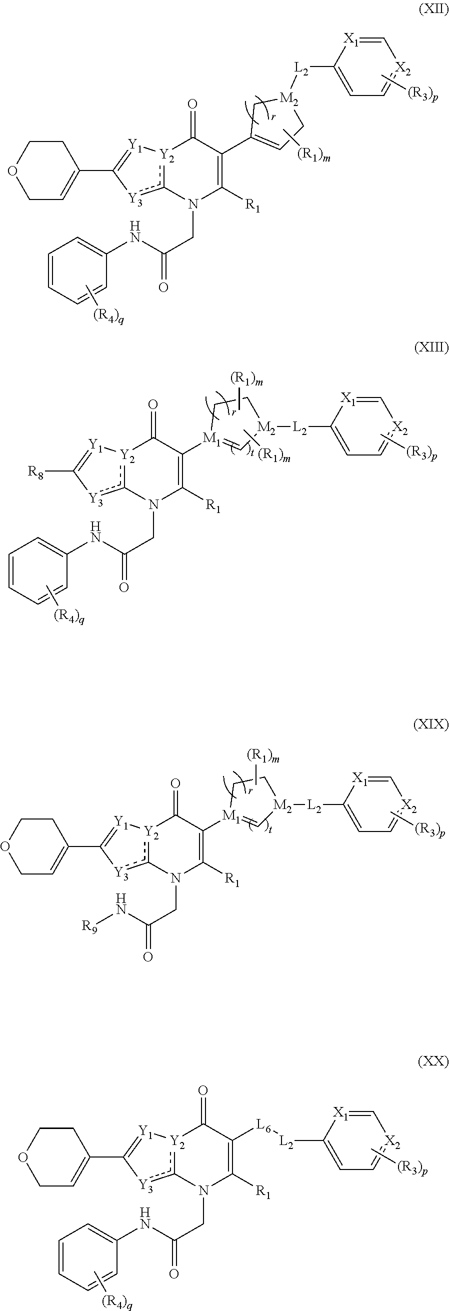 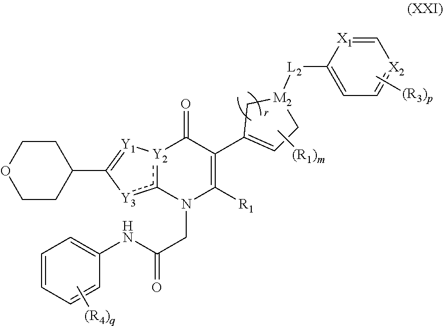
R₈ is C₃-C₁₂ fused cycloalkyl, C₂-C₁₀ fused heterocyclyl, wherein the fused cycloalkyl and fused heterocyclyl are each optionally substituted with one or more substituents selected from hydrogen, deuterium, halogen, amino, cyano, hydroxyl, C₁₋₆ alkyl, C₂₋₆ alkenyl, C₂₋₆ alkynyl, C₁₋₆ alkoxy, oxo;
R₉ is bridged C₃-C₁₀ cycloalkyl or bridged C₂-C₁₀ heterocyclyl wherein the bridged cycloalkyl and bridged heterocyclyl are each optionally substituted with one or more substituents selected from hydrogen, deuterium, halogen, amino, cyano, hydroxyl, C₁₋₅ alkyl, C₂₋₆ alkenyl, C₂₋₆ alkynyl, C₁₋₆ alkoxy, oxo, C₁₋₆ haloalkyl, C₃₋₅ cycloalkyl;
L₆ is bridged C₃-C₁₀ cycloalkyl or bridged C₂-C₁₀ heterocyclyl wherein the bridged cycloalkyl and bridged heterocyclyl are each optionally substituted with one or more substituents selected from hydrogen, deuterium, halogen, amino, cyano, hydroxyl, C₁₋₆ alkyl, C₂₋₆ alkenyl, C₂₋₆ alkynyl, C₁₋₆ alkoxy, oxo, C₁₋₆ haloalkyl, C₃₋₅ cycloalkyl;
Y₁, Y₂, Y₃, M₁, M₂, L₂, X₁, X₂, R₁, R₃, R₄, m, p, q, r, t are as defined in claim 1 and claim 24.

26. The compound of claim 1, or a tautomer, cis- or trans-isomer, mesomer, racemate, enantiomer, diastereomer, or mixture thereof, or a pharmaceutically acceptable salt, solvate, or prodrug thereof, the compound is of formulas (XXII)-(XXIII) 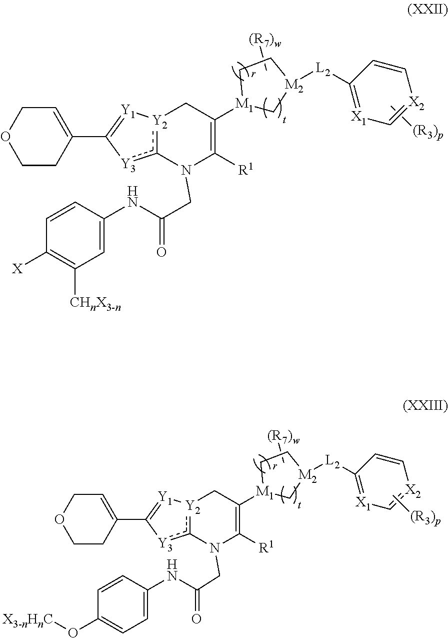
each X is independently selected from F, Cl, Br, I;
Y₁, Y₂, Y₃, M₁, M₂, L₂, X₁, X₂, R₁, R₃, R₇, m, n, p, r, t, w are as defined in claims 1 and 24.

27. The compound of claim 24, or a tautomer, cis- or trans-isomer, mesomer, racemate, enantiomer, diastereomer, or mixture thereof, or a pharmaceutically acceptable salt, solvate, or prodrug thereof, wherein,
(i) ring A is C₆-C₁₄ aryl or C₅-C₁₂ heteroaryl containing 1 to 4 heteroatoms selected from the group consisting of O, S and N; or,
(ii) R₅ and R₆ are each independently selected from the group consisting of hydrogen, deuterium, halogen, amino, cyano, oxo, hydroxy, C₁-C₃ alkyl, C₁-C₃ alkoxy, C₁-C₃ haloalkyl and C₁-C₃ hydroxyalkyl; or, R₅ and R₆ together with atom to which they are bound with form 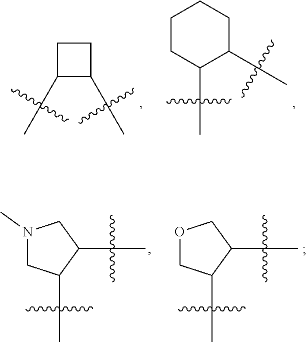
or,
(iii) Rₐ and R_{b} are each independently selected from the group consisting of hydrogen, deuterium, halogen, amino, cyano, oxo, hydroxy, C₁-C₃ alkyl, C₁-C₃ alkoxy, C₁-C₃ haloalkyl and C₁-C₃ hydroxyalkyl; or,
(iv) L₁ is bond, O, C₁-C₃ alkyl, C₂-C₃ alkenyl, —C₁-C₃ alkyl-NH—, —C₂-C₃ alkenyl-NH—, C₂-C₃ alkynyl, C₁-C₃ alkoxy, C₁-C₃ alkylthio, C₁-C₃ haloalkyl, —C₃-C₆ cycloalkyl-CONH—, —C₁-C₃ alkyl-C₃-C₆ cycloalkyl-CONH, —C₁-C₃ haloalkyl-NH—, —C₃-C₆-heterocyclic-NH-alkyl, —C₃-C₆ cycloalkyl-NH—, —C₁-C₆ alkyl-C(NH)—NH, —C₆-C₁₀ heteroaryl-C₁-C₆ alkyl, —S(O)₂—NH— and —C₁-C₆ alkyl-S(O)₂—NH—; or,
(v) ring A is selected from the group consisting of 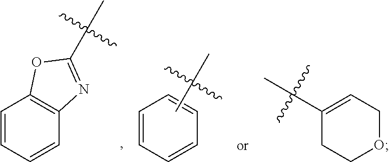
or,
(vi) ring B is selected from the group consisting of phenyl, pyridyl, pyrrolyl or pyrimidinyl; or,
(vii) R₁, R₃ and R₄ are each independently selected from the group consisting of hydrogen, deuterium, halogen, amino, cyano, oxo, hydroxy, C₁-C₆ alkyl, C₁-C₆ alkoxy, C₁-C₆ haloalkyl, C₁-C₆ hydroxyalkyl and C₂-C₆ alkenyl;
preferably, R₁, R₃ and R₄ are each independently selected from the group consisting of hydrogen, deuterium, halogen, amino, cyano, oxo, hydroxy, C₁-C₃ alkyl, —CF₃; or,
(viii) R₇ is selected from the group consisting of hydrogen, deuterium, halogen, amino, cyano, oxo, hydroxy, C₁-C₃ alkyl, C₁-C₃ alkoxy, C₁-C₃ haloalkyl and C₁-C₃ hydroxyalkyl;
or, two of R₇ together with atom to which they are bound with form a bridged C₃-C₁₀ cycloalkyl or bridged C₃-C₁₀ heterocyclyl, preferably, 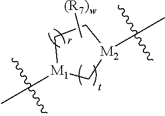
represents 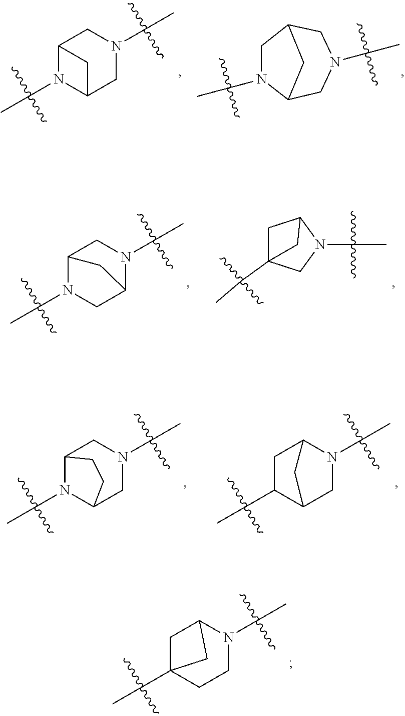
or,
(ix) L₂ is bond, O, —C(O), —CONH—, C₁-C₆ alkyl, C₂-C₆ alkenyl, —C₁-C₃ alkyl-NH—, —C₂-C₃ alkenyl-NH—, C₂-C₃ alkynyl, C₁-C₃ alkoxy, C₁-C₃ alkylthio, C₁-C₃ haloalkyl and —CON(R_{c})—; or,
R_{c} is selected from the group consisting of hydrogen, deuterium, halogen, amino, cyano, oxo, hydroxy, C₁-C₃ alkyl, C₁-C₃ alkoxy, C₁-C₃ haloalkyl and C₁-C₃ hydroxyalkyl.

28. The compound of claim 26, or a tautomer, cis- or trans-isomer, mesomer, racemate, enantiomer, diastereomer, or mixture thereof, or a pharmaceutically acceptable salt, solvate, or prodrug thereof, wherein, R₁, R₃ and R₄ are each independently selected from the group consisting of hydrogen, deuterium, halogen, amino, cyano, oxo, hydroxy, C₁-C₆ alkyl, C₁-C₆ alkoxy, C₁-C₆ haloalkyl, C₁-C₆ hydroxyalkyl and C₂-C₆ alkenyl;
preferably, R₁, R₃ and R₄ are each independently selected from the group consisting of hydrogen, deuterium, halogen, amino, cyano, oxo, hydroxy, C₁-C₃ alkyl, —CF₃.

29. The compound of claim 1, or a tautomer, cis- or trans-isomer, mesomer, racemate, enantiomer, diastereomer, or mixture thereof, or a pharmaceutically acceptable salt, solvate, or prodrug thereof, wherein, (i) two of R₂ together with atom to which they are bound with form a bridged C₃-C₁₀ cycloalkyl or bridged C₃-C₁₀ heterocyclyl;
preferably, 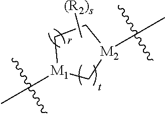
represents 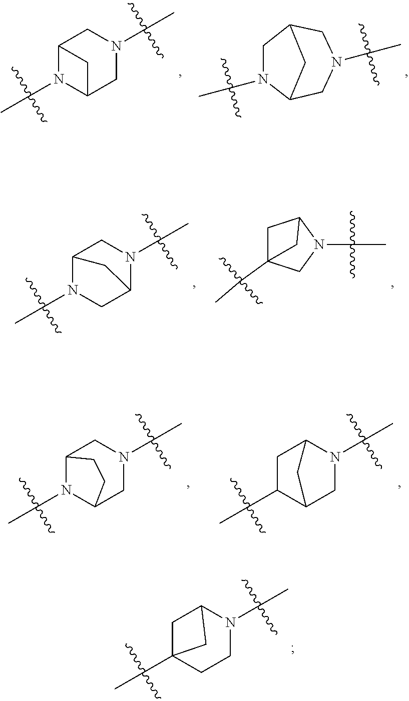
or,
(ii) R₁, R₃ and R₄ are each independently selected from the group consisting of hydrogen, deuterium, halogen, amino, cyano, oxo, hydroxy, C₁-C₆ alkyl, C₁-C₆ alkoxy, C₁-C₆ haloalkyl, C₁-C₆ hydroxyalkyl and C₂-C₆ alkenyl;
preferably, R₁, R₃ and R₄ are each independently selected from the group consisting of hydrogen, deuterium, halogen, amino, cyano, oxo, hydroxy, C₁-C₃ alkyl, —CF₃; or,
(iii) L₂ is bond, O, —C(O), —CONH—, C₁-C₆ alkyl, C₂-C₆ alkenyl, —C₁-C₃ alkyl-NH—, —C₂-C₃ alkenyl-NH—, C₂-C₃ alkynyl, C₁-C₃ alkoxy, C₁-C₃ alkylthio, C₁-C₃ haloalkyl and —CON(R_{c})—;
R_{c} is selected from the group consisting of hydrogen, deuterium, halogen, amino, cyano, oxo, hydroxy, C₁-C₃ alkyl, C₁-C₃ alkoxy, C₁-C₃ haloalkyl and C₁-C₃ hydroxyalkyl.

30. The compound of claim 26, or a tautomer, cis- or trans-isomer, mesomer, racemate, enantiomer, diastereomer, or mixture thereof, or a pharmaceutically acceptable salt, solvate, or prodrug thereof, wherein, R₇ is selected from the group consisting of hydrogen, deuterium, halogen, amino, cyano, oxo, hydroxy, C₁-C₃ alkyl, C₁-C₃ alkoxy, C₁-C₃ haloalkyl and C₁-C₃ hydroxyalkyl;
or, two of R₇ together with atom to which they are bound with form a bridged C₃-C₁₀ cycloalkyl or bridged C₃-C₁₀ heterocyclyl, preferably, 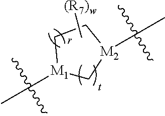
represents

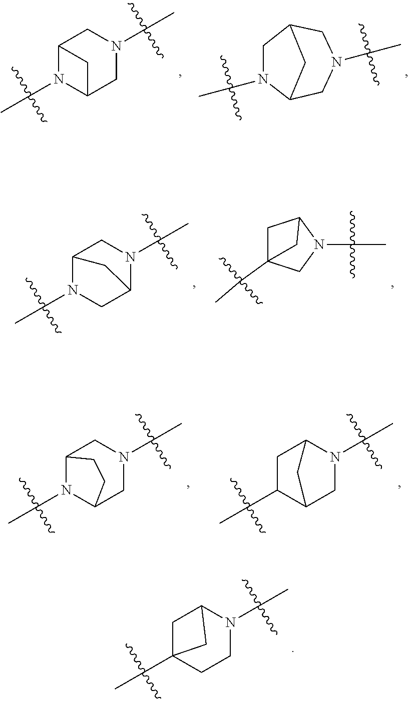

31. The compound of claim 26, or a tautomer, cis- or trans-isomer, mesomer, racemate, enantiomer, diastereomer, or mixture thereof, or a pharmaceutically acceptable salt, solvate, or prodrug thereof, wherein, L₂ is bond, O, —C(O), —CONH—, C₁-C₆ alkyl, C₂-C₆ alkenyl, —C₁-C₃ alkyl-NH—, —C₂-C₃ alkenyl-NH—, C₂-C₃ alkynyl, C₁-C₃ alkoxy, C₁-C₃ alkylthio, C₁-C₃ haloalkyl and —CON(R_{c})—;
R_{c} is selected from the group consisting of hydrogen, deuterium, halogen, amino, cyano, oxo, hydroxy, C₁-C₃ alkyl, C₁-C₃ alkoxy, C₁-C₃ haloalkyl and C₁-C₃ hydroxyalkyl.

32. The compound of claim 25, or a tautomer, cis- or trans-isomer, mesomer, racemate, enantiomer, diastereomer, or mixture thereof, or a pharmaceutically acceptable salt, solvate, or prodrug thereof, wherein,
(i) 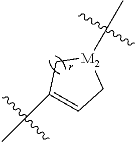
is 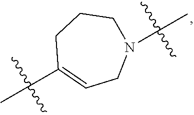 [SMILES: *C1=CCN(*)CCC1]
or,
(ii) in the formula (XIII), R8 is selected from the group consisting of 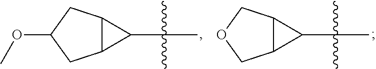
(iii) R9 is 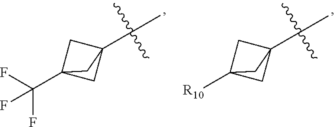
R10 is C₁₋₅ alkyl, C₃₋₅ cycloalkyl;
(iv) L6 is 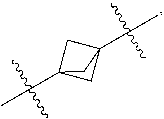 [SMILES: *C12CC(*)(C1)C2]
or L6-L2 is 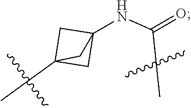 [SMILES: *C(=O)NC12CC(*)(C1)C2]
or,
(v) L₂ is bond, O, —C(O), —CONH—, C₁-C₆ alkyl, C₂-C₆ alkenyl, —C₁-C₃ alkyl-NH—, —C₂-C₃ alkenyl-NH—, C₂-C₃ alkynyl, C₁-C₃ alkoxy, C₁-C₃ alkylthio, C₁-C₃ haloalkyl and —CON(R_{c})—;
R_{c} is selected from the group consisting of hydrogen, deuterium, halogen, amino, cyano, oxo, hydroxy, C₁-C₃ alkyl, C₁-C₃ alkoxy, C₁-C₃ haloalkyl and C₁-C₃ hydroxyalkyl;
(vi) R₁, R₃ and R₄ are each independently selected from the group consisting of hydrogen, deuterium, halogen, amino, cyano, oxo, hydroxy, C₁-C₆ alkyl, C₁-C₆ alkoxy, C₁-C₆ haloalkyl, C₁-C₆ hydroxyalkyl and C₂-C₆ alkenyl;
preferably, R₁, R₃ and R₄ are each independently selected from the group consisting of hydrogen, deuterium, halogen, amino, cyano, oxo, hydroxy, C₁-C₃ alkyl, —CF₃.

33. The compound of claim 26, or a tautomer, cis- or trans-isomer, mesomer, racemate, enantiomer, diastereomer, or mixture thereof, or a pharmaceutically acceptable salt, solvate, or prodrug thereof, wherein, in the formula (XXII), X linked to the phenyl is Cl, and/or CHₙX₃₋ₙ is CF₃; in the formula (XXIII), CHₙX₃₋ₙ is CF₃.

34. The compound of claim 1 is selected from the group consisting of: 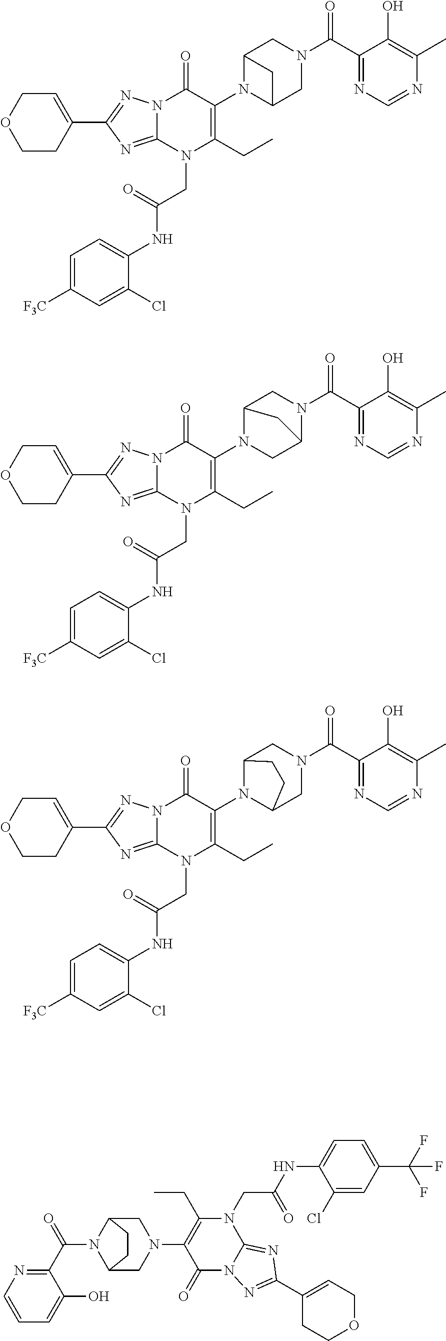 [SMILES: CCc1c(N2C3CC2CN(C(=O)c2ncnc(C)c2O)C3)c(=O)n2nc(C3=CCOCC3)nc2n1CC(=O)Nc1ccc(C(F)(F)F)cc1Cl ; CCc1c(N2CC3CC2CN3C(=O)c2ncnc(C)c2O)c(=O)n2nc(C3=CCOCC3)nc2n1CC(=O)Nc1ccc(C(F)(F)F)cc1Cl ; CCc1c(N2C3CCC2CN(C(=O)c2ncnc(C)c2O)C3)c(=O)n2nc(C3=CCOCC3)nc2n1CC(=O)Nc1ccc(C(F)(F)F)cc1Cl ; CCc1c(N2CC3CCC(C2)N3C(=O)c2ncccc2O)c(=O)n2nc(C3=CCOCC3)nc2n1CC(=O)Nc1ccc(C(F)(F)F)cc1Cl] 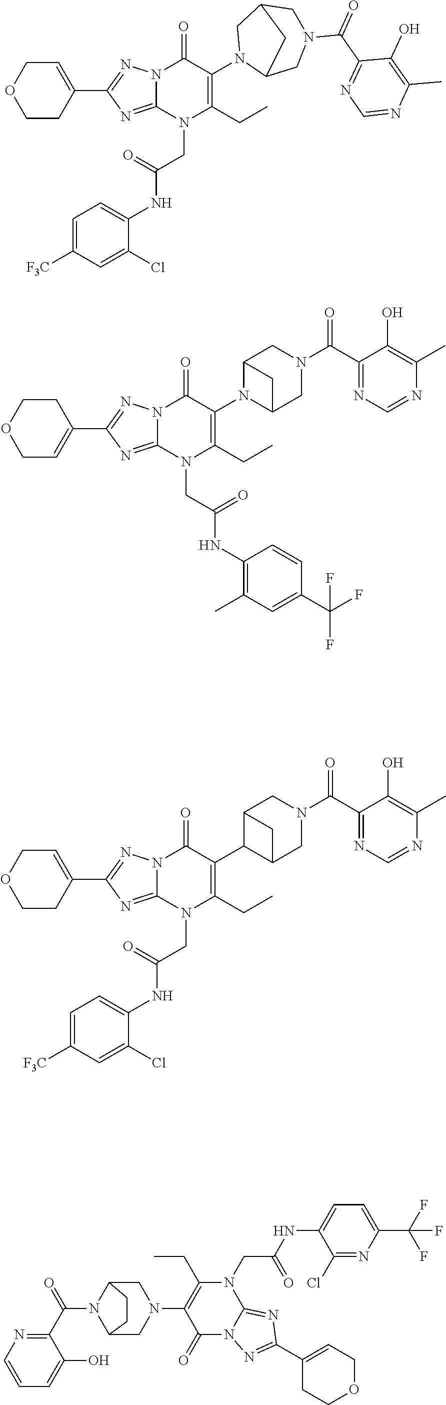 [SMILES: CCc1c(N2CC3CC2CN(C(=O)c2ncnc(C)c2O)C3)c(=O)n2nc(C3=CCOCC3)nc2n1CC(=O)Nc1ccc(C(F)(F)F)cc1Cl ; CCc1c(N2C3CC2CN(C(=O)c2ncnc(C)c2O)C3)c(=O)n2nc(C3=CCOCC3)nc2n1CC(=O)Nc1ccc(C(F)(F)F)cc1C ; CCc1c(C2C3CC2CN(C(=O)c2ncnc(C)c2O)C3)c(=O)n2nc(C3=CCOCC3)nc2n1CC(=O)Nc1ccc(C(F)(F)F)cc1Cl ; CCc1c(N2CC3CCC(C2)N3C(=O)c2ncccc2O)c(=O)n2nc(C3=CCOCC3)nc2n1CC(=O)Nc1ccc(C(F)(F)F)nc1Cl] 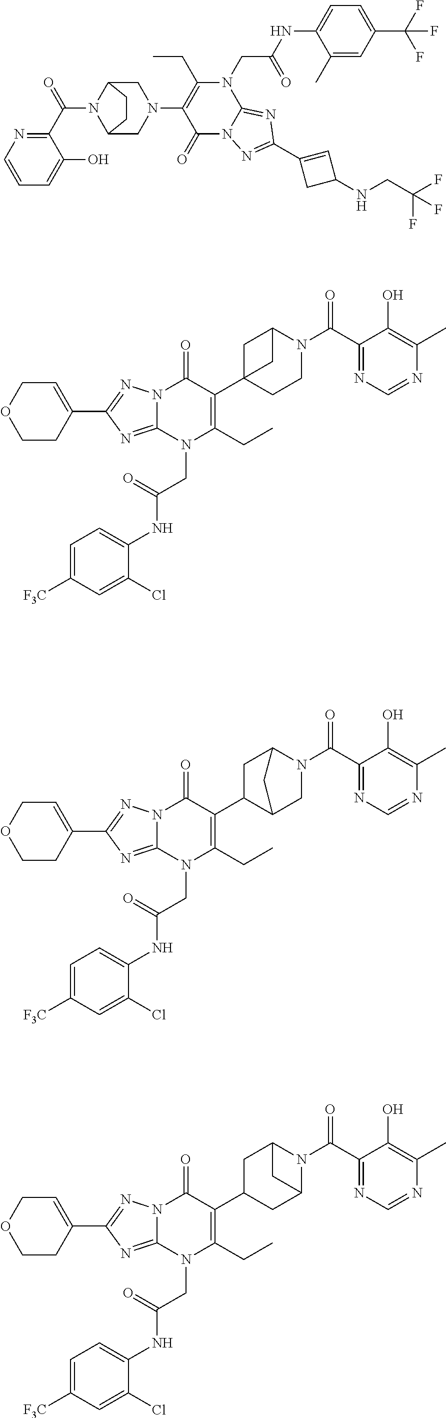 [SMILES: CCc1c(N2CC3CCC(C2)N3C(=O)c2ncccc2O)c(=O)n2nc(C3=CC(NCC(F)(F)F)C3)nc2n1CC(=O)Nc1ccc(C(F)(F)F)cc1C ; CCc1c(C23CCN(C(=O)c4ncnc(C)c4O)C(C2)C3)c(=O)n2nc(C3=CCOCC3)nc2n1CC(=O)Nc1ccc(C(F)(F)F)cc1Cl ; CCc1c(C2CC3CC2CN3C(=O)c2ncnc(C)c2O)c(=O)n2nc(C3=CCOCC3)nc2n1CC(=O)Nc1ccc(C(F)(F)F)cc1Cl ; CCc1c(C2CC3CC(C2)N3C(=O)c2ncnc(C)c2O)c(=O)n2nc(C3=CCOCC3)nc2n1CC(=O)Nc1ccc(C(F)(F)F)cc1Cl] 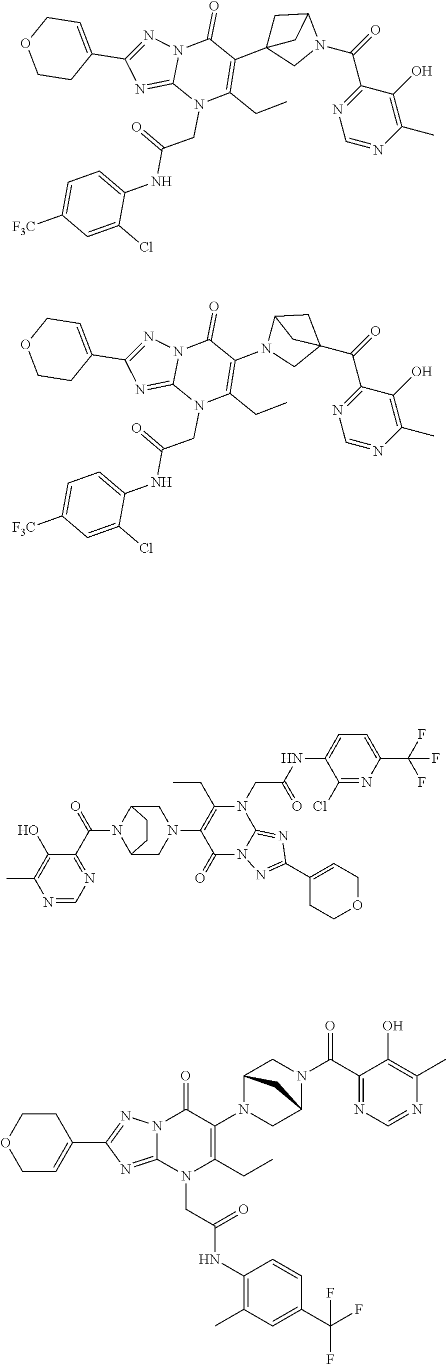 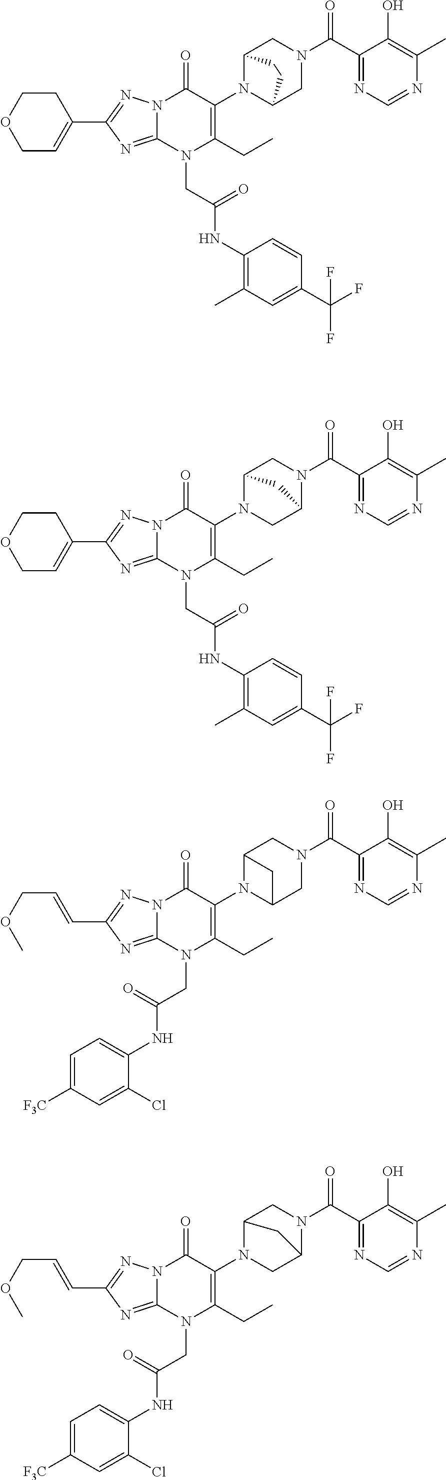 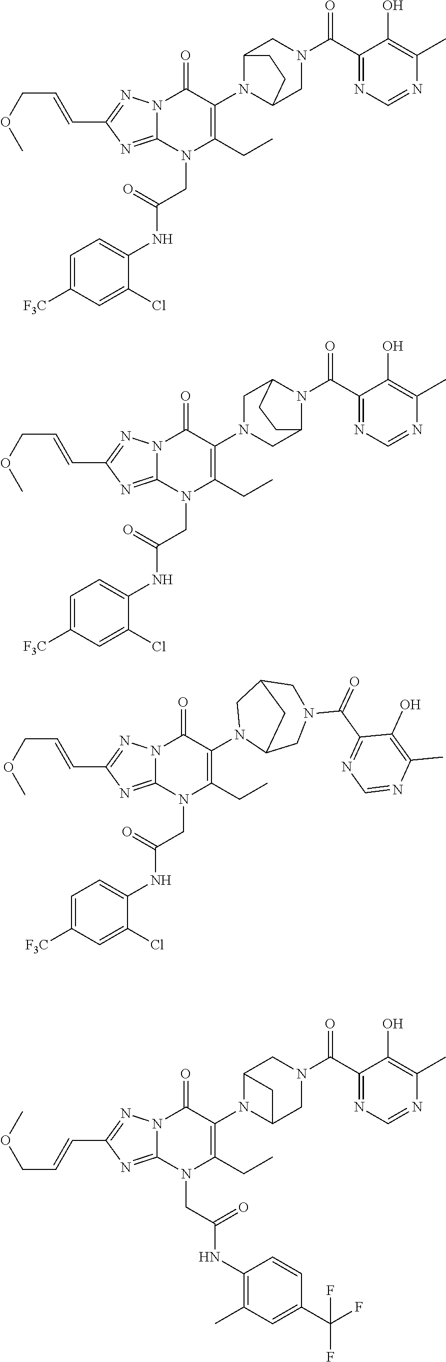 [SMILES: CCc1c(N2C3CCC2CN(C(=O)c2ncnc(C)c2O)C3)c(=O)n2nc(/C=C/COC)nc2n1CC(=O)Nc1ccc(C(F)(F)F)cc1Cl ; CCc1c(N2CC3CCC(C2)N3C(=O)c2ncnc(C)c2O)c(=O)n2nc(/C=C/COC)nc2n1CC(=O)Nc1ccc(C(F)(F)F)cc1Cl ; CCc1c(N2CC3CC2CN(C(=O)c2ncnc(C)c2O)C3)c(=O)n2nc(/C=C/COC)nc2n1CC(=O)Nc1ccc(C(F)(F)F)cc1Cl ; CCc1c(N2C3CC2CN(C(=O)c2ncnc(C)c2O)C3)c(=O)n2nc(/C=C/COC)nc2n1CC(=O)Nc1ccc(C(F)(F)F)cc1C] 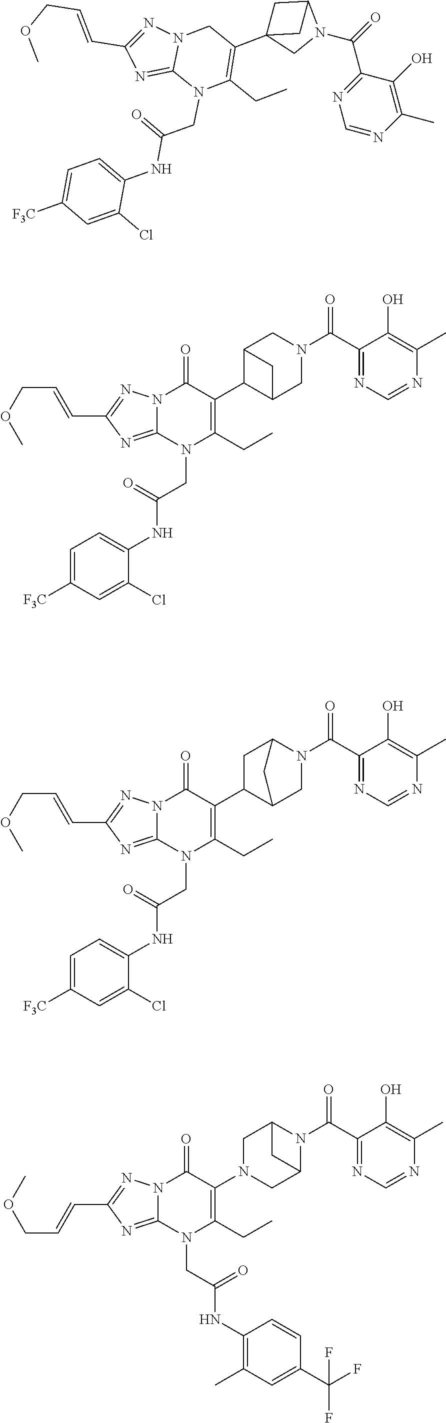 [SMILES: CCC1=C(C23CC(C2)N(C(=O)c2ncnc(C)c2O)C3)Cn2nc(/C=C/COC)nc2N1CC(=O)Nc1ccc(C(F)(F)F)cc1Cl ; CCc1c(C2C3CC2CN(C(=O)c2ncnc(C)c2O)C3)c(=O)n2nc(/C=C/COC)nc2n1CC(=O)Nc1ccc(C(F)(F)F)cc1Cl ; CCc1c(C2CC3CC2CN3C(=O)c2ncnc(C)c2O)c(=O)n2nc(/C=C/COC)nc2n1CC(=O)Nc1ccc(C(F)(F)F)cc1Cl ; CCc1c(N2CC3CC(C2)N3C(=O)c2ncnc(C)c2O)c(=O)n2nc(/C=C/COC)nc2n1CC(=O)Nc1ccc(C(F)(F)F)cc1C] 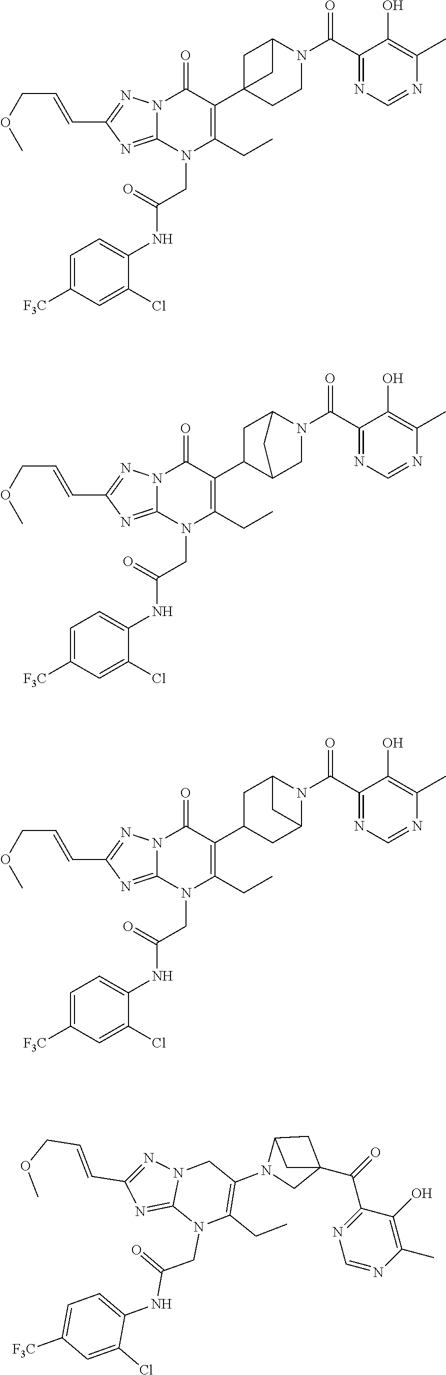 [SMILES: CCc1c(C23CCN(C(=O)c4ncnc(C)c4O)C(C2)C3)c(=O)n2nc(/C=C/COC)nc2n1CC(=O)Nc1ccc(C(F)(F)F)cc1Cl ; CCc1c(C2CC3CC2CN3C(=O)c2ncnc(C)c2O)c(=O)n2nc(/C=C/COC)nc2n1CC(=O)Nc1ccc(C(F)(F)F)cc1Cl ; CCc1c(C2CC3CC(C2)N3C(=O)c2ncnc(C)c2O)c(=O)n2nc(/C=C/COC)nc2n1CC(=O)Nc1ccc(C(F)(F)F)cc1Cl ; CCC1=C(N2CC3(C(=O)c4ncnc(C)c4O)CC2C3)Cn2nc(/C=C/COC)nc2N1CC(=O)Nc1ccc(C(F)(F)F)cc1Cl] 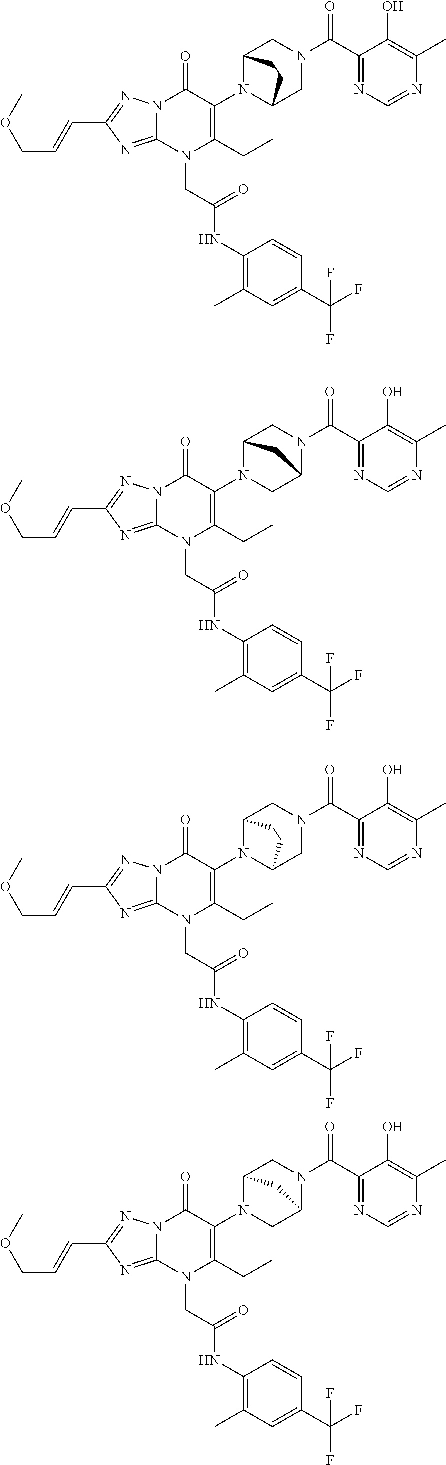 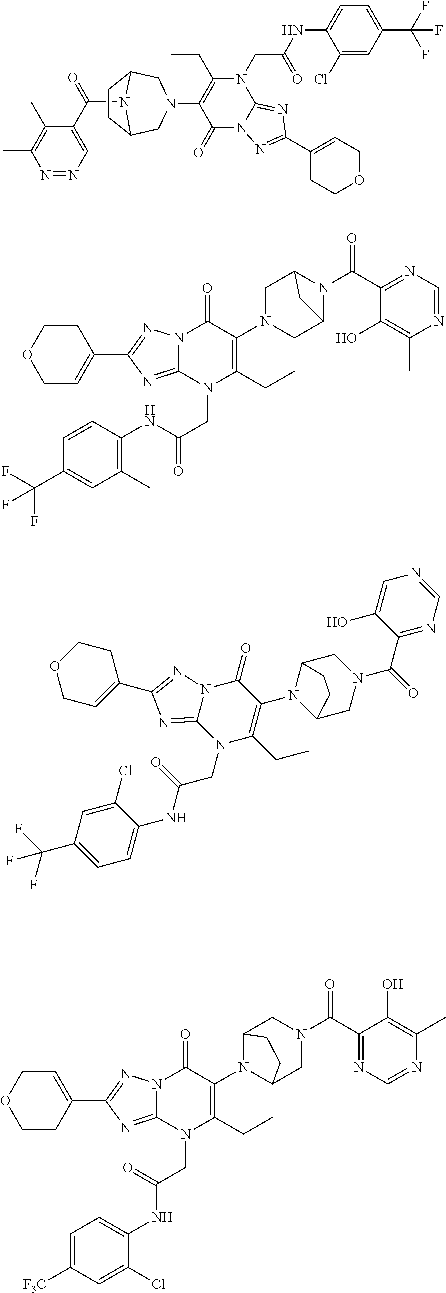 [SMILES: CCc1c(N2CC3CCC(C2)N3C(=O)c2cnnc(C)c2C)c(=O)n2nc(C3=CCOCC3)nc2n1CC(=O)Nc1ccc(C(F)(F)F)cc1Cl ; CCc1c(N2CC3CC(C2)N3C(=O)c2ncnc(C)c2O)c(=O)n2nc(C3=CCOCC3)nc2n1CC(=O)Nc1ccc(C(F)(F)F)cc1C ; CCc1c(N2C3CCC2CN(C(=O)c2ncncc2O)C3)c(=O)n2nc(C3=CCOCC3)nc2n1CC(=O)Nc1ccc(C(F)(F)F)cc1Cl ; CCc1c(N2C3CCC2CN(C(=O)c2ncnc(C)c2O)C3)c(=O)n2nc(C3=CCOCC3)nc2n1CC(=O)Nc1ccc(C(F)(F)F)cc1Cl] 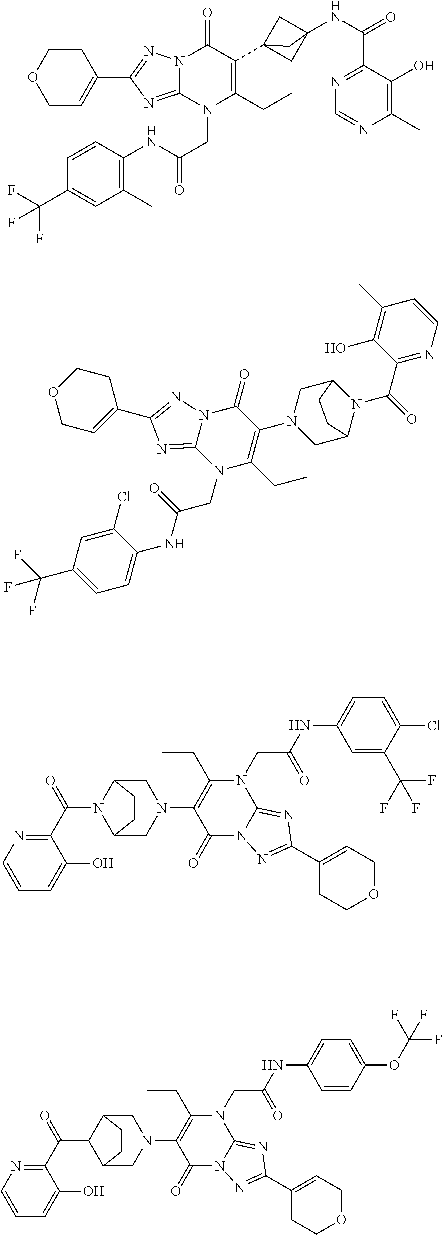 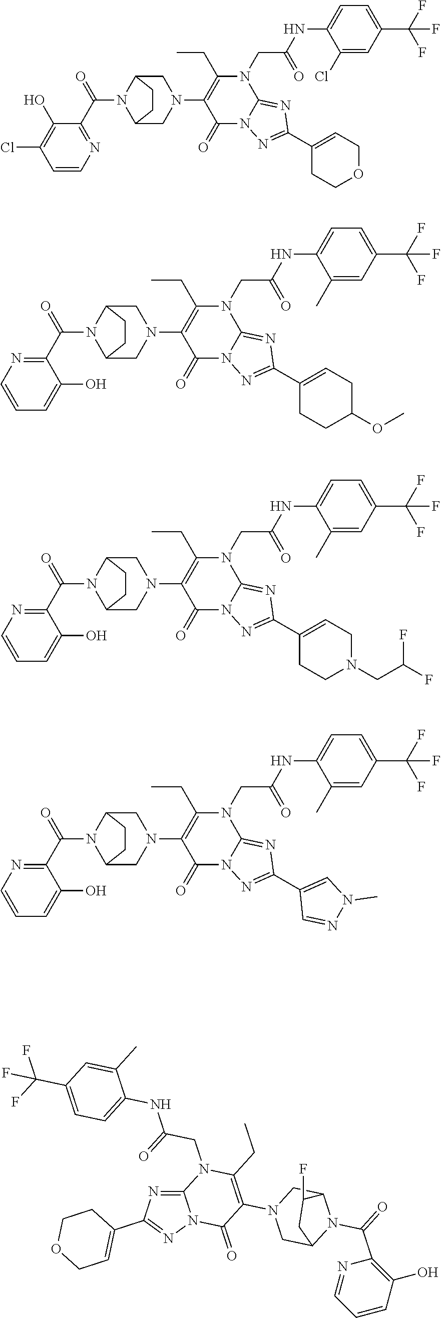 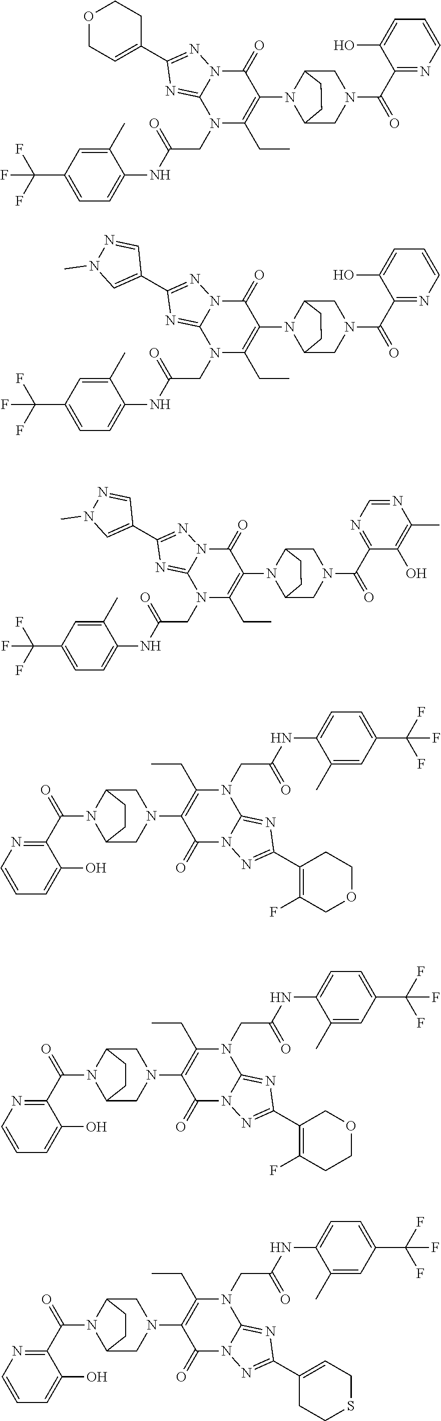 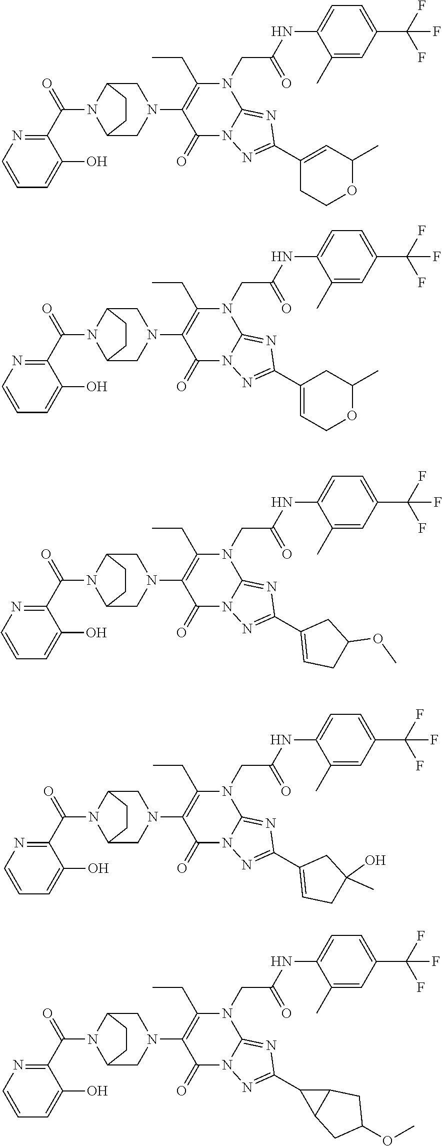 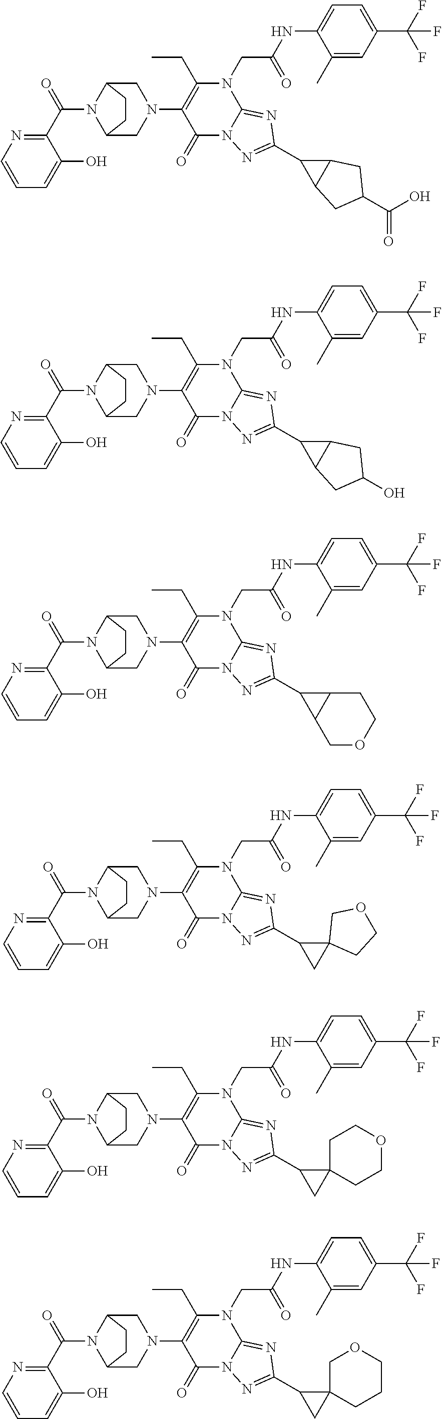 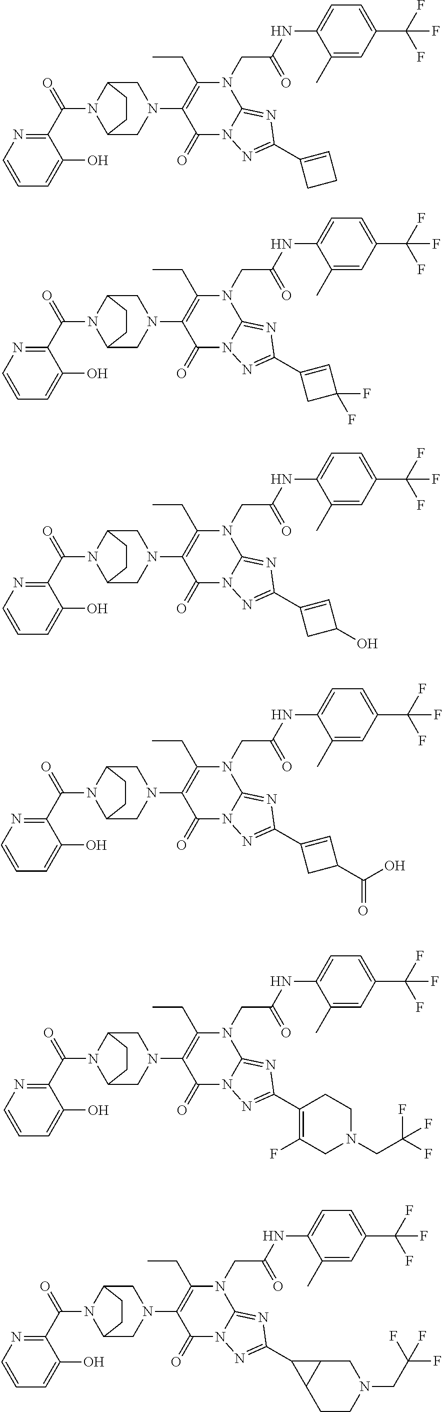 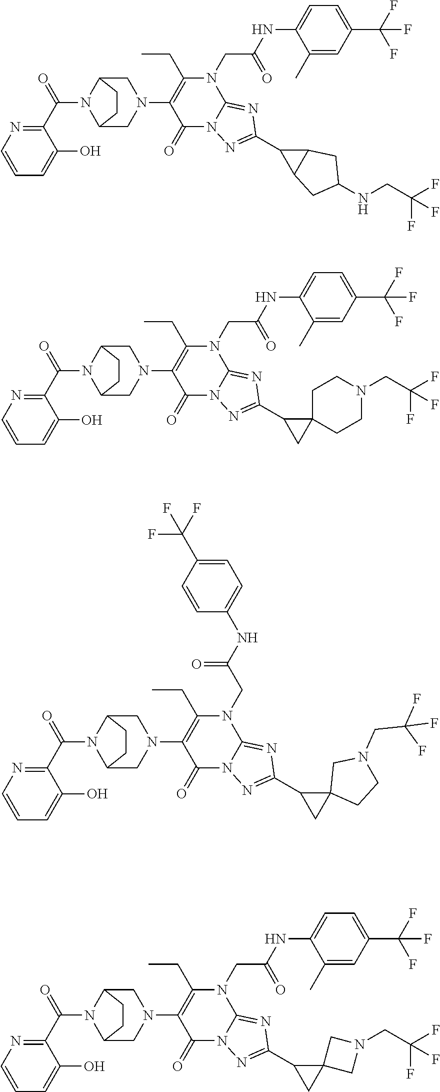 [SMILES: CCc1c(N2CC3CCC(C2)N3C(=O)c2ncccc2O)c(=O)n2nc(C3C4CC(NCC(F)(F)F)CC43)nc2n1CC(=O)Nc1ccc(C(F)(F)F)cc1C ; CCc1c(N2CC3CCC(C2)N3C(=O)c2ncccc2O)c(=O)n2nc(C3CC34CCN(CC(F)(F)F)CC4)nc2n1CC(=O)Nc1ccc(C(F)(F)F)cc1C ; CCc1c(N2CC3CCC(C2)N3C(=O)c2ncccc2O)c(=O)n2nc(C3CC34CCN(CC(F)(F)F)C4)nc2n1CC(=O)Nc1ccc(C(F)(F)F)cc1 ; CCc1c(N2CC3CCC(C2)N3C(=O)c2ncccc2O)c(=O)n2nc(C3CC34CN(CC(F)(F)F)C4)nc2n1CC(=O)Nc1ccc(C(F)(F)F)cc1C]
or a tautomer, cis- or trans-isomer, mesomer, racemate, enantiomer, diastereomer, or mixture thereof, or a pharmaceutically acceptable salt, solvate, or prodrug thereof.

35. The compound of claim 24 selected from the group consisting of: 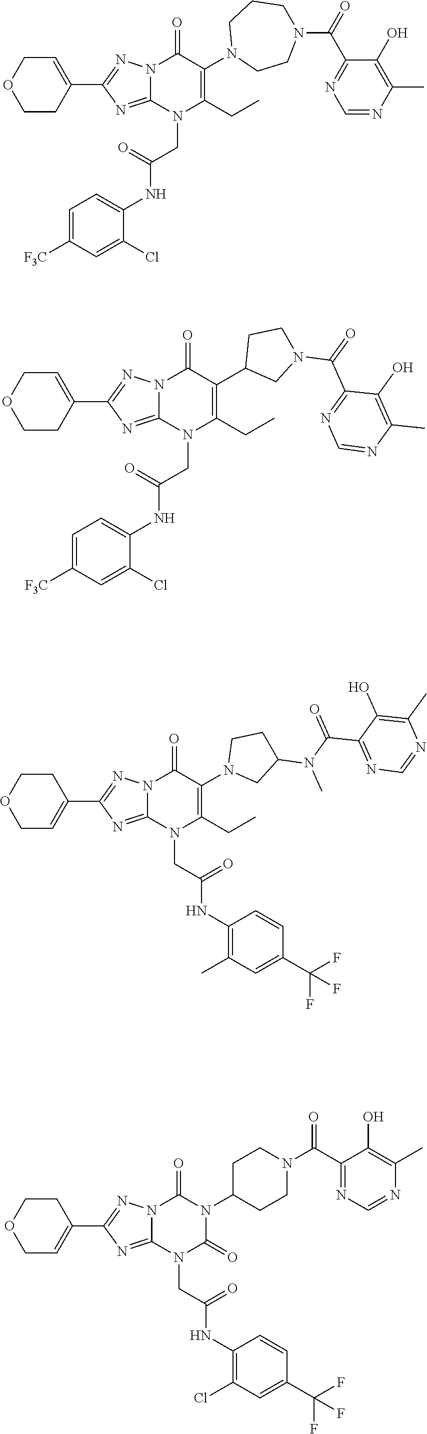 [SMILES: CCc1c(N2CCCN(C(=O)c3ncnc(C)c3O)CC2)c(=O)n2nc(C3=CCOCC3)nc2n1CC(=O)Nc1ccc(C(F)(F)F)cc1Cl ; CCc1c(C2CCN(C(=O)c3ncnc(C)c3O)C2)c(=O)n2nc(C3=CCOCC3)nc2n1CC(=O)Nc1ccc(C(F)(F)F)cc1Cl ; CCc1c(N2CCC(N(C)C(=O)c3ncnc(C)c3O)C2)c(=O)n2nc(C3=CCOCC3)nc2n1CC(=O)Nc1ccc(C(F)(F)F)cc1C ; Cc1ncnc(C(=O)N2CCC(n3c(=O)n(CC(=O)Nc4ccc(C(F)(F)F)cc4Cl)c4nc(C5=CCOCC5)nn4c3=O)CC2)c1O] 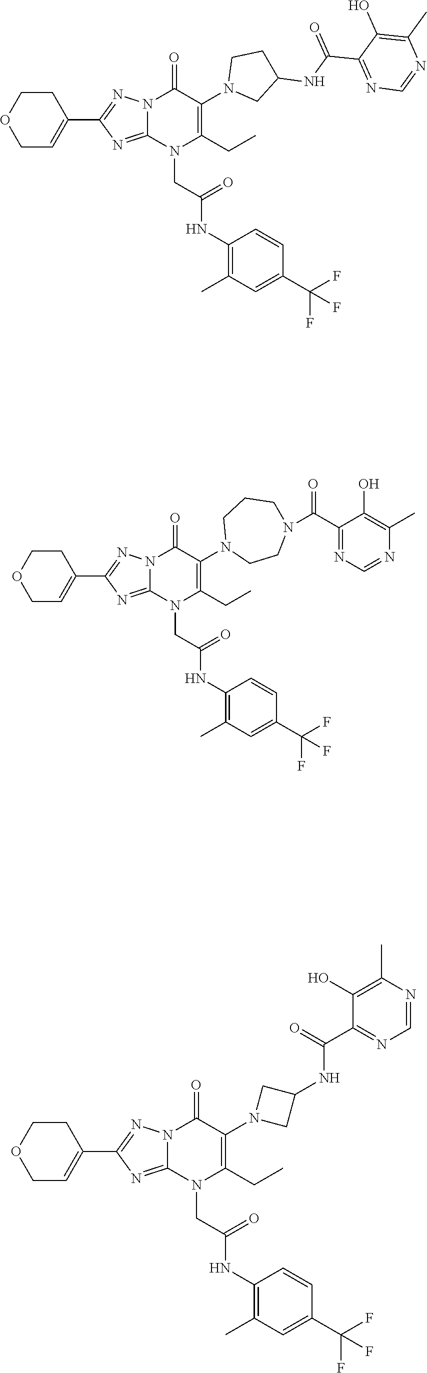 [SMILES: CCc1c(N2CCC(NC(=O)c3ncnc(C)c3O)C2)c(=O)n2nc(C3=CCOCC3)nc2n1CC(=O)Nc1ccc(C(F)(F)F)cc1C ; CCc1c(N2CCCN(C(=O)c3ncnc(C)c3O)CC2)c(=O)n2nc(C3=CCOCC3)nc2n1CC(=O)Nc1ccc(C(F)(F)F)cc1C ; CCc1c(N2CC(NC(=O)c3ncnc(C)c3O)C2)c(=O)n2nc(C3=CCOCC3)nc2n1CC(=O)Nc1ccc(C(F)(F)F)cc1C] 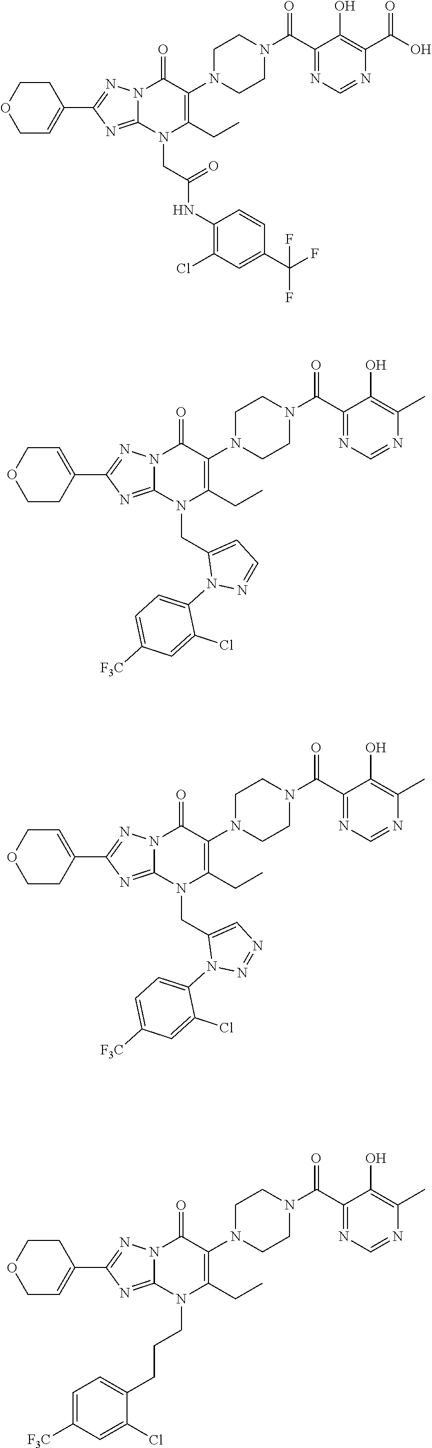 [SMILES: CCc1c(N2CCN(C(=O)c3ncnc(C(=O)O)c3O)CC2)c(=O)n2nc(C3=CCOCC3)nc2n1CC(=O)Nc1ccc(C(F)(F)F)cc1Cl ; CCc1c(N2CCN(C(=O)c3ncnc(C)c3O)CC2)c(=O)n2nc(C3=CCOCC3)nc2n1Cc1ccnn1-c1ccc(C(F)(F)F)cc1Cl ; CCc1c(N2CCN(C(=O)c3ncnc(C)c3O)CC2)c(=O)n2nc(C3=CCOCC3)nc2n1Cc1cnnn1-c1ccc(C(F)(F)F)cc1Cl ; CCc1c(N2CCN(C(=O)c3ncnc(C)c3O)CC2)c(=O)n2nc(C3=CCOCC3)nc2n1CCCc1ccc(C(F)(F)F)cc1Cl] 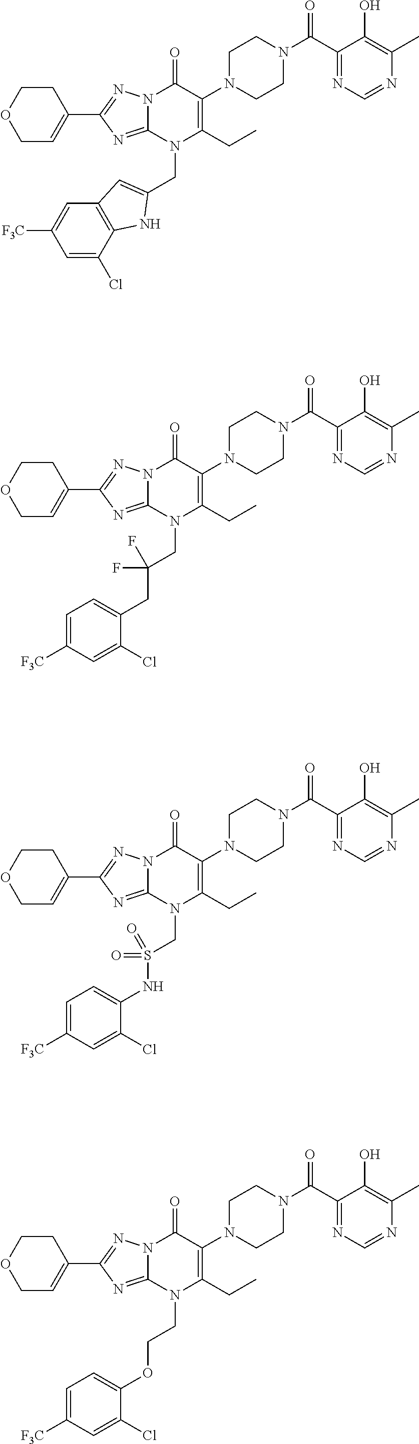 [SMILES: CCc1c(N2CCN(C(=O)c3ncnc(C)c3O)CC2)c(=O)n2nc(C3=CCOCC3)nc2n1Cc1cc2cc(C(F)(F)F)cc(Cl)c2[nH]1 ; CCc1c(N2CCN(C(=O)c3ncnc(C)c3O)CC2)c(=O)n2nc(C3=CCOCC3)nc2n1CC(F)(F)Cc1ccc(C(F)(F)F)cc1Cl ; CCc1c(N2CCN(C(=O)c3ncnc(C)c3O)CC2)c(=O)n2nc(C3=CCOCC3)nc2n1CS(=O)(=O)Nc1ccc(C(F)(F)F)cc1Cl ; CCc1c(N2CCN(C(=O)c3ncnc(C)c3O)CC2)c(=O)n2nc(C3=CCOCC3)nc2n1CCOc1ccc(C(F)(F)F)cc1Cl] 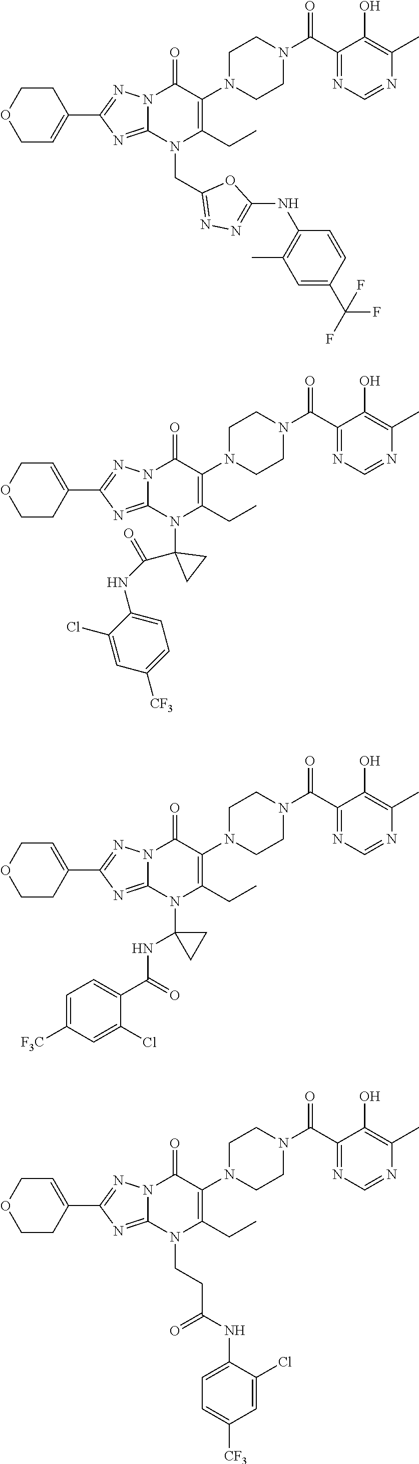 [SMILES: CCc1c(N2CCN(C(=O)c3ncnc(C)c3O)CC2)c(=O)n2nc(C3=CCOCC3)nc2n1Cc1nnc(Nc2ccc(C(F)(F)F)cc2C)o1 ; CCc1c(N2CCN(C(=O)c3ncnc(C)c3O)CC2)c(=O)n2nc(C3=CCOCC3)nc2n1C1(C(=O)Nc2ccc(C(F)(F)F)cc2Cl)CC1 ; CCc1c(N2CCN(C(=O)c3ncnc(C)c3O)CC2)c(=O)n2nc(C3=CCOCC3)nc2n1C1(NC(=O)c2ccc(C(F)(F)F)cc2Cl)CC1 ; CCc1c(N2CCN(C(=O)c3ncnc(C)c3O)CC2)c(=O)n2nc(C3=CCOCC3)nc2n1CCC(=O)Nc1ccc(C(F)(F)F)cc1Cl] 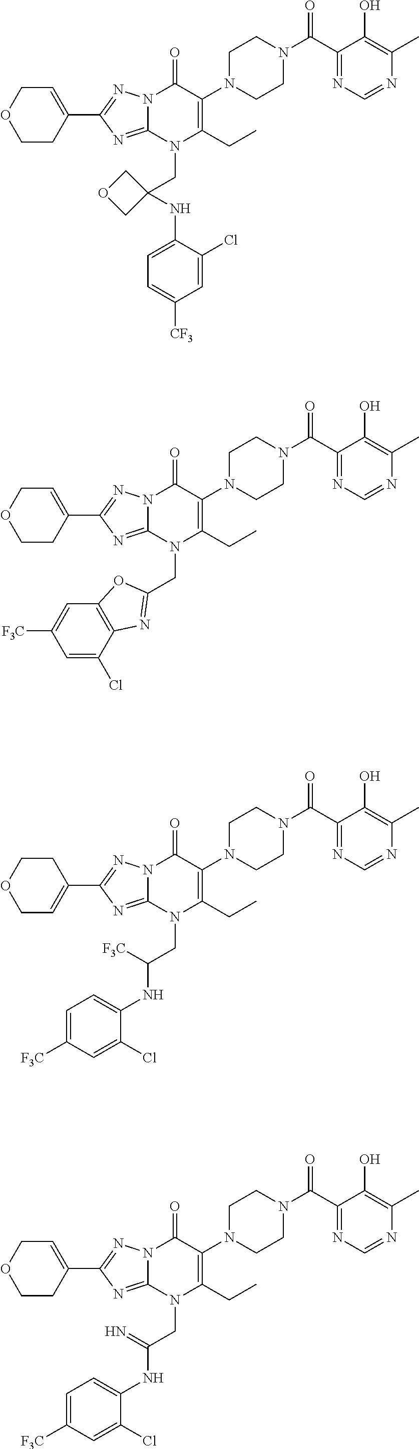 [SMILES: CCc1c(N2CCN(C(=O)c3ncnc(C)c3O)CC2)c(=O)n2nc(C3=CCOCC3)nc2n1CC1(Nc2ccc(C(F)(F)F)cc2Cl)COC1 ; CCc1c(N2CCN(C(=O)c3ncnc(C)c3O)CC2)c(=O)n2nc(C3=CCOCC3)nc2n1Cc1nc2c(Cl)cc(C(F)(F)F)cc2o1 ; CCc1c(N2CCN(C(=O)c3ncnc(C)c3O)CC2)c(=O)n2nc(C3=CCOCC3)nc2n1CC(Nc1ccc(C(F)(F)F)cc1Cl)C(F)(F)F ; CCc1c(N2CCN(C(=O)c3ncnc(C)c3O)CC2)c(=O)n2nc(C3=CCOCC3)nc2n1CC(=N)Nc1ccc(C(F)(F)F)cc1Cl] 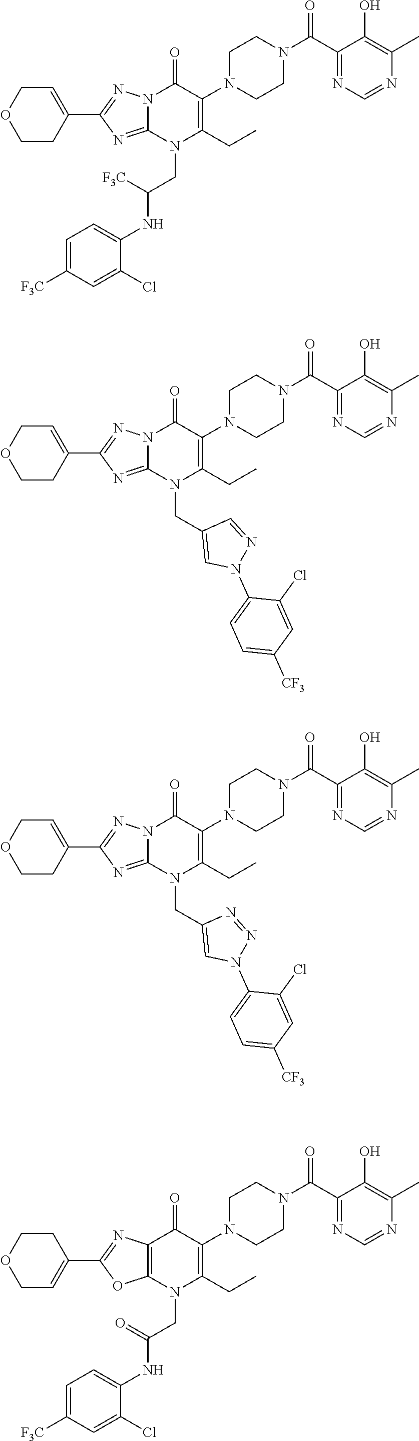 [SMILES: CCc1c(N2CCN(C(=O)c3ncnc(C)c3O)CC2)c(=O)n2nc(C3=CCOCC3)nc2n1CC(Nc1ccc(C(F)(F)F)cc1Cl)C(F)(F)F ; CCc1c(N2CCN(C(=O)c3ncnc(C)c3O)CC2)c(=O)n2nc(C3=CCOCC3)nc2n1Cc1cnn(-c2ccc(C(F)(F)F)cc2Cl)c1 ; CCc1c(N2CCN(C(=O)c3ncnc(C)c3O)CC2)c(=O)n2nc(C3=CCOCC3)nc2n1Cc1cn(-c2ccc(C(F)(F)F)cc2Cl)nn1 ; CCc1c(N2CCN(C(=O)c3ncnc(C)c3O)CC2)c(=O)c2nc(C3=CCOCC3)oc2n1CC(=O)Nc1ccc(C(F)(F)F)cc1Cl] 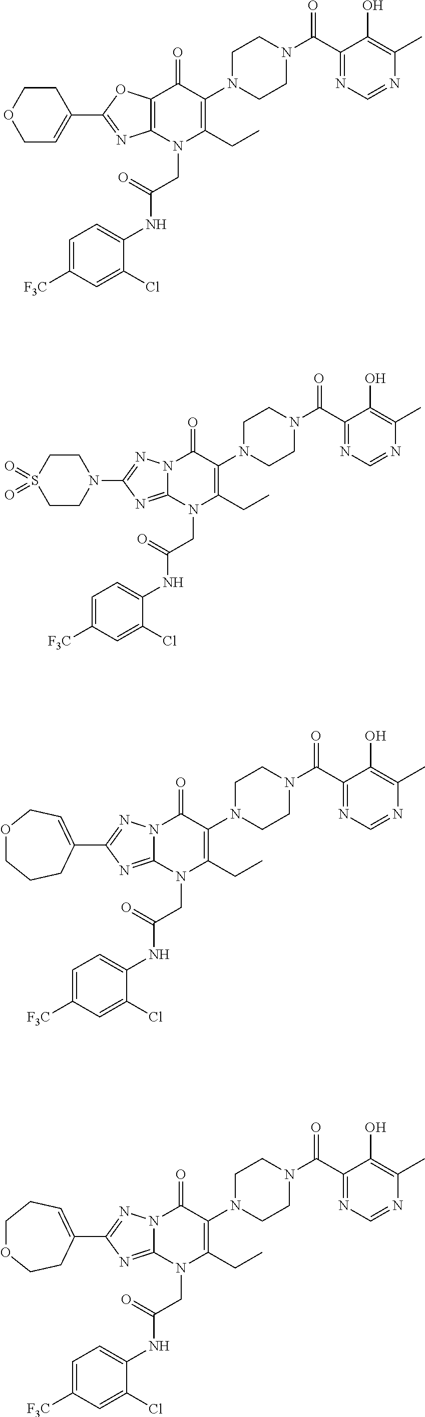 [SMILES: CCc1c(N2CCN(C(=O)c3ncnc(C)c3O)CC2)c(=O)c2oc(C3=CCOCC3)nc2n1CC(=O)Nc1ccc(C(F)(F)F)cc1Cl ; CCc1c(N2CCN(C(=O)c3ncnc(C)c3O)CC2)c(=O)n2nc(N3CCS(=O)(=O)CC3)nc2n1CC(=O)Nc1ccc(C(F)(F)F)cc1Cl ; CCc1c(N2CCN(C(=O)c3ncnc(C)c3O)CC2)c(=O)n2nc(C3=CCOCCC3)nc2n1CC(=O)Nc1ccc(C(F)(F)F)cc1Cl ; CCc1c(N2CCN(C(=O)c3ncnc(C)c3O)CC2)c(=O)n2nc(C3=CCCOCC3)nc2n1CC(=O)Nc1ccc(C(F)(F)F)cc1Cl] 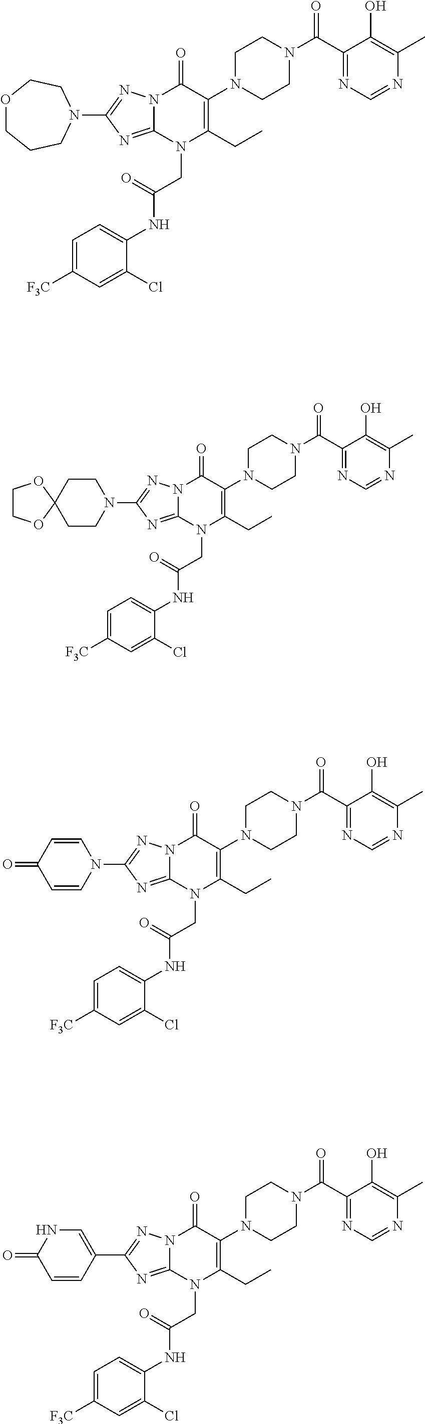 [SMILES: CCc1c(N2CCN(C(=O)c3ncnc(C)c3O)CC2)c(=O)n2nc(N3CCCOCC3)nc2n1CC(=O)Nc1ccc(C(F)(F)F)cc1Cl ; CCc1c(N2CCN(C(=O)c3ncnc(C)c3O)CC2)c(=O)n2nc(N3CCC4(CC3)OCCO4)nc2n1CC(=O)Nc1ccc(C(F)(F)F)cc1Cl ; CCc1c(N2CCN(C(=O)c3ncnc(C)c3O)CC2)c(=O)n2nc(-n3ccc(=O)cc3)nc2n1CC(=O)Nc1ccc(C(F)(F)F)cc1Cl ; CCc1c(N2CCN(C(=O)c3ncnc(C)c3O)CC2)c(=O)n2nc(-c3ccc(=O)[nH]c3)nc2n1CC(=O)Nc1ccc(C(F)(F)F)cc1Cl] 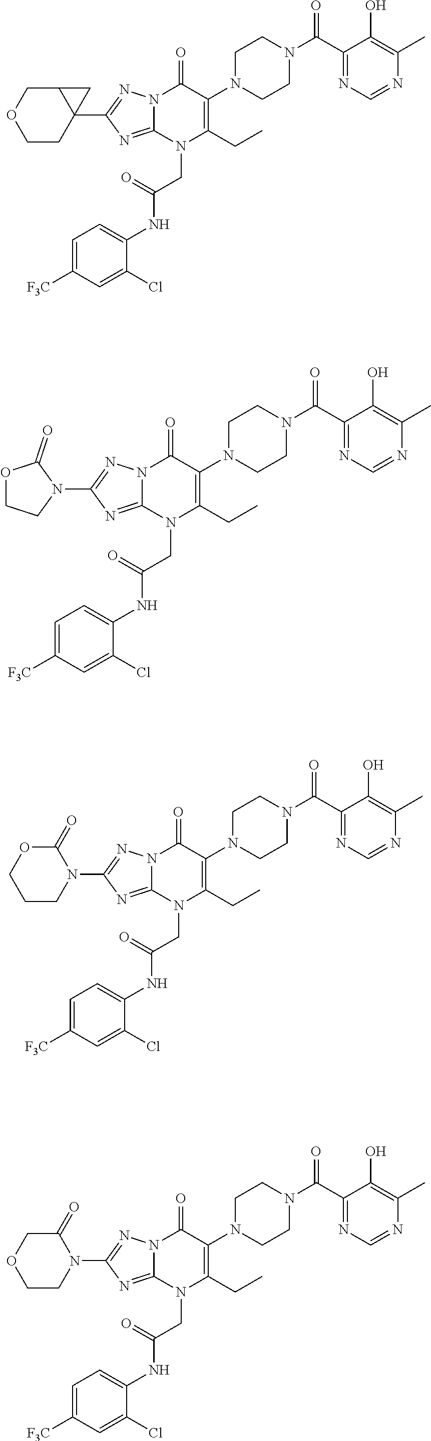 [SMILES: CCc1c(N2CCN(C(=O)c3ncnc(C)c3O)CC2)c(=O)n2nc(C34CCOCC3C4)nc2n1CC(=O)Nc1ccc(C(F)(F)F)cc1Cl ; CCc1c(N2CCN(C(=O)c3ncnc(C)c3O)CC2)c(=O)n2nc(N3CCOC3=O)nc2n1CC(=O)Nc1ccc(C(F)(F)F)cc1Cl ; CCc1c(N2CCN(C(=O)c3ncnc(C)c3O)CC2)c(=O)n2nc(N3CCCOC3=O)nc2n1CC(=O)Nc1ccc(C(F)(F)F)cc1Cl ; CCc1c(N2CCN(C(=O)c3ncnc(C)c3O)CC2)c(=O)n2nc(N3CCOCC3=O)nc2n1CC(=O)Nc1ccc(C(F)(F)F)cc1Cl] 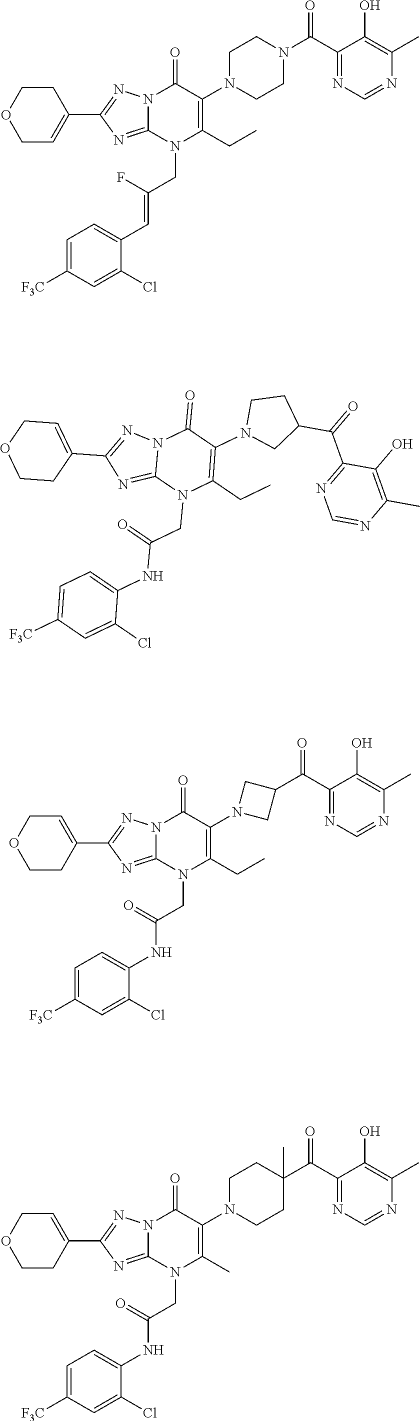 [SMILES: CCc1c(N2CCN(C(=O)c3ncnc(C)c3O)CC2)c(=O)n2nc(C3=CCOCC3)nc2n1C/C(F)=C/c1ccc(C(F)(F)F)cc1Cl ; CCc1c(N2CCC(C(=O)c3ncnc(C)c3O)C2)c(=O)n2nc(C3=CCOCC3)nc2n1CC(=O)Nc1ccc(C(F)(F)F)cc1Cl ; CCc1c(N2CC(C(=O)c3ncnc(C)c3O)C2)c(=O)n2nc(C3=CCOCC3)nc2n1CC(=O)Nc1ccc(C(F)(F)F)cc1Cl ; Cc1ncnc(C(=O)C2(C)CCN(c3c(C)n(CC(=O)Nc4ccc(C(F)(F)F)cc4Cl)c4nc(C5=CCOCC5)nn4c3=O)CC2)c1O] 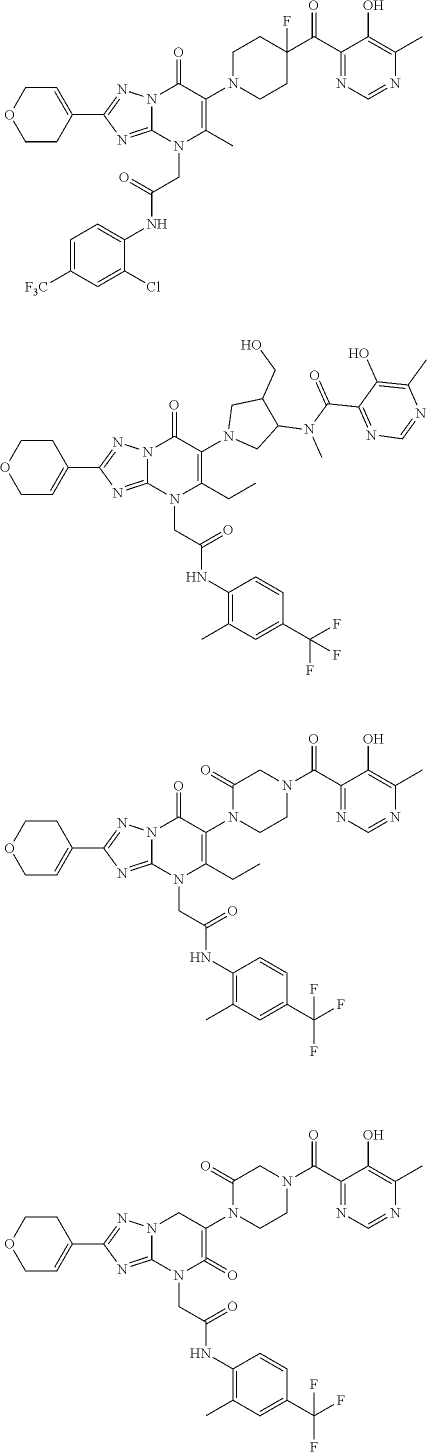 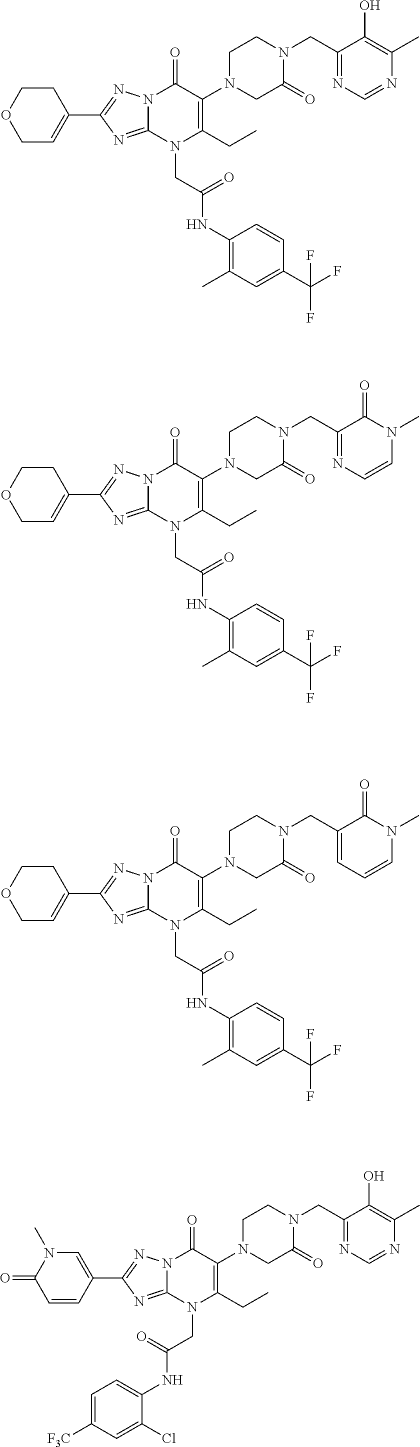 [SMILES: CCc1c(N2CCN(Cc3ncnc(C)c3O)C(=O)C2)c(=O)n2nc(C3=CCOCC3)nc2n1CC(=O)Nc1ccc(C(F)(F)F)cc1C ; CCc1c(N2CCN(Cc3nccn(C)c3=O)C(=O)C2)c(=O)n2nc(C3=CCOCC3)nc2n1CC(=O)Nc1ccc(C(F)(F)F)cc1C ; CCc1c(N2CCN(Cc3cccn(C)c3=O)C(=O)C2)c(=O)n2nc(C3=CCOCC3)nc2n1CC(=O)Nc1ccc(C(F)(F)F)cc1C ; CCc1c(N2CCN(Cc3ncnc(C)c3O)C(=O)C2)c(=O)n2nc(-c3ccc(=O)n(C)c3)nc2n1CC(=O)Nc1ccc(C(F)(F)F)cc1Cl] 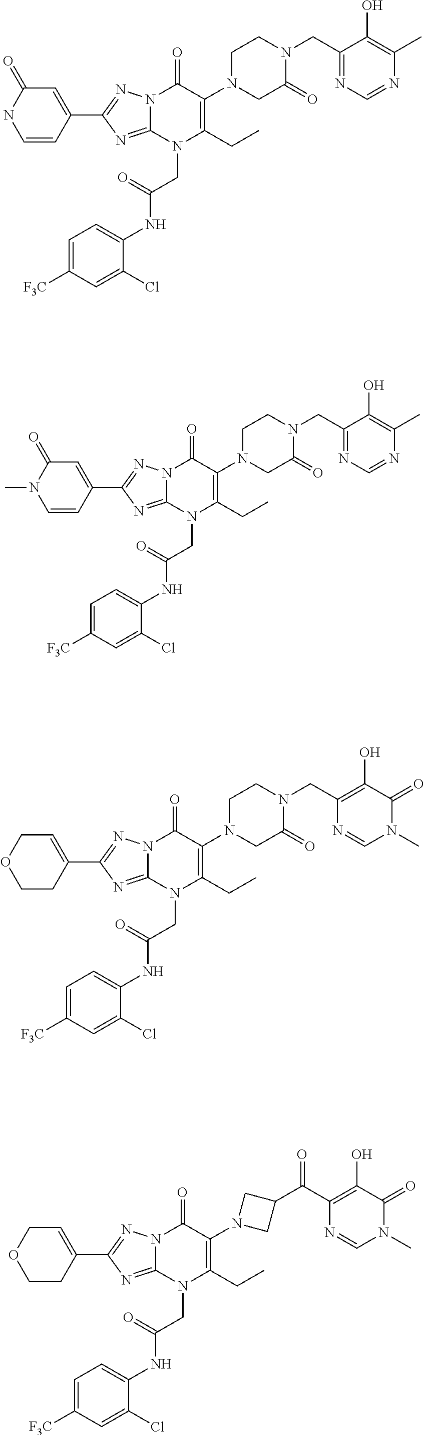 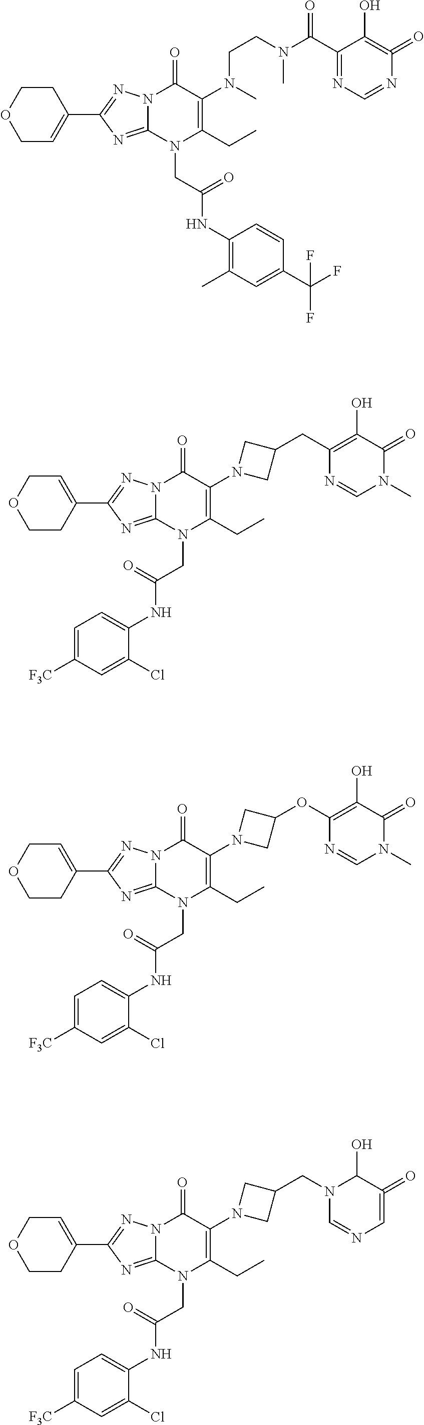 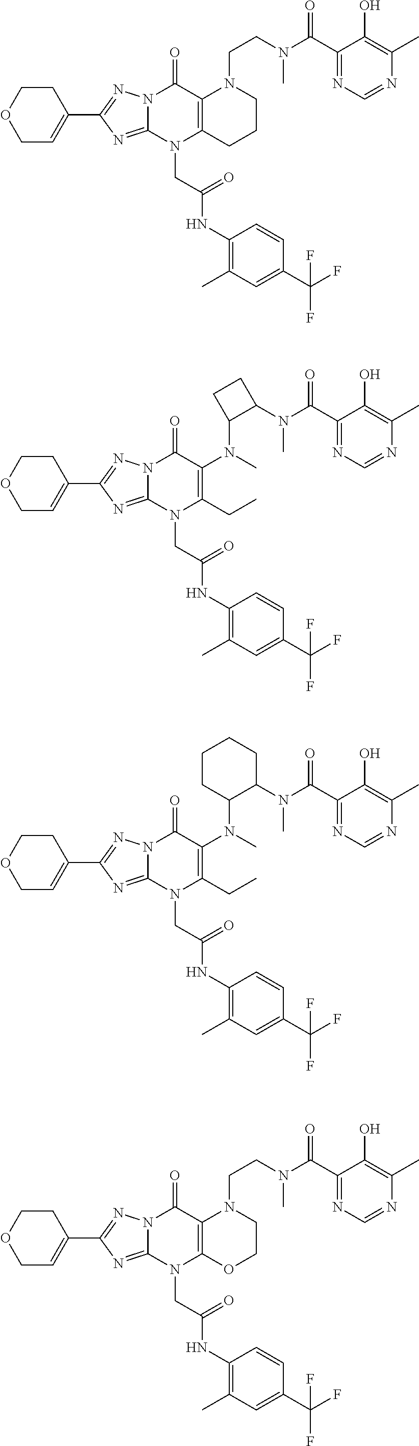 [SMILES: Cc1cc(C(F)(F)F)ccc1NC(=O)Cn1c2c(c(=O)n3nc(C4=CCOCC4)nc13)N(CCN(C)C(=O)c1ncnc(C)c1O)CCC2 ; CCc1c(N(C)C2CCC2N(C)C(=O)c2ncnc(C)c2O)c(=O)n2nc(C3=CCOCC3)nc2n1CC(=O)Nc1ccc(C(F)(F)F)cc1C ; CCc1c(N(C)C2CCCCC2N(C)C(=O)c2ncnc(C)c2O)c(=O)n2nc(C3=CCOCC3)nc2n1CC(=O)Nc1ccc(C(F)(F)F)cc1C ; Cc1cc(C(F)(F)F)ccc1NC(=O)Cn1c2c(c(=O)n3nc(C4=CCOCC4)nc13)N(CCN(C)C(=O)c1ncnc(C)c1O)CCO2] 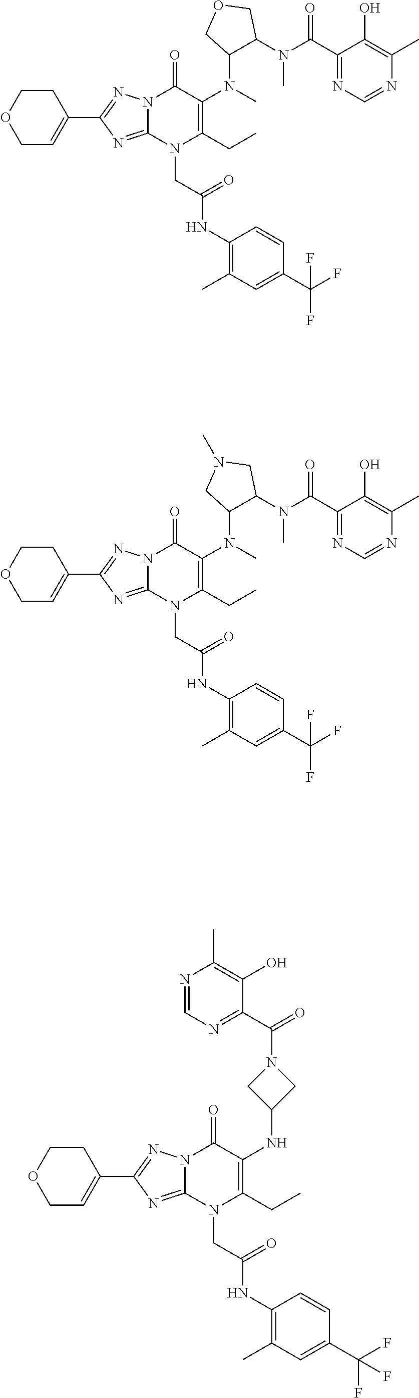 [SMILES: CCc1c(N(C)C2COCC2N(C)C(=O)c2ncnc(C)c2O)c(=O)n2nc(C3=CCOCC3)nc2n1CC(=O)Nc1ccc(C(F)(F)F)cc1C ; CCc1c(N(C)C2CN(C)CC2N(C)C(=O)c2ncnc(C)c2O)c(=O)n2nc(C3=CCOCC3)nc2n1CC(=O)Nc1ccc(C(F)(F)F)cc1C ; CCc1c(NC2CN(C(=O)c3ncnc(C)c3O)C2)c(=O)n2nc(C3=CCOCC3)nc2n1CC(=O)Nc1ccc(C(F)(F)F)cc1C] 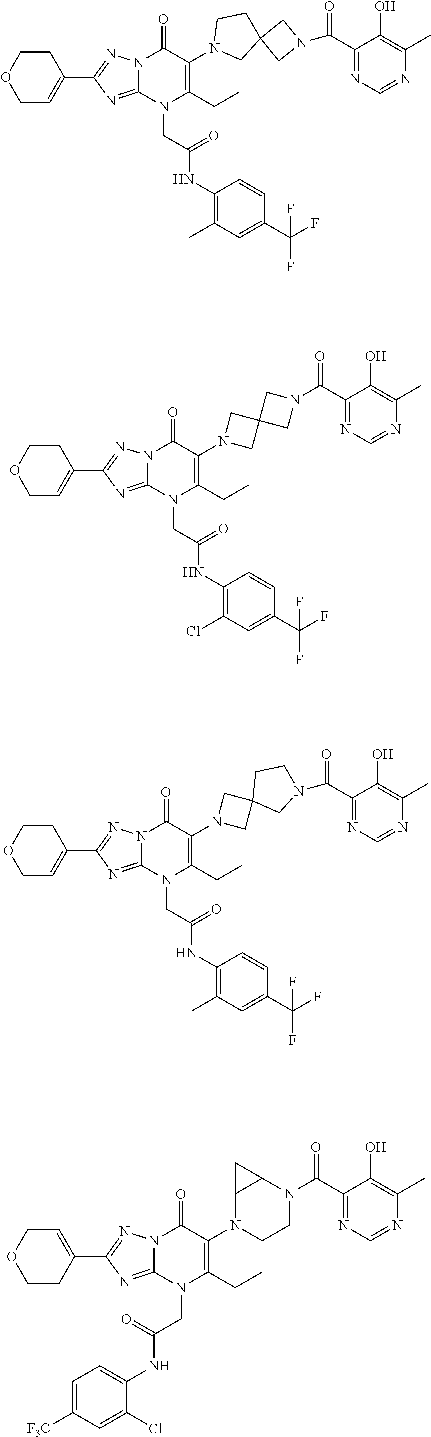 [SMILES: CCc1c(N2CCC3(CN(C(=O)c4ncnc(C)c4O)C3)C2)c(=O)n2nc(C3=CCOCC3)nc2n1CC(=O)Nc1ccc(C(F)(F)F)cc1C ; CCc1c(N2CC3(CN(C(=O)c4ncnc(C)c4O)C3)C2)c(=O)n2nc(C3=CCOCC3)nc2n1CC(=O)Nc1ccc(C(F)(F)F)cc1Cl ; CCc1c(N2CC3(CCN(C(=O)c4ncnc(C)c4O)C3)C2)c(=O)n2nc(C3=CCOCC3)nc2n1CC(=O)Nc1ccc(C(F)(F)F)cc1C ; CCc1c(N2CCN(C(=O)c3ncnc(C)c3O)C3CC32)c(=O)n2nc(C3=CCOCC3)nc2n1CC(=O)Nc1ccc(C(F)(F)F)cc1Cl] 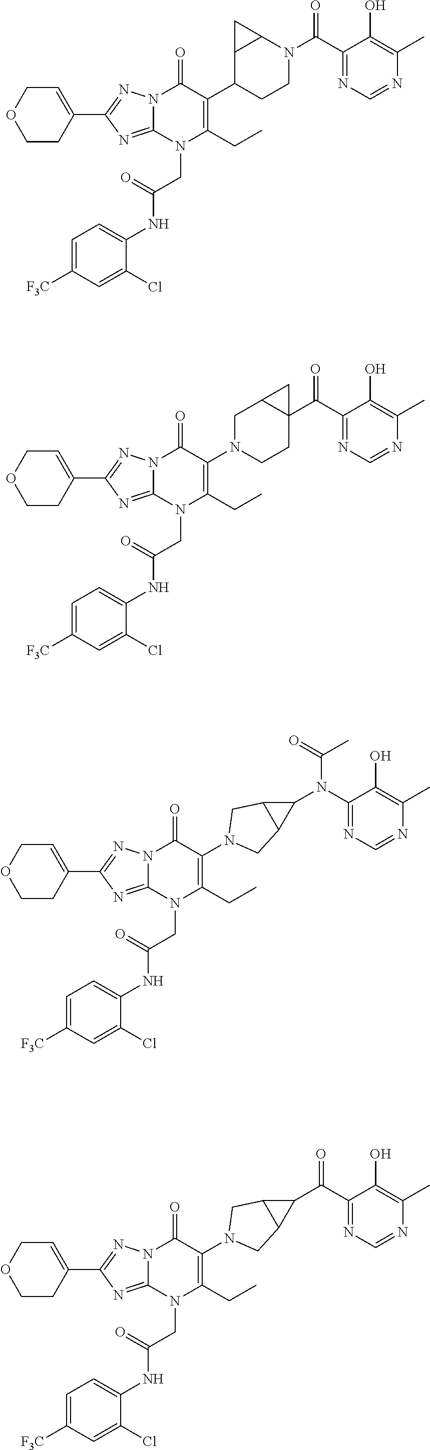 [SMILES: CCc1c(C2CCN(C(=O)c3ncnc(C)c3O)C3CC23)c(=O)n2nc(C3=CCOCC3)nc2n1CC(=O)Nc1ccc(C(F)(F)F)cc1Cl ; CCc1c(N2CCC3(C(=O)c4ncnc(C)c4O)CC3C2)c(=O)n2nc(C3=CCOCC3)nc2n1CC(=O)Nc1ccc(C(F)(F)F)cc1Cl ; CCc1c(N2CC3C(C2)C3N(C(C)=O)c2ncnc(C)c2O)c(=O)n2nc(C3=CCOCC3)nc2n1CC(=O)Nc1ccc(C(F)(F)F)cc1Cl ; CCc1c(N2CC3C(C2)C3C(=O)c2ncnc(C)c2O)c(=O)n2nc(C3=CCOCC3)nc2n1CC(=O)Nc1ccc(C(F)(F)F)cc1Cl] 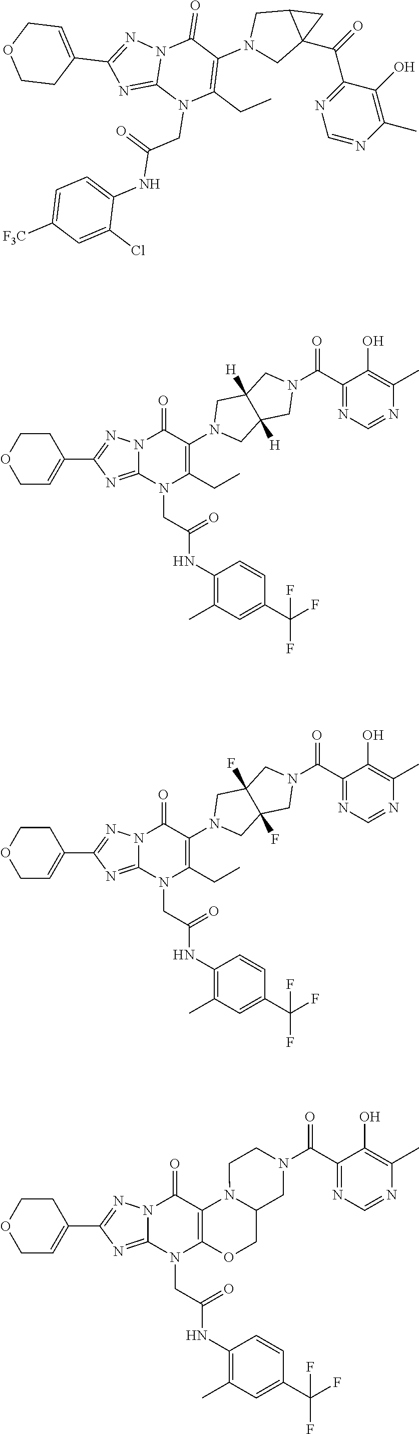 [SMILES: CCc1c(N2CC3CC3(C(=O)c3ncnc(C)c3O)C2)c(=O)n2nc(C3=CCOCC3)nc2n1CC(=O)Nc1ccc(C(F)(F)F)cc1Cl ; CCc1c(N2C[C@H]3CN(C(=O)c4ncnc(C)c4O)C[C@H]3C2)c(=O)n2nc(C3=CCOCC3)nc2n1CC(=O)Nc1ccc(C(F)(F)F)cc1C ; CCc1c(N2C[C@]3(F)CN(C(=O)c4ncnc(C)c4O)C[C@]3(F)C2)c(=O)n2nc(C3=CCOCC3)nc2n1CC(=O)Nc1ccc(C(F)(F)F)cc1C ; Cc1cc(C(F)(F)F)ccc1NC(=O)Cn1c2c(c(=O)n3nc(C4=CCOCC4)nc13)N1CCN(C(=O)c3ncnc(C)c3O)CC1CO2] 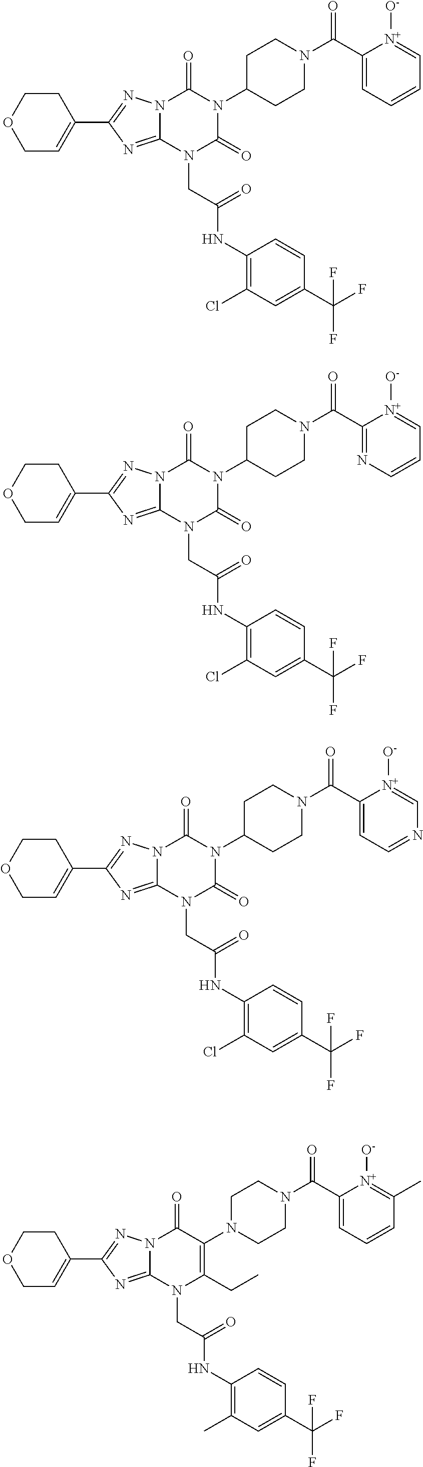 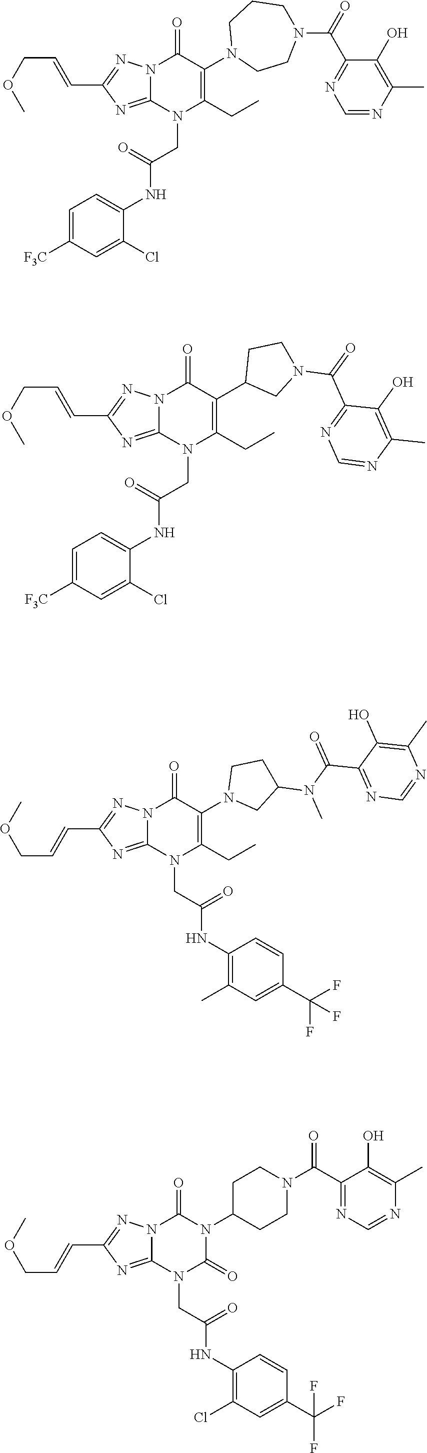 [SMILES: CCc1c(N2CCCN(C(=O)c3ncnc(C)c3O)CC2)c(=O)n2nc(/C=C/COC)nc2n1CC(=O)Nc1ccc(C(F)(F)F)cc1Cl ; CCc1c(C2CCN(C(=O)c3ncnc(C)c3O)C2)c(=O)n2nc(/C=C/COC)nc2n1CC(=O)Nc1ccc(C(F)(F)F)cc1Cl ; CCc1c(N2CCC(N(C)C(=O)c3ncnc(C)c3O)C2)c(=O)n2nc(/C=C/COC)nc2n1CC(=O)Nc1ccc(C(F)(F)F)cc1C ; COC/C=C/c1nc2n(CC(=O)Nc3ccc(C(F)(F)F)cc3Cl)c(=O)n(C3CCN(C(=O)c4ncnc(C)c4O)CC3)c(=O)n2n1] 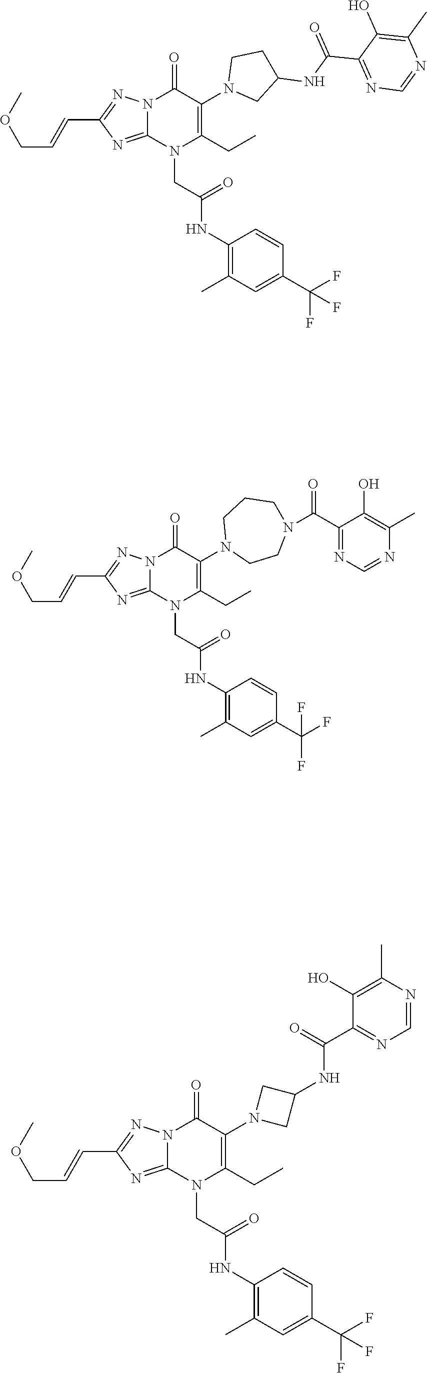 [SMILES: CCc1c(N2CCC(NC(=O)c3ncnc(C)c3O)C2)c(=O)n2nc(/C=C/COC)nc2n1CC(=O)Nc1ccc(C(F)(F)F)cc1C ; CCc1c(N2CCCN(C(=O)c3ncnc(C)c3O)CC2)c(=O)n2nc(/C=C/COC)nc2n1CC(=O)Nc1ccc(C(F)(F)F)cc1C ; CCc1c(N2CC(NC(=O)c3ncnc(C)c3O)C2)c(=O)n2nc(/C=C/COC)nc2n1CC(=O)Nc1ccc(C(F)(F)F)cc1C] 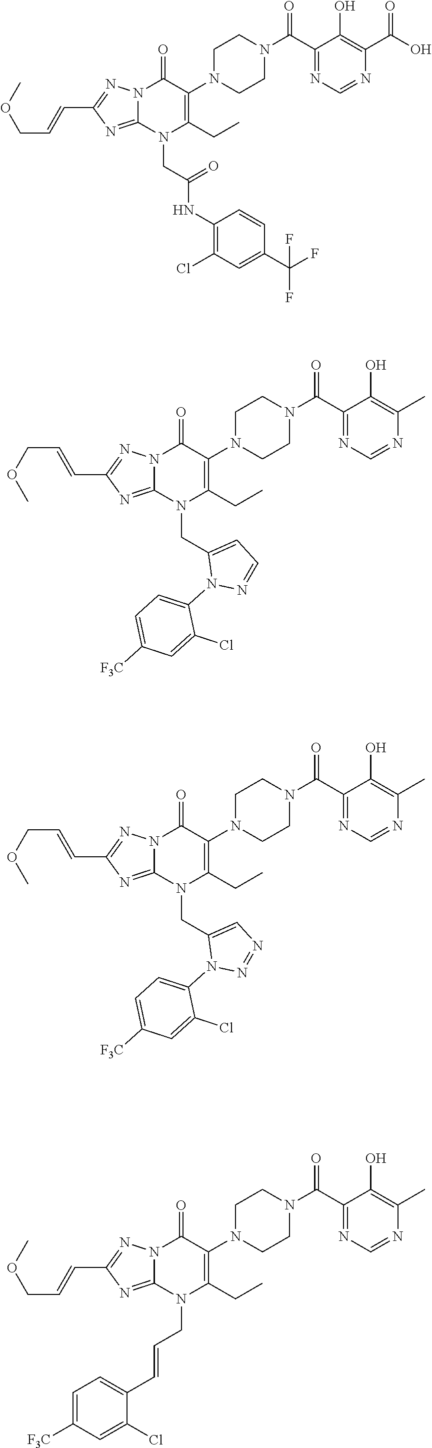 [SMILES: CCc1c(N2CCN(C(=O)c3ncnc(C(=O)O)c3O)CC2)c(=O)n2nc(/C=C/COC)nc2n1CC(=O)Nc1ccc(C(F)(F)F)cc1Cl ; CCc1c(N2CCN(C(=O)c3ncnc(C)c3O)CC2)c(=O)n2nc(/C=C/COC)nc2n1Cc1ccnn1-c1ccc(C(F)(F)F)cc1Cl ; CCc1c(N2CCN(C(=O)c3ncnc(C)c3O)CC2)c(=O)n2nc(/C=C/COC)nc2n1Cc1cnnn1-c1ccc(C(F)(F)F)cc1Cl ; CCc1c(N2CCN(C(=O)c3ncnc(C)c3O)CC2)c(=O)n2nc(/C=C/COC)nc2n1C/C=C/c1ccc(C(F)(F)F)cc1Cl] 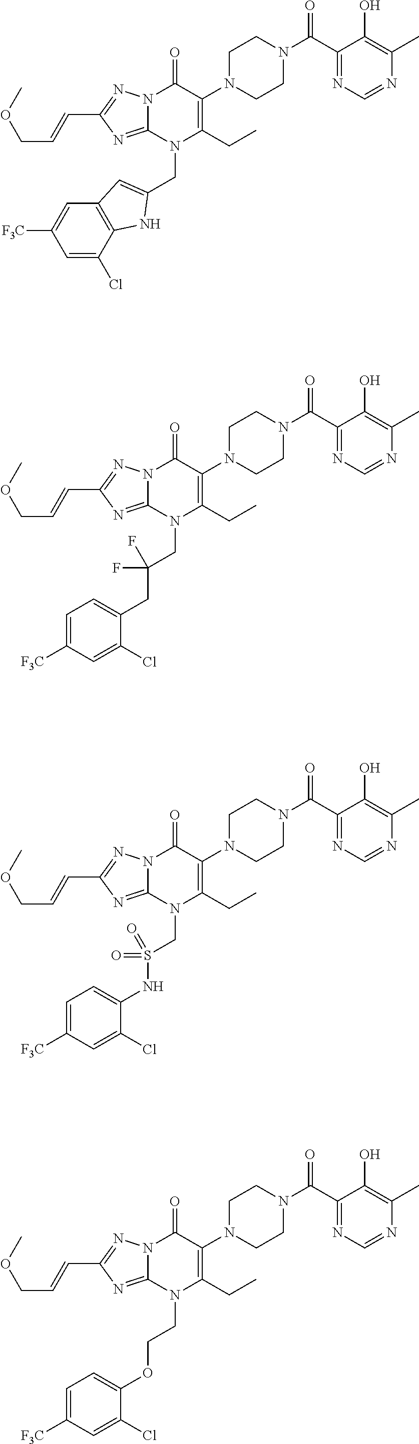 [SMILES: CCc1c(N2CCN(C(=O)c3ncnc(C)c3O)CC2)c(=O)n2nc(/C=C/COC)nc2n1Cc1cc2cc(C(F)(F)F)cc(Cl)c2[nH]1 ; CCc1c(N2CCN(C(=O)c3ncnc(C)c3O)CC2)c(=O)n2nc(/C=C/COC)nc2n1CC(F)(F)Cc1ccc(C(F)(F)F)cc1Cl ; CCc1c(N2CCN(C(=O)c3ncnc(C)c3O)CC2)c(=O)n2nc(/C=C/COC)nc2n1CS(=O)(=O)Nc1ccc(C(F)(F)F)cc1Cl ; CCc1c(N2CCN(C(=O)c3ncnc(C)c3O)CC2)c(=O)n2nc(/C=C/COC)nc2n1CCOc1ccc(C(F)(F)F)cc1Cl] 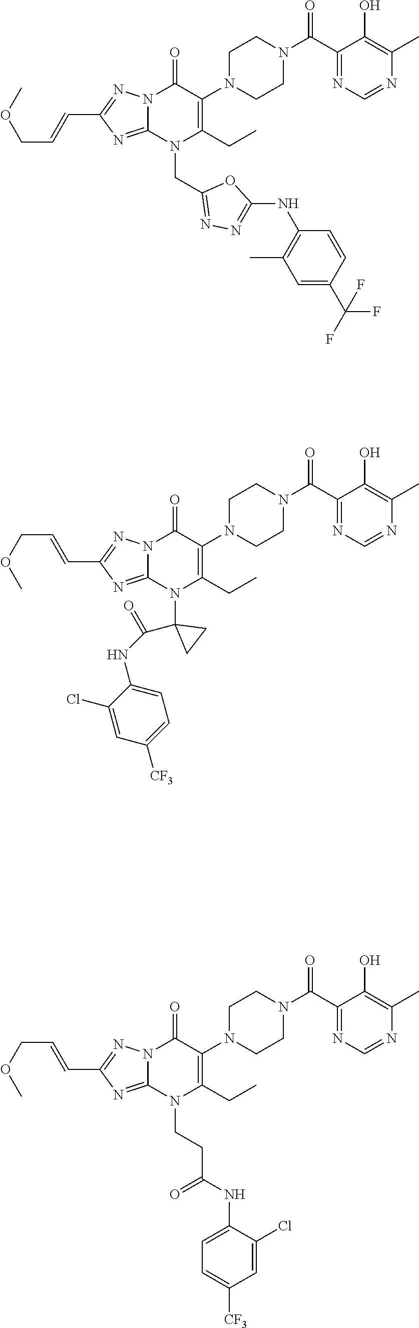 [SMILES: CCc1c(N2CCN(C(=O)c3ncnc(C)c3O)CC2)c(=O)n2nc(/C=C/COC)nc2n1Cc1nnc(Nc2ccc(C(F)(F)F)cc2C)o1 ; CCc1c(N2CCN(C(=O)c3ncnc(C)c3O)CC2)c(=O)n2nc(/C=C/COC)nc2n1C1(C(=O)Nc2ccc(C(F)(F)F)cc2Cl)CC1 ; CCc1c(N2CCN(C(=O)c3ncnc(C)c3O)CC2)c(=O)n2nc(/C=C/COC)nc2n1CCC(=O)Nc1ccc(C(F)(F)F)cc1Cl] 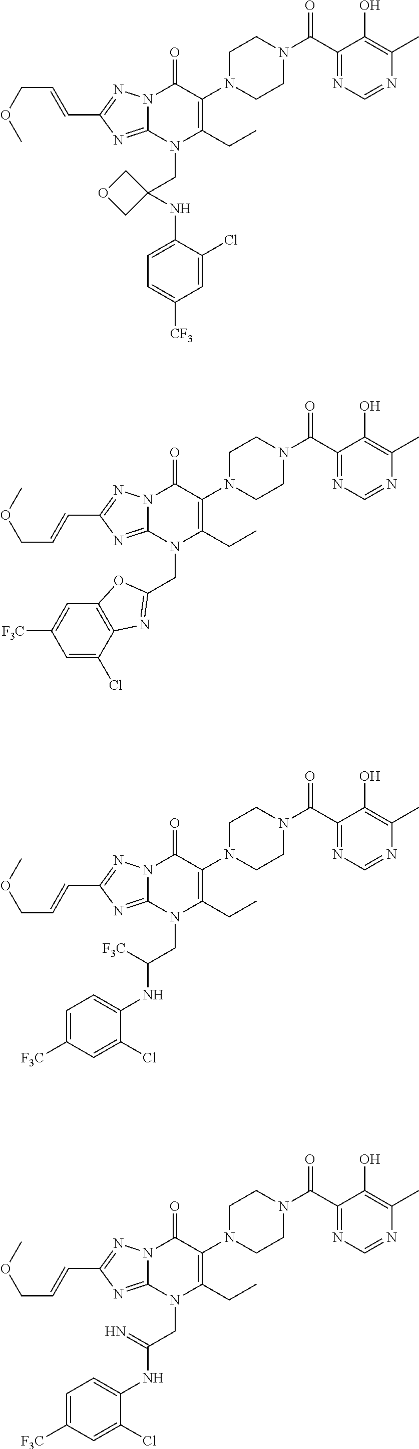 [SMILES: CCc1c(N2CCN(C(=O)c3ncnc(C)c3O)CC2)c(=O)n2nc(/C=C/COC)nc2n1CC1(Nc2ccc(C(F)(F)F)cc2Cl)COC1 ; CCc1c(N2CCN(C(=O)c3ncnc(C)c3O)CC2)c(=O)n2nc(/C=C/COC)nc2n1Cc1nc2c(Cl)cc(C(F)(F)F)cc2o1 ; CCc1c(N2CCN(C(=O)c3ncnc(C)c3O)CC2)c(=O)n2nc(/C=C/COC)nc2n1CC(Nc1ccc(C(F)(F)F)cc1Cl)C(F)(F)F ; CCc1c(N2CCN(C(=O)c3ncnc(C)c3O)CC2)c(=O)n2nc(/C=C/COC)nc2n1CC(=N)Nc1ccc(C(F)(F)F)cc1Cl] 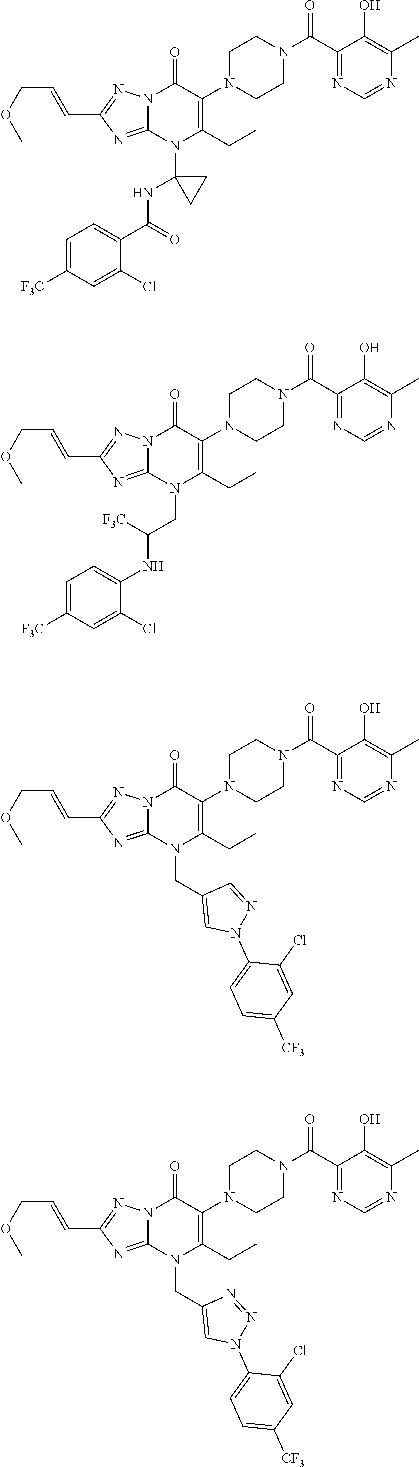 [SMILES: CCc1c(N2CCN(C(=O)c3ncnc(C)c3O)CC2)c(=O)n2nc(/C=C/COC)nc2n1C1(NC(=O)c2ccc(C(F)(F)F)cc2Cl)CC1 ; CCc1c(N2CCN(C(=O)c3ncnc(C)c3O)CC2)c(=O)n2nc(/C=C/COC)nc2n1CC(Nc1ccc(C(F)(F)F)cc1Cl)C(F)(F)F ; CCc1c(N2CCN(C(=O)c3ncnc(C)c3O)CC2)c(=O)n2nc(/C=C/COC)nc2n1Cc1cnn(-c2ccc(C(F)(F)F)cc2Cl)c1 ; CCc1c(N2CCN(C(=O)c3ncnc(C)c3O)CC2)c(=O)n2nc(/C=C/COC)nc2n1Cc1cn(-c2ccc(C(F)(F)F)cc2Cl)nn1] 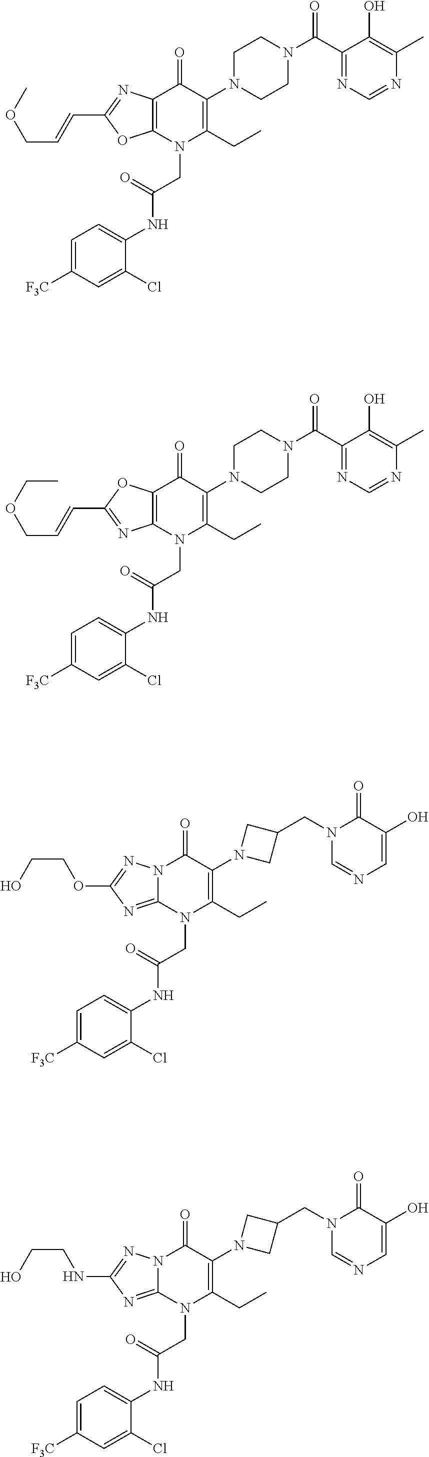 [SMILES: CCc1c(N2CCN(C(=O)c3ncnc(C)c3O)CC2)c(=O)c2nc(/C=C/COC)oc2n1CC(=O)Nc1ccc(C(F)(F)F)cc1Cl ; CCOC/C=C/c1nc2c(o1)c(=O)c(N1CCN(C(=O)c3ncnc(C)c3O)CC1)c(CC)n2CC(=O)Nc1ccc(C(F)(F)F)cc1Cl ; CCc1c(N2CC(Cn3cncc(O)c3=O)C2)c(=O)n2nc(OCCO)nc2n1CC(=O)Nc1ccc(C(F)(F)F)cc1Cl ; CCc1c(N2CC(Cn3cncc(O)c3=O)C2)c(=O)n2nc(NCCO)nc2n1CC(=O)Nc1ccc(C(F)(F)F)cc1Cl] 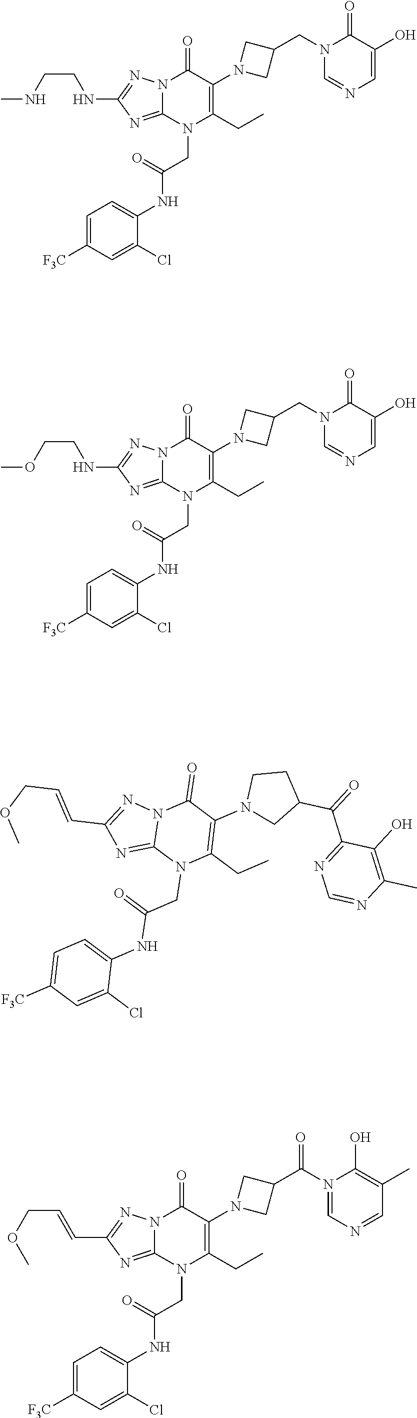 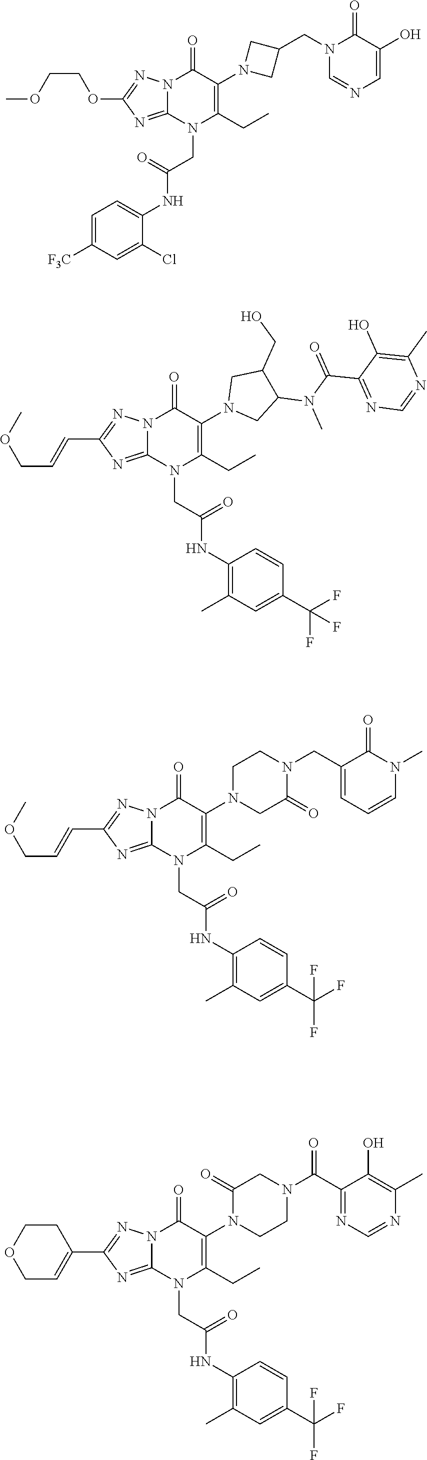 [SMILES: CCc1c(N2CC(Cn3cncc(O)c3=O)C2)c(=O)n2nc(OCCOC)nc2n1CC(=O)Nc1ccc(C(F)(F)F)cc1Cl ; CCc1c(N2CC(CO)C(N(C)C(=O)c3ncnc(C)c3O)C2)c(=O)n2nc(/C=C/COC)nc2n1CC(=O)Nc1ccc(C(F)(F)F)cc1C ; CCc1c(N2CCN(Cc3cccn(C)c3=O)C(=O)C2)c(=O)n2nc(/C=C/COC)nc2n1CC(=O)Nc1ccc(C(F)(F)F)cc1C ; CCc1c(N2CCN(C(=O)c3ncnc(C)c3O)CC2=O)c(=O)n2nc(C3=CCOCC3)nc2n1CC(=O)Nc1ccc(C(F)(F)F)cc1C] 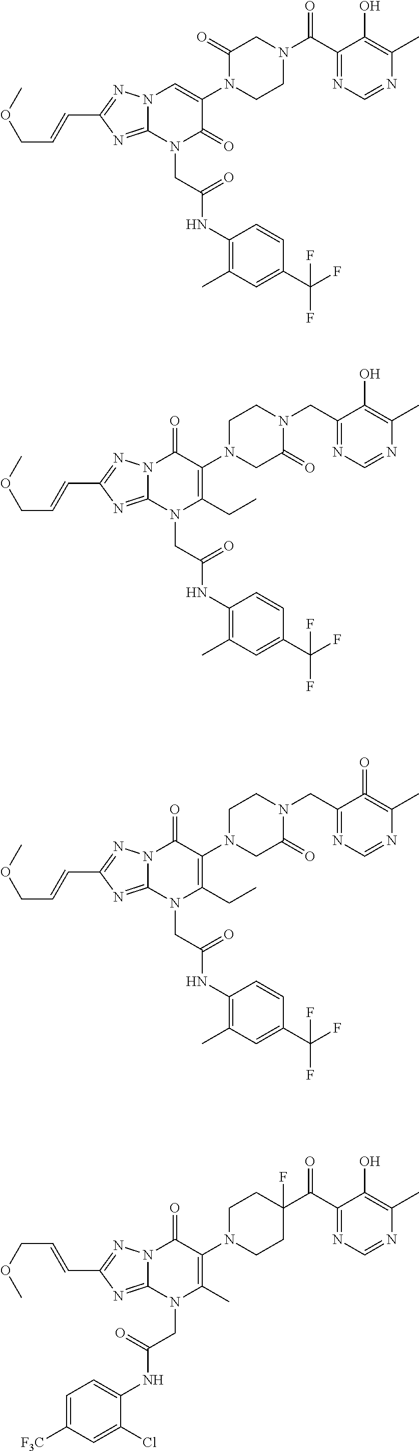 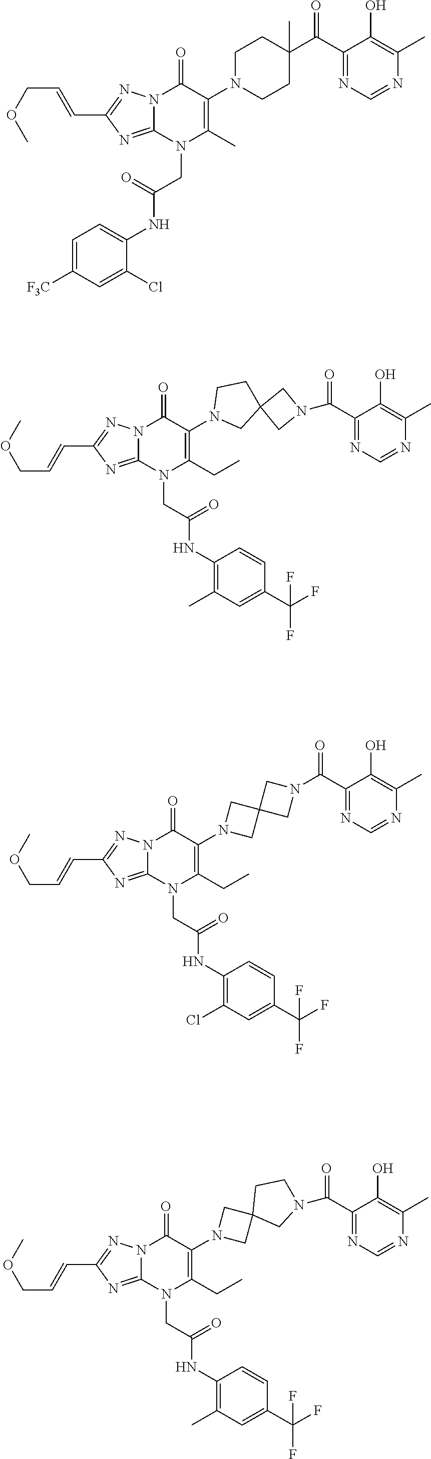 [SMILES: COC/C=C/c1nc2n(CC(=O)Nc3ccc(C(F)(F)F)cc3Cl)c(C)c(N3CCC(C)(C(=O)c4ncnc(C)c4O)CC3)c(=O)n2n1 ; CCc1c(N2CCC3(CN(C(=O)c4ncnc(C)c4O)C3)C2)c(=O)n2nc(/C=C/COC)nc2n1CC(=O)Nc1ccc(C(F)(F)F)cc1C ; CCc1c(N2CC3(CN(C(=O)c4ncnc(C)c4O)C3)C2)c(=O)n2nc(/C=C/COC)nc2n1CC(=O)Nc1ccc(C(F)(F)F)cc1Cl ; CCc1c(N2CC3(CCN(C(=O)c4ncnc(C)c4O)C3)C2)c(=O)n2nc(/C=C/COC)nc2n1CC(=O)Nc1ccc(C(F)(F)F)cc1C] 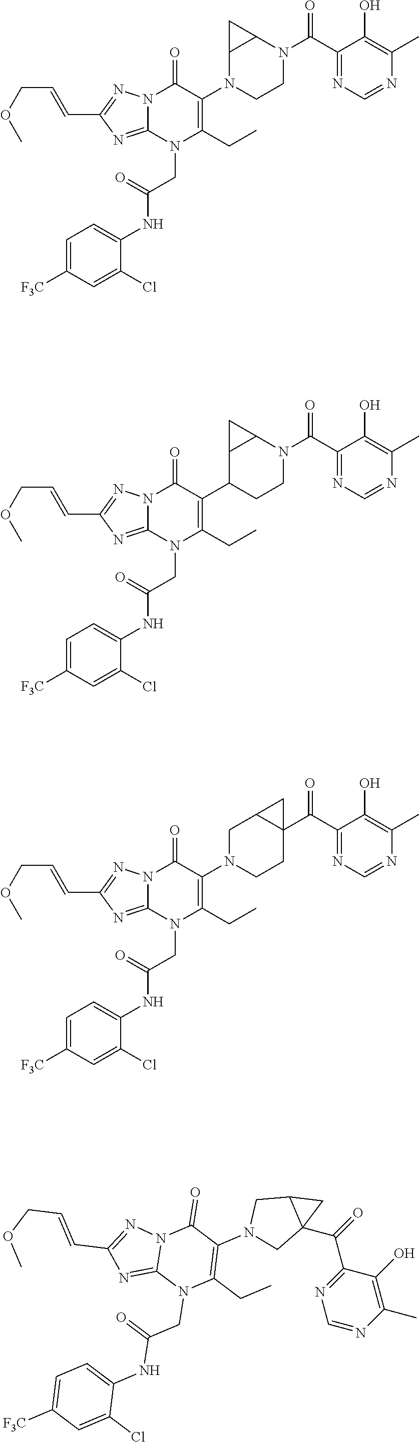 [SMILES: CCc1c(N2CCN(C(=O)c3ncnc(C)c3O)C3CC32)c(=O)n2nc(/C=C/COC)nc2n1CC(=O)Nc1ccc(C(F)(F)F)cc1Cl ; CCc1c(C2CCN(C(=O)c3ncnc(C)c3O)C3CC23)c(=O)n2nc(/C=C/COC)nc2n1CC(=O)Nc1ccc(C(F)(F)F)cc1Cl ; CCc1c(N2CCC3(C(=O)c4ncnc(C)c4O)CC3C2)c(=O)n2nc(/C=C/COC)nc2n1CC(=O)Nc1ccc(C(F)(F)F)cc1Cl ; CCc1c(N2CC3CC3(C(=O)c3ncnc(C)c3O)C2)c(=O)n2nc(/C=C/COC)nc2n1CC(=O)Nc1ccc(C(F)(F)F)cc1Cl] 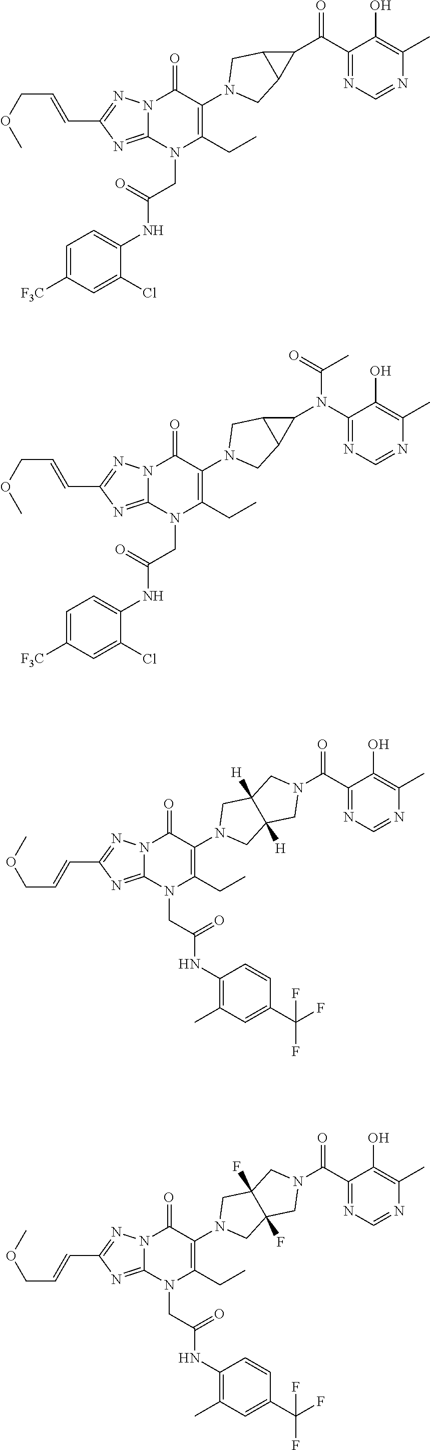 [SMILES: CCc1c(N2CC3C(C2)C3C(=O)c2ncnc(C)c2O)c(=O)n2nc(/C=C/COC)nc2n1CC(=O)Nc1ccc(C(F)(F)F)cc1Cl ; CCc1c(N2CC3C(C2)C3N(C(C)=O)c2ncnc(C)c2O)c(=O)n2nc(/C=C/COC)nc2n1CC(=O)Nc1ccc(C(F)(F)F)cc1Cl ; CCc1c(N2C[C@H]3CN(C(=O)c4ncnc(C)c4O)C[C@H]3C2)c(=O)n2nc(/C=C/COC)nc2n1CC(=O)Nc1ccc(C(F)(F)F)cc1C ; CCc1c(N2C[C@]3(F)CN(C(=O)c4ncnc(C)c4O)C[C@]3(F)C2)c(=O)n2nc(/C=C/COC)nc2n1CC(=O)Nc1ccc(C(F)(F)F)cc1C] 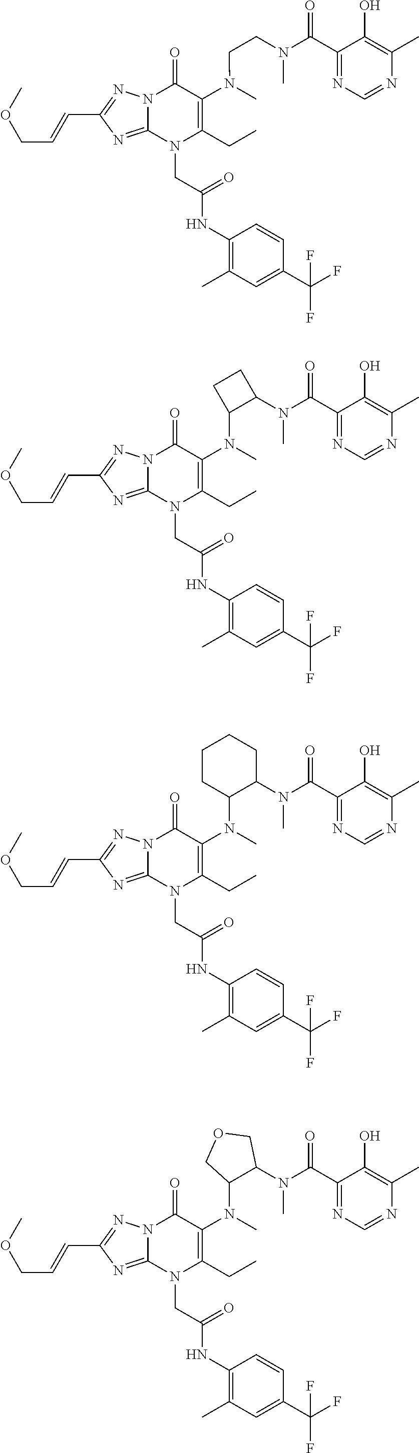 [SMILES: CCc1c(N(C)CCN(C)C(=O)c2ncnc(C)c2O)c(=O)n2nc(/C=C/COC)nc2n1CC(=O)Nc1ccc(C(F)(F)F)cc1C ; CCc1c(N(C)C2CCC2N(C)C(=O)c2ncnc(C)c2O)c(=O)n2nc(/C=C/COC)nc2n1CC(=O)Nc1ccc(C(F)(F)F)cc1C ; CCc1c(N(C)C2CCCCC2N(C)C(=O)c2ncnc(C)c2O)c(=O)n2nc(/C=C/COC)nc2n1CC(=O)Nc1ccc(C(F)(F)F)cc1C ; CCc1c(N(C)C2COCC2N(C)C(=O)c2ncnc(C)c2O)c(=O)n2nc(/C=C/COC)nc2n1CC(=O)Nc1ccc(C(F)(F)F)cc1C] 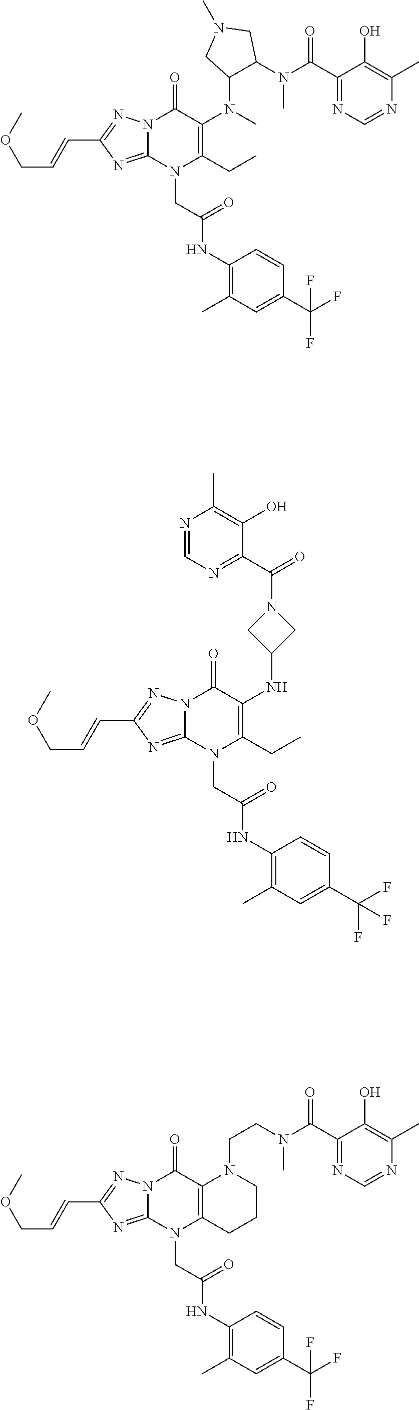 [SMILES: CCc1c(N(C)C2CN(C)CC2N(C)C(=O)c2ncnc(C)c2O)c(=O)n2nc(/C=C/COC)nc2n1CC(=O)Nc1ccc(C(F)(F)F)cc1C ; CCc1c(NC2CN(C(=O)c3ncnc(C)c3O)C2)c(=O)n2nc(/C=C/COC)nc2n1CC(=O)Nc1ccc(C(F)(F)F)cc1C ; COC/C=C/c1nc2n(CC(=O)Nc3ccc(C(F)(F)F)cc3C)c3c(c(=O)n2n1)N(CCN(C)C(=O)c1ncnc(C)c1O)CCC3] 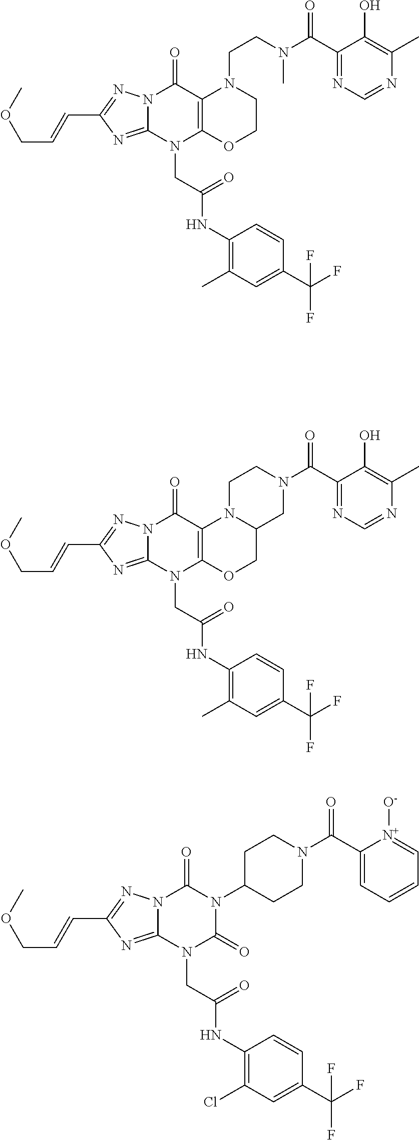 [SMILES: COC/C=C/c1nc2n(CC(=O)Nc3ccc(C(F)(F)F)cc3C)c3c(c(=O)n2n1)N(CCN(C)C(=O)c1ncnc(C)c1O)CCO3 ; COC/C=C/c1nc2n(CC(=O)Nc3ccc(C(F)(F)F)cc3C)c3c(c(=O)n2n1)N1CCN(C(=O)c2ncnc(C)c2O)CC1CO3 ; COC/C=C/c1nc2n(CC(=O)Nc3ccc(C(F)(F)F)cc3Cl)c(=O)n(C3CCN(C(=O)c4cccc[n+]4[O-])CC3)c(=O)n2n1] 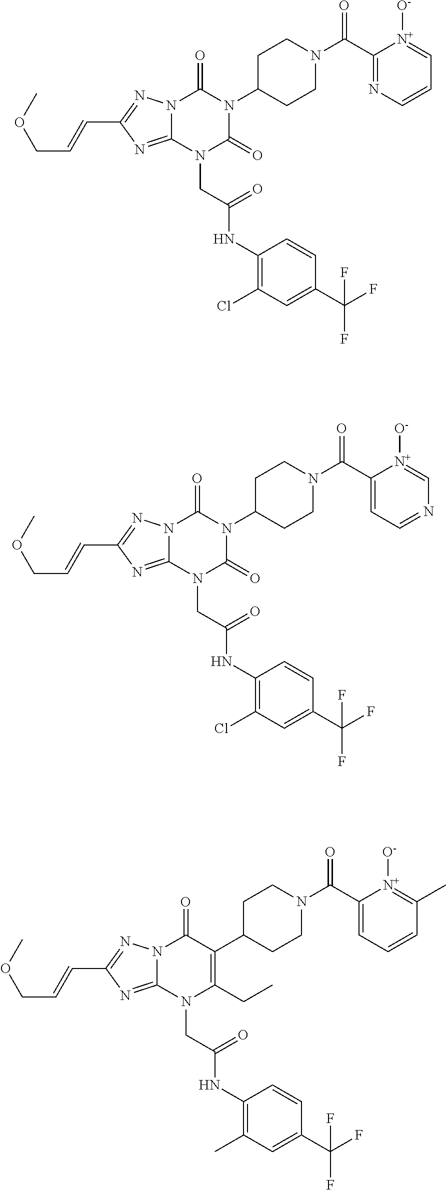 [SMILES: COC/C=C/c1nc2n(CC(=O)Nc3ccc(C(F)(F)F)cc3Cl)c(=O)n(C3CCN(C(=O)c4nccc[n+]4[O-])CC3)c(=O)n2n1 ; COC/C=C/c1nc2n(CC(=O)Nc3ccc(C(F)(F)F)cc3Cl)c(=O)n(C3CCN(C(=O)c4ccnc[n+]4[O-])CC3)c(=O)n2n1 ; CCc1c(C2CCN(C(=O)c3cccc(C)[n+]3[O-])CC2)c(=O)n2nc(/C=C/COC)nc2n1CC(=O)Nc1ccc(C(F)(F)F)cc1C] 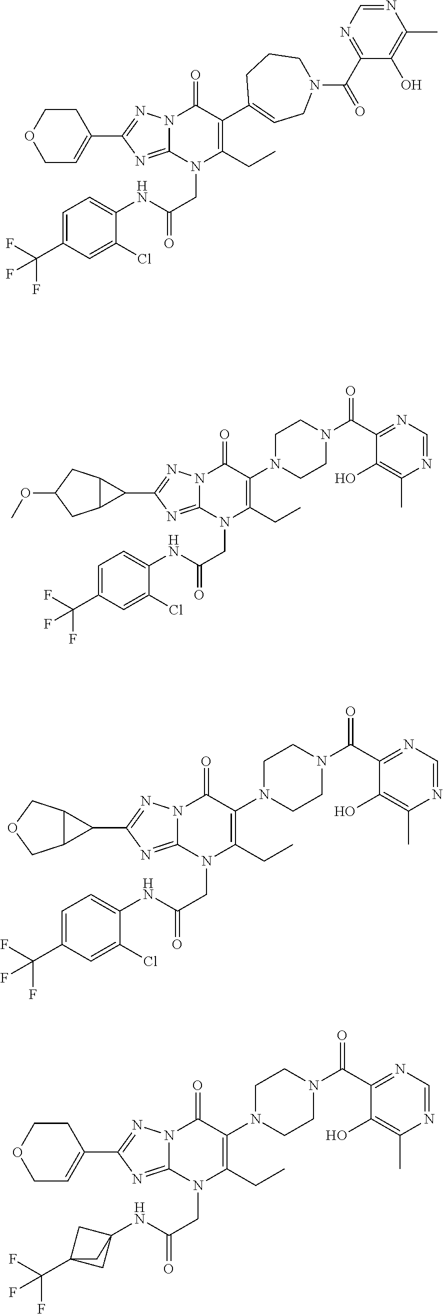 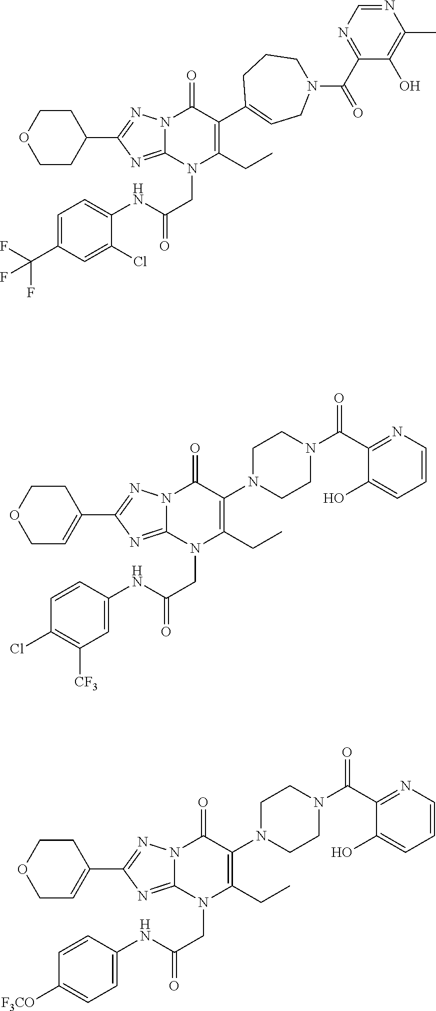 [SMILES: CCc1c(C2=CCN(C(=O)c3ncnc(C)c3O)CCC2)c(=O)n2nc(C3CCOCC3)nc2n1CC(=O)Nc1ccc(C(F)(F)F)cc1Cl ; CCc1c(N2CCN(C(=O)c3ncccc3O)CC2)c(=O)n2nc(C3=CCOCC3)nc2n1CC(=O)Nc1ccc(Cl)c(C(F)(F)F)c1 ; CCc1c(N2CCN(C(=O)c3ncccc3O)CC2)c(=O)n2nc(C3=CCOCC3)nc2n1CC(=O)Nc1ccc(OC(F)(F)F)cc1]
or a tautomer, cis- or trans-isomer, mesomer, racemate, enantiomer, diastereomer, or mixture thereof, or a pharmaceutically acceptable salt, solvate, or prodrug thereof.

36. A pharmaceutical composition comprising a therapeutically effective amount of the compound of claim 1 or a tautomer, cis- or trans-isomer, mesomer, racemate, enantiomer, diastereomer, or mixture thereof, or a pharmaceutically acceptable salt solvate, or prodrug thereof, and a pharmaceutically acceptable carrier.

37. A method of treating cancer in a subject in need thereof, comprising administering to the subject an effective amount of the compound of claim 1, or a tautomer, cis- or trans-isomer, mesomer, racemate, enantiomer, diastereomer, or mixture thereof, or a pharmaceutically acceptable salt solvate, or prodrug thereof.

38. A method of treating a disorder or disease which can be treated by WRN inhibition in a subject, comprising administering to the subject a therapeutically effective amount of the compound of claim 1, or a tautomer, cis- or trans-isomer, mesomer, racemate, enantiomer, diastereomer, or mixture thereof, or a pharmaceutically acceptable salt solvate, or prodrug thereof.

39. A method for treating cancer in a subject in need thereof, comprising administering to the subject an effective amount of a compound of claim 1, or a tautomer, cis- or trans-isomer, mesomer, racemate, enantiomer, diastereomer, or mixture thereof, or a pharmaceutically acceptable salt solvate, or prodrug thereof, wherein the cancer is selected from the group consisting of microsatellite instability-high (MSI-H) or mismatch repair deficient (dMMR), preferably the microsatellite instability-high (MSI-H) or mismatch repair deficient (dMMR) is selected from colorectal, gastric, prostate, endometrial, adrenocortical, uterine, cervical, esophageal, breast, kidney and ovarian cancer.
